(19) United States
(12) Patent Application Publication
(10) Pub. No.: US 2024/0349527 A1
(43) Pub. Date: Oct. 17, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Chi-Sik KIM, Gyeonggi-do (KR); Young-Kwang KIM, Gyeonggi-do (KR); Yoo-Jin DOH, Gyeonggi-do (KR); HaeYeon KIM, Gyeonggi-do (KR)

(21) Appl. No.: 18/590,075

(22) Filed: Feb. 28, 2024

(30) Foreign Application Priority Data
Apr. 4, 2023 (KR) 10-2023-0044282

(51) Int. Cl. H10K 50/11; H10K 85/60

(52) U.S. Cl. CPC H10K 50/11; H10K 85/626; H10K 85/6574

(57) ABSTRACT

The present disclosure relates to a plurality of host materials comprising a first host material comprising a compound represented by formula 1 and a second host material comprising a compound represented by formula 2, an organic electroluminescent compound represented by formula 3 and formula 4, and an organic electroluminescent device comprising the same.

### TECHNICAL FIELD

The present disclosure relates to a plurality of host materials, organic electroluminescent compound, and an organic electroluminescent device comprising the same.

### BACKGROUND ART

The TPD/Alq₃ bilayer small molecule organic electroluminescent device (OLED) consisting of a light-emitting layer and a charge transport layer, was first developed by Tang, et al., of Eastman Kodak in 1987. Thereafter, the studies on an OLED have been rapidly effected, and OLEDs have been commercialized. At present, organic electroluminescent devices mainly use phosphorescent materials having excellent luminous efficiency in panel realization. For prolonged use of a display and high resolution, a OLED having low driving voltage and/or high luminous efficiency and/or long lifespan is required.

Various materials or concepts have been proposed for the organic layer of the organic electroluminescent device in order to improve luminous efficiency and/or driving voltage and/or lifespan, but they have not been satisfactory for practical use. Accordingly, there is a continuing need to develop an organic electroluminescent device having more improved performance, for example, improved driving voltage and/or luminous efficiency and/or power efficiency and/or lifespan characteristics, compared to the organic electroluminescent device disclosed in the related art.

Chinese Patent Application Laid-open CN 110294663 A discloses an organic electroluminescent device using an anthracene-phenanthrofuran-based compound as a host. However, said reference does not specifically disclose an organic electroluminescent device using a plurality of host materials of a specific combination claimed in the present disclosure, and it is still required to develop a host material for improving the performance of OLED.

### DISCLOSURE OF THE INVENTION

### Problems to be Solved

The object of the present disclosure is to provide a plurality of improved host materials capable of providing an organic electroluminescent device with improved driving voltage and/or luminous efficiency and/or lifespan characteristics.

Another object of the present disclosure is to provide an organic electroluminescent compound having a novel structure suitable for application to an organic electroluminescent device.

Still another object of the present disclosure is to provide an organic electroluminescent device having improved driving voltage and/or luminous efficiency and/or lifespan characteristics by comprising the compound of the present disclosure or a specific combination of compounds.

### Solution to Problem

As a result of extensive research to solve the above technical problems, the present inventors have completed the present disclosure by discovering that said objects are achieved by a plurality of host materials, comprising one or more first host compounds and one or more second host compounds, wherein the first host compound is represented by the following formula 1:

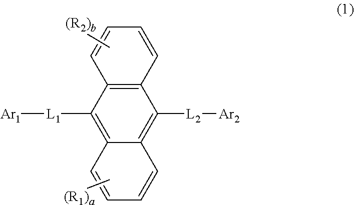

wherein,
Ar₁ and Ar₂ each independently represent a substituted or unsubstituted (C6-C30)aryl, or a substituted or unsubstituted (3- to 30-membered)heteroaryl;
L₁ and L₂ each independently represent a single bond, a substituted or unsubstituted (C6-C30)arylene, or a substituted or unsubstituted (3- to 30-membered)heteroarylene;
R₁ and R₂ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —N—(Ar₇)(Ar₈);
Ar₇ and Ar₈ each independently represent hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
a and b each independently represent an integer of 1 to 4, where if a and b each independently represent an integer of 2 or more, each of R₁ and R₂ may be the same or different, and
wherein the second host compound is represented by the following formula 2:

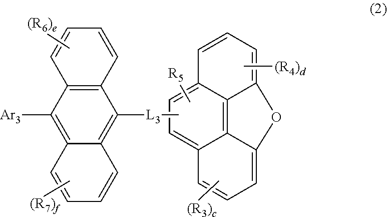

wherein,
Ar₃ represents a substituted or unsubstituted (C6-C30)aryl, or a substituted or unsubstituted (3- to 30-membered)heteroaryl;
L₃ represents a single bond, a substituted or unsubstituted (C6-C30)arylene, or a substituted or unsubstituted (3- to 30-membered)heteroarylene;
R₃ to R₇ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —N—(Ar₉)(Ar₁₀);
Ar₉ and Ar₁₀ each independently represent hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
c and d each independently represent an integer of 1 to 3; and e and f each independently represent an integer of 1 to 4, where if c to f are an integer of 2 or more, each of R₃, R₄, R₆, and R₇ may be the same or different.

In addition, the present inventors have completed the present invention by discovering that said object is achieved by the compound represented by the following formula 3 or 4:

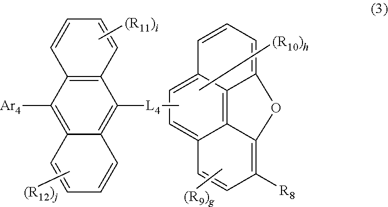

wherein,
Ar₄ represents a substituted or unsubstituted (C6-C30)aryl, or a substituted or unsubstituted (3- to 30-membered)heteroaryl;
L₄ represents a single bond, a substituted or unsubstituted (C6-C30)arylene, or a substituted or unsubstituted (3- to 30-membered)heteroarylene;
R₈ represents a substituted or unsubstituted (C6-C30)aryl, or a substituted or unsubstituted (3- to 30-membered)heteroaryl;
R₉ to R₁₂ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or—N—(Ar₁₁)(Ar₁₂);
Ar₁₁ and Ar₁₂ each independently represent hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
g represents an integer of 1 or 2, and h, i and j each independently represent an integer of 1 to 4, where if g to j are an integer of 2 or more, each of R₉ to R₁₂ may be the same or different,

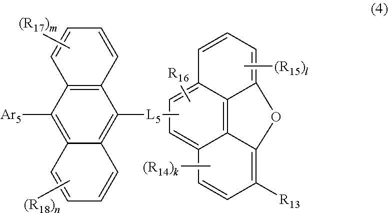

wherein,
Ar₅ represents a substituted or unsubstituted (C6-C30)aryl, or a substituted or unsubstituted (3- to 30-membered)heteroaryl;
L₅ represents a single bond, a substituted or unsubstituted (C6-C30)arylene, or a substituted or unsubstituted (3- to 30-membered)heteroarylene;
R₁₃ represents hydrogen or deuterium;
R₁₄ to R₁₈ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or—N—(Ar₁₃)(Ar₁₄);
Ar₁₃ and Ar₁₄ each independently represent hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
k represents an integer of 1 or 2, I represents an integer of 1 to 3, and m and n each independently represent an integer of 1 to 4, where if k to n are an integer of 2 or more, each of R₁₄, R₁₅, R₁₇, and R₁₈ may be the same or different;
provided that, at least one of R₁₄, R₁₅ and R₁₆ represents a substituted or unsubstituted (C6-C30)aryl, or a substituted or unsubstituted (3- to 30-membered)heteroaryl; and at least one of R₁₃ to R₁₈ represents deuterium.

### Advantageous Effects of Invention

The plurality of host materials and the organic electroluminescent compound according to the present disclosure exhibit suitable performance for use in an organic electroluminescent device.

In addition, by comprising the compound according to the present disclosure as an organic electroluminescent material, or a specific combination of the compounds according to the present disclosure as a plurality of host materials, an organic electroluminescent device having a lower driving voltage and/or higher luminous efficiency and/or long lifespan characteristics than a conventional organic electroluminescent device is provided, and a display device or a lighting device using the same could be manufactured.

### EMBODIMENTS OF THE INVENTION

Hereinafter, the present disclosure will be described in detail. However, the following description is intended to explain the present disclosure, and is not meant to restrict the scope of the present disclosure.

Herein, the term “organic electroluminescent compound” in the present disclosure refers to a compound that may be used in an organic electroluminescent device, and may be comprised in any layer consists the organic electroluminescent device, as needed.

Herein, the term “organic electroluminescent material” in the present disclosure refers to a material that may be used in an organic electroluminescent device, may comprise one or more compounds, and may be comprised in any layer consists an organic electroluminescent device, as needed. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

Herein, the term “a plurality of host materials” in the present disclosure refers to a host material comprising a combination of two or more compounds that may be comprised in any light-emitting layer consisting an organic electroluminescent device, and may refer to both materials before (e.g., before deposition) and after (e.g., after deposition) being comprised in the organic electroluminescent device. In one embodiment, the plurality of host materials of the present disclosure may be a combination of two or more host materials, and may optionally further include a conventional material comprised in the organic electroluminescent material. The two or more types of compounds comprised in the plurality of host materials of the present disclosure may be comprised together in one light-emitting layer or may be included in different light-emitting layers through methods used in the art. For example, the two or more types of host materials may be mixed-deposited, co-deposited, or individually deposited.

Herein, the term “(C1-C30)alkyl” in the present disclosure refers to a linear or branched alkyl having 1 to 30 carbon atoms consisting the chain, in which the number of carbon atoms is preferably 1 to 10, more preferably 1 to 6. Specific examples of the alkyl may include methyl, ethyl, n-propyl, isopropyl, n-butyl, isobutyl, tert-butyl, sec-butyl, etc.

Herein, the term “(C3-C30)cycloalkyl” or “(C3-C30)cycloalkylene” in the present disclosure refers to a monocyclic or polycyclic hydrocarbon having 3 to 30 ring backbone carbon atoms, preferably having 3 to 20 carbon atoms, and more preferably having 3 to 7 carbon atoms. Examples of the cycloalkyl may include cyclopropyl, cyclobutyl, cyclopentyl, cyclohexyl, cyclopentylmethyl, cyclohexylmethyl, and the like.

Herein, the term “(3- to 7-membered)heterocycloalkyl” in the present disclosure refers to a cycloalkyl having 3 to 7 ring backbone atoms and comprising at least one heteroatom selected from the group consisting of B, N, O, S, Si, and P, preferably at least one heteroatom selected from the group consisting of O, S, and N, for example, tetrahydrofuran, pyrrolidine, thiolane, tetrahydropyran, etc.

Herein, the term “(C6-C30)aryl” or “(C6-C30)arylene” in the present disclosure refers to a monocyclic or fused ring-based radical derived from an aromatic hydrocarbon having 6 to 30 ring backbone carbon atoms, and may be partially saturated. The aryl and arylene include those having a spiro structure.

The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, spiro[cyclopentene-fluorene]yl, spiro[dihydroindene-fluorene]yl, azulenyl, tetramethyldihydrophenanthrenyl, etc. Specifically, examples of the aryl include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylenyl, 3-biphenylenyl, 4-biphenylenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the term “(3- to 30-membered)heteroaryl” or “(3- to 30-membered)heteroarylene” refers to an aryl or arylene group having 3 to 30 ring backbone atoms and comprising at least one heteroatom selected from the group consisting of B, N, O, S, Si, and P. The number of heteroatoms is preferably 1 to 4, and may be a single ring system or a fused ring system fused with one or more benzene rings, and may be partially saturated. In addition, the heteroaryl or heteroarylene herein also includes a form in which one or more heteroaryl groups or aryl groups are linked to the heteroaryl group by a single bond, and also includes those having a spiro structure. Examples of the heteroaryl may include monocyclic heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc; fused heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazindolyl, benzopyrazindolyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazoyl, phenoxazinyl, benzodioxolyl, benzotriazolinyl, hydroacridazolyl, phenothidazolyl, quinazolinyl, quinazolinyl, quinoxalinyl, benzoimidazolyl, benzocarbazolyl, benzocarbazolyl, phenothidazolyl, benzotriazolinolinazoyl, phenazolinoindolyl fused cyclic heteroaryl such as linyl, dimethylbenzopyrimidinyl, indolocarbazolyl, indenocarbazolyl, etc. More specifically, examples of the heteroaryl include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrole-1-yl, 2-methylpyrrole-3-yl, 2-methylpyrrole-4-yl, 2-methylpyrrole-5-yl, 3-methylpyrrole-1-yl, 3-methylpyrrole-2-yl, 3-methylpyrrole-4-yl, 3-methylpyrrole-5-yl, 3-methylpyrrole-5-yl, 2-tert-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-Benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-Benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germanfluorenyl, 2-germanfluorenyl, 3-germanfluorenyl, 4-germanfluorenyl, 1-dibenzoselenophenyl, 2-dibenzoselenophenyl, 3-dibenzoselenophenyl, 4-dibenzoselenophenyl. Herein, the term “halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other; for example, when two substituents in a benzene derivative occupy positions 1 and 2, it is called an ortho position. Meta indicates that two substituents are at positions 1 and 3; for example, when two substituents in a benzene derivative occupy positions 1 and 3, it is called a meta position. Para indicates that two substituents are at positions 1 and 4; for example, when two substituents in a benzene derivative occupy positions 1 and 4, it is called a para position.

In addition, “substituted” in the expression “substituted or unsubstituted” refers to a hydrogen atom in a functional group that is replaced with another atom or another functional group (i.e., a substituent). Herein, the substituents of substituents of the substituted alkyl, the substituted cycloalkyl, the substituted aryl, the substituted heteroaryl, and the substituted alkoxy are each independently selected from the group consisting deuterium; halogen; cyano; carboxyl; nitro; hydroxy; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3-7 membered)heterocycloalkyl; (C6-C30)aryloxy; (C6-C30)arylthio; (C6-C30)aryl substituted or unsubstituted with at least one of (C1-C30)alkyl and di(C6-C30)arylamino; (3-30 membered)heteroaryl substituted or unsubstituted with at least one (C6-C30)aryl; tri(C1-C30)alkylsilyl; tri(C6-C30)arylsilyl; di(C1-C30)alkyl(C6-C30)arylsilyl; (C1-C30)alkyldi(C6-C30)arylsilyl; fused ring of (C3-C30) aliphatic ring and (C6-C30) aromatic ring; amino; mono- or di-(C1-C30)alkylamino; mono- or di-(C2-C30)alkenylamino; mono- or di-(C6-C30)arylamino substituted or unsubstituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl (C6-C30)arylamino; (C2-C30)alkenyl (3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. According to one embodiment of the present disclosure, the substituents each independently are at least one selected from the group consisting of (C1-C30)alkyl; (C6-C30)aryl substituted or unsubstituted with at least one of (C1-C30)alkyl and di(C6-C30)arylamino; (3- to 30-membered)heteroaryl substituted or unsubstituted with at least one (C6-C30)aryl; and substituted or unsubstituted di(C6-C30)arylamino. According to another embodiment of the present disclosure, the substituents each independently are at least one selected from the group consisting of (C1-C6)alkyl; (C6-C20)aryl substituted or unsubstituted with at least one of (C1-C6)alkyl and di(C6-C12)arylamino; (5- to 15-membered)heteroaryl substituted or unsubstituted with at least one (C6-C12)aryl; and a substituted or unsubstituted di(C6-C18)arylamino. For example, the substituents each independently may be methyl; phenyl substituted unsubstituted with naphthyl or diphenylamino; naphthyl; biphenyl; phenanthrenyl; terphenyl; dimethylfluorenyl; pyrimidinyl substituted with one or more phenyl; triazinyl substituted with one or more of phenyl, naphthyl, and biphenyl; dibenzofuranyl; dibenzothiophenyl; carbazolyl substituted with phenyl; or amino substituted with one or more of phenyl, naphthyl, biphenyl, and phenanthrenyl, etc.

Herein, when a ring is formed by being linked with an adjacent substituent or when two adjacent substituents are linked with each other to form a ring, said ring may be a substituted or unsubstituted (3- to 30-membered) mono- or polycyclic, alicyclic or aromatic ring, or a combination thereof, formed by being linked with two or more adjacent substituents. In addition, the formed ring may comprise one or more heteroatoms selected from B, N, O, S, Si and P, preferably one or more heteroatoms selected from N, O and S. According to an embodiment of the present disclosure, the number of ring backbone atoms is (5-20 membered), and according to another embodiment of the present disclosure, the number of ring backbone atoms is (5-15 membered). For example, the fused ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring.

Herein, the heteroaryl or heteroarylene may each independently comprise at least one heteroatom selected from B, N, O, S, Si, and P. In addition, the heteroatom may be bonded to at least one selected from the group consisting of hydrogen, deuterium, halogen, cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

The compounds represented by formula 1 may be described in more detail as follows.

In formula 1, Ar₁ and Ar₂ may each independently be, a substituted or unsubstituted (C6-C30)aryl, or a substituted or unsubstituted (3-30)membered heteroaryl, preferably a substituted or unsubstituted (C6-C25)aryl, or a substituted or unsubstituted (3-25)membered heteroaryl, more preferably a substituted or unsubstituted (C6-C20)aryl, or a substituted or unsubstituted (3-20)membered heteroaryl. In one embodiment of the present disclosure, Ar₁ and Ar₂ of formula 1 may each independently be, a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted triphenylenyl, a substituted or unsubstituted fluorenyl, a substituted or unsubstituted dibenzofluorenyl, a substituted or unsubstituted dibenzofuranyl, a substituted or unsubstituted dibenzothiophenyl, a substituted or unsubstituted carbazolyl, or a substituted or unsubstituted dibenzoselenophenyl. For example, in formula 1, Ar₁ and Ar₂ may each independently be, a substituted or unsubstituted phenyl, a substituted or unsubstituted biphenyl, a substituted or unsubstituted p-terphenyl, a substituted or unsubstituted m-terphenyl, a substituted or unsubstituted o-terphenyl, a substituted or unsubstituted naphthyl, a substituted or unsubstituted phenylnaphthyl, a substituted or unsubstituted phenanthrenyl, or a substituted or unsubstituted phenylphenanthrenyl, and the hydrogen in each substituent may be substituted with deuterium.

In formula 1, L₁ and L₂ may each independently be, a single bond, a substituted or unsubstituted (C6-C30)arylene, or a substituted or unsubstituted (3- to 30-membered)heteroarylene, preferably, a single bond, a substituted or unsubstituted (C6-C25)arylene, or a substituted or unsubstituted (3- to 25-membered)heteroarylene, more preferably, a single bond, a substituted or unsubstituted (C6-C25)arylene, or a substituted or unsubstituted (3- to 25-membered)heteroarylene. For example, in formula 1, L₁ and L₂ may each independently be, a single bond, a substituted or unsubstituted phenylene, a substituted or unsubstituted p-terphenylene, a substituted or unsubstituted m-terphenylene, a substituted or unsubstituted o-terphenylene, a substituted or unsubstituted naphthylene, a substituted or unsubstituted phenylnaphthylene, or a substituted or unsubstituted phenanthrenylene, and the hydrogen in each substituent may be substituted with deuterium.

In formula 1, R₁ and R₂ may each independently be, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. For example, in formula 1, R₁ and R₂ may each independently be, hydrogen or deuterium.

In formula 1, a and b each independently, may be an integer of 1 to 4, for example, 1, 2, 3, or 4.

The compounds represented by formula 1 may be selected from the following compounds, but are not limited thereto.

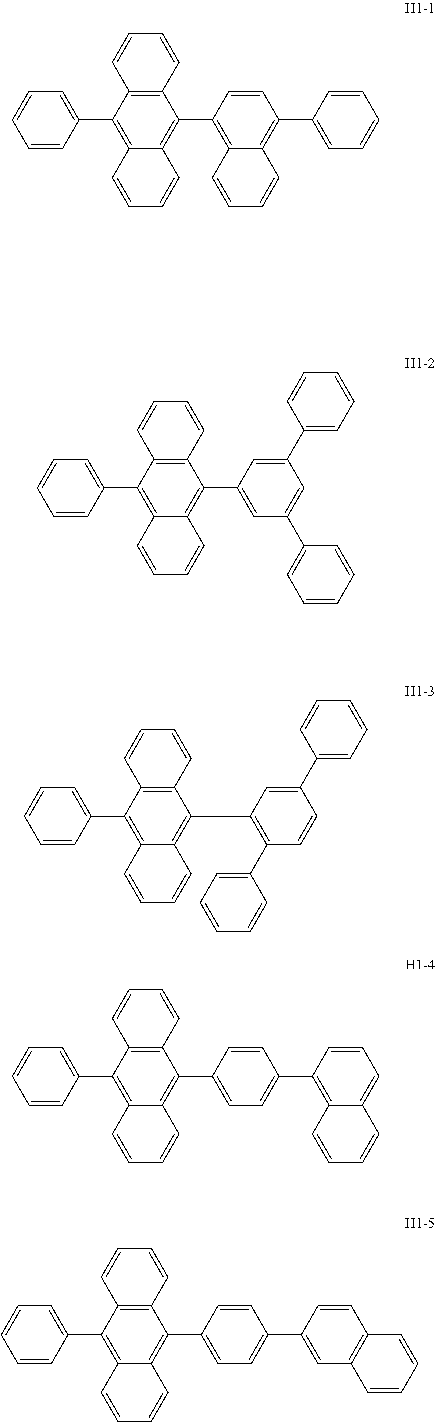

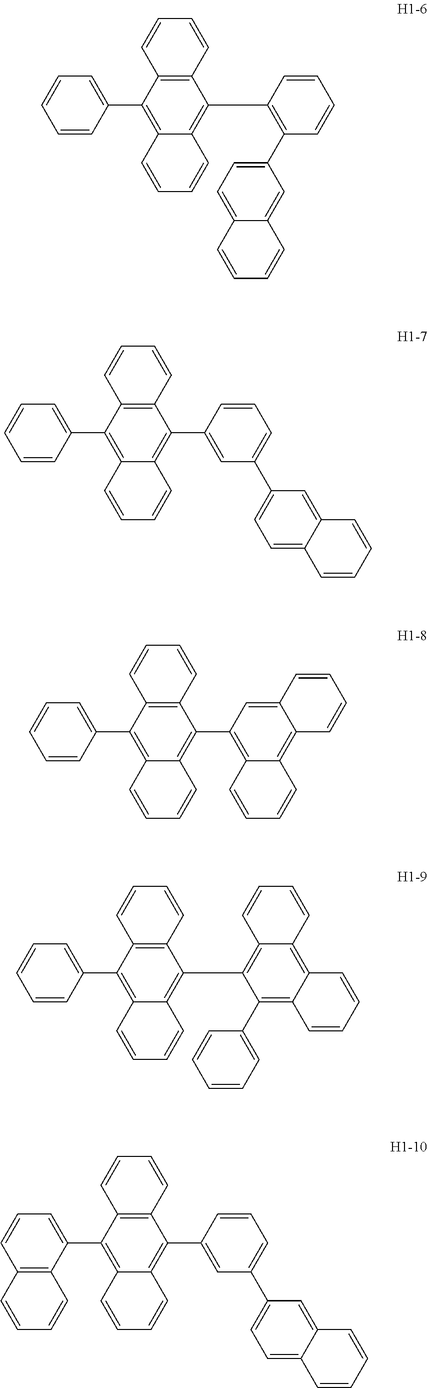

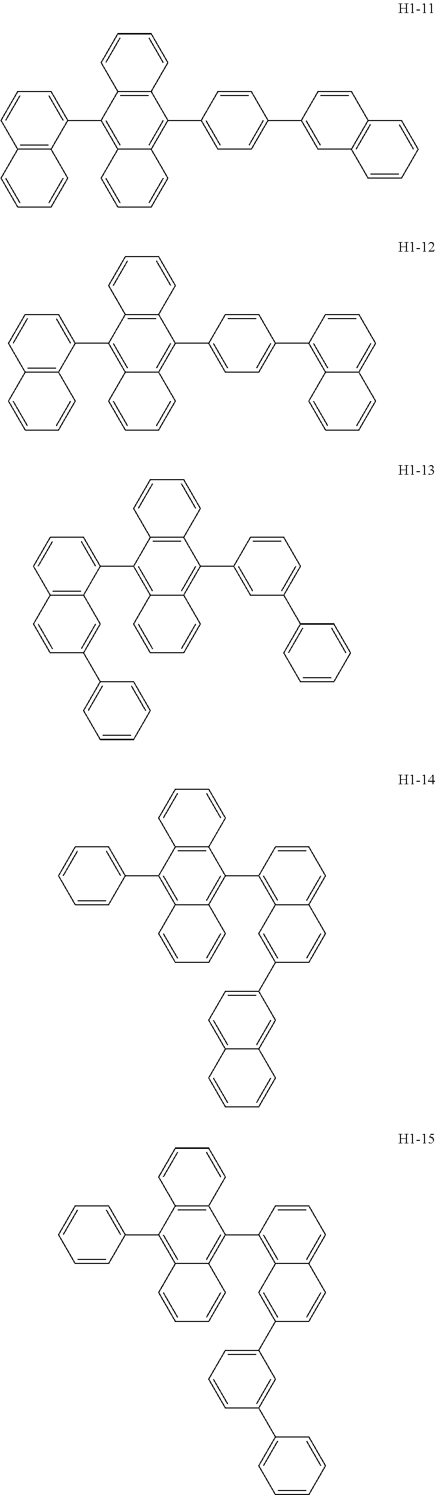

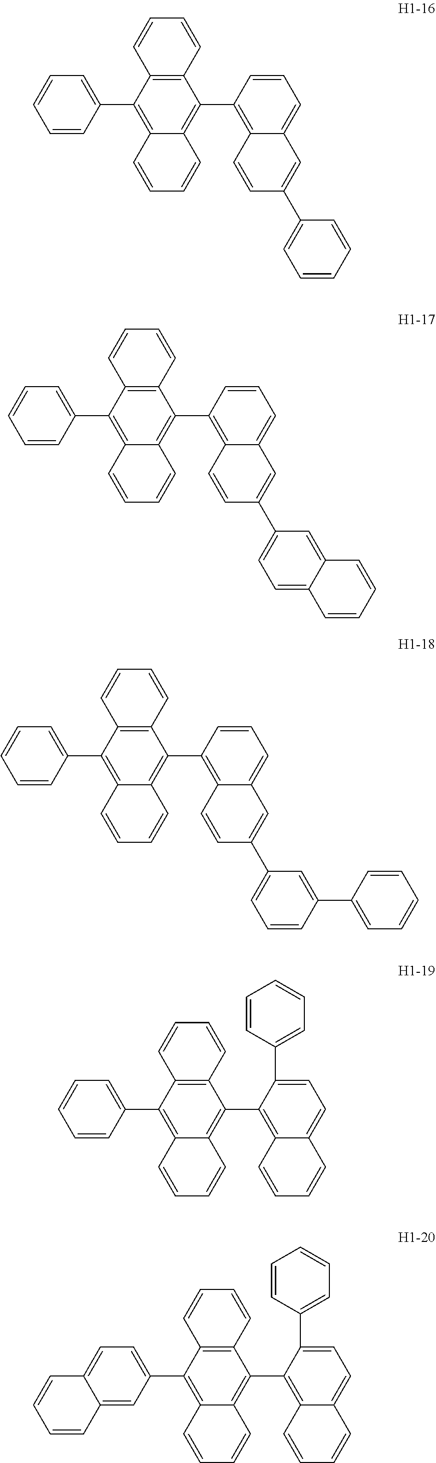

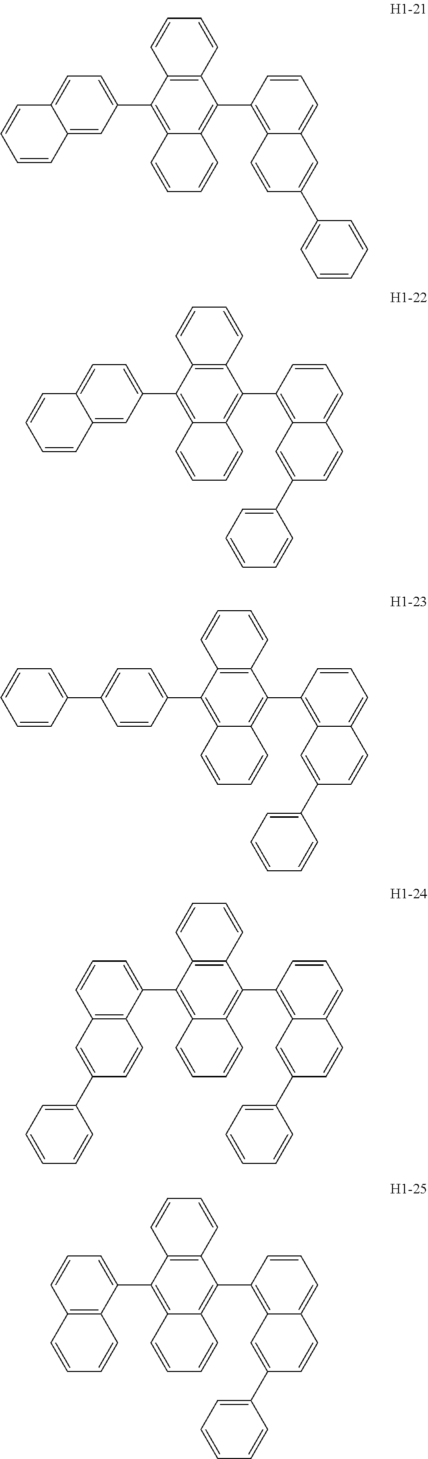

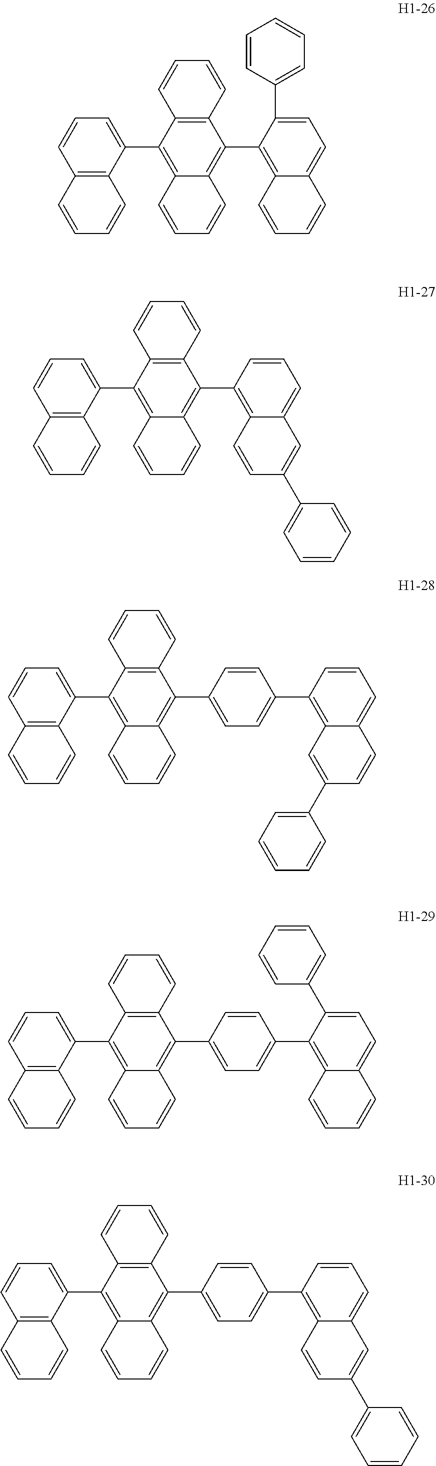

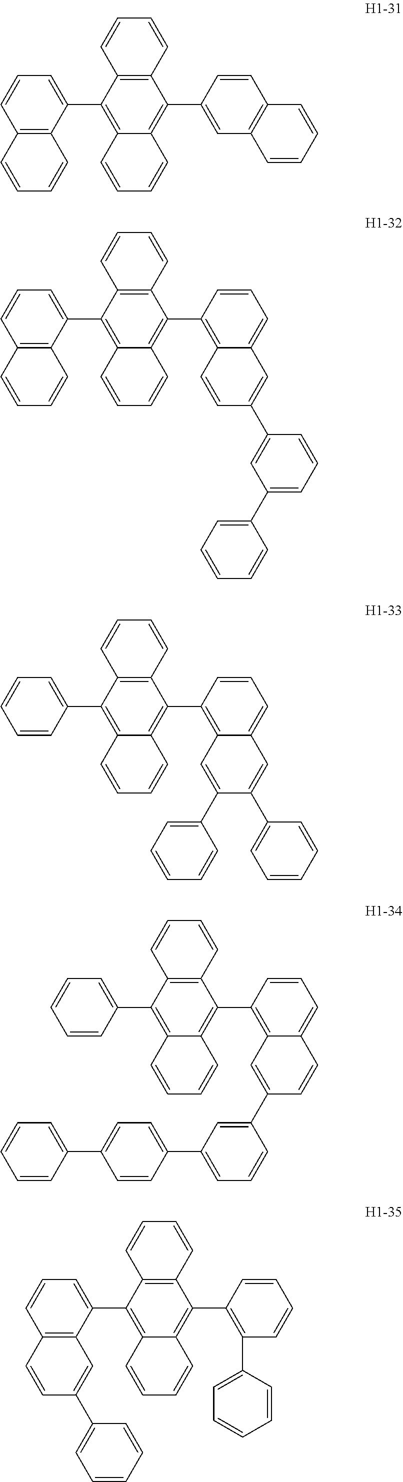

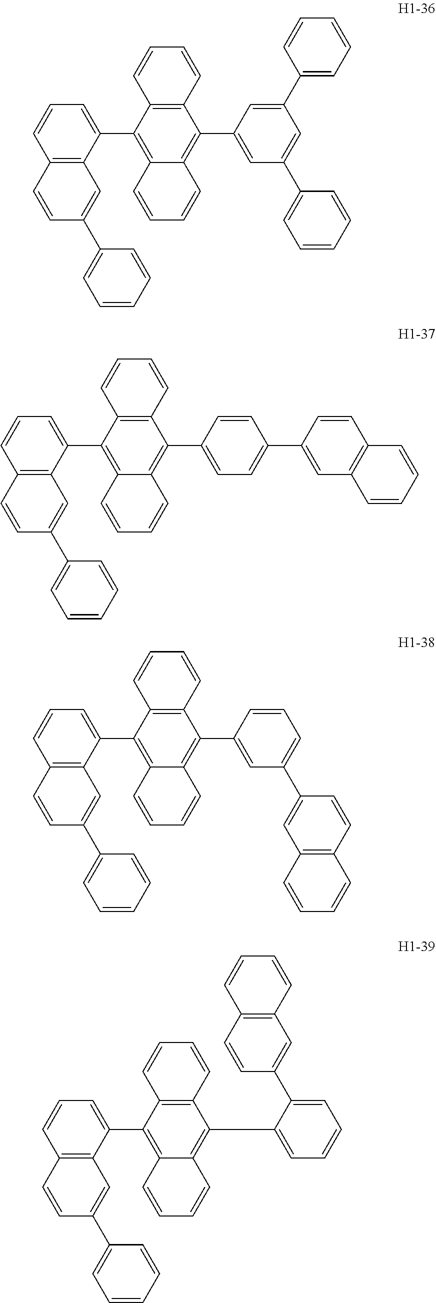

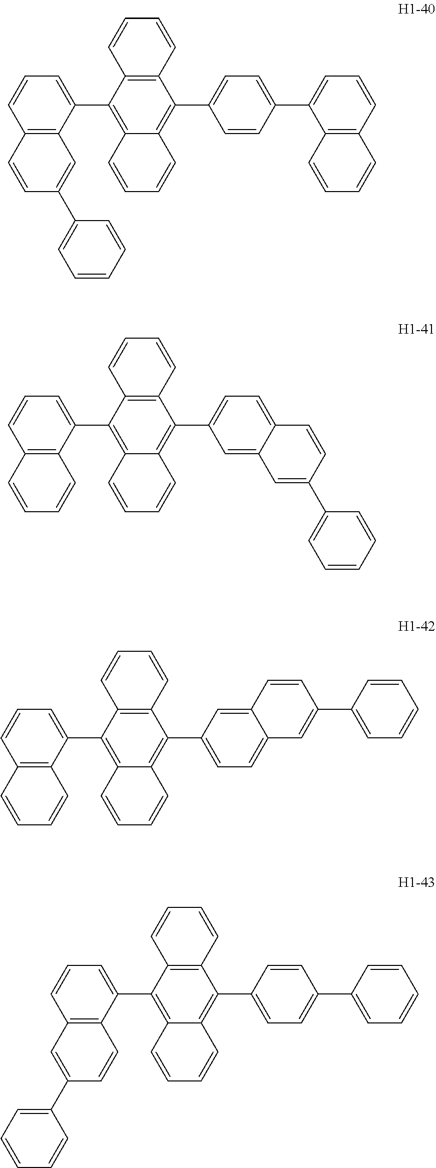

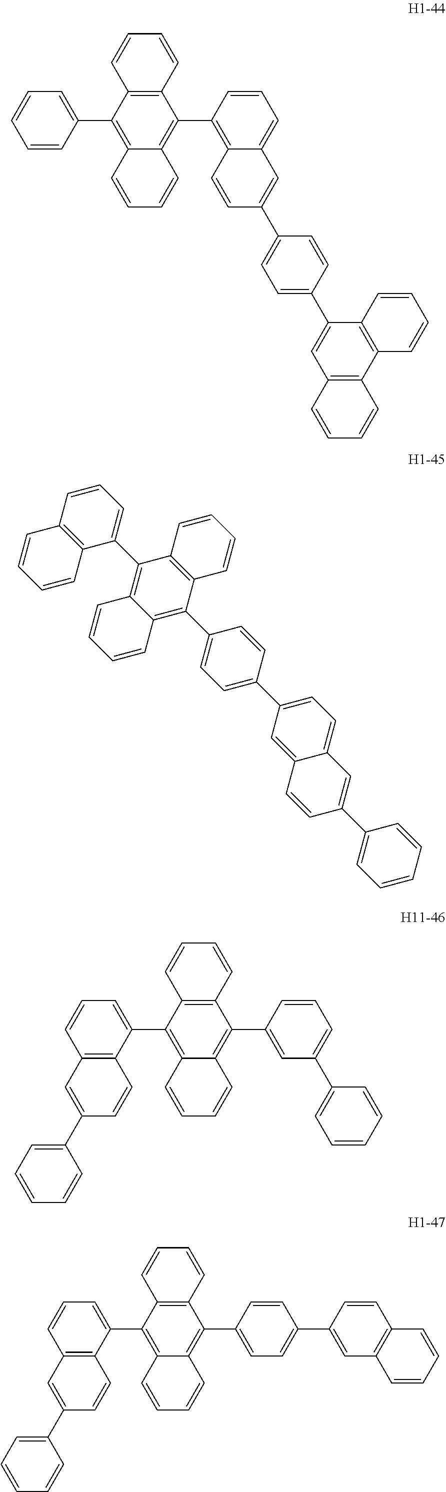

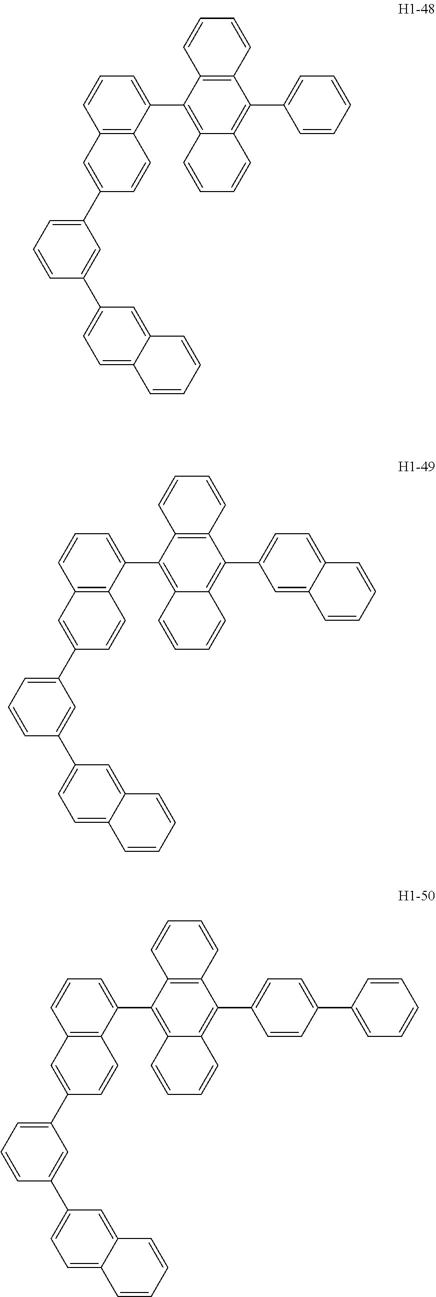

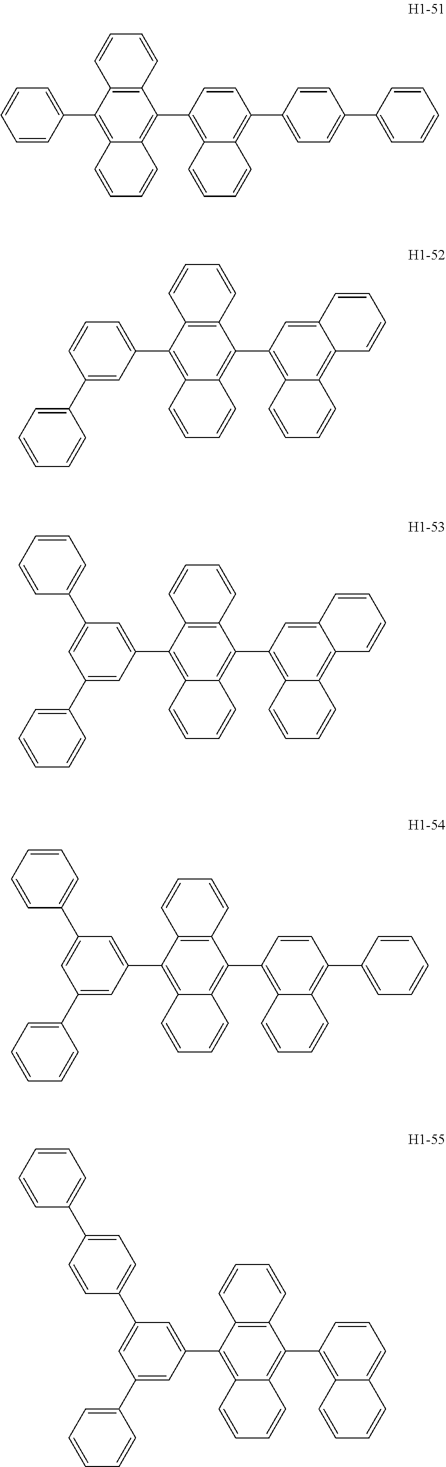

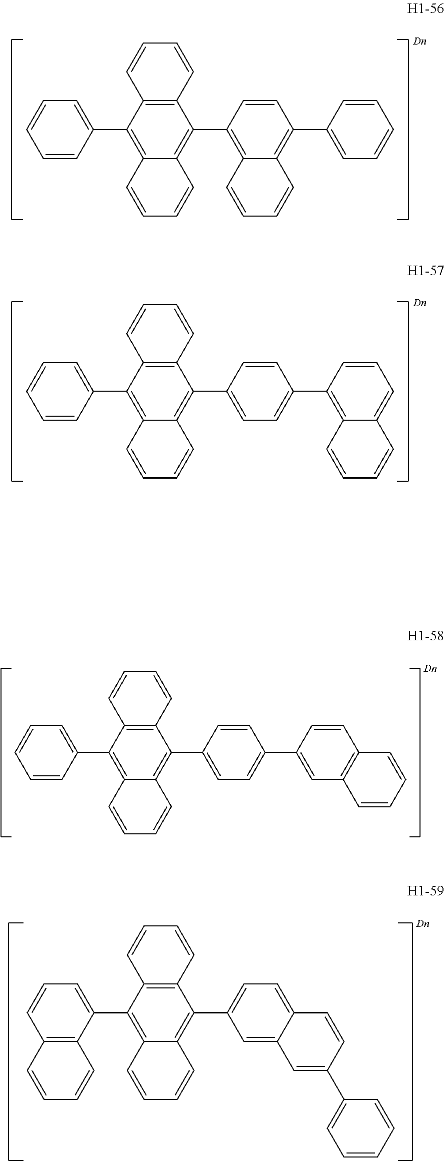

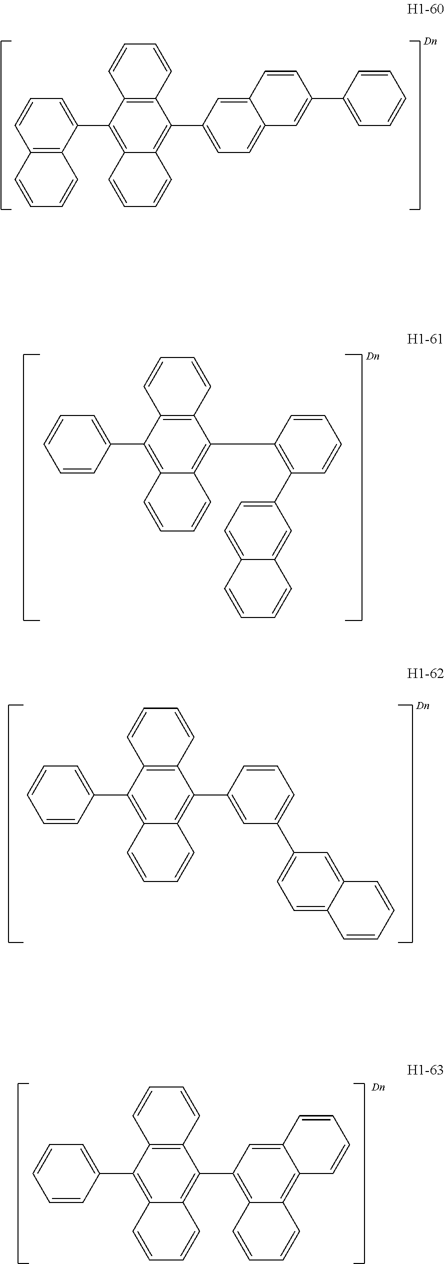

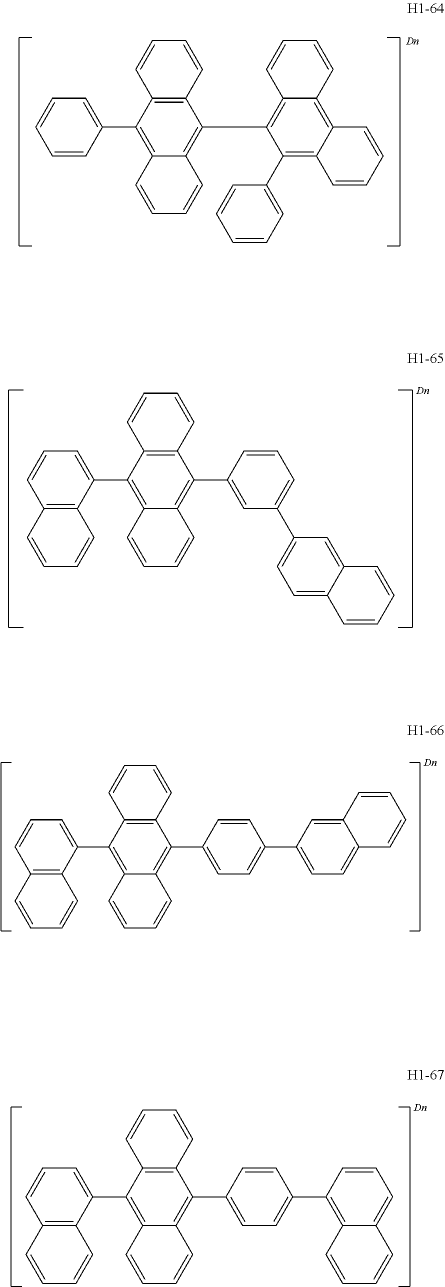

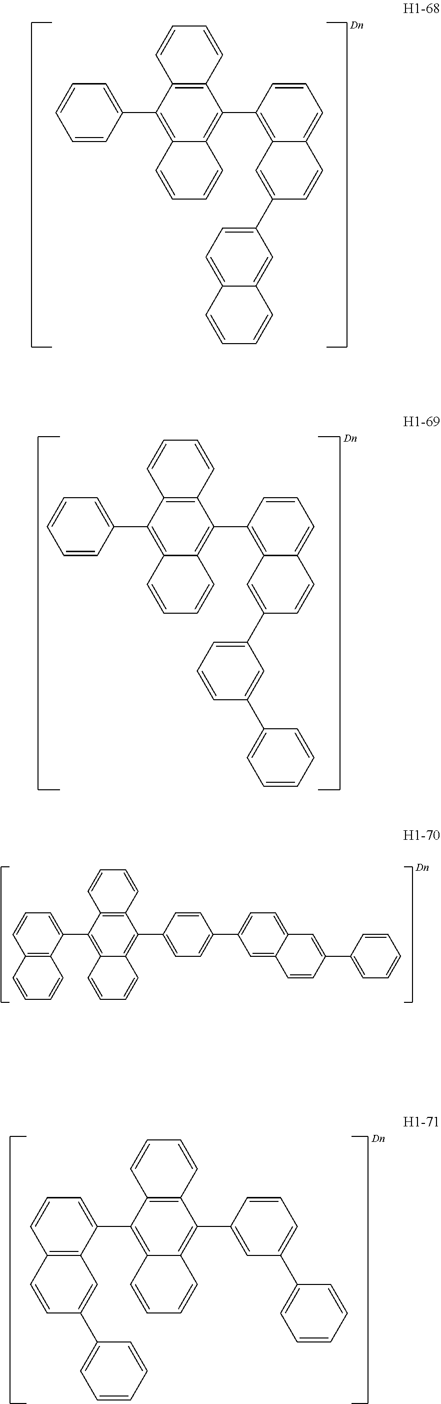

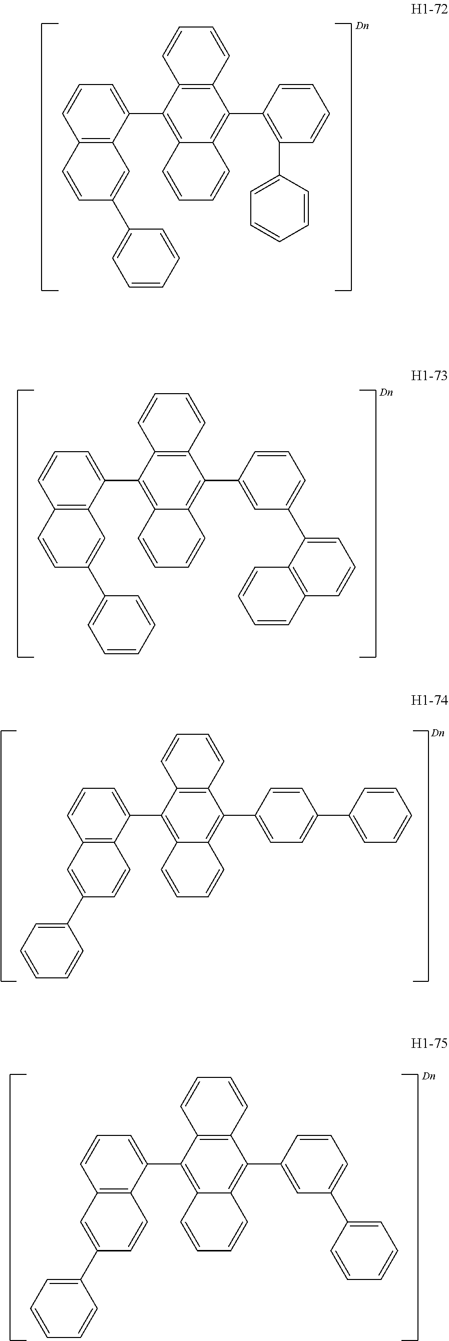

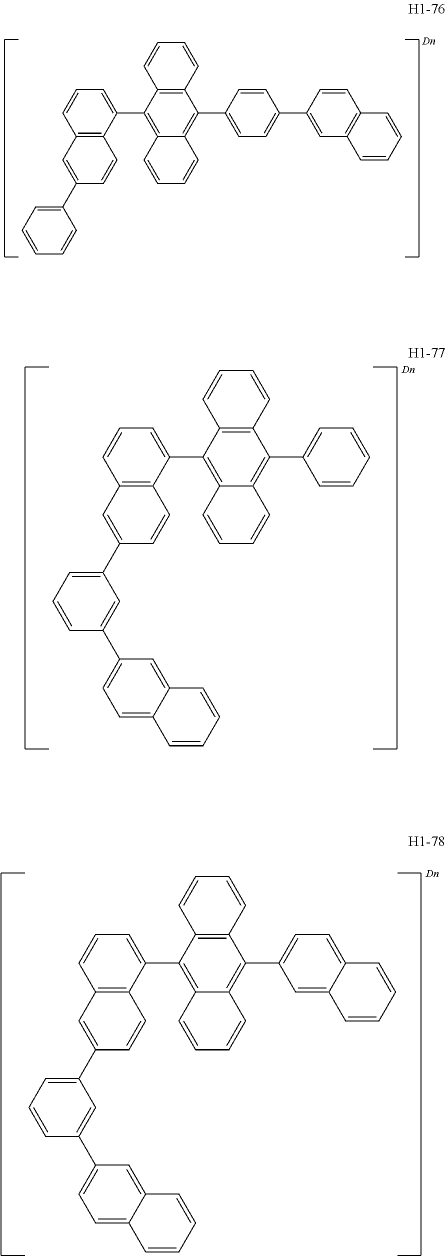

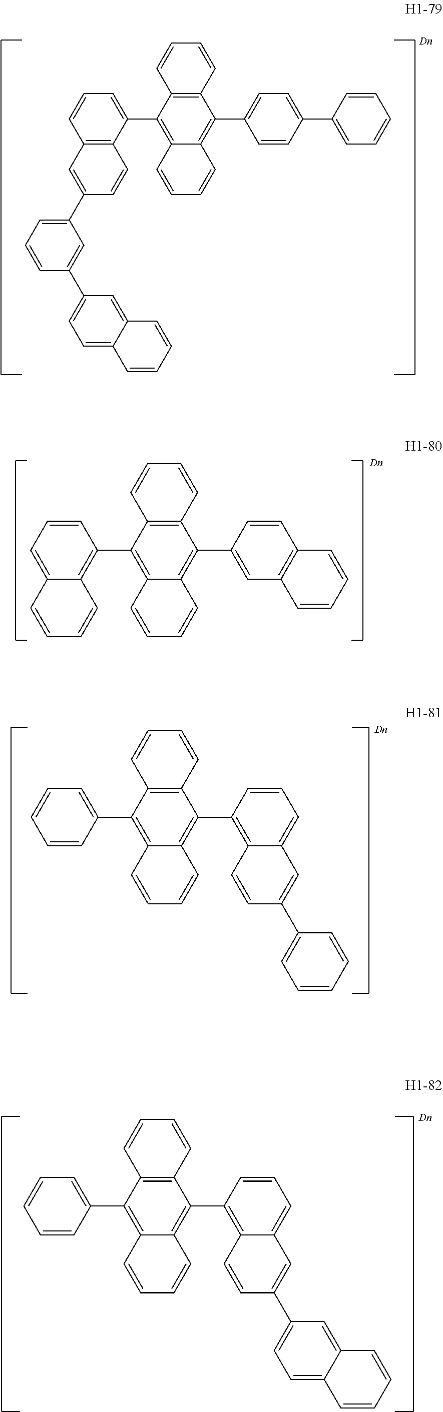

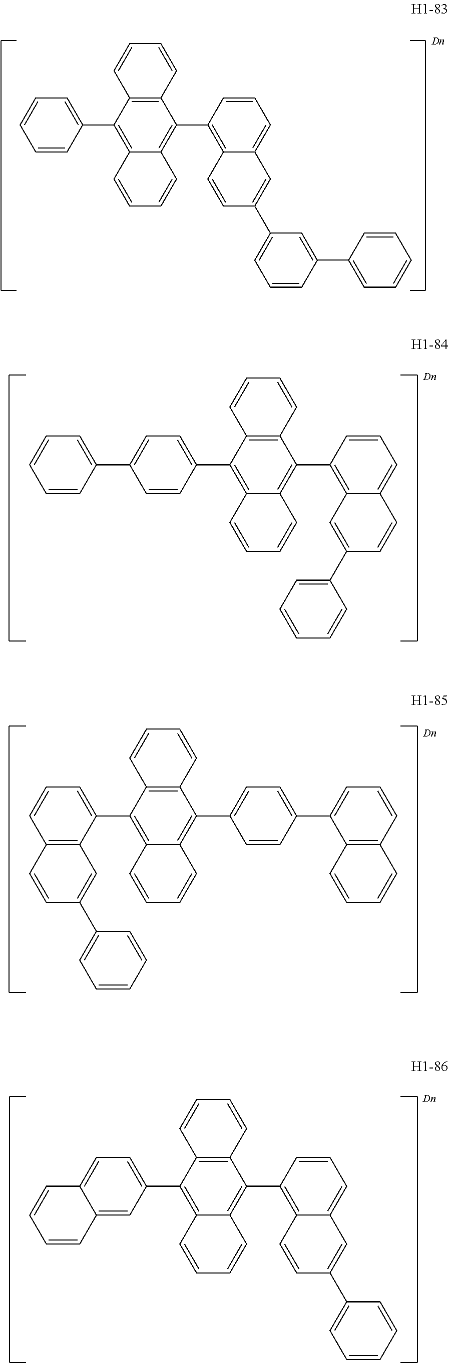

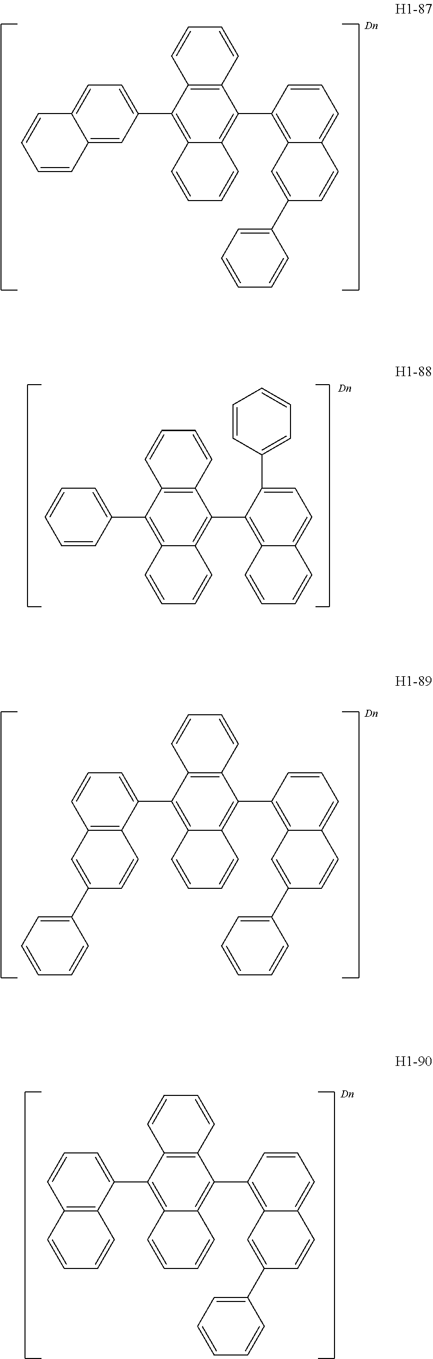

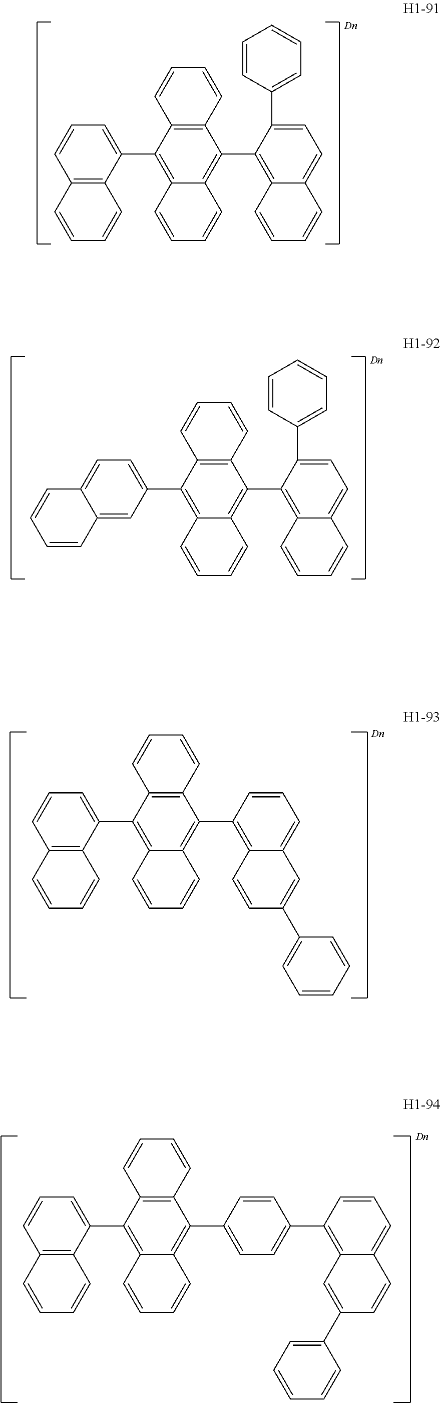

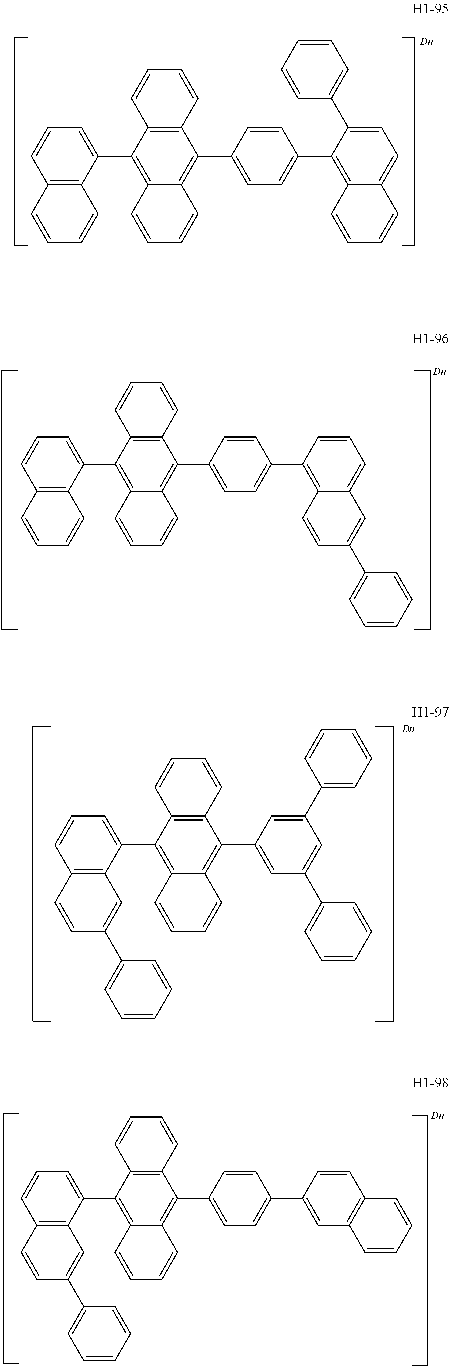

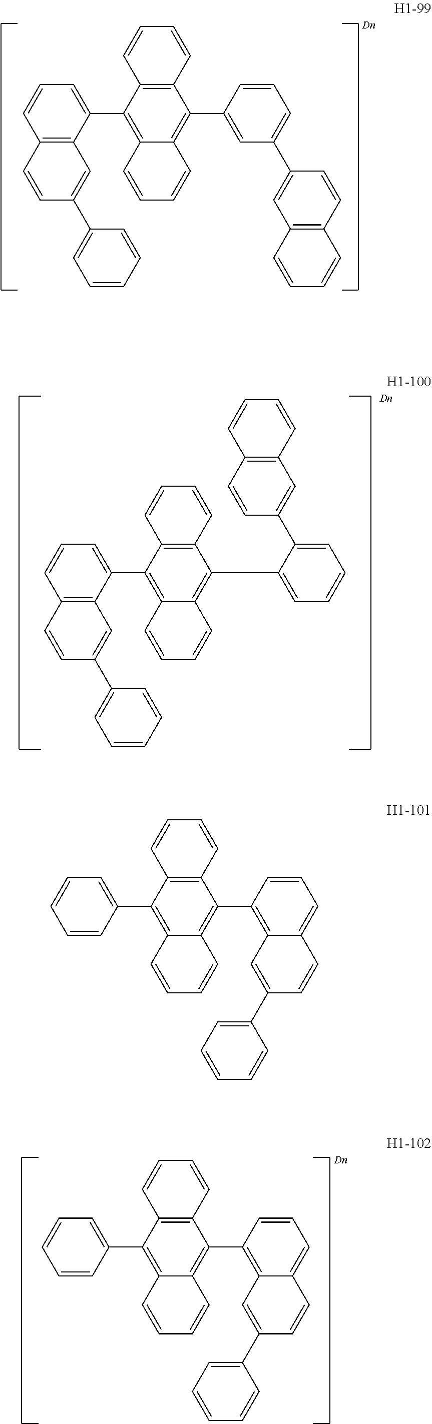

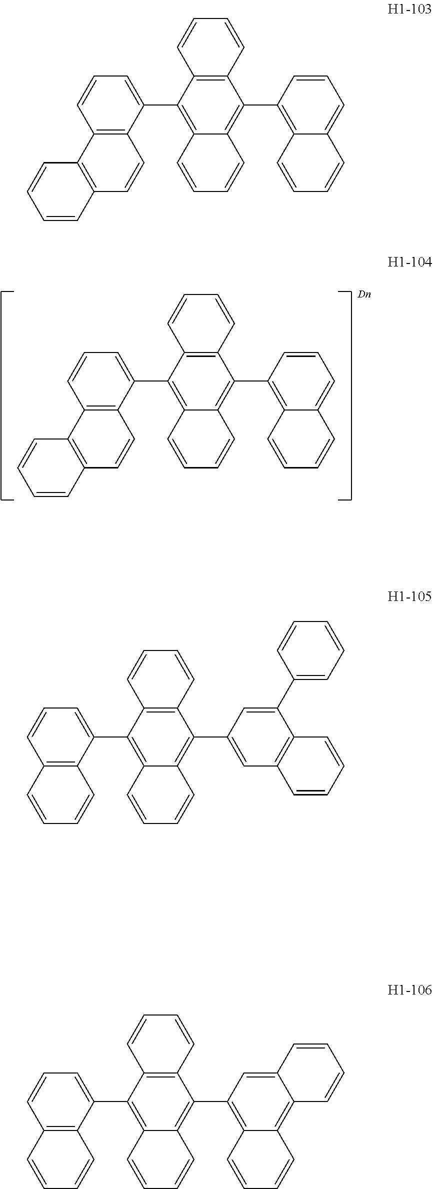

Dn of compounds H1-56 to H1-100, H1-102, and H1-104 means that n hydrogens is replaced by deuteriums, and n is between 1 and the maximum number of hydrogens in each compound.

The compounds represented by formula 2 may be described in more detail as follows.

In formula 2, Ar₃ may be a substituted or unsubstituted (C6-C30)aryl, or a substituted or unsubstituted (3- to 30-membered)heteroaryl, preferably a substituted or unsubstituted (C6-C25)aryl, or a substituted or unsubstituted (3- to 25-membered)heteroaryl, more preferably a substituted or unsubstituted (C6-C20)aryl, or a substituted or unsubstituted (3- to 20-membered)heteroaryl. In one embodiment of the present disclosure, Ar₃ of formula 2 may be a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted triphenylenyl, a substituted or unsubstituted fluorenyl, a substituted or unsubstituted dibenzofluorenyl, a substituted or unsubstituted dibenzofuranyl, a substituted or unsubstituted dibenzothiophenyl, a substituted or unsubstituted carbazolyl, a substituted or unsubstituted dibenzoselenophenyl, or a substituted or unsubstituted naphthobenzofuranyl. For example, in formula 2, Ar₃ may be a substituted or unsubstituted phenyl, a substituted or unsubstituted biphenyl, a substituted or unsubstituted p-terphenyl, a substituted or unsubstituted m-terphenyl, a substituted or unsubstituted o-terphenyl, a substituted or unsubstituted naphthyl, a substituted or unsubstituted phenylnaphthyl, a substituted or unsubstituted phenanthrenyl, a substituted or unsubstituted phenylphenanthrenyl, a substituted or unsubstituted naphthylphenanthrenyl, a substituted or unsubstituted dibenzofuranyl, a substituted or unsubstituted naphthobenzofuranyl, or a substituted or unsubstituted carbazolyl, and the hydrogen in each substituent may be substituted with deuterium.

In formula 2, L₃ may be a single bond, a substituted or unsubstituted (C6-C30)arylene, or a substituted or unsubstituted (3- to 30-membered)heteroarylene, preferably a single bond, a substituted or unsubstituted (C6-C25)arylene, or a substituted or unsubstituted (3- to 25-membered)heteroarylene, more preferably a single bond, a substituted or unsubstituted (C6-C25)arylene, or a substituted or unsubstituted (3- to 25-membered)heteroarylene. For example, L₃ may be a single bond.

In formula 2, R₃ to R₇ may each independently be, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. For example, in formula 2, R₃ to R₇ may each independently be, hydrogen, deuterium, a substituted or unsubstituted phenyl, or a substituted or unsubstituted biphenyl, and the hydrogen in each substituent may be substituted with deuterium.

In formula 2, c and d each independently, may be an integer of 1 to 3, for example, 1, 2, or 3.

In formula 2, e and f each independently, may be an integer of 1 to 4, for example, 1, 2, 3, or 4.

The compound represented by formula 2 may be selected from the following compounds, but are not limited thereto.

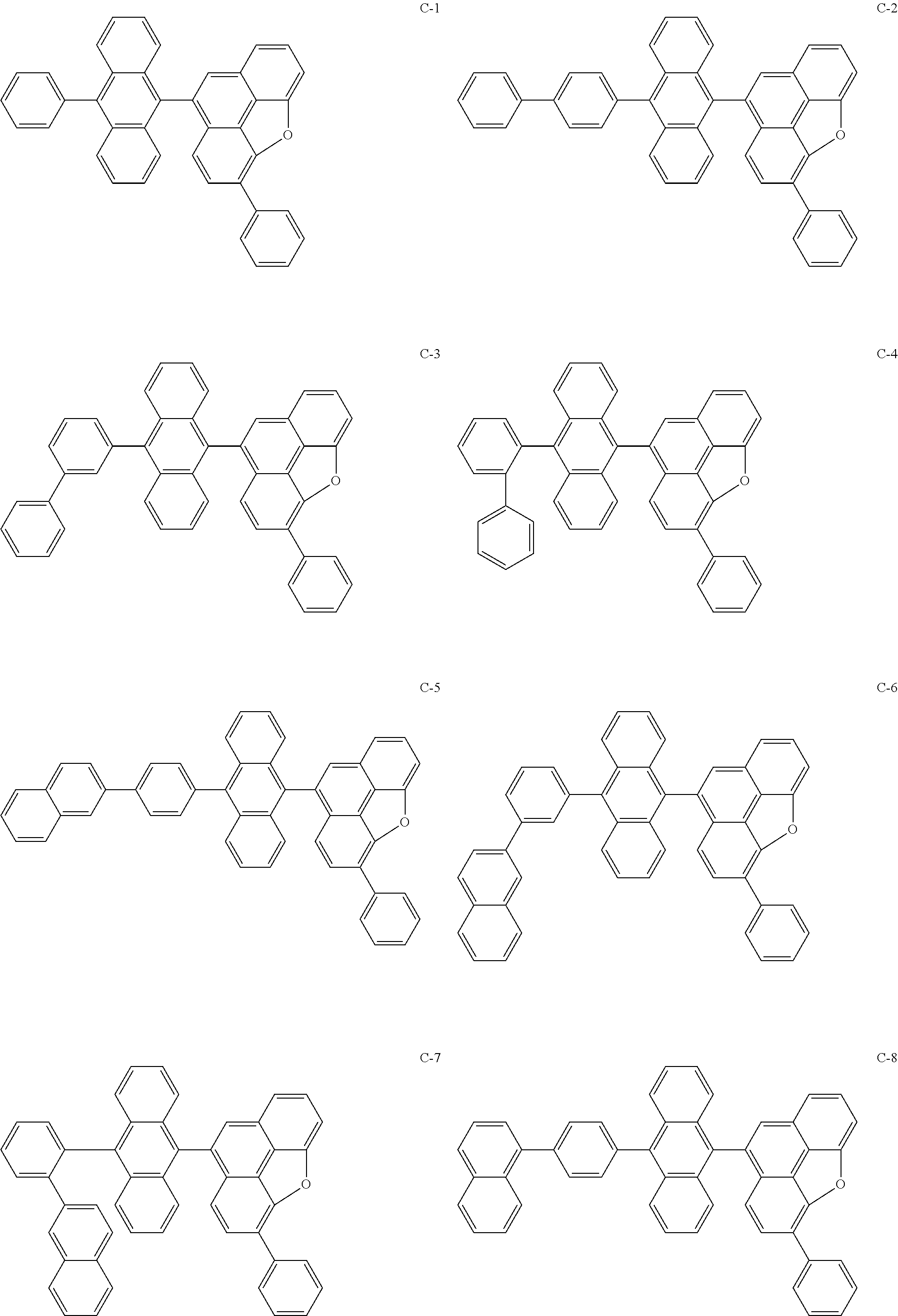

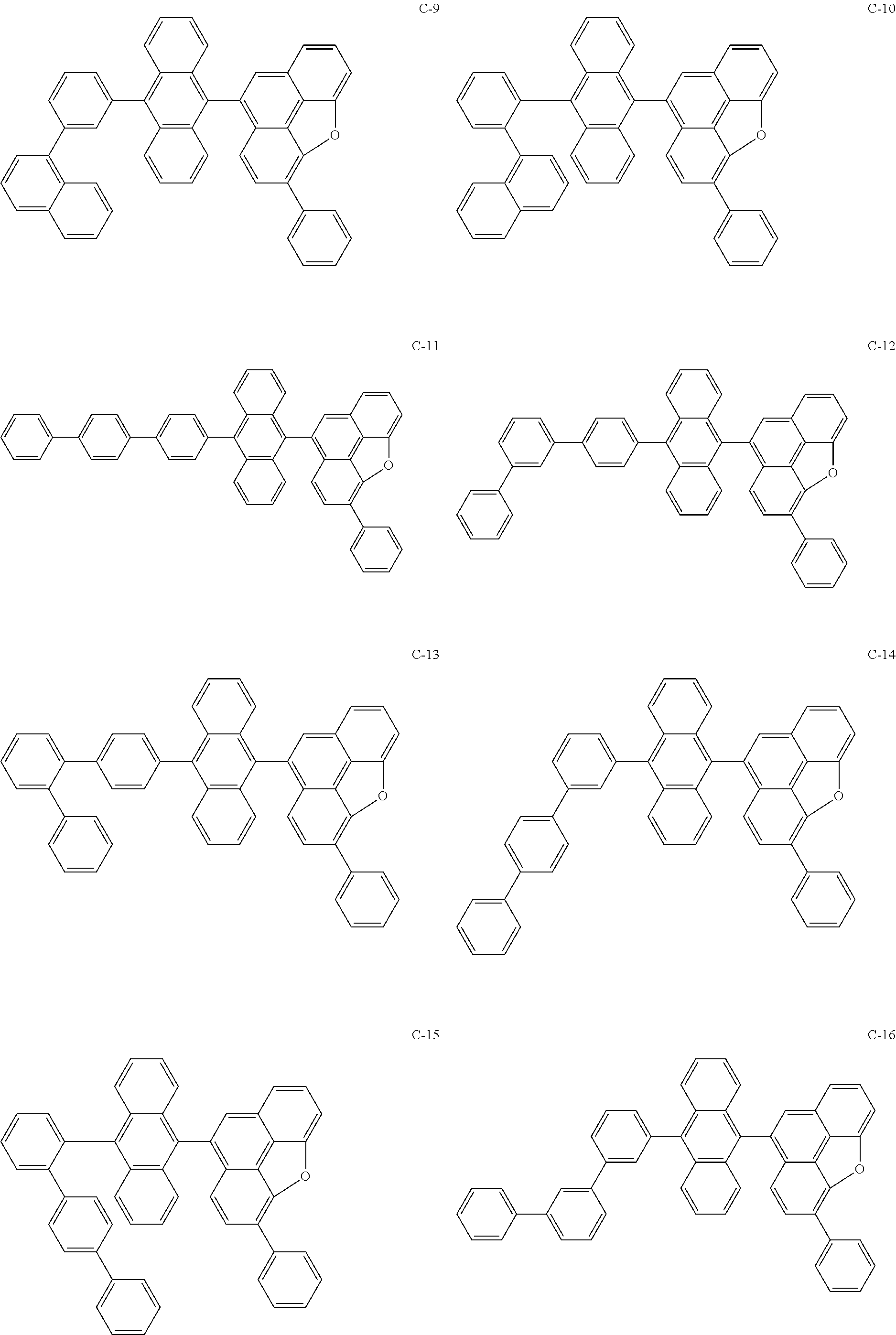

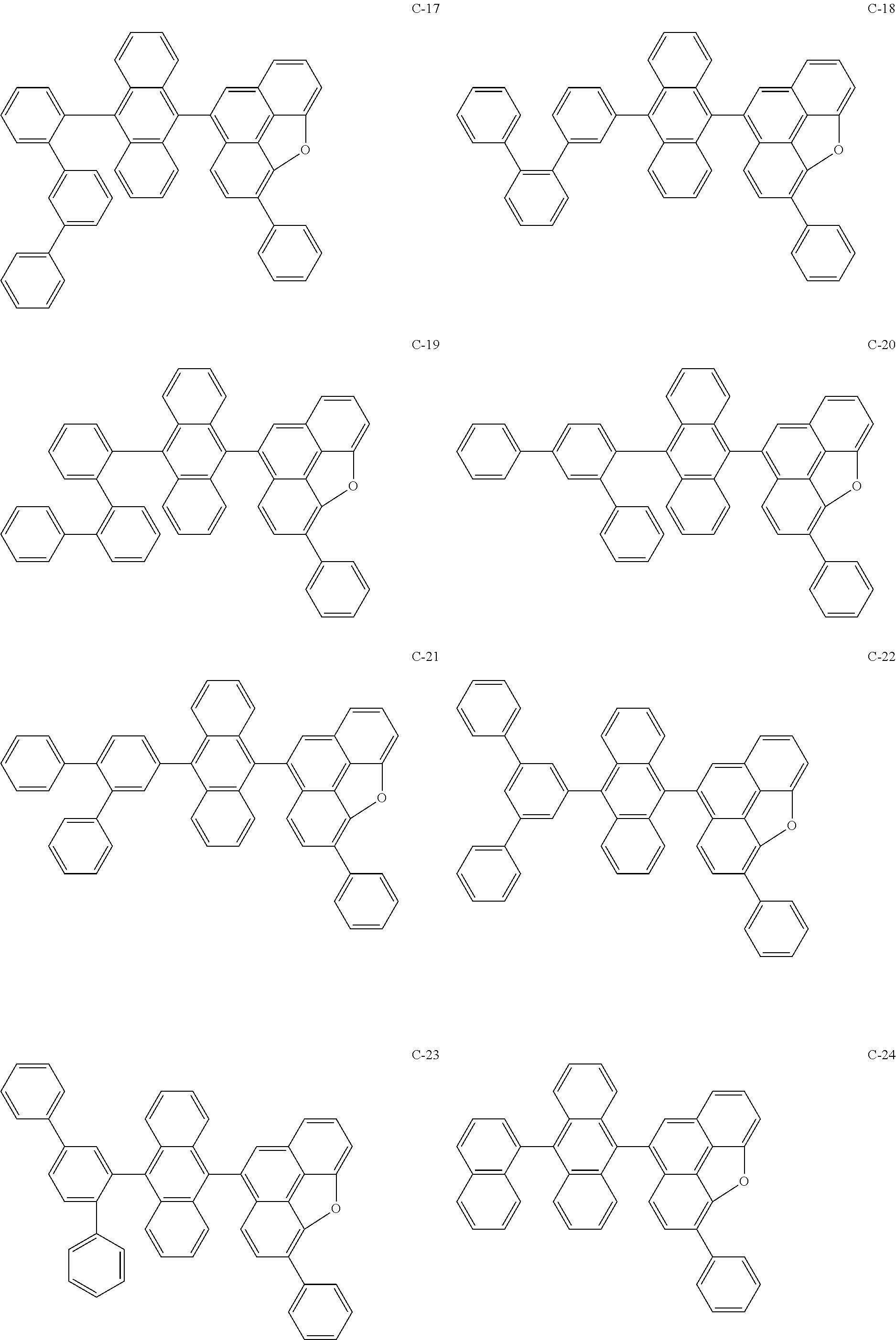

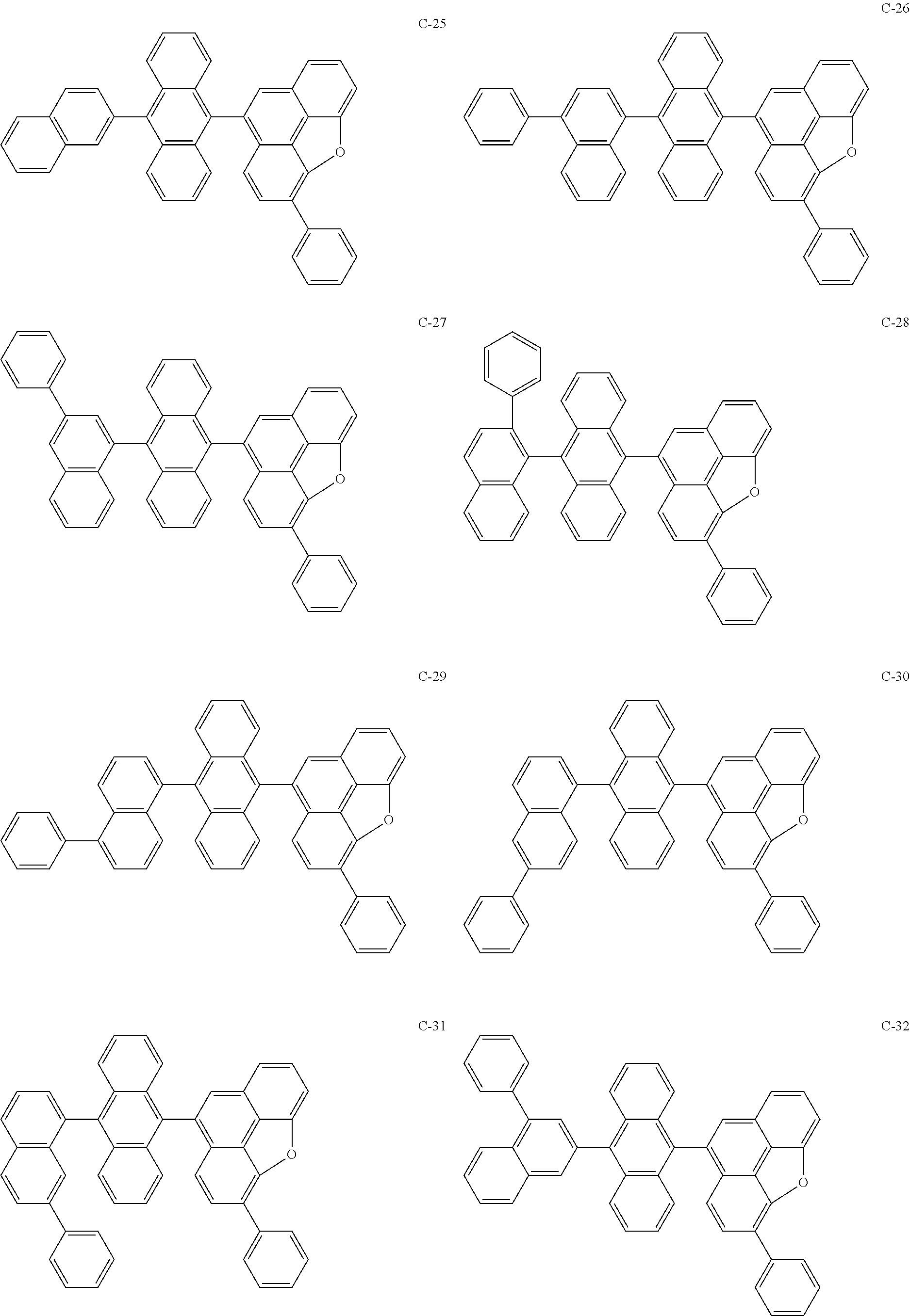

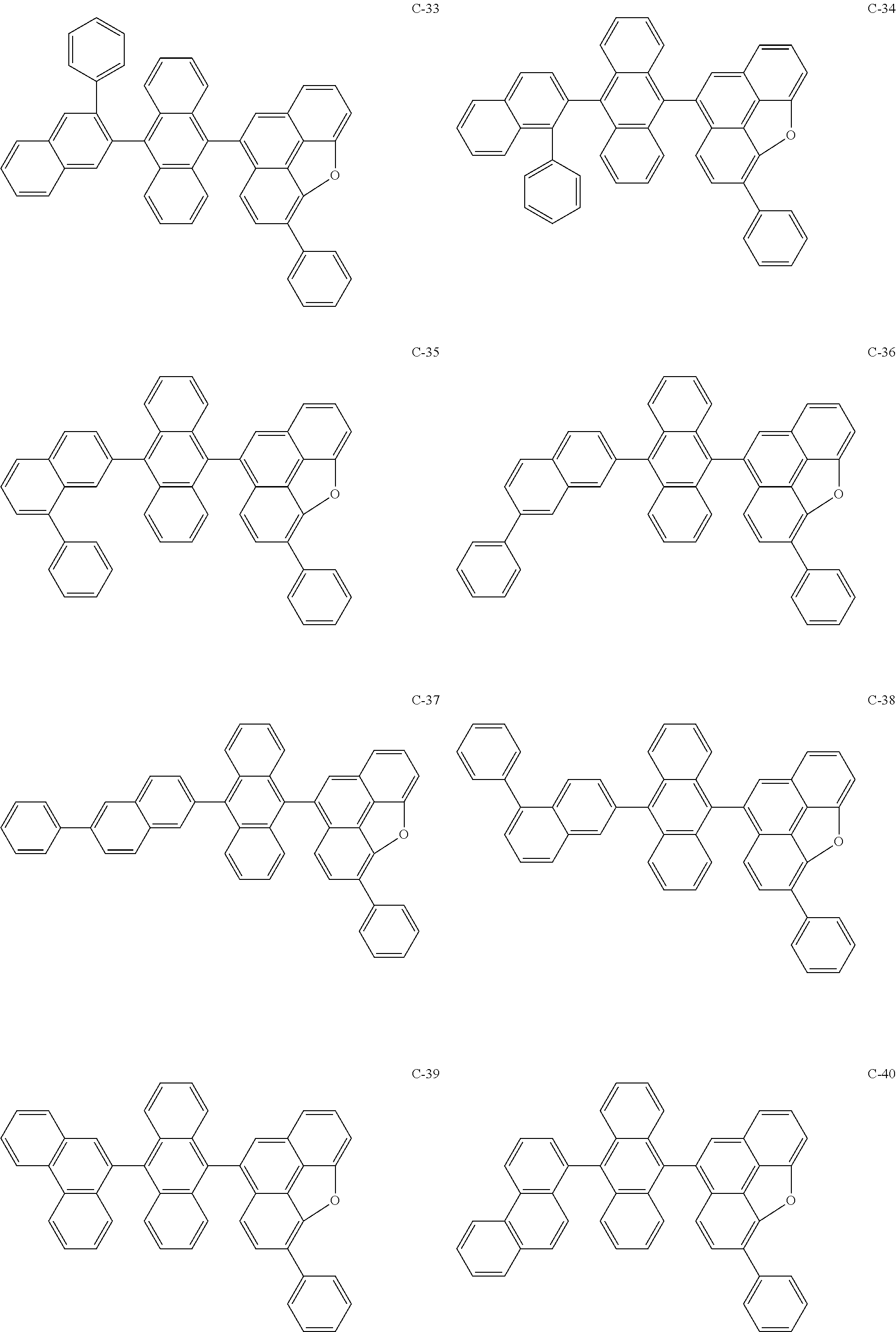

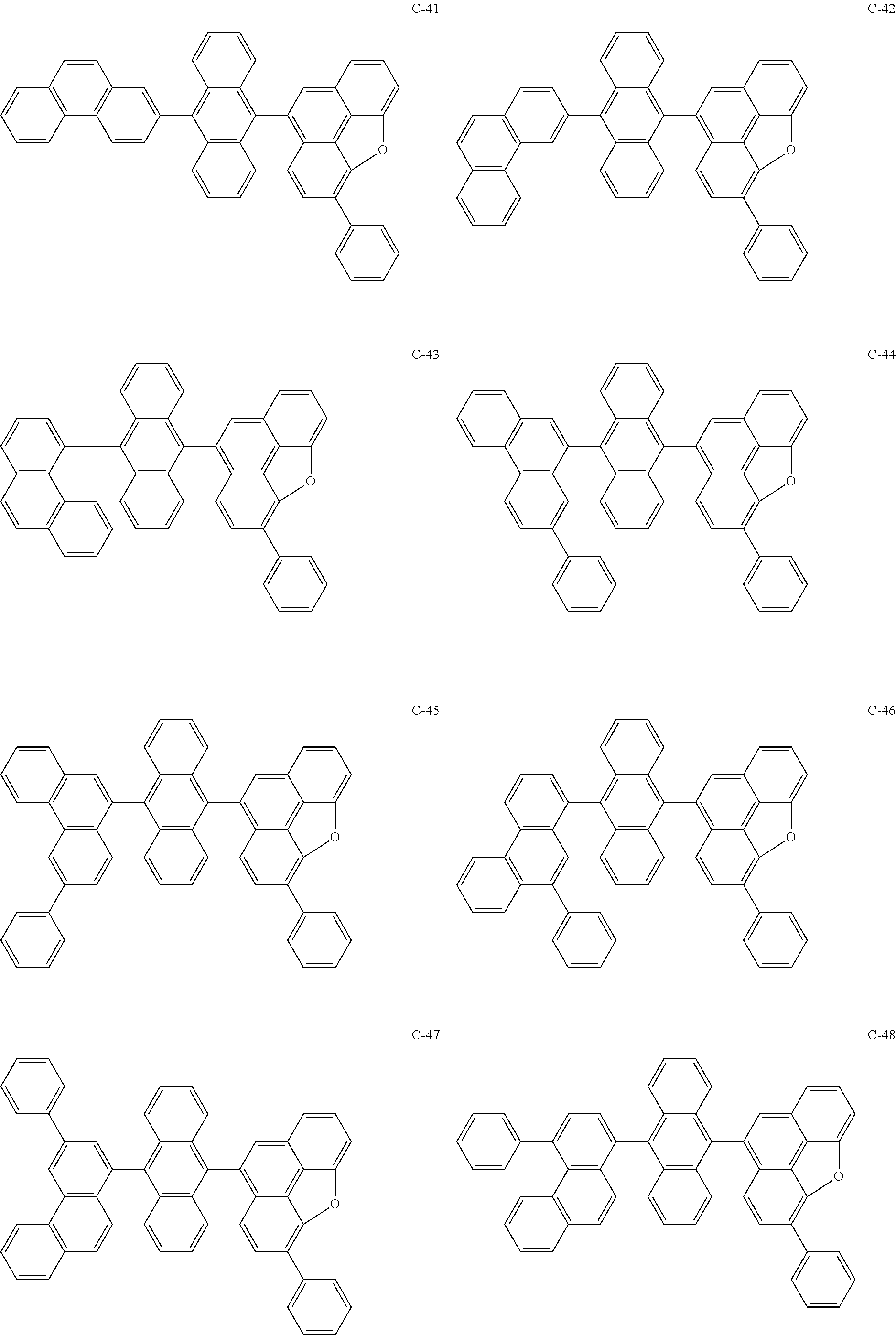

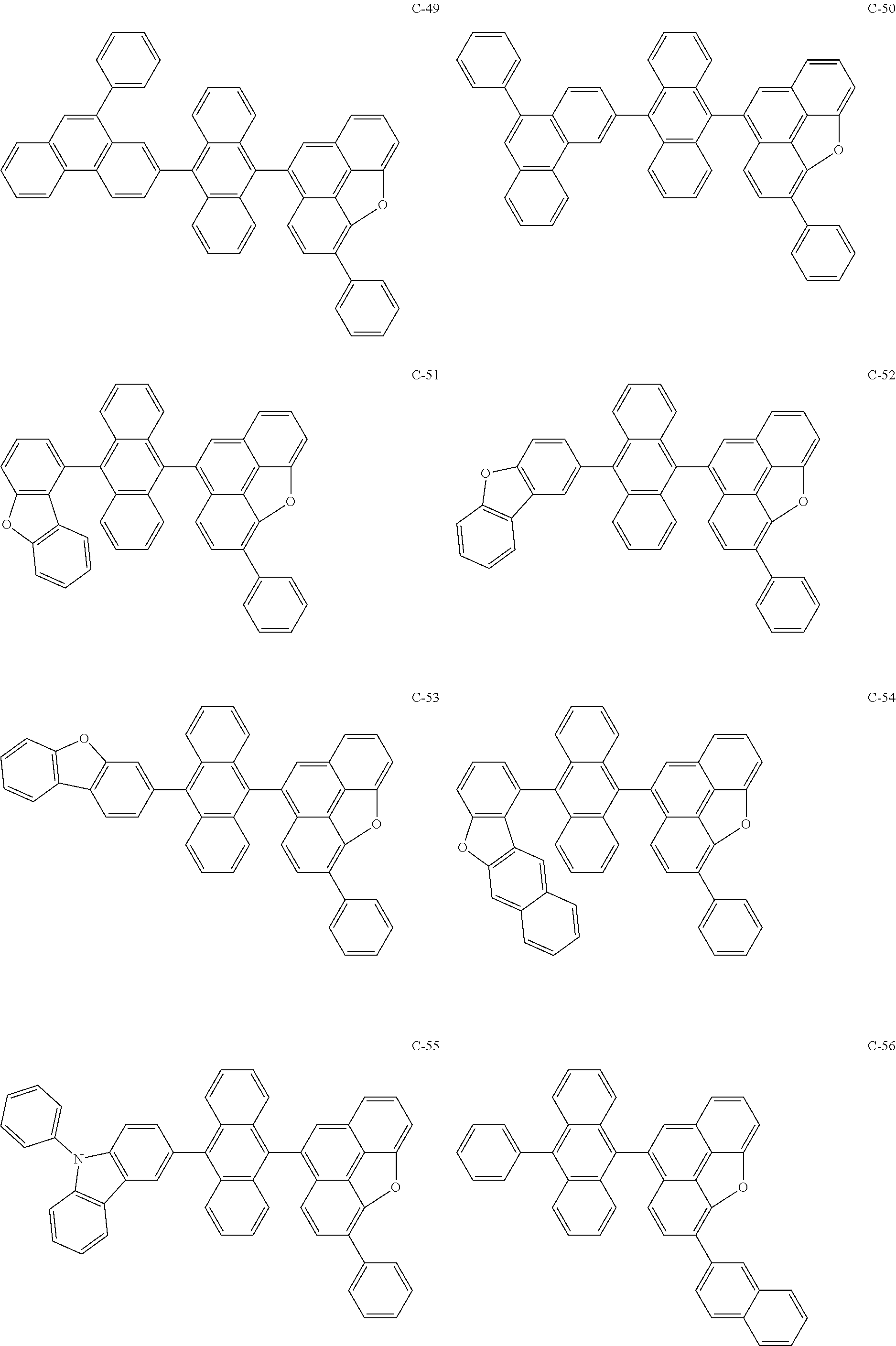

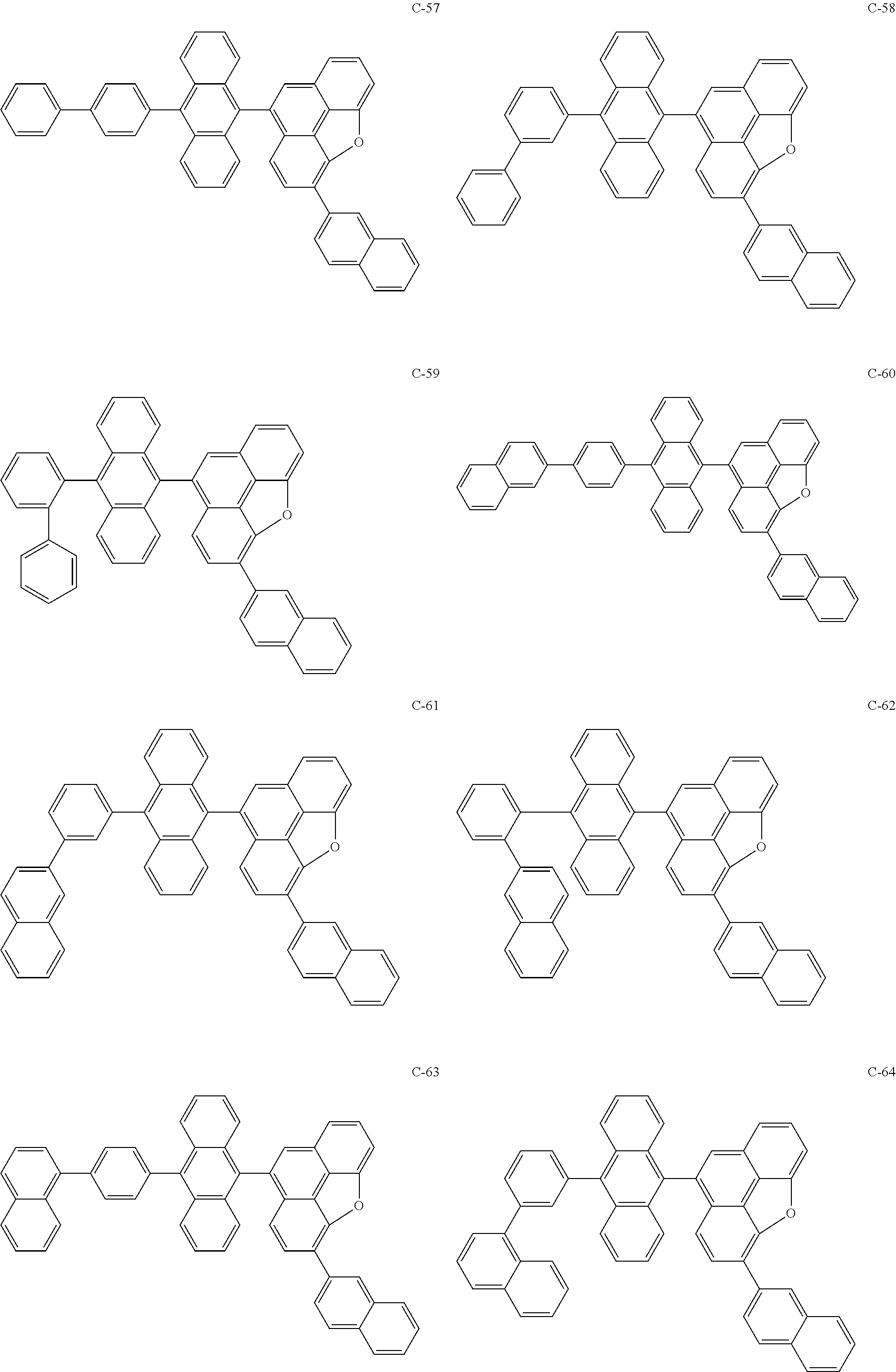

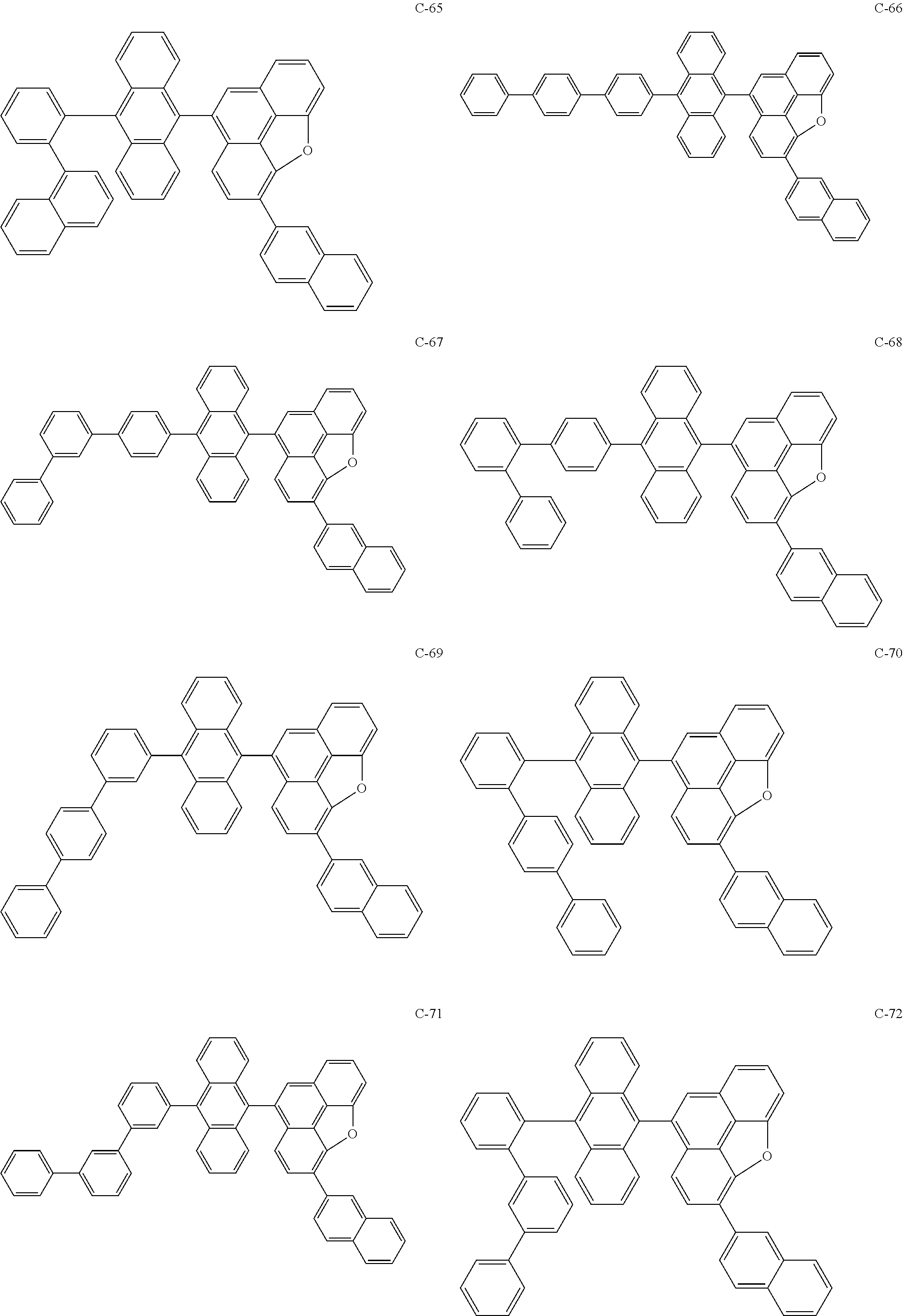

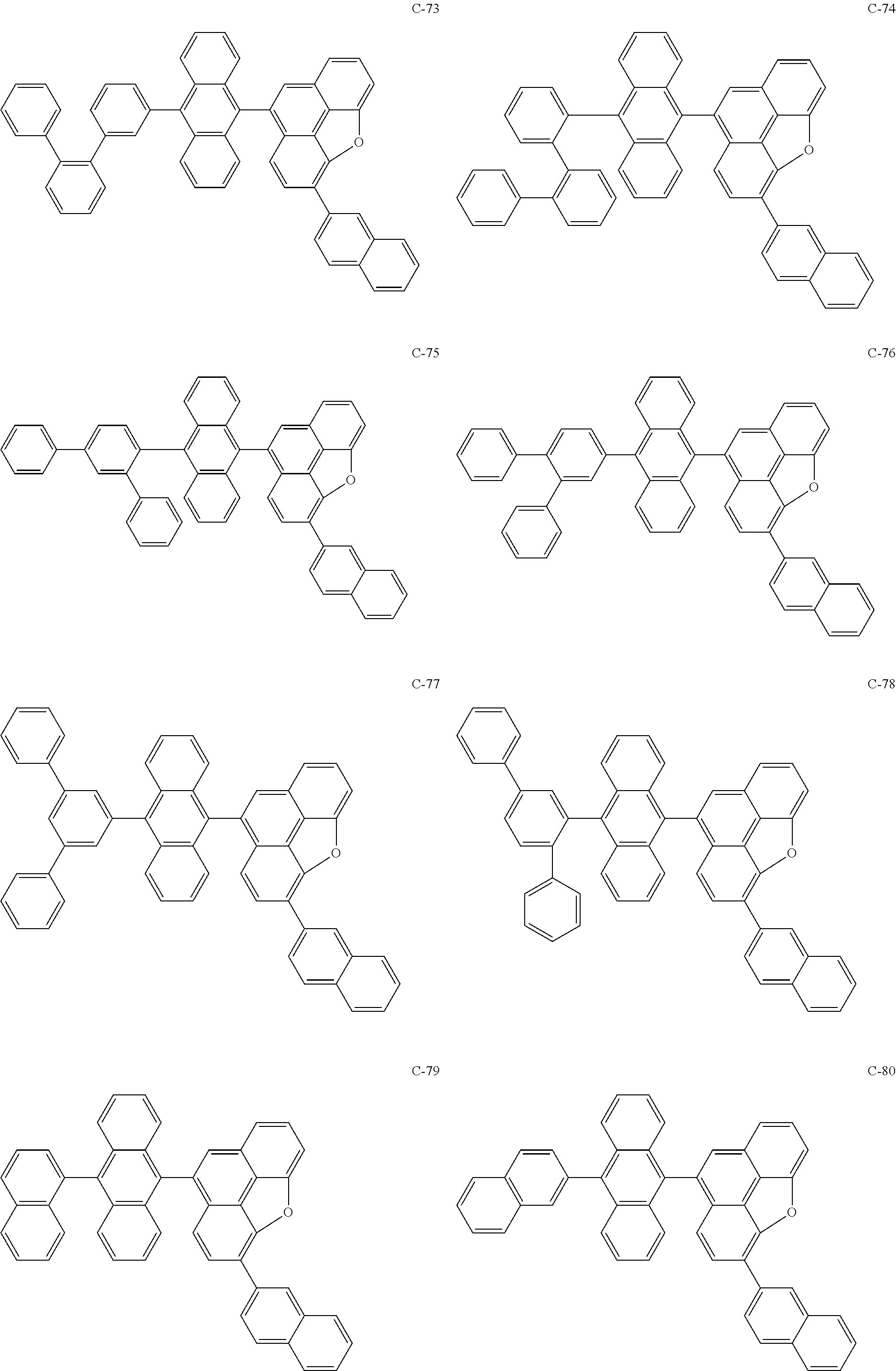

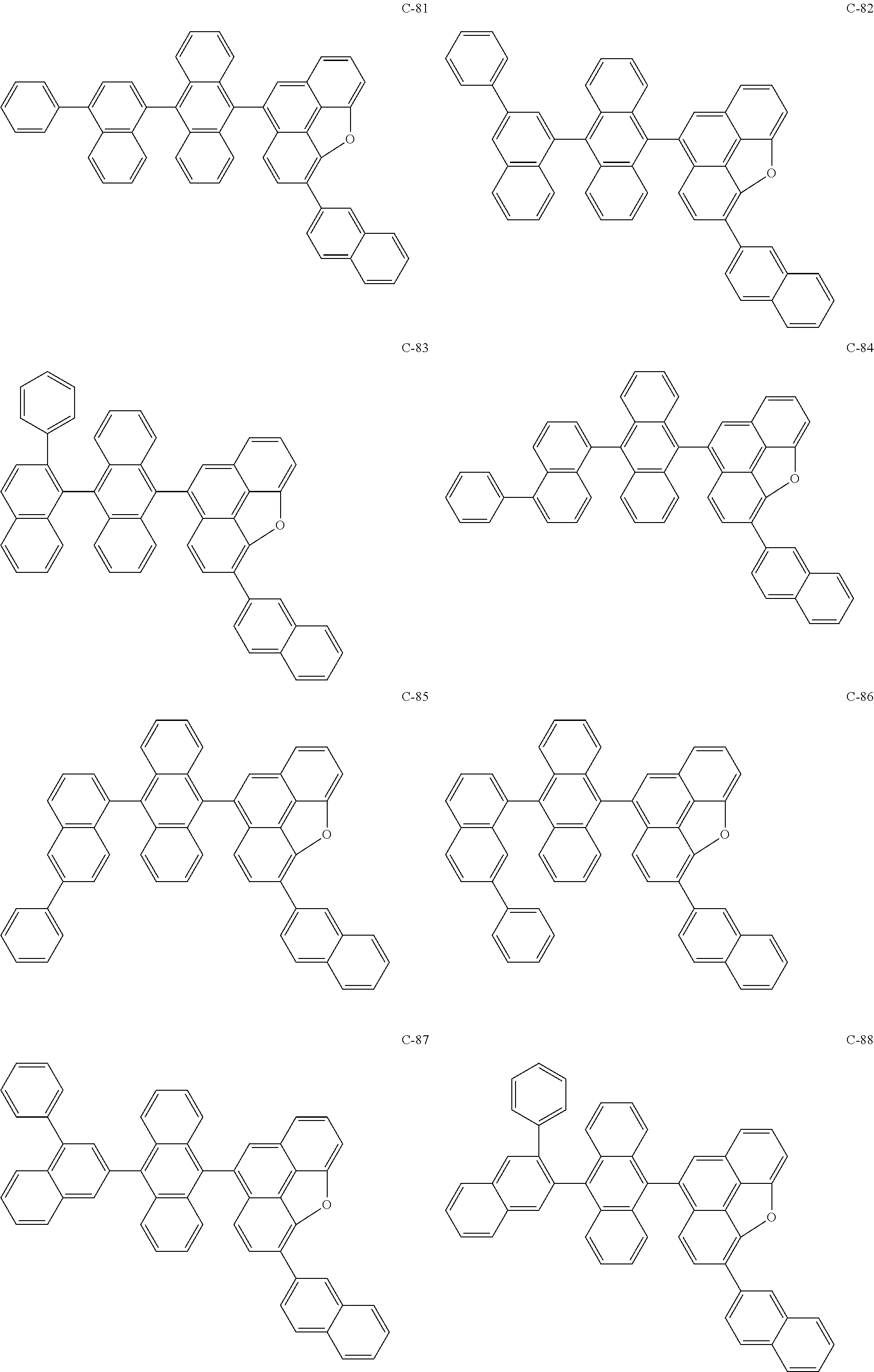

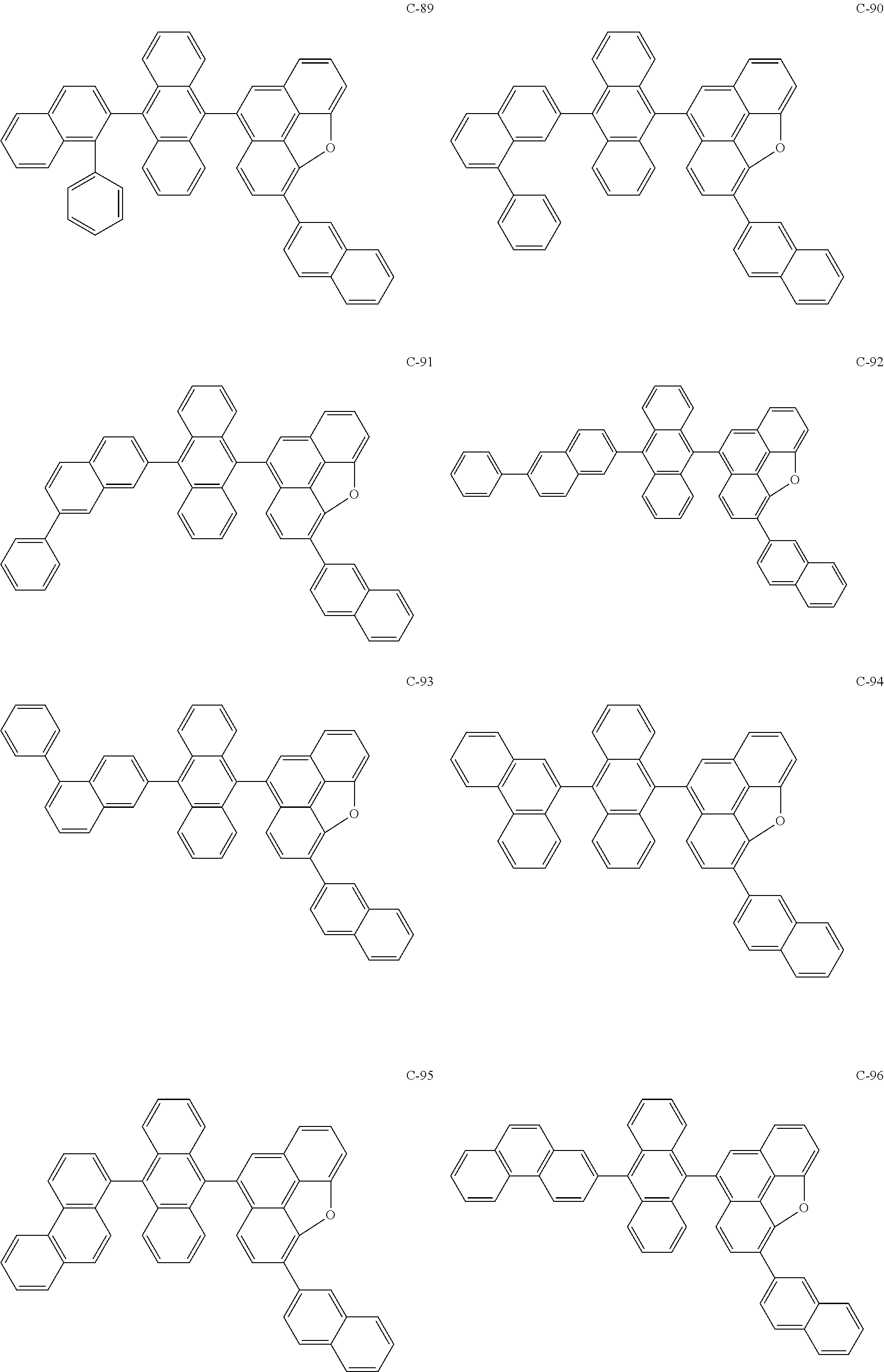

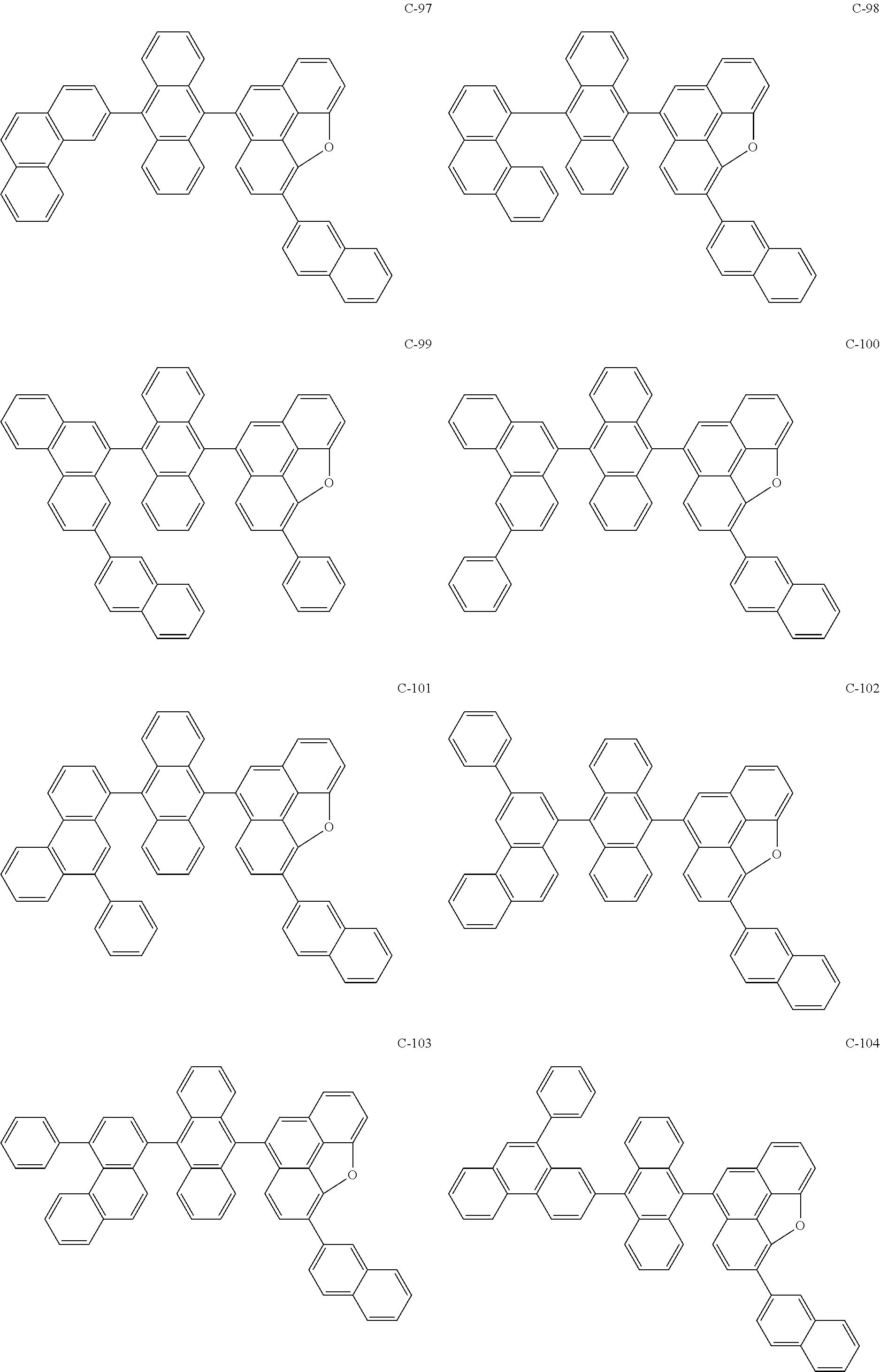

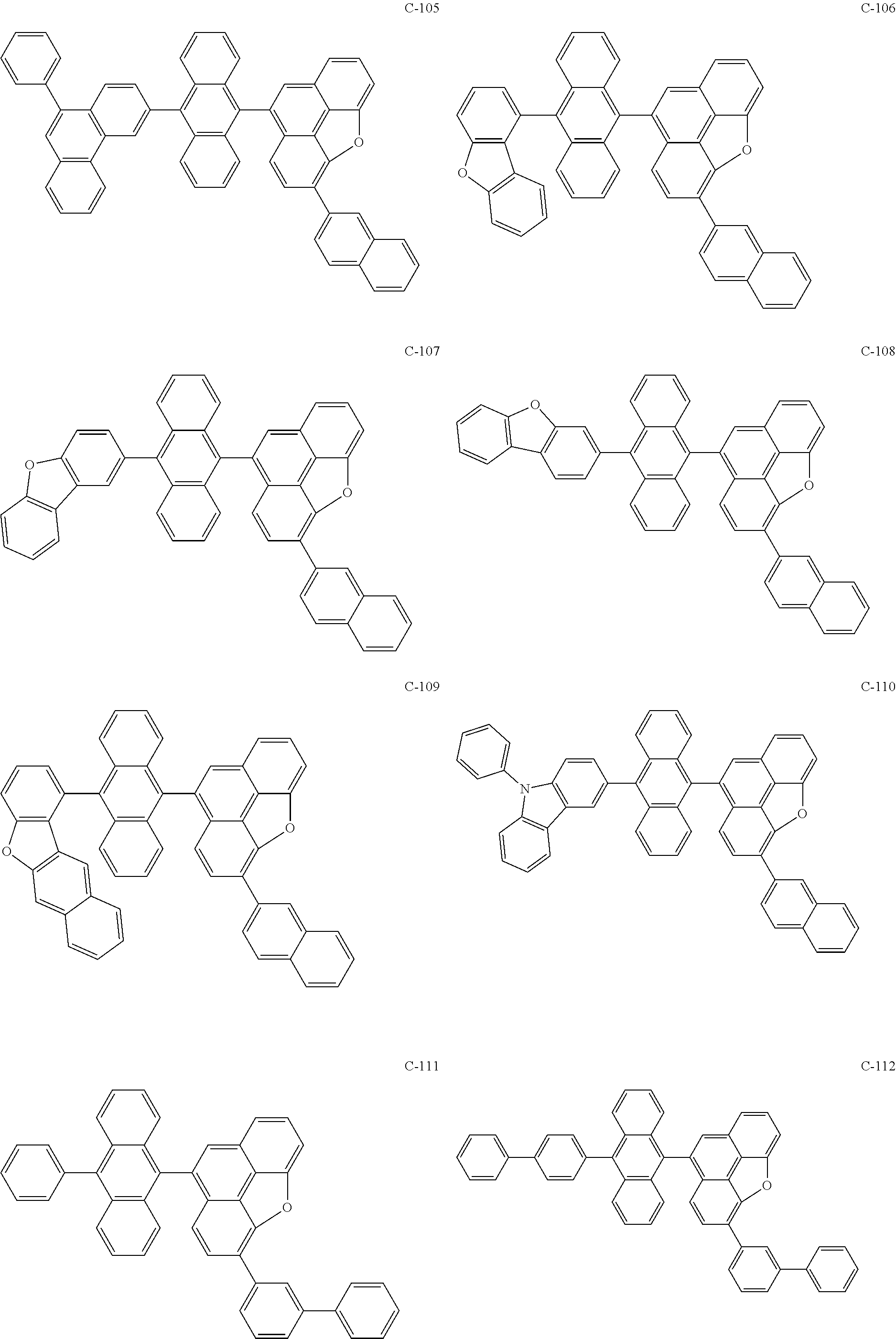

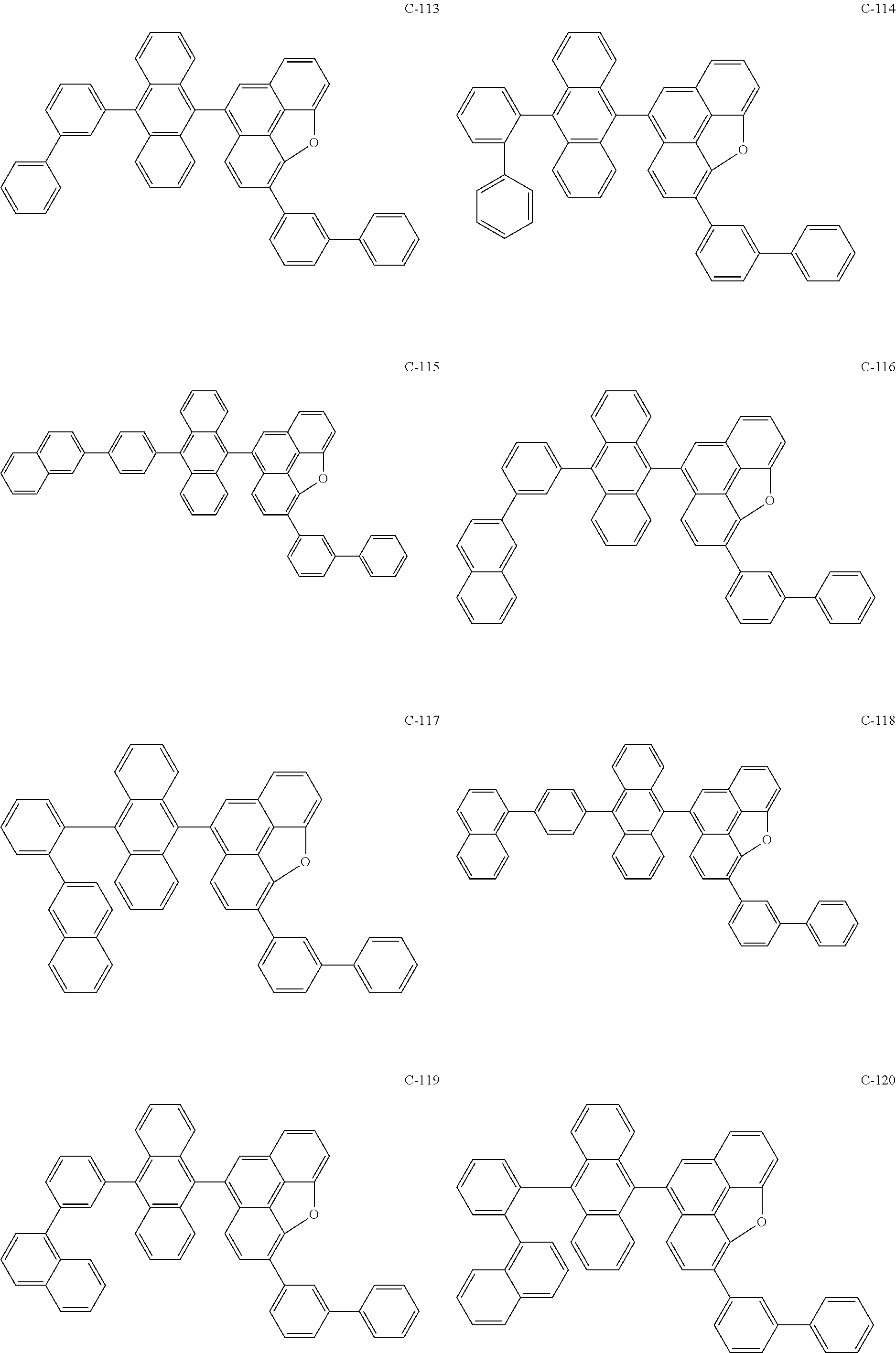

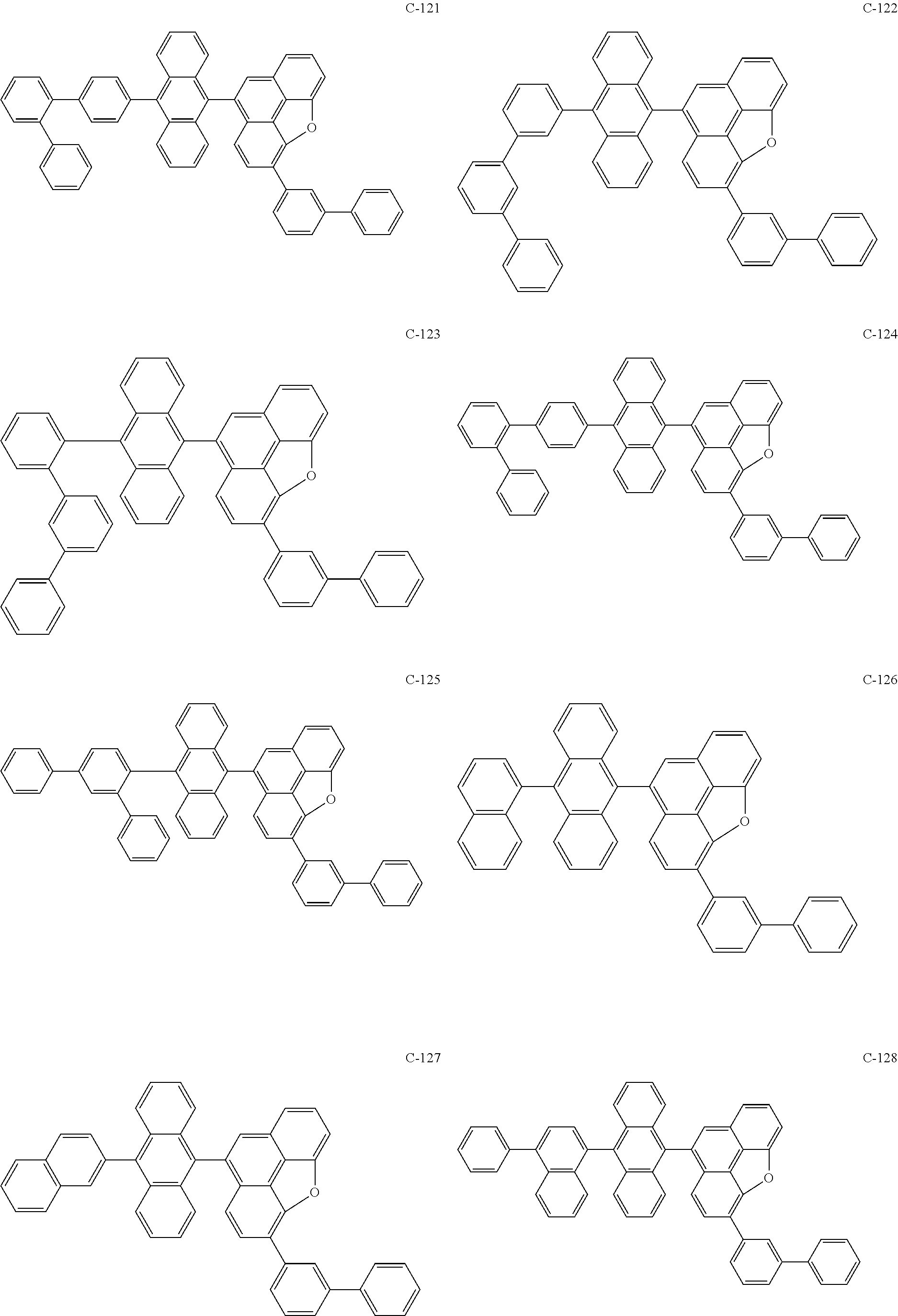

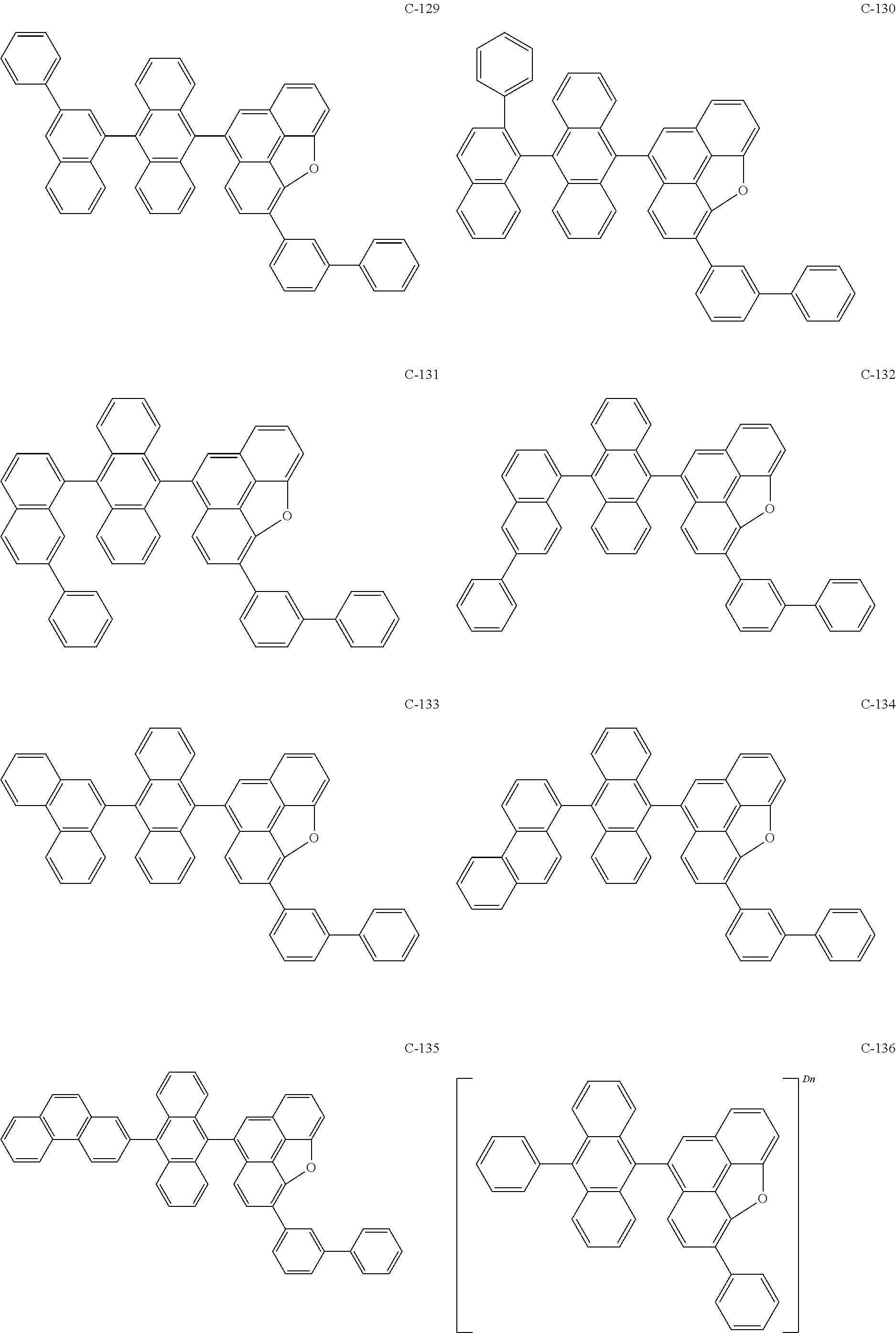

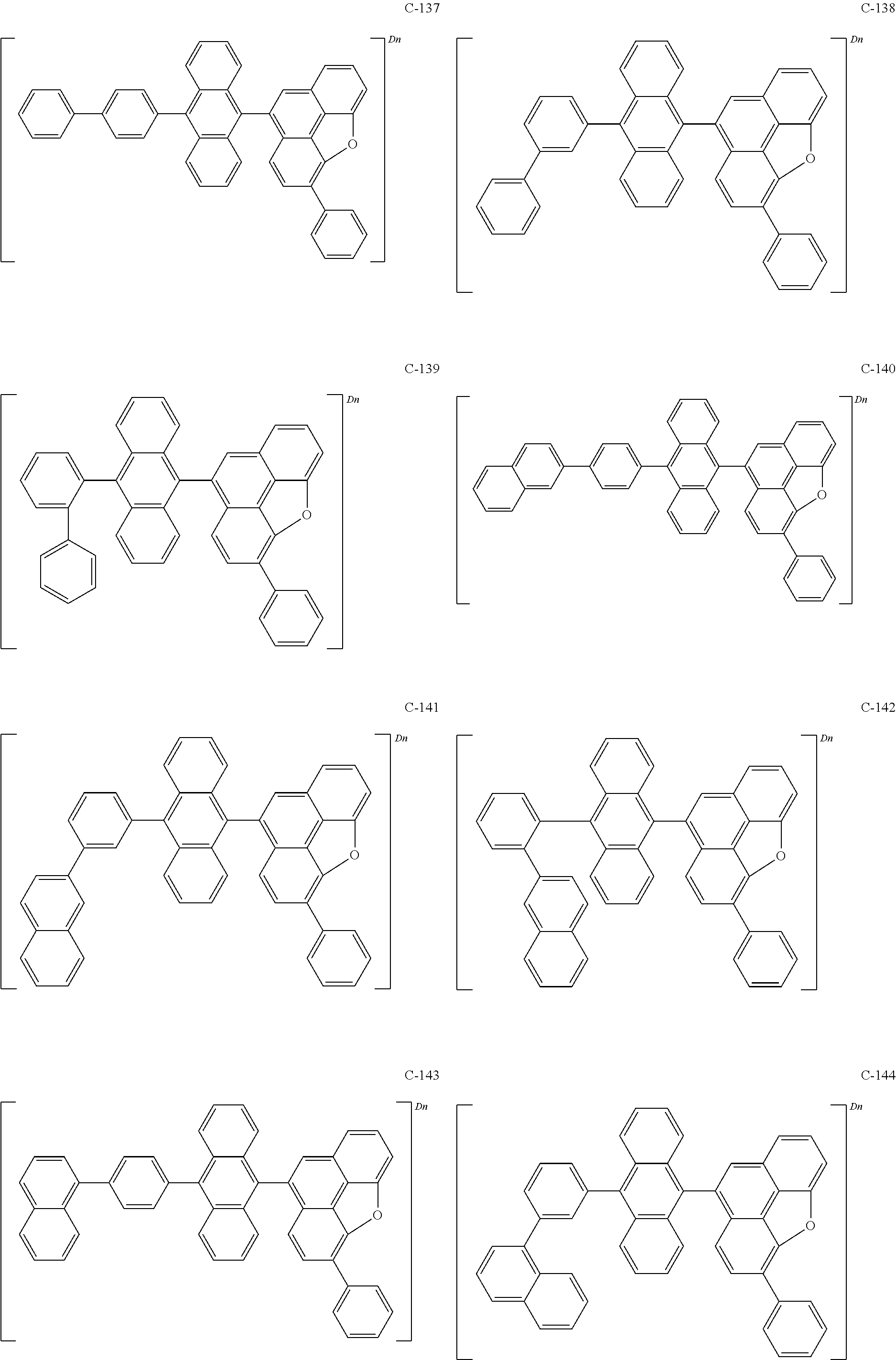

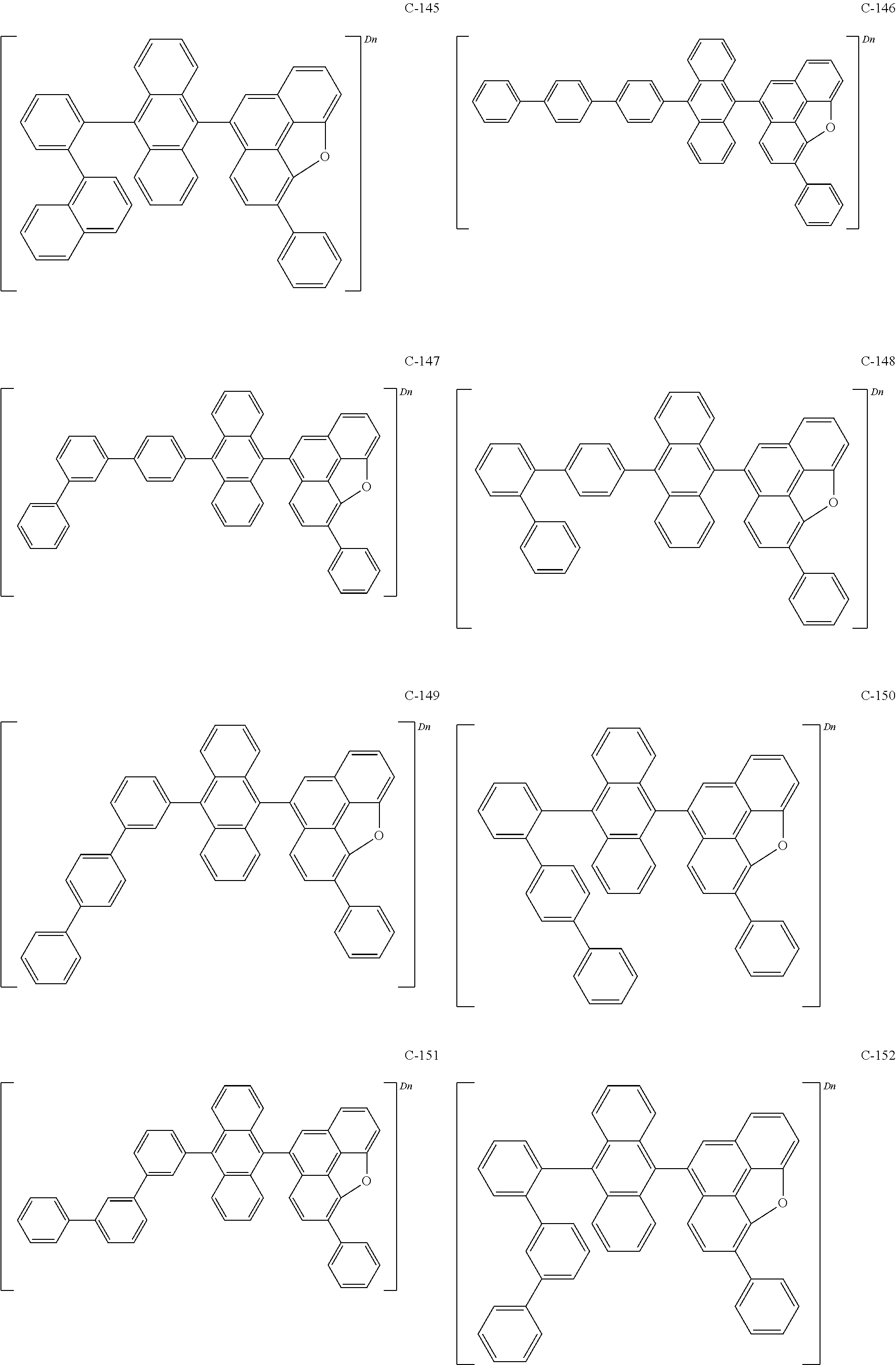

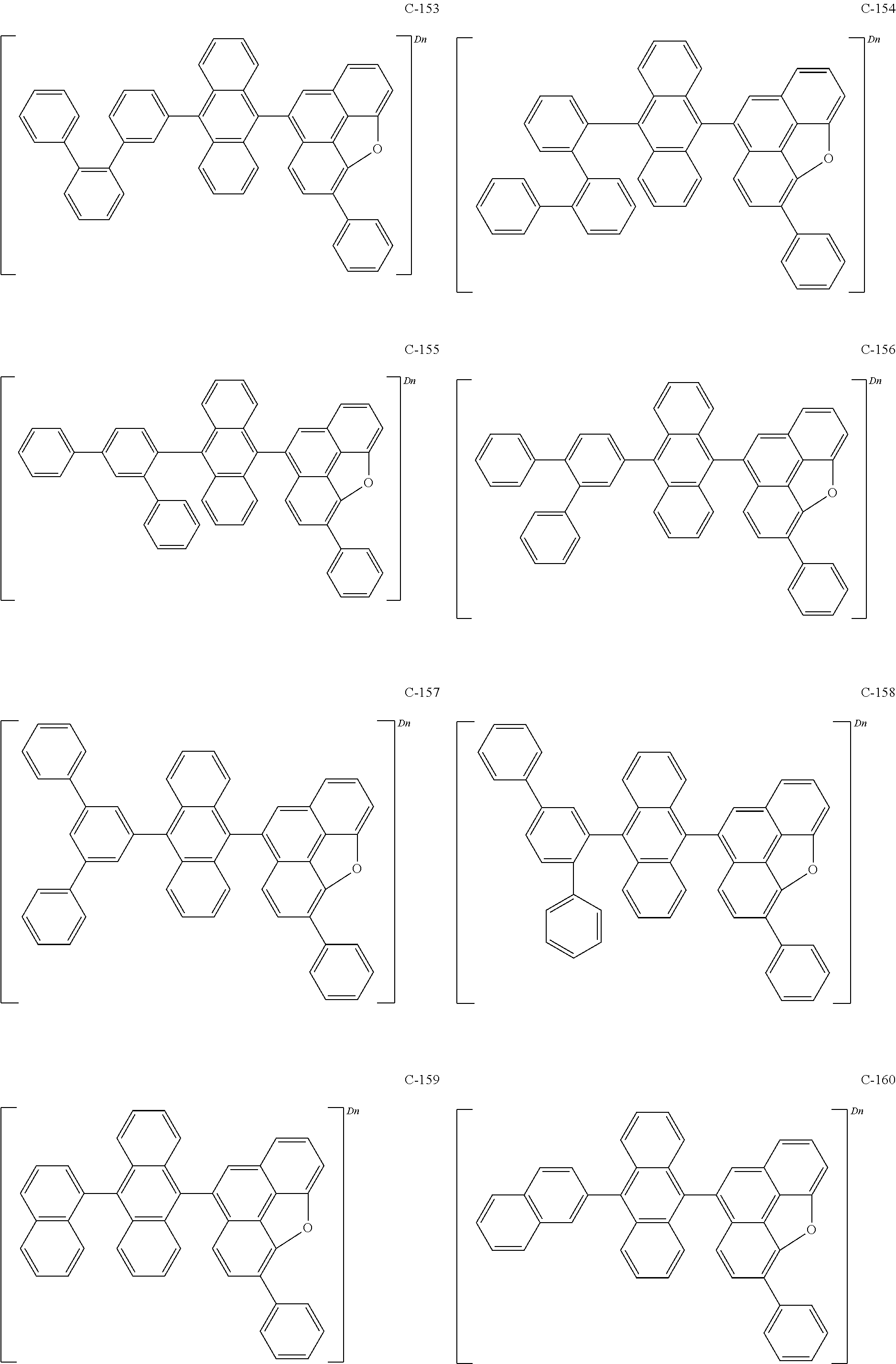

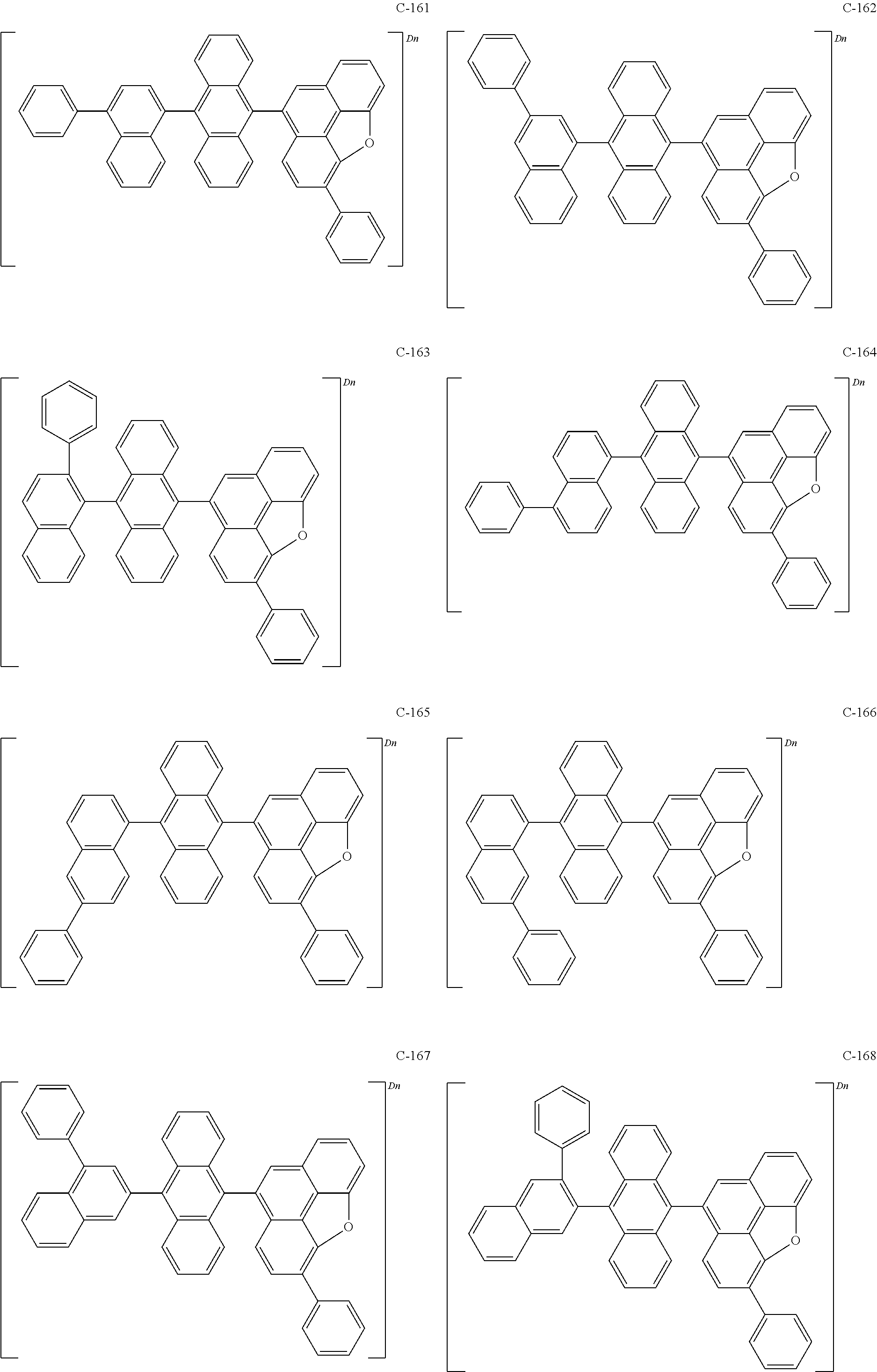

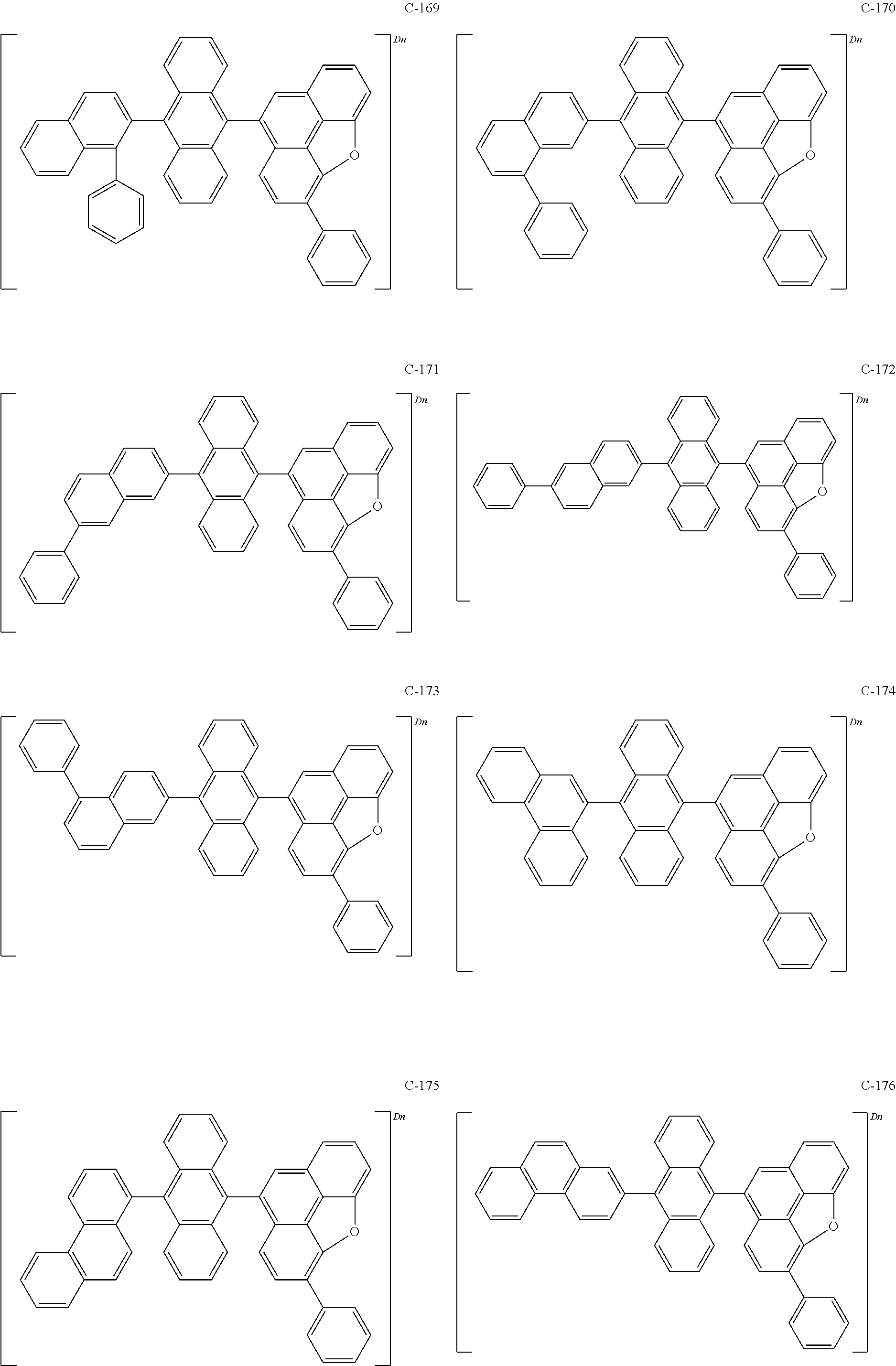

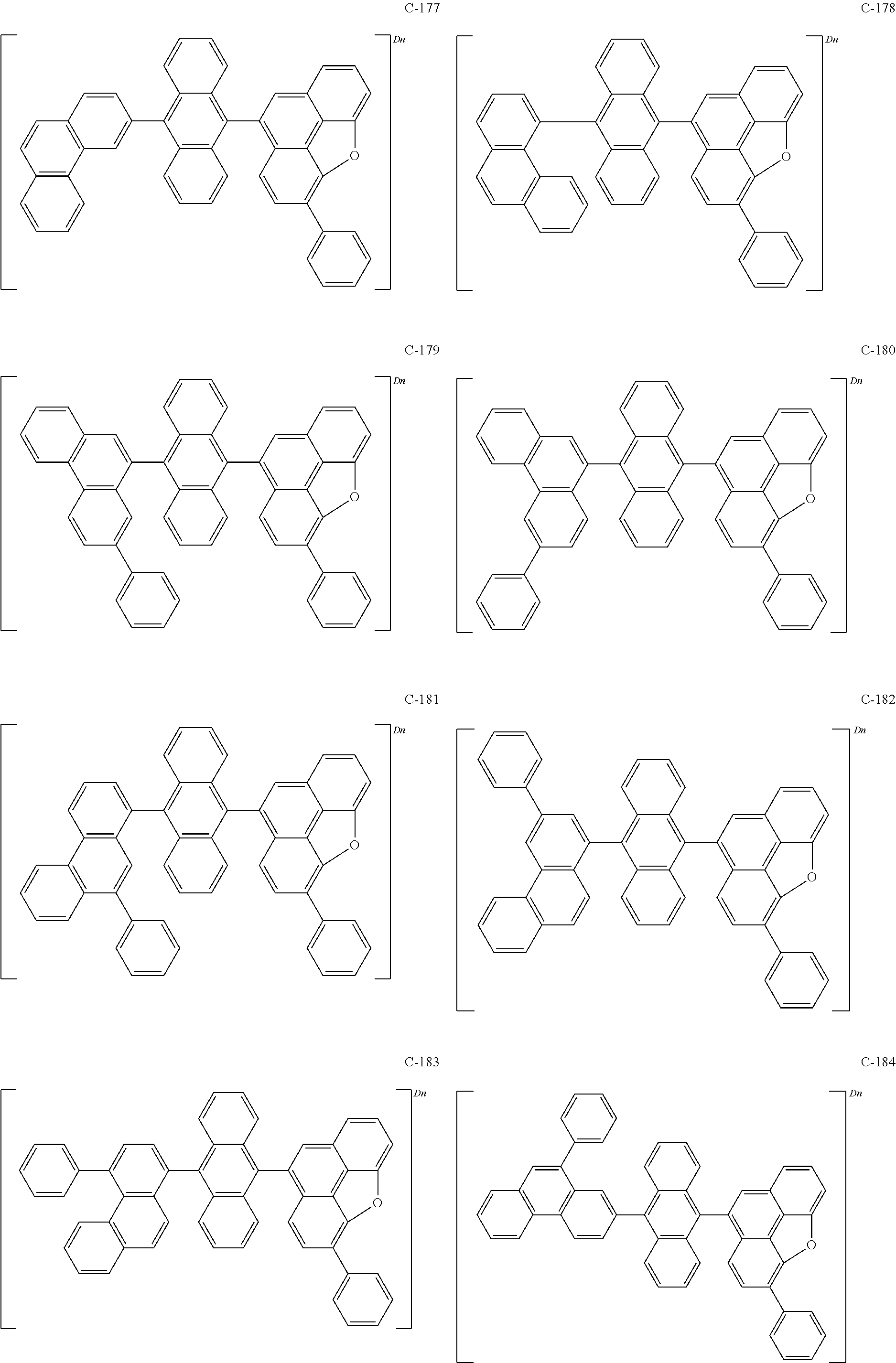

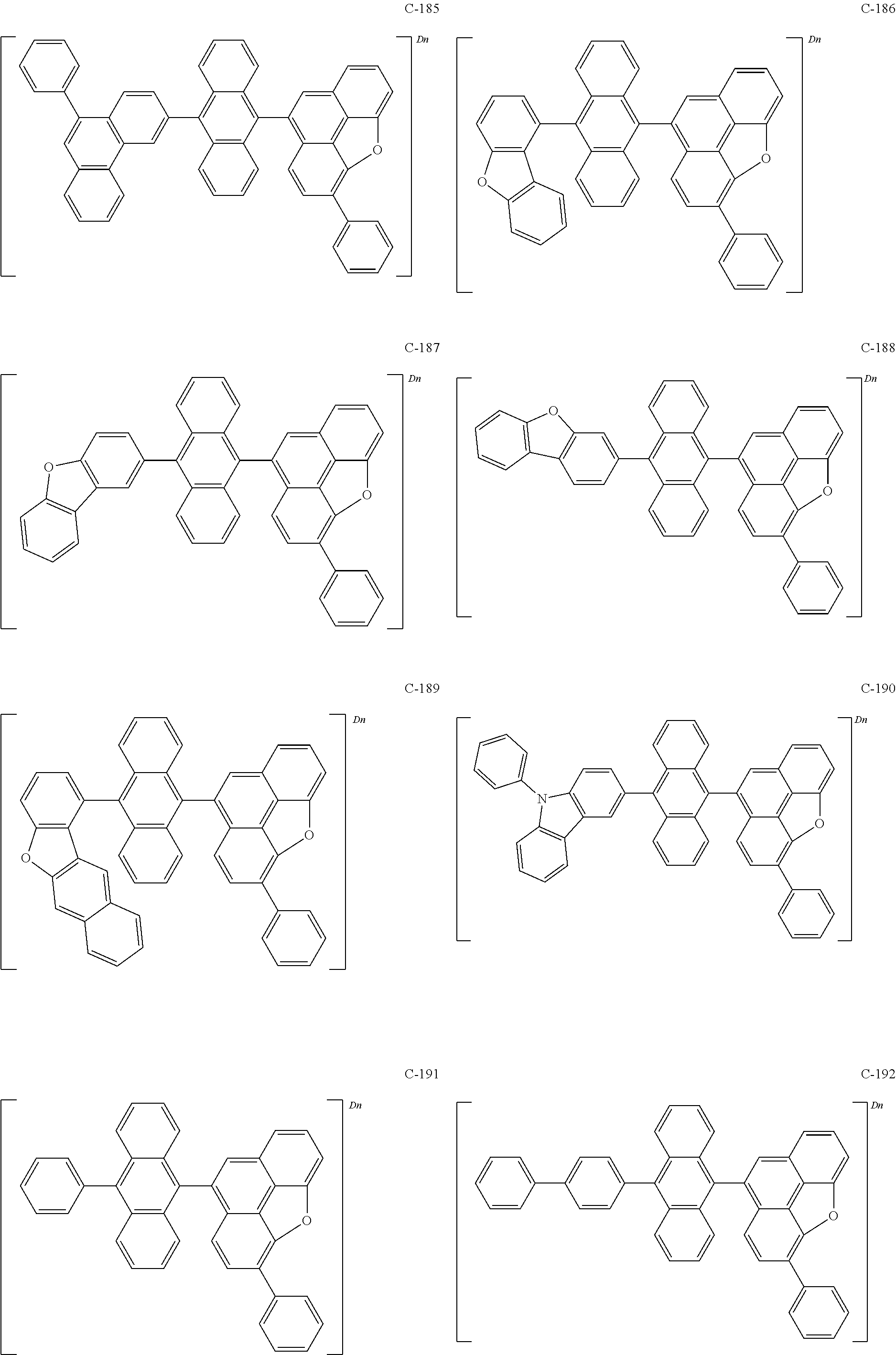

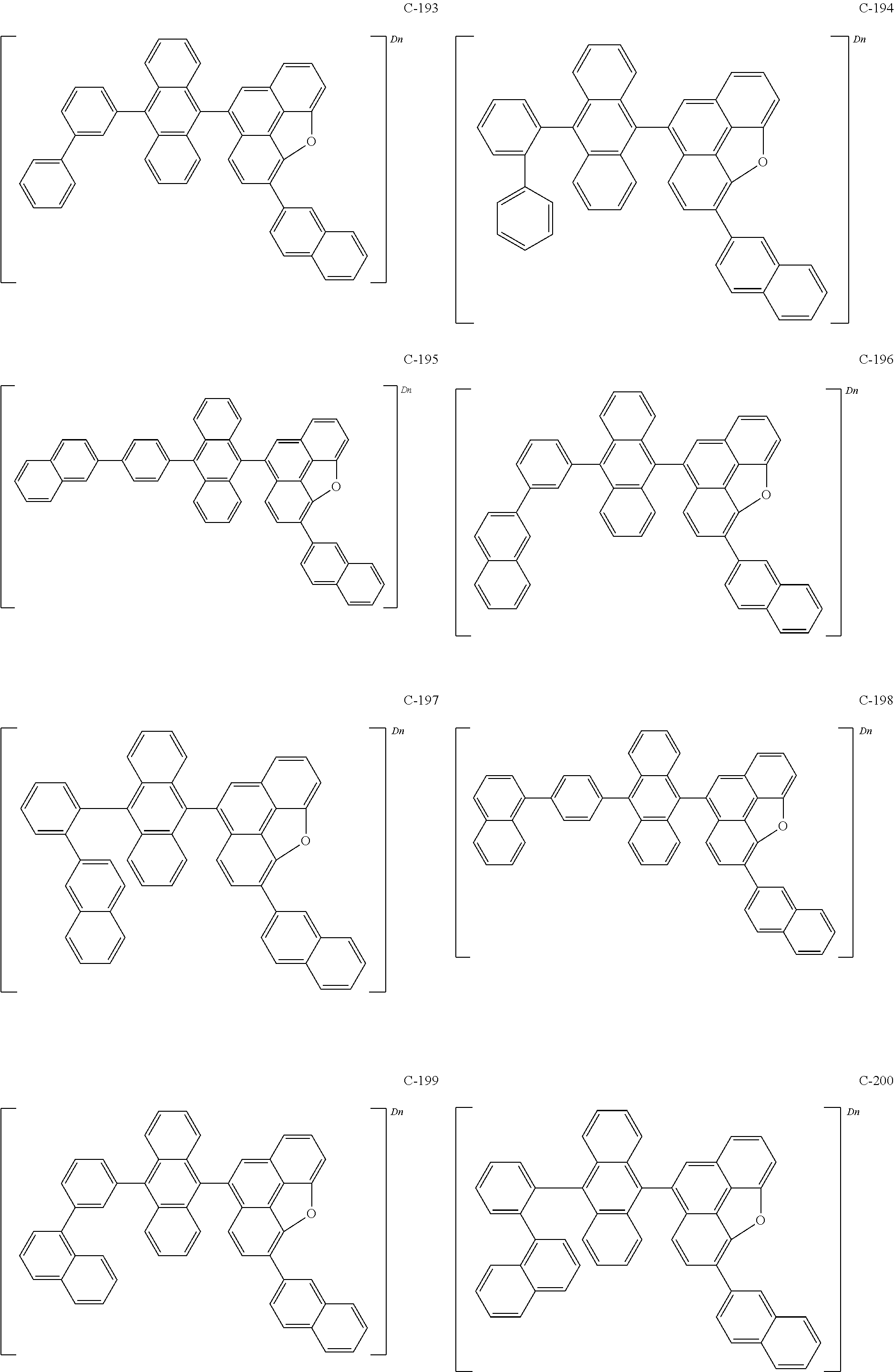

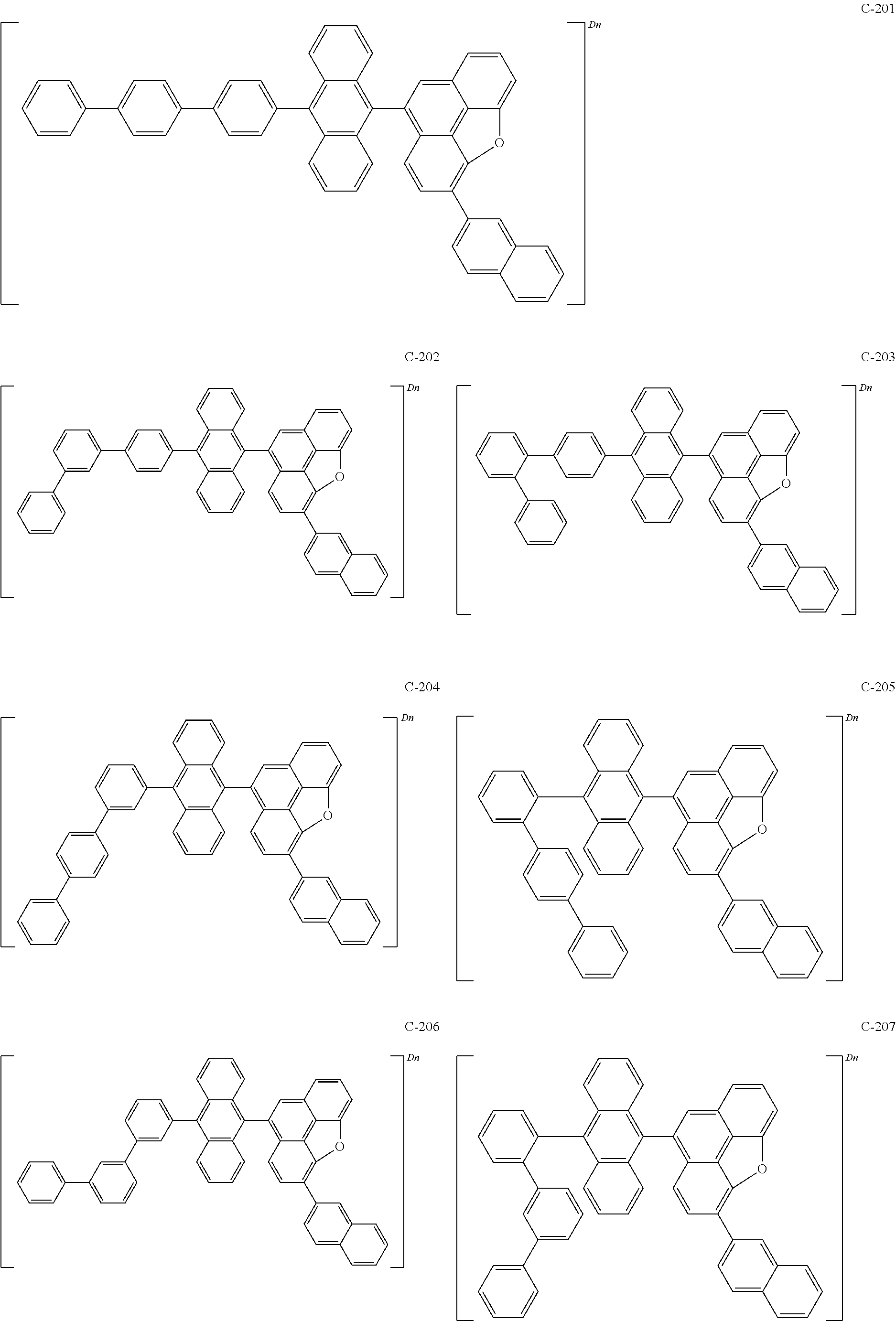

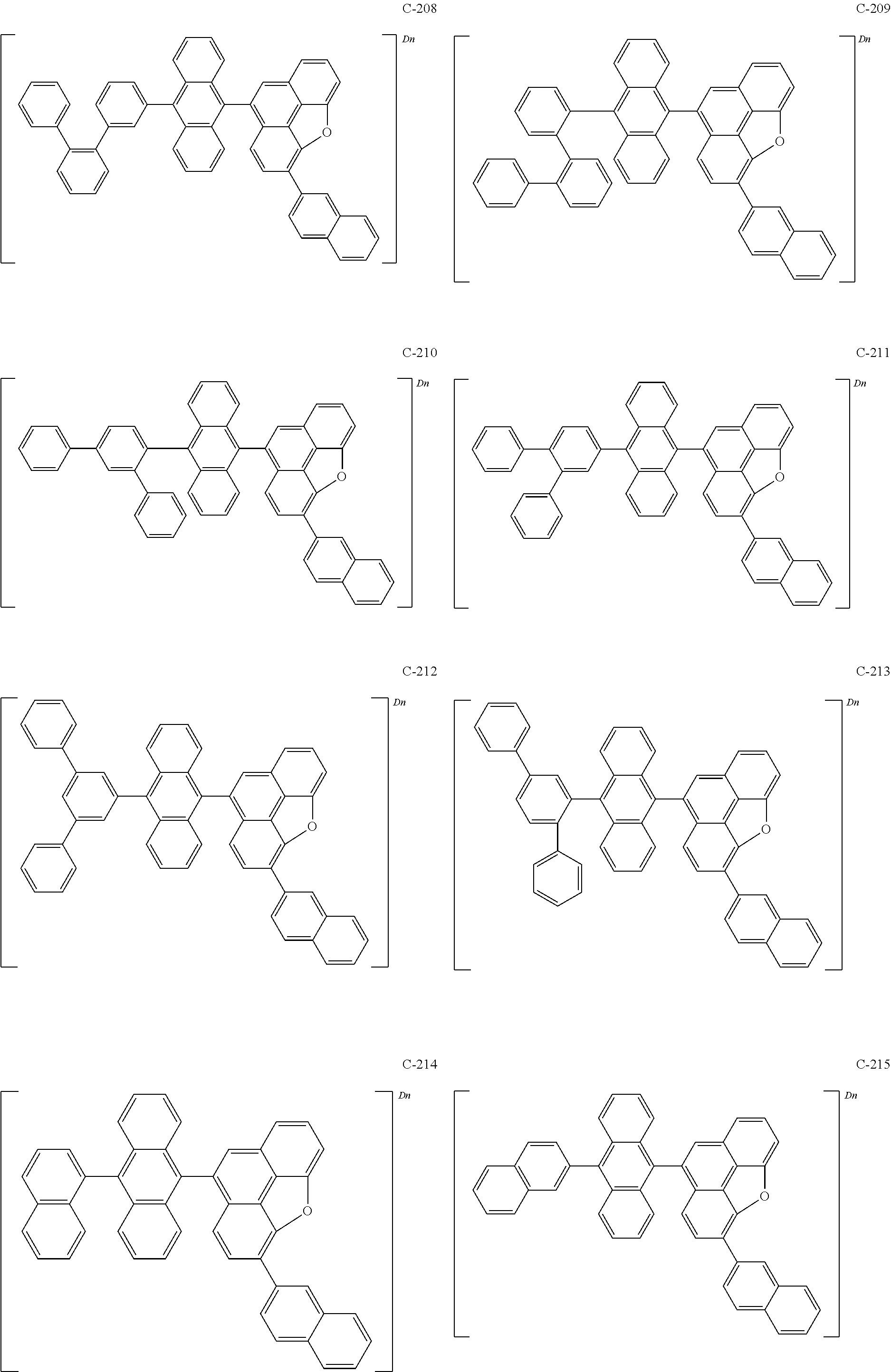

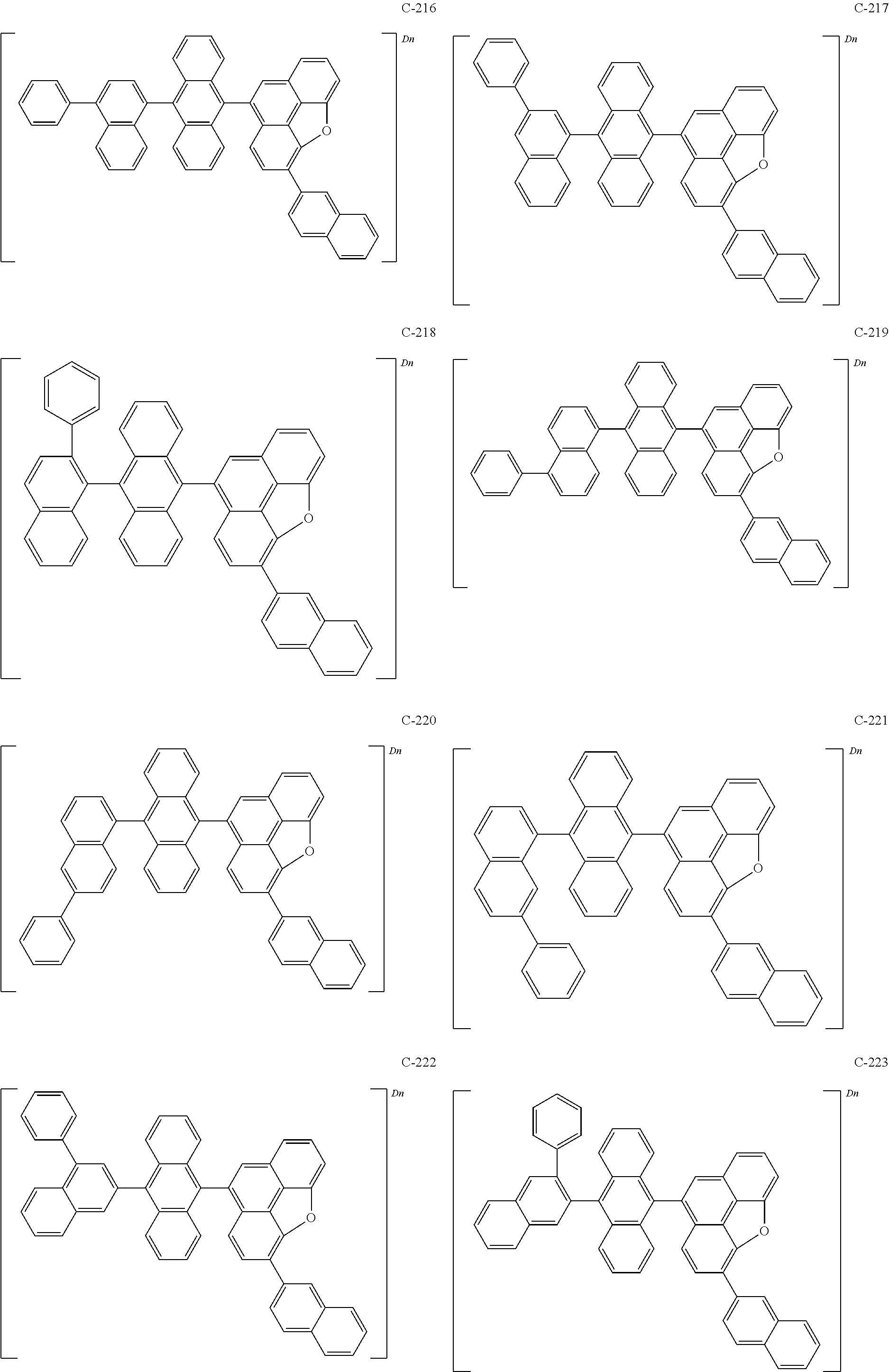

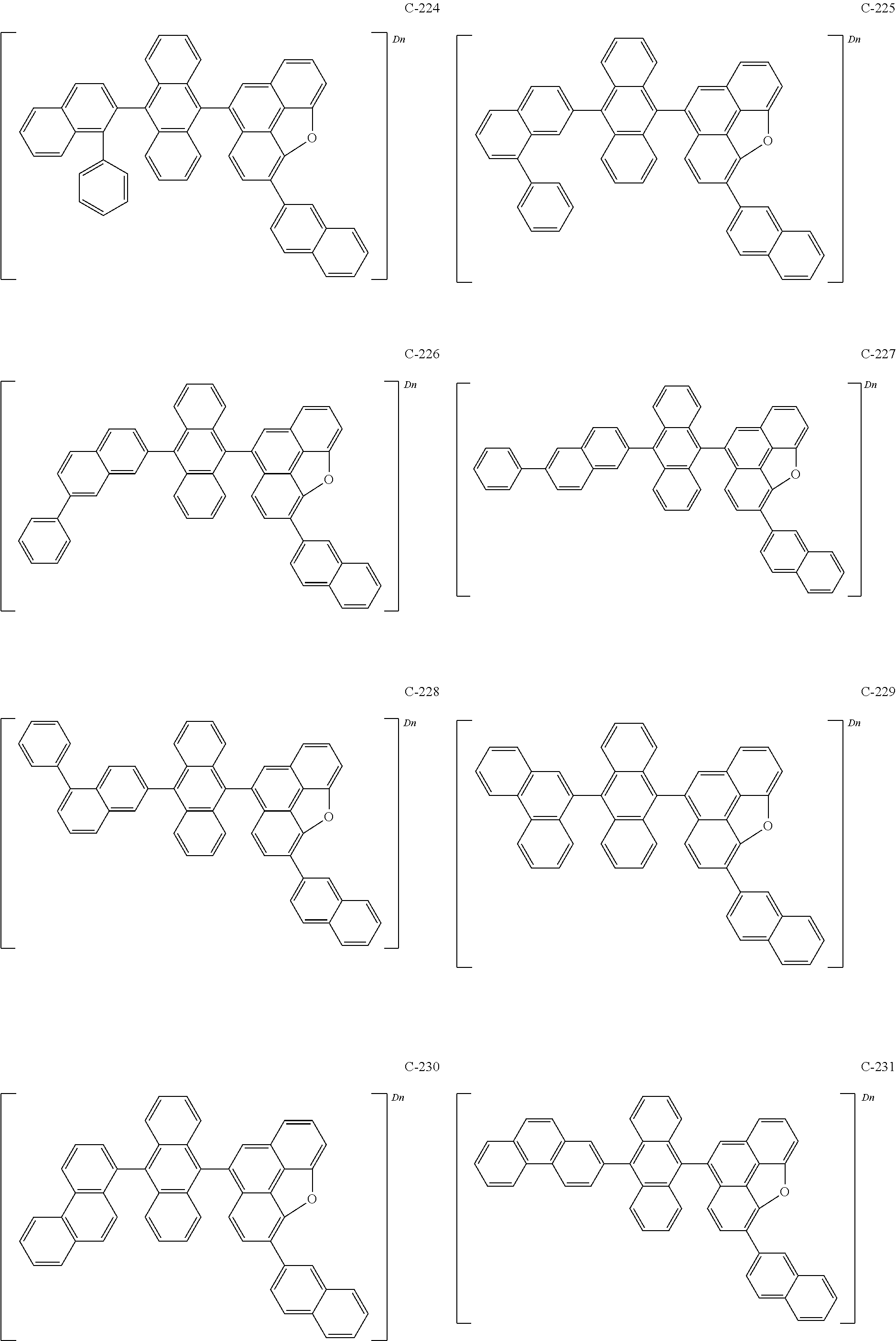

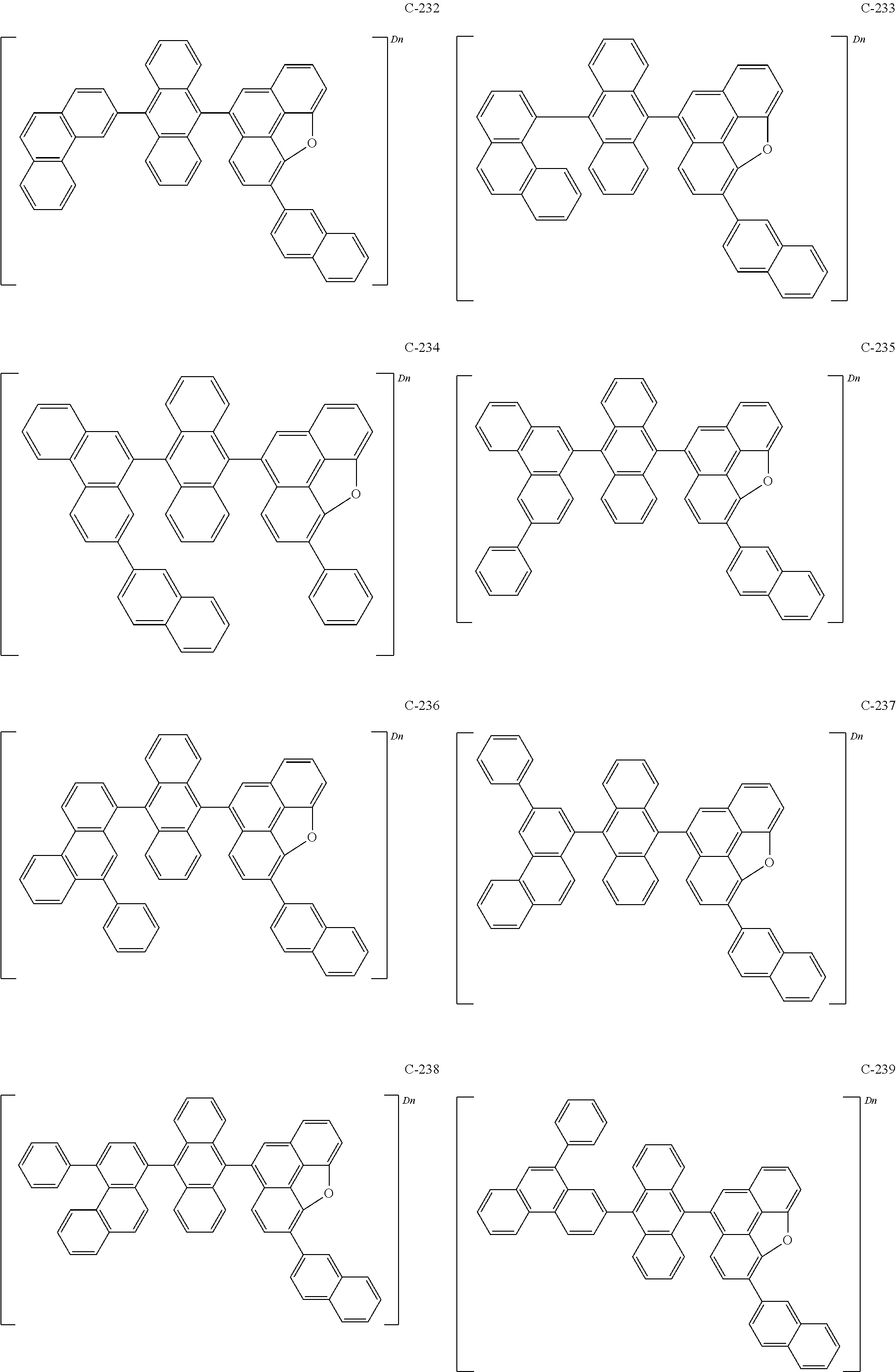

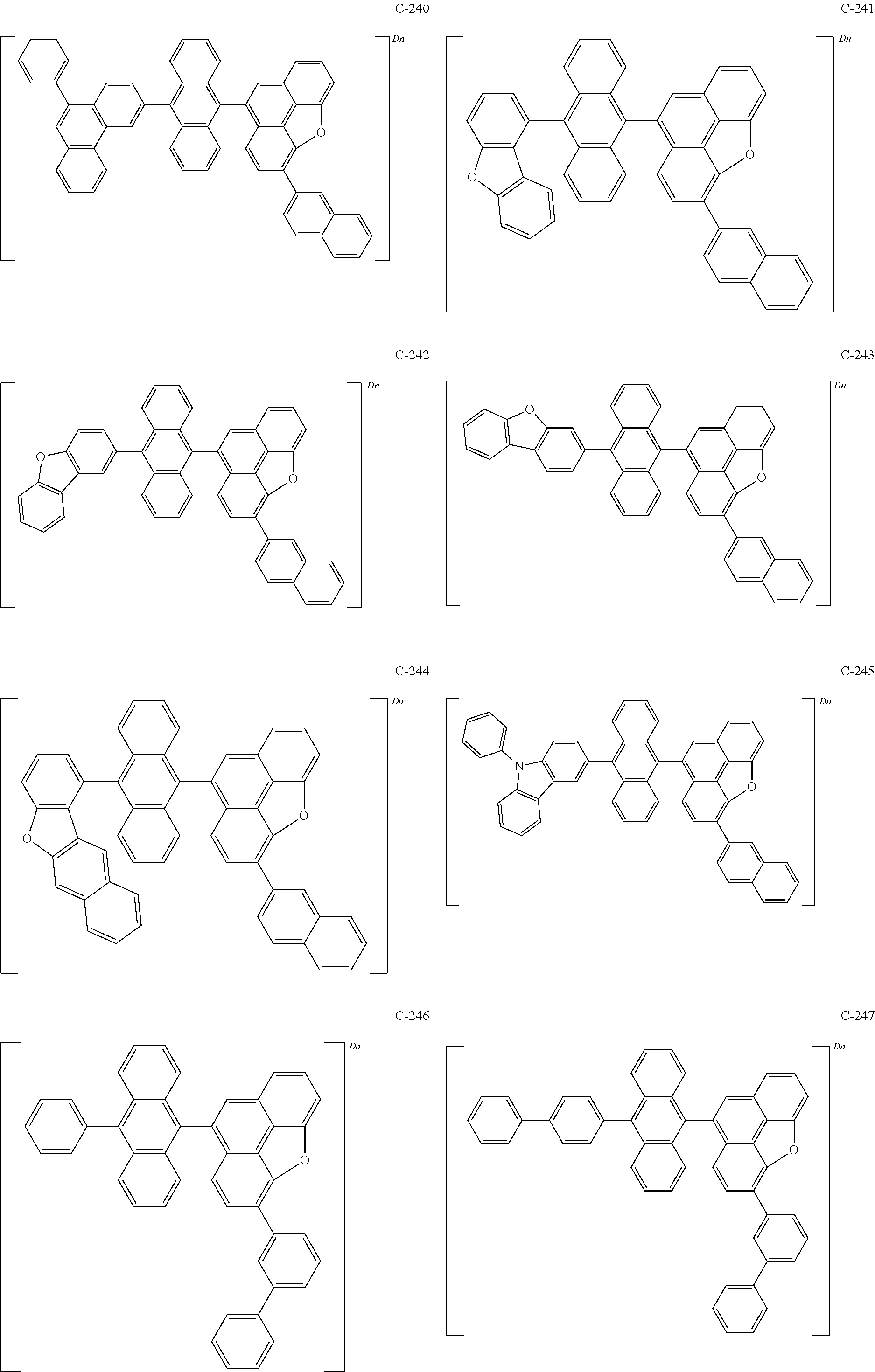

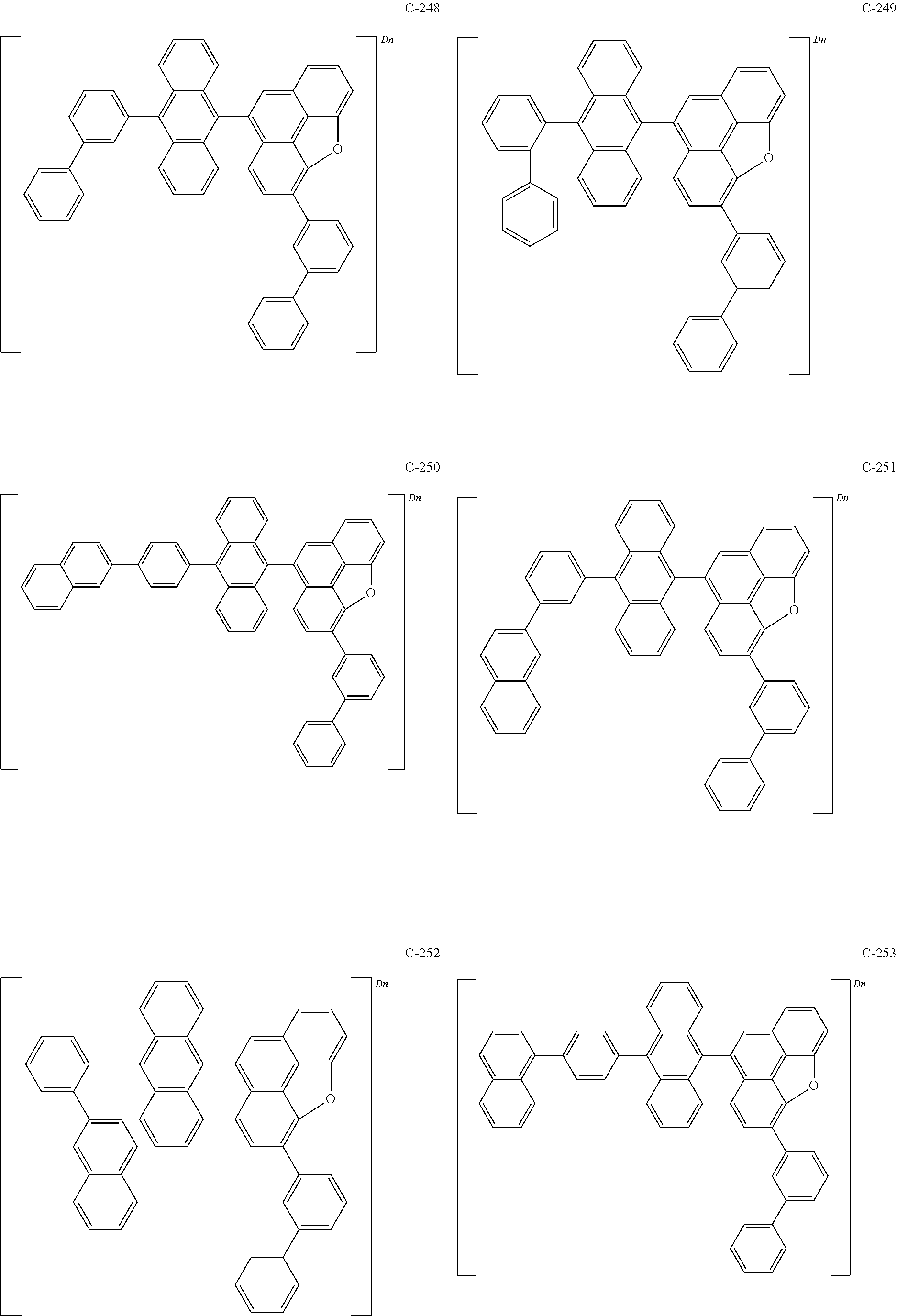

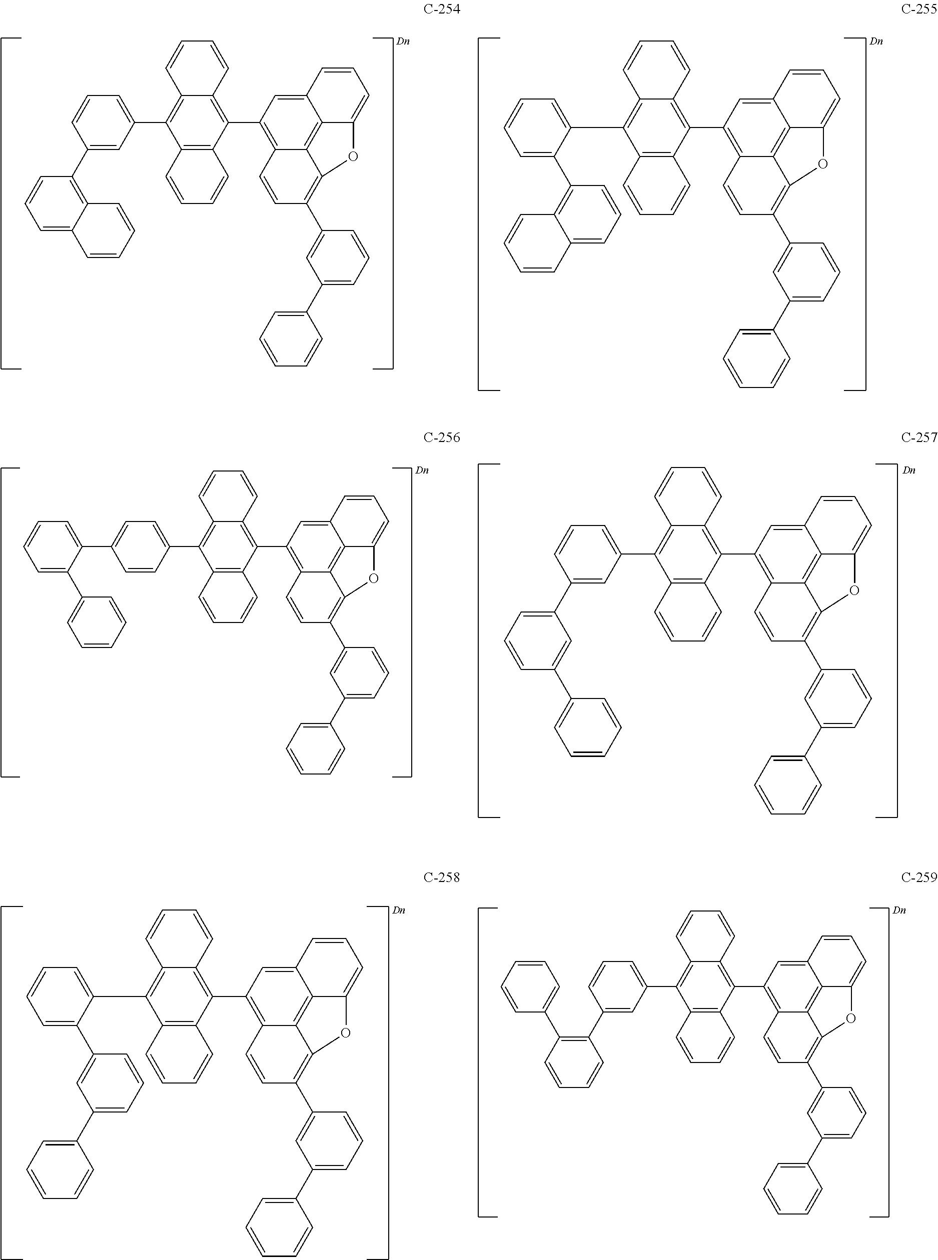

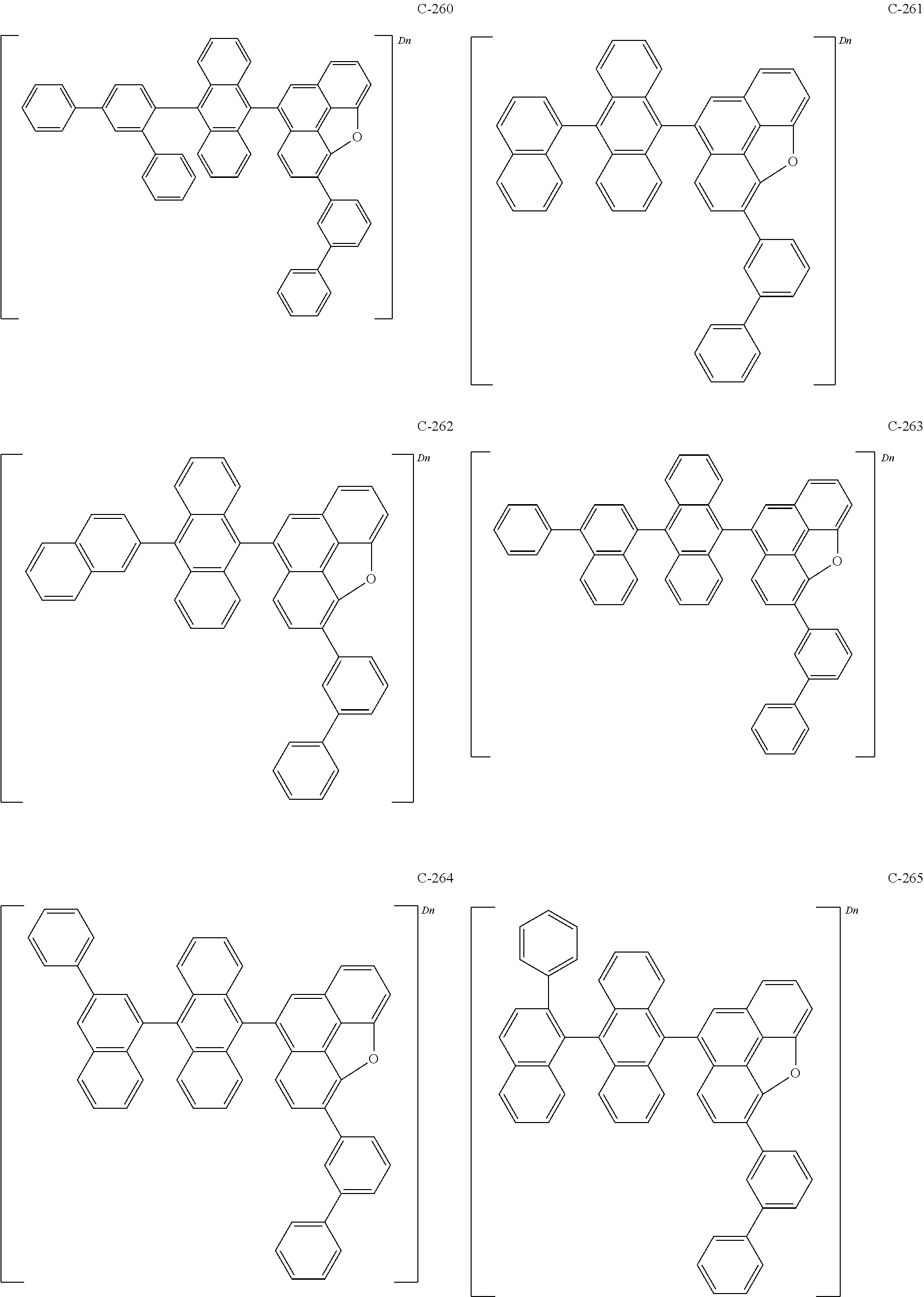

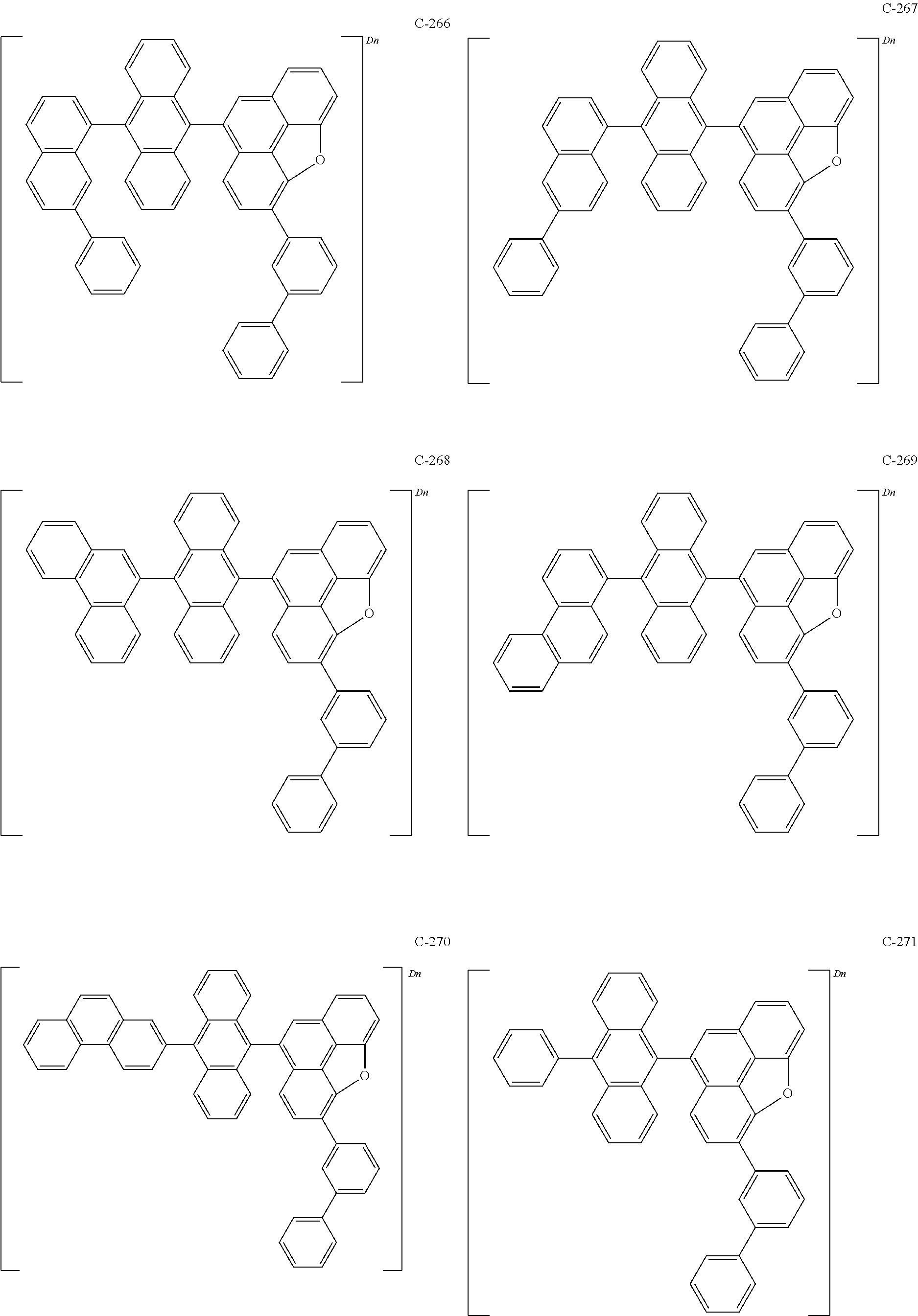

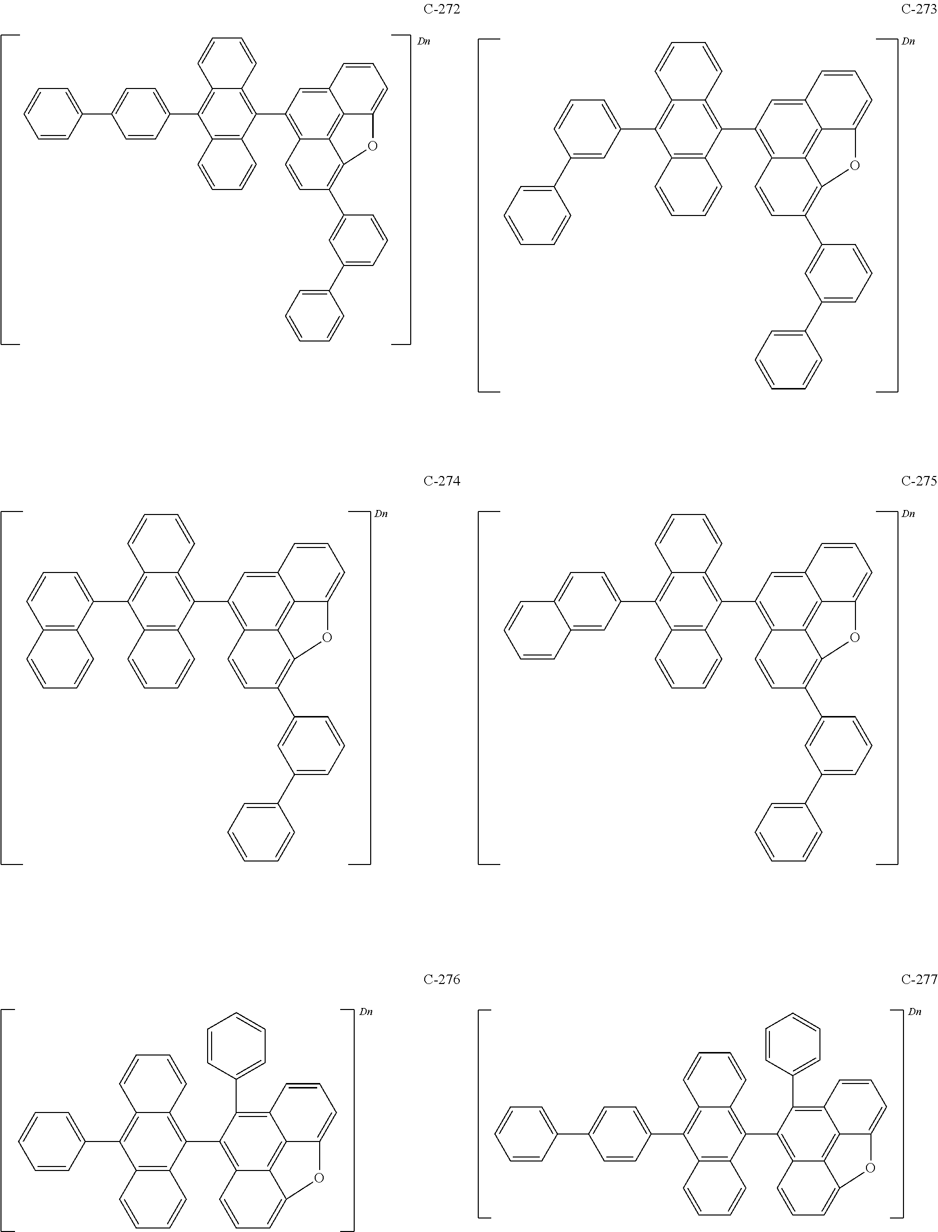

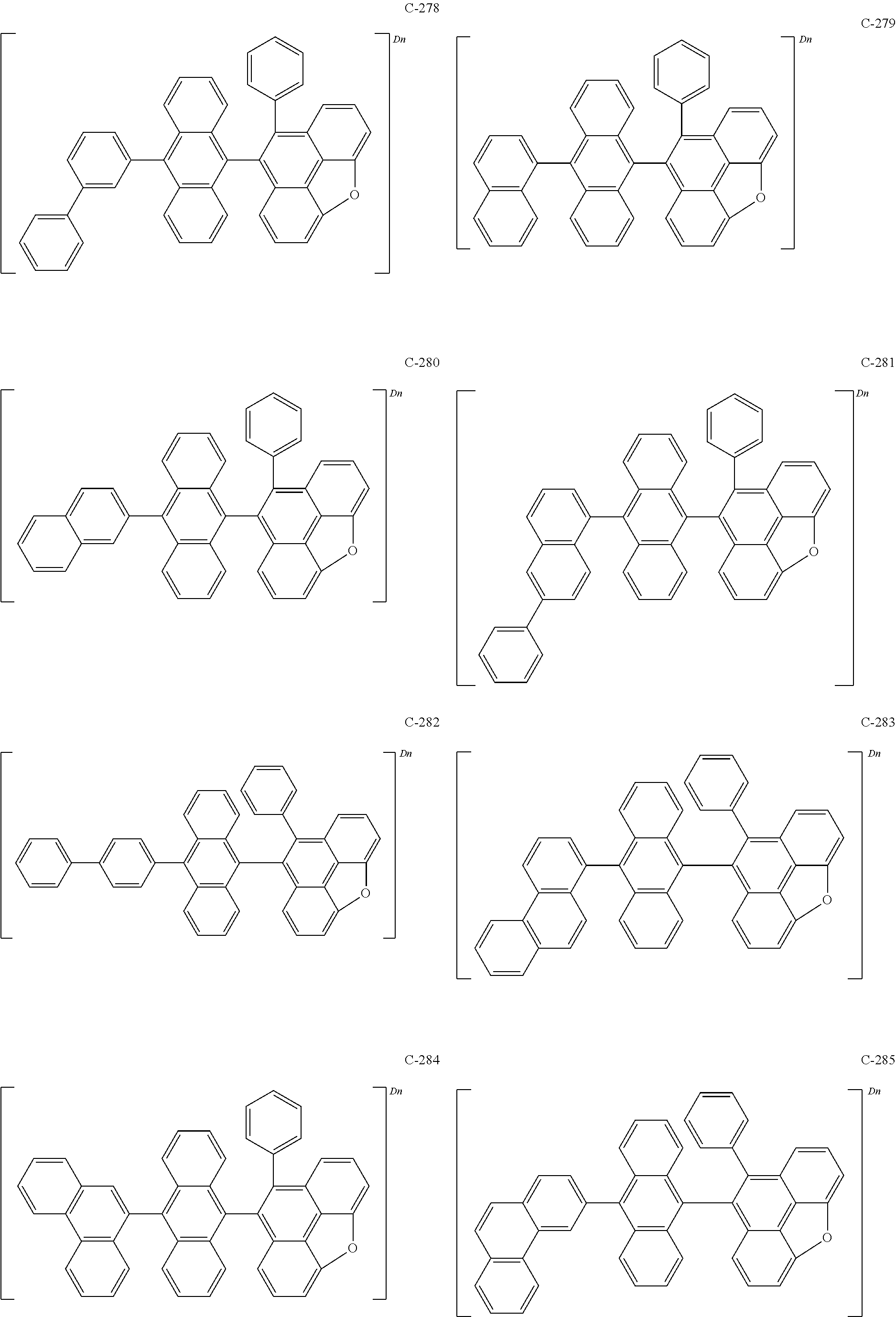

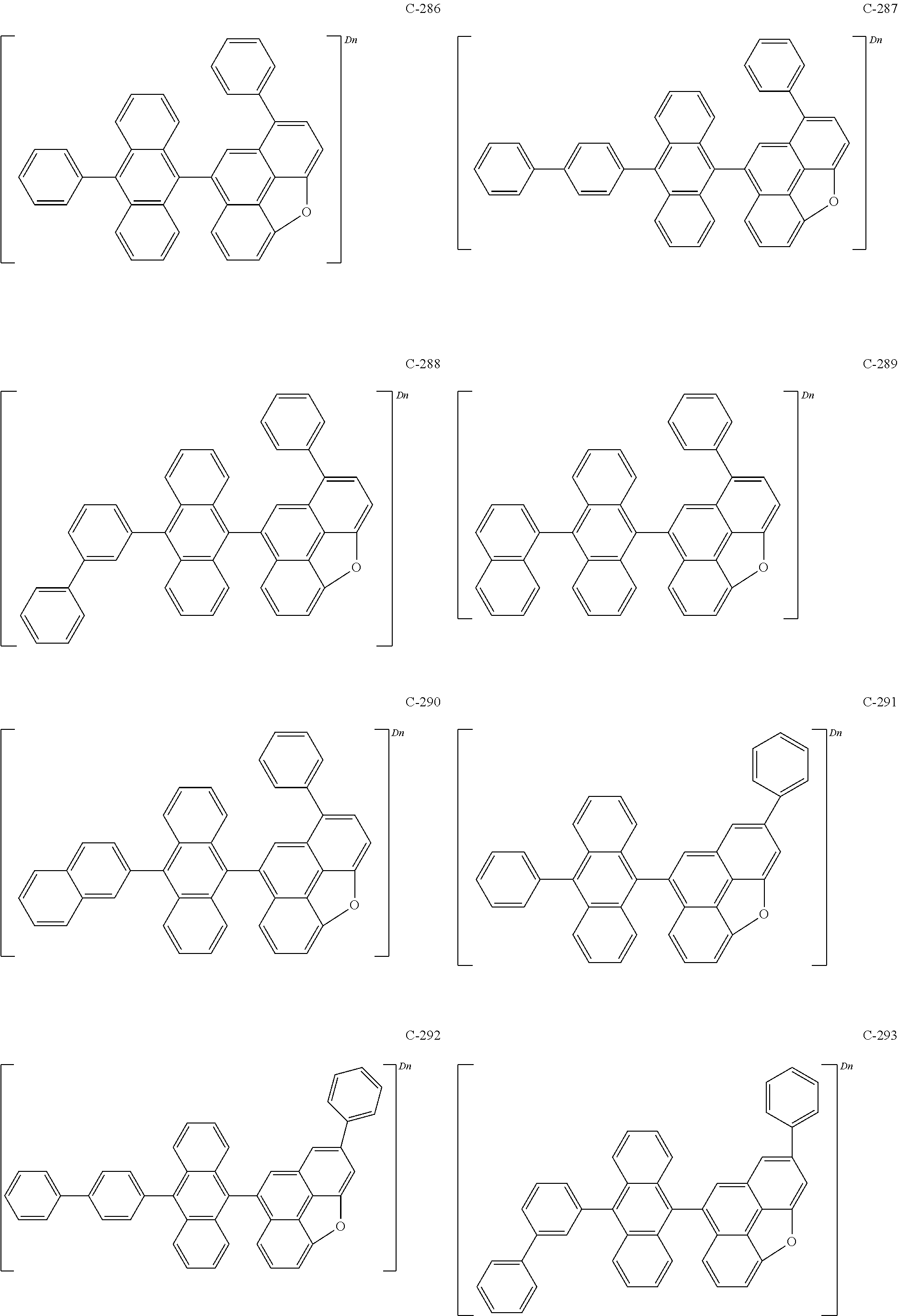

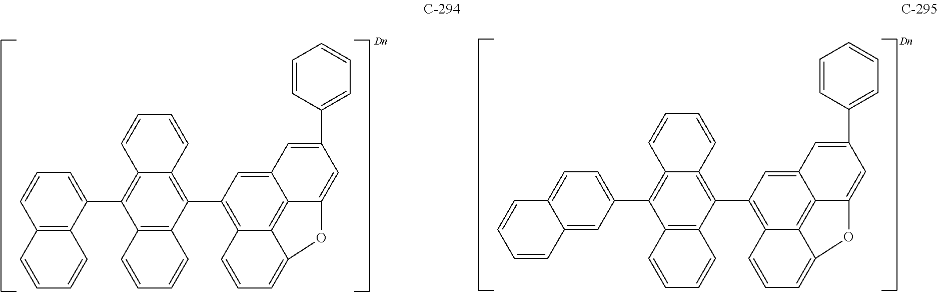

Dn of compounds C-136 to C-295 means that n hydrogens is replaced by deuteriums, and n is between 1 and the maximum number of hydrogens in each compound.

Substituents of the substituted alkyl, the substituted aryl, the substituted arylene, the substituted heteroaryl, and the substituted heteroarylene included in formulas 1 and 2 may each independently be selected from the group consisting of deuterium; halogen; cyano; carboxyl; nitro; hydroxy; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl substituted or unsubstituted with at least one of deuterium and (C6-C30)aryl; (C6-C30)aryl substituted or unsubstituted with at least one of deuterium and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C2-C30)alkenylamino; mono- or di-(C6-C30)arylamino; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphine; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

The compounds represented by formula 3 may be described in more detail as follows.

In formula 3, Ar₄ may be a substituted or unsubstituted (C6-C30)aryl, or a substituted or unsubstituted (3- to 30-membered)heteroaryl, preferably a substituted or unsubstituted (C6-C25)aryl, or a substituted or unsubstituted (3- to 25-membered)heteroaryl, more preferably a substituted or unsubstituted (C6-C20)aryl, or a substituted or unsubstituted (3- to 20-membered)heteroaryl. In one embodiment of the present disclosure, Ar₄ of formula 3 may be a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted triphenylenyl, a substituted or unsubstituted fluorenyl, a substituted or unsubstituted dibenzofluorenyl, a substituted or unsubstituted dibenzofuranyl, a substituted or unsubstituted dibenzothiophenyl, a substituted or unsubstituted carbazolyl, a substituted or unsubstituted dibenzoselenophenyl, or a substituted or unsubstituted naphthobenzofuranyl. For example, in formula 3, Ar₄ may be a substituted or unsubstituted phenyl, a substituted or unsubstituted biphenyl, a substituted or unsubstituted p-terphenyl, a substituted or unsubstituted m-terphenyl, a substituted or unsubstituted o-terphenyl, a substituted or unsubstituted naphthyl, a substituted or unsubstituted phenylnaphthyl, a substituted or unsubstituted phenanthrenyl, a substituted or unsubstituted phenylphenanthrenyl, a substituted or unsubstituted naphthylphenanthrenyl, a substituted or unsubstituted dibenzofuranyl, a substituted or unsubstituted naphthobenzofuranyl, or a substituted or unsubstituted carbazolyl, and the hydrogen in each substituent may be substituted with deuterium.

In formula 3, L₄ may be a single bond, a substituted or unsubstituted (C6-C30)arylene, or a substituted or unsubstituted (3- to 30-membered)heteroarylene, preferably a single bond, a substituted or unsubstituted (C6-C25)arylene, or a substituted or unsubstituted (3- to 25-membered)heteroarylene, more preferably a single bond, a substituted or unsubstituted (C6-C25)arylene, or a substituted or unsubstituted (3- to 25-membered)heteroarylene. For example, L₄ may be a single bond.

In formula 3, R₈ represents a substituted or unsubstituted (C6-C30)aryl, or a substituted or unsubstituted (3- to 30-membered)heteroaryl, preferably a substituted or unsubstituted (C6-C25)aryl, or a substituted or unsubstituted (3- to 25-membered)heteroaryl, more preferably a substituted or unsubstituted (C6-C25)aryl, or a substituted or unsubstituted (3- to 25-membered)heteroaryl. For example, in formula 3, R₈ may be a substituted or unsubstituted phenyl, a substituted or unsubstituted biphenyl, or a substituted or unsubstituted naphthyl, and the hydrogen in each substituent may be substituted with deuterium.

In formula 3, R₉ to R₁₂ may each independently be, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)heteroarylamino, or a substituted or unsubstituted (C6-C30)aryl(3- to 30-membered)heteroarylamino. For example, in formula 3, R₉ to R₁₂ may each independently be, hydrogen or deuterium.

In formula 3, g is an integer of 1 or 2, and may be, for example, 1 or 2.

In formula 3, h, i, and j are each independently an integer of 1 to 4, and may be, for example, 1, 2, 3, or 4.

According to one embodiment of the present disclosure, formula 3 may be represented by the following formula 3-1 or 3-2.

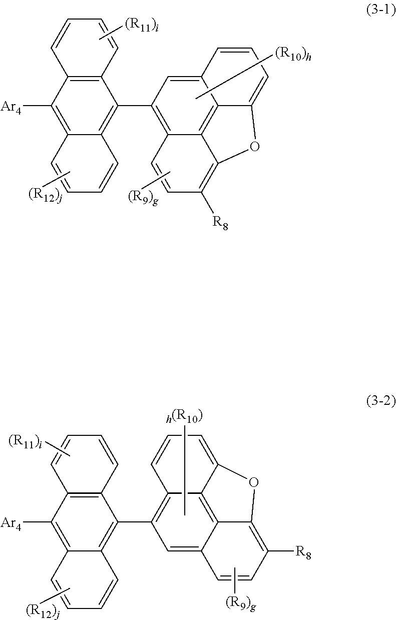

In formulas 3-1 and 3-2,

Ar₄, R₈ to R₁₂, and g to j are the same as defined in formula 3.

The compound represented by formula 3 may be selected from the following compounds, but are not limited thereto.

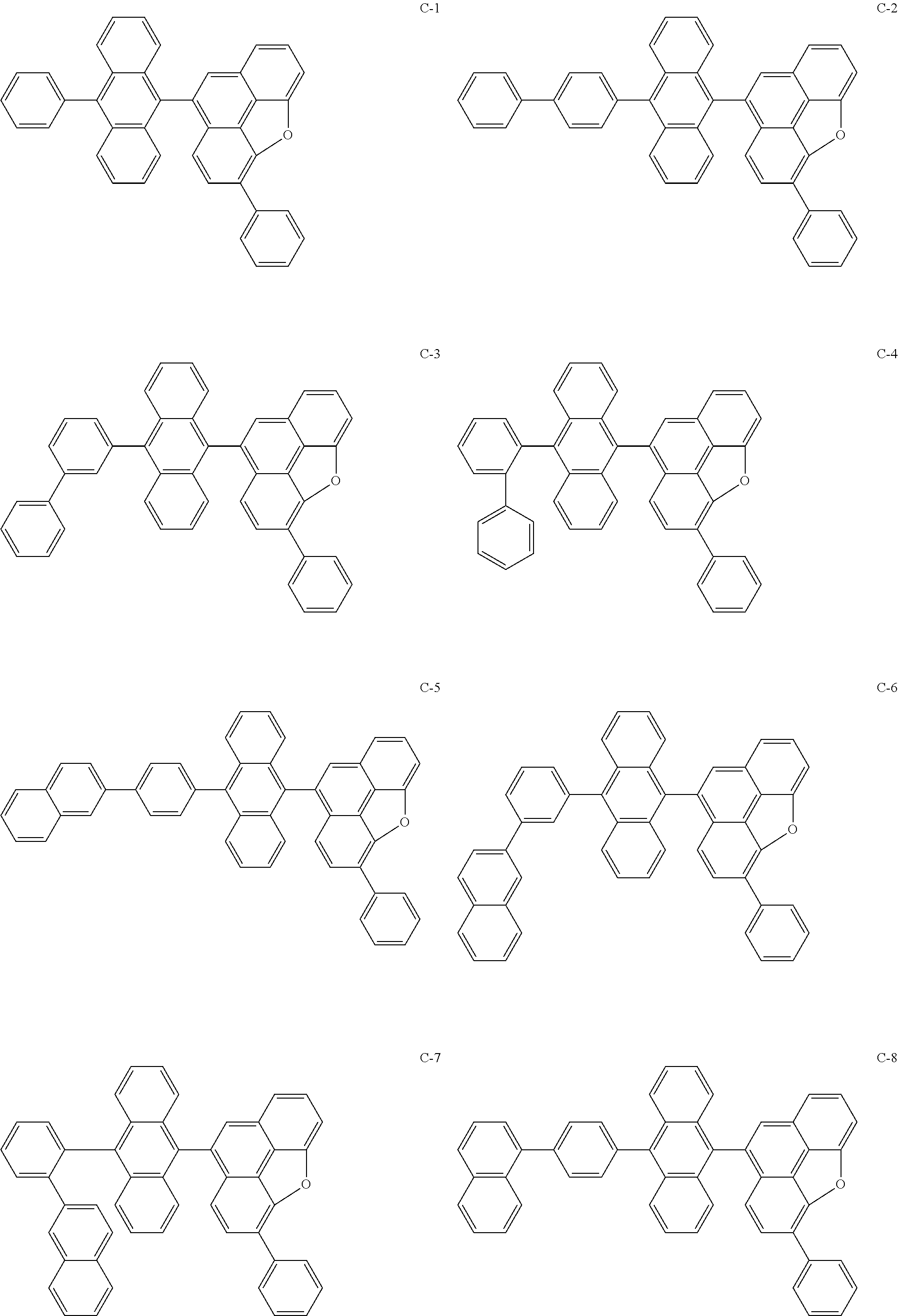

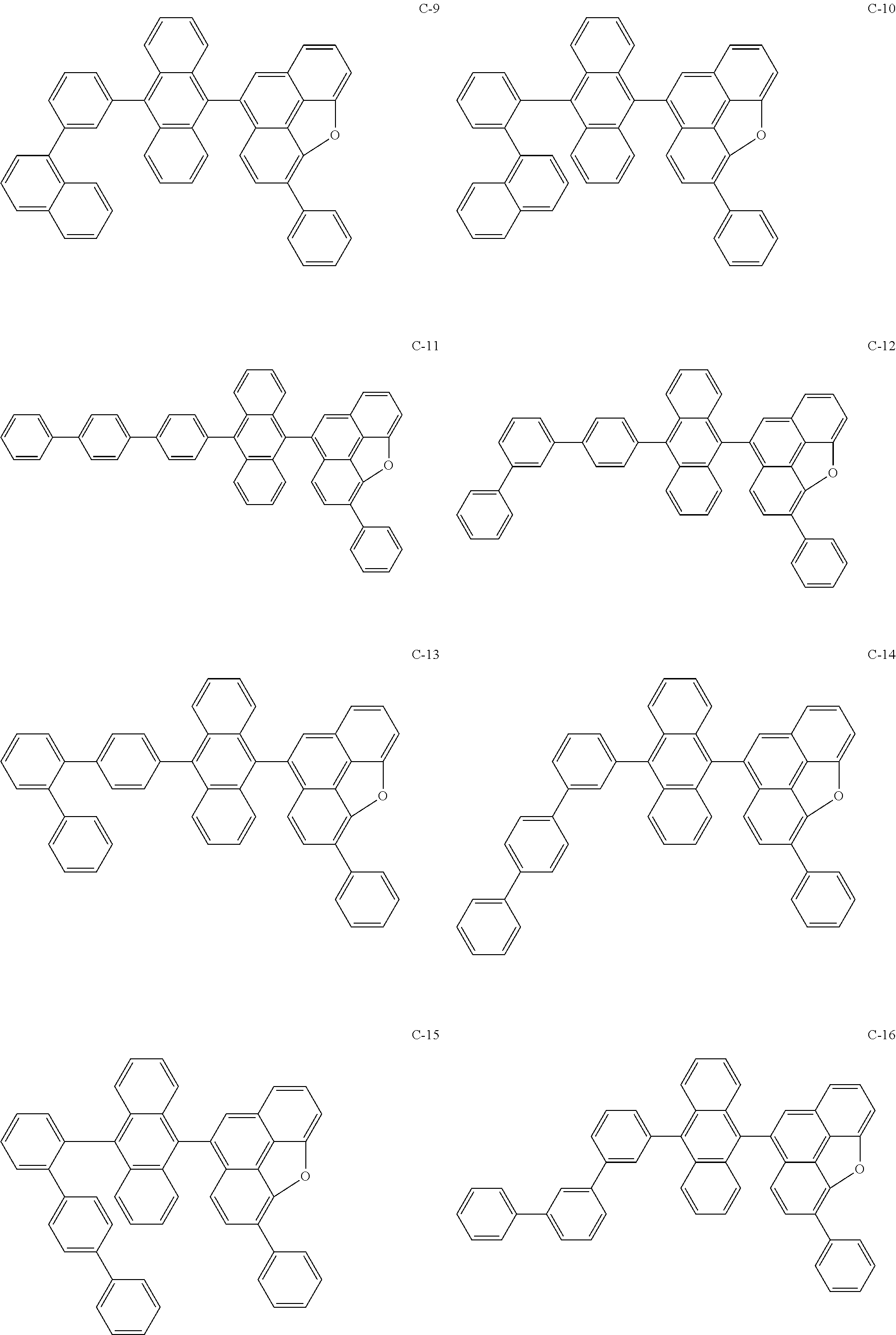

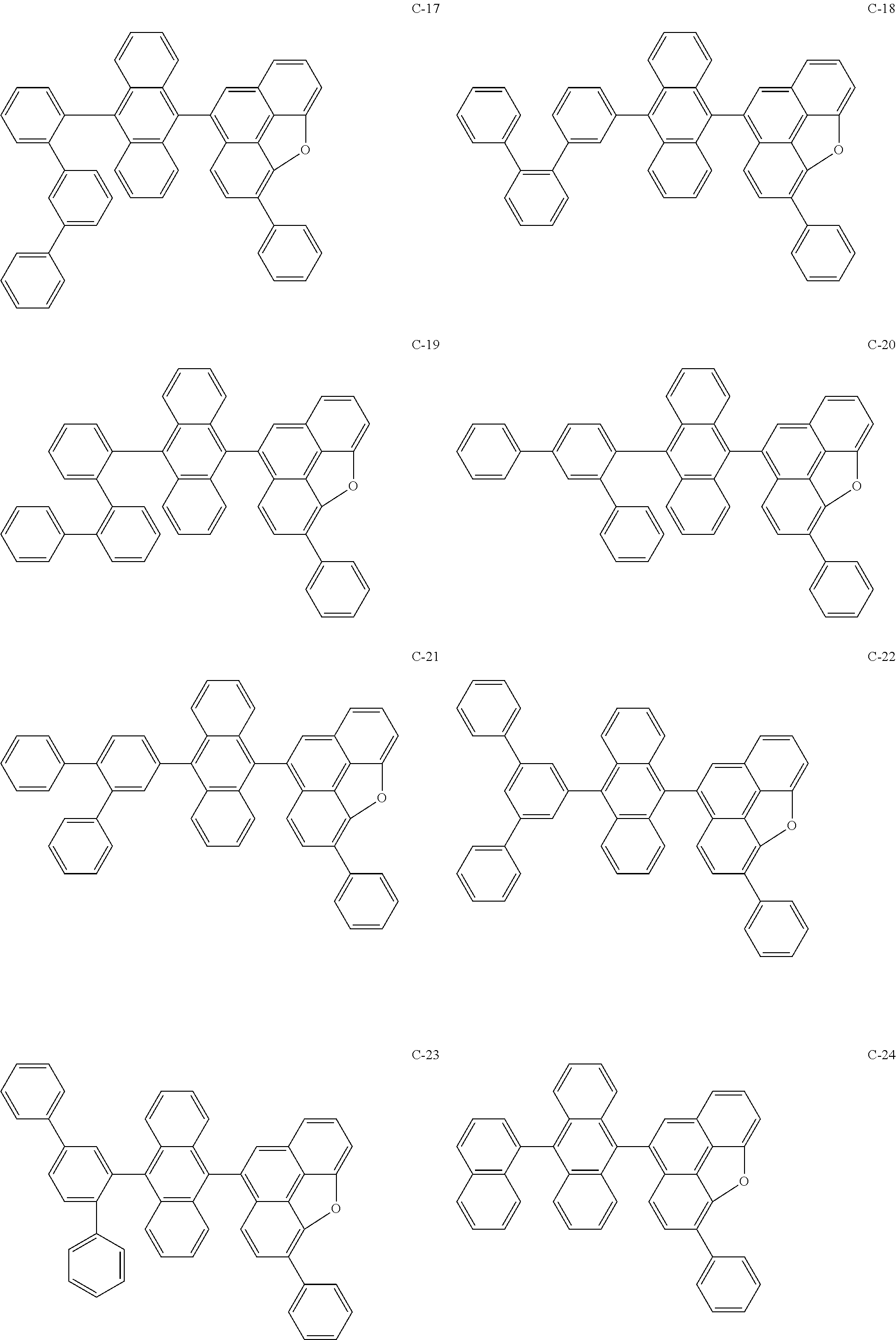

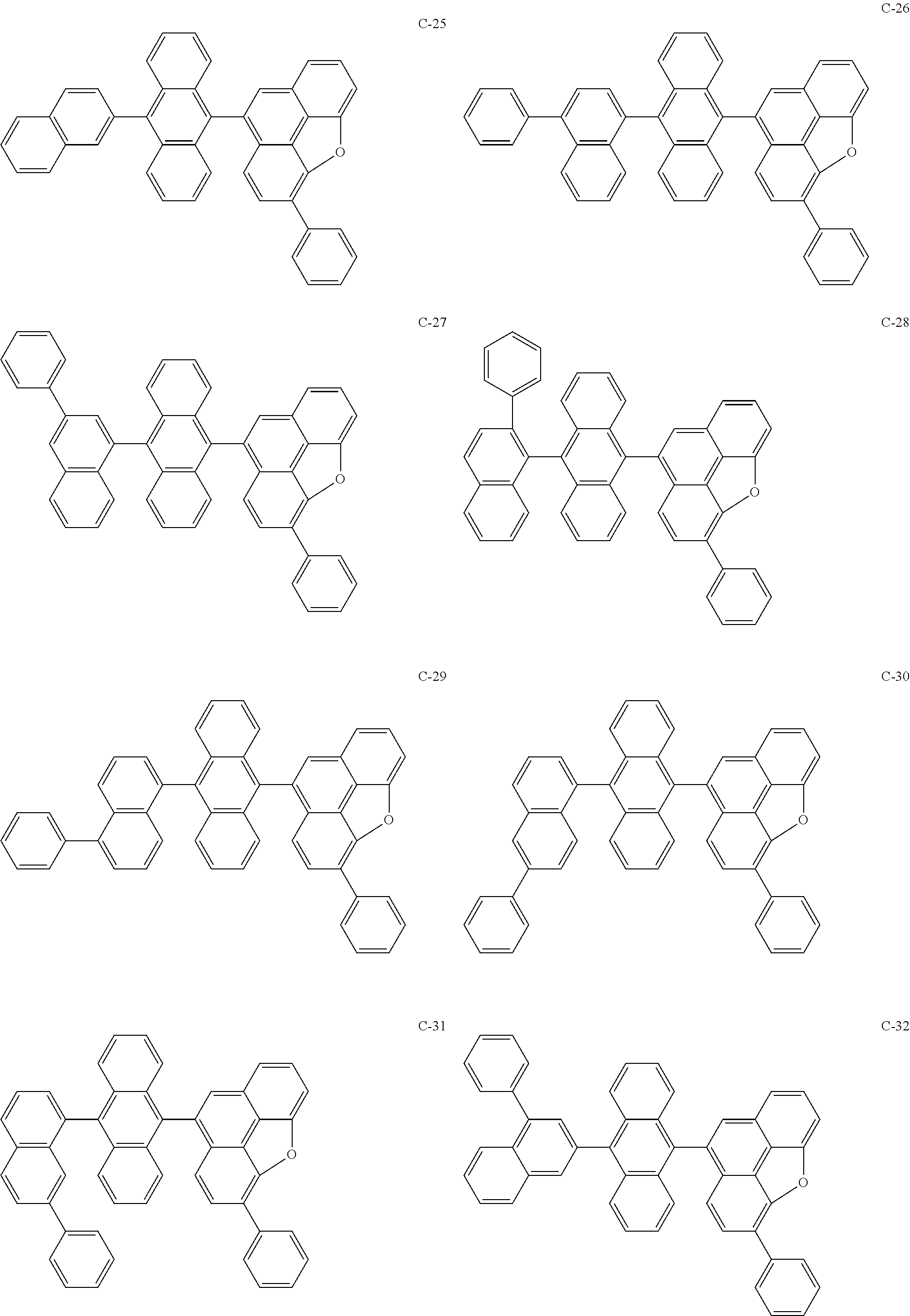

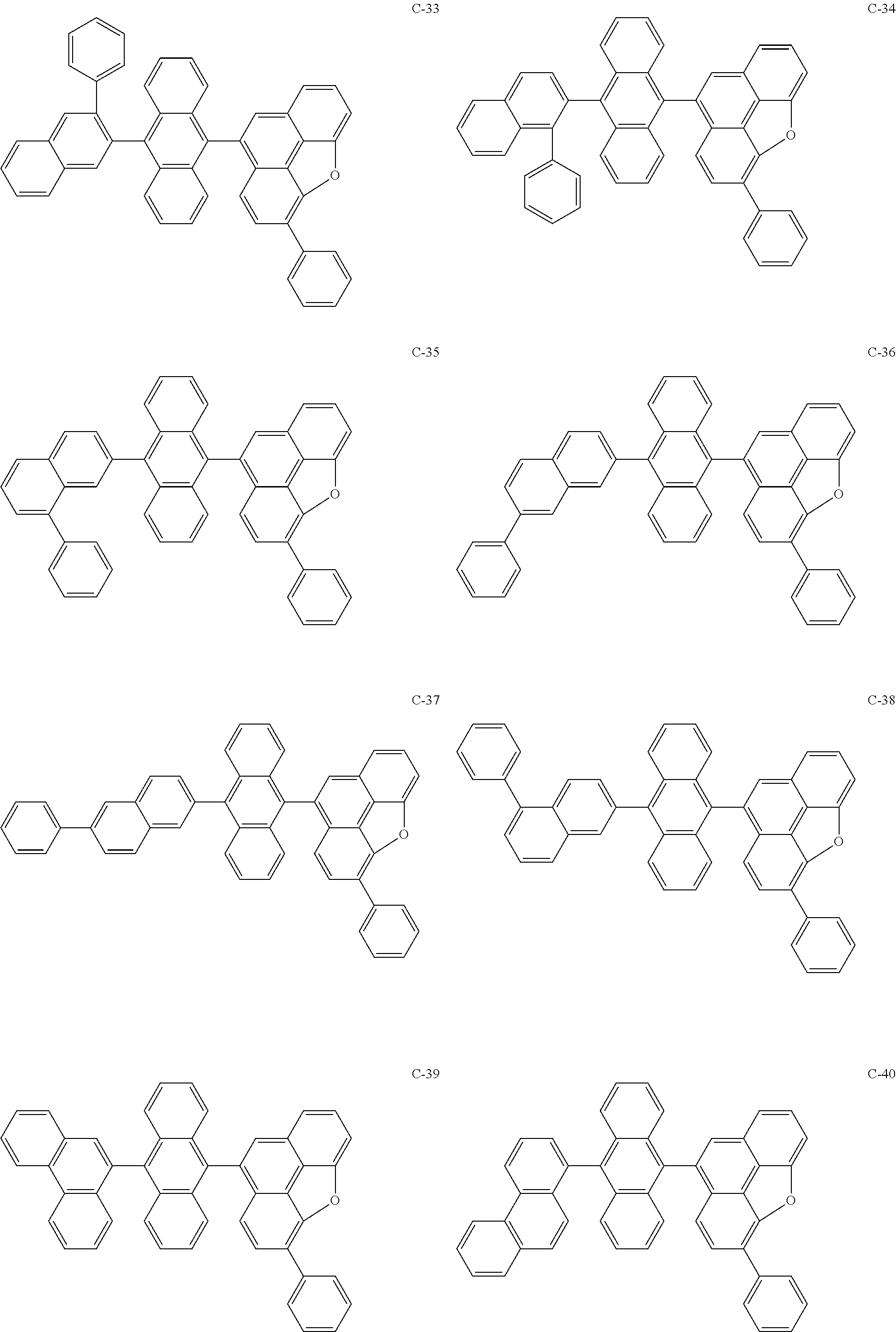

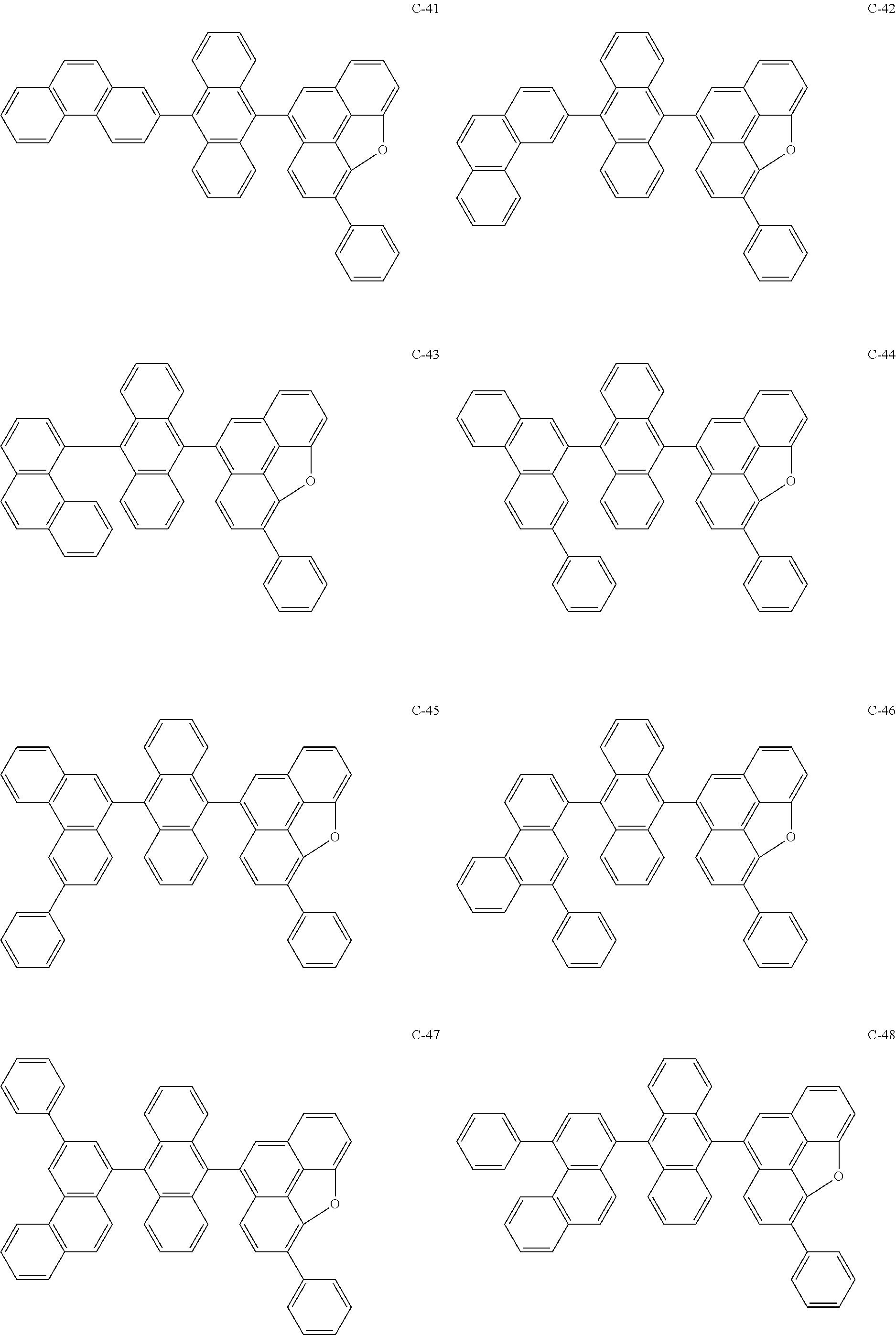

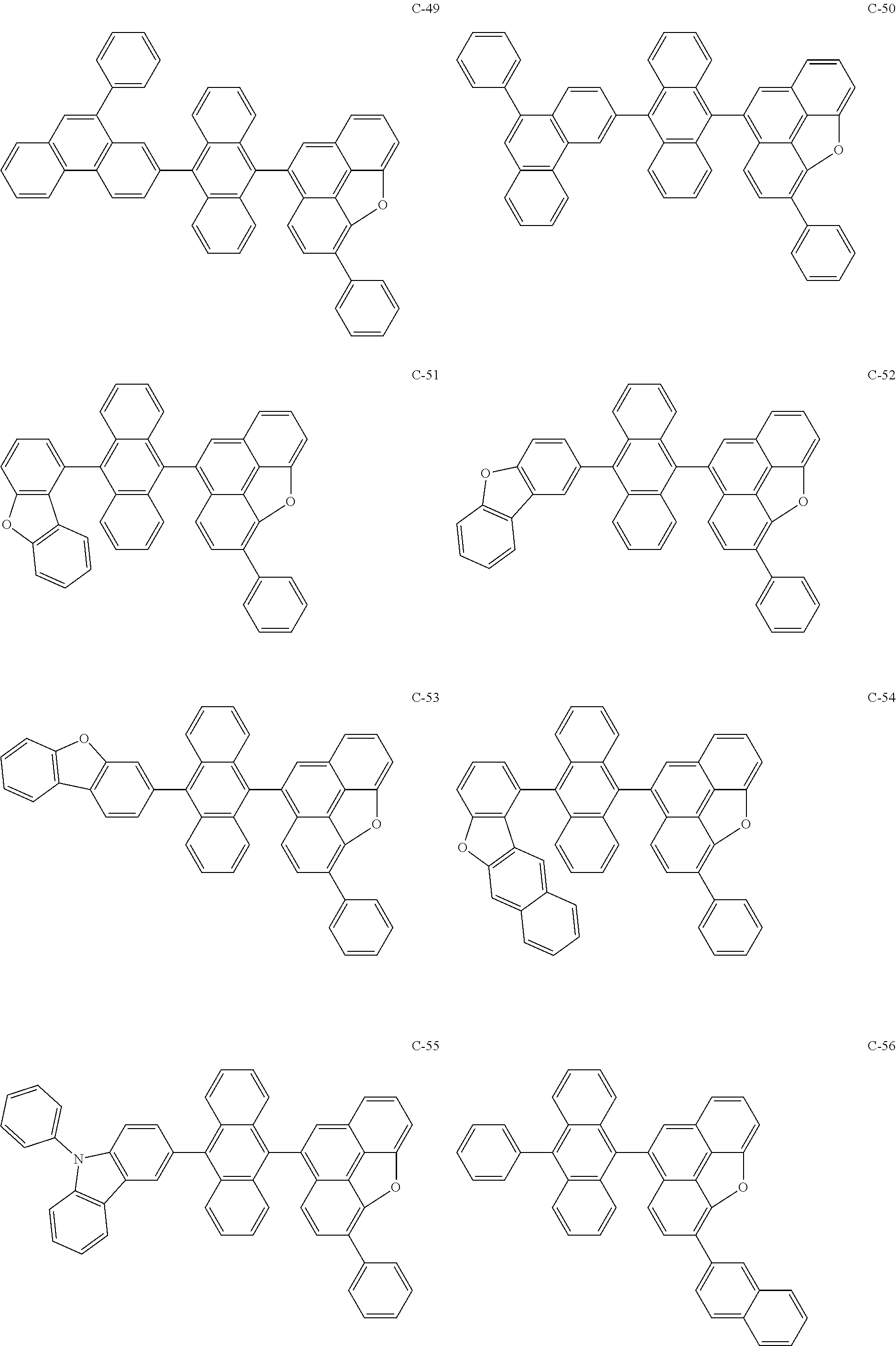

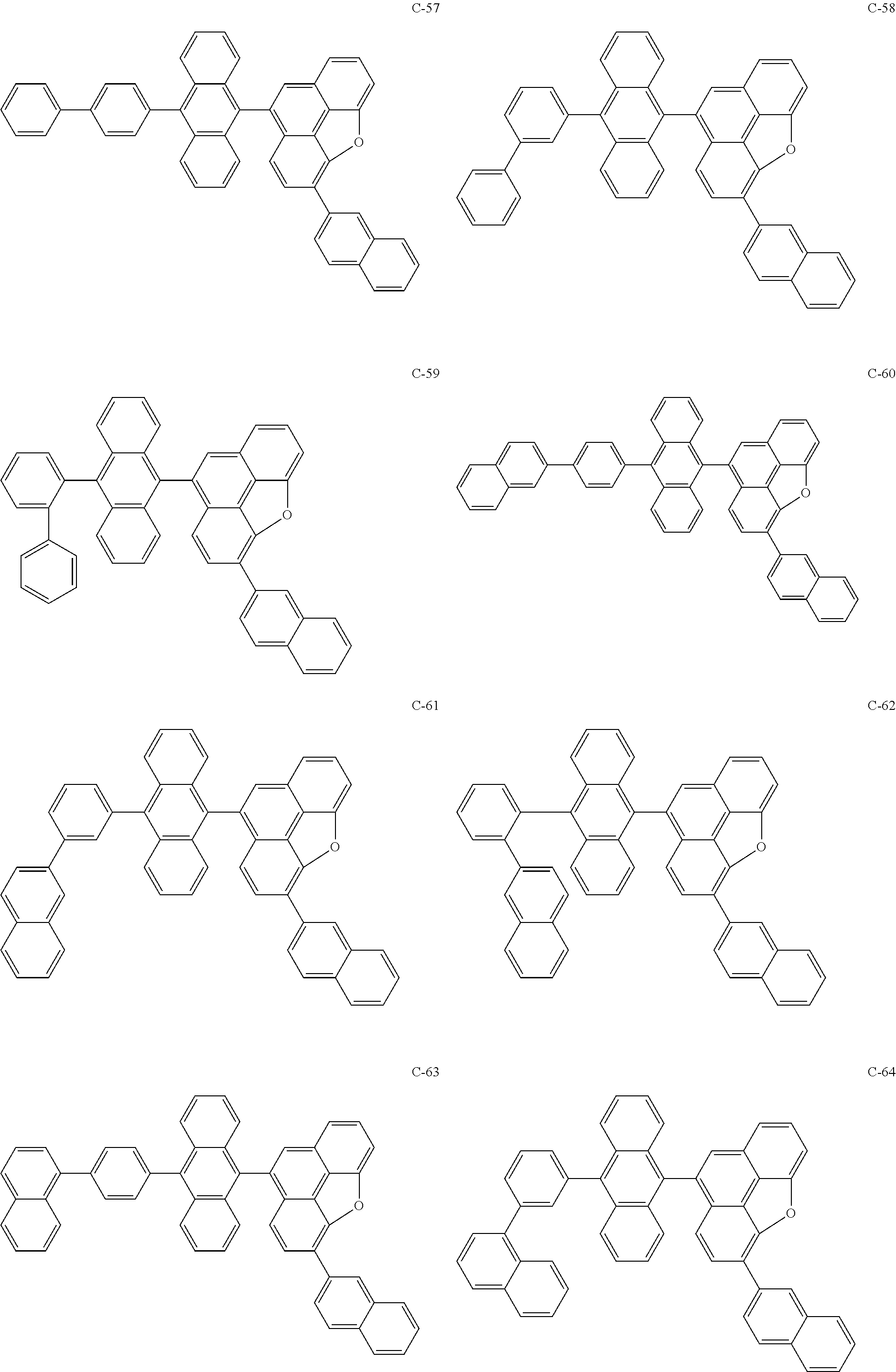

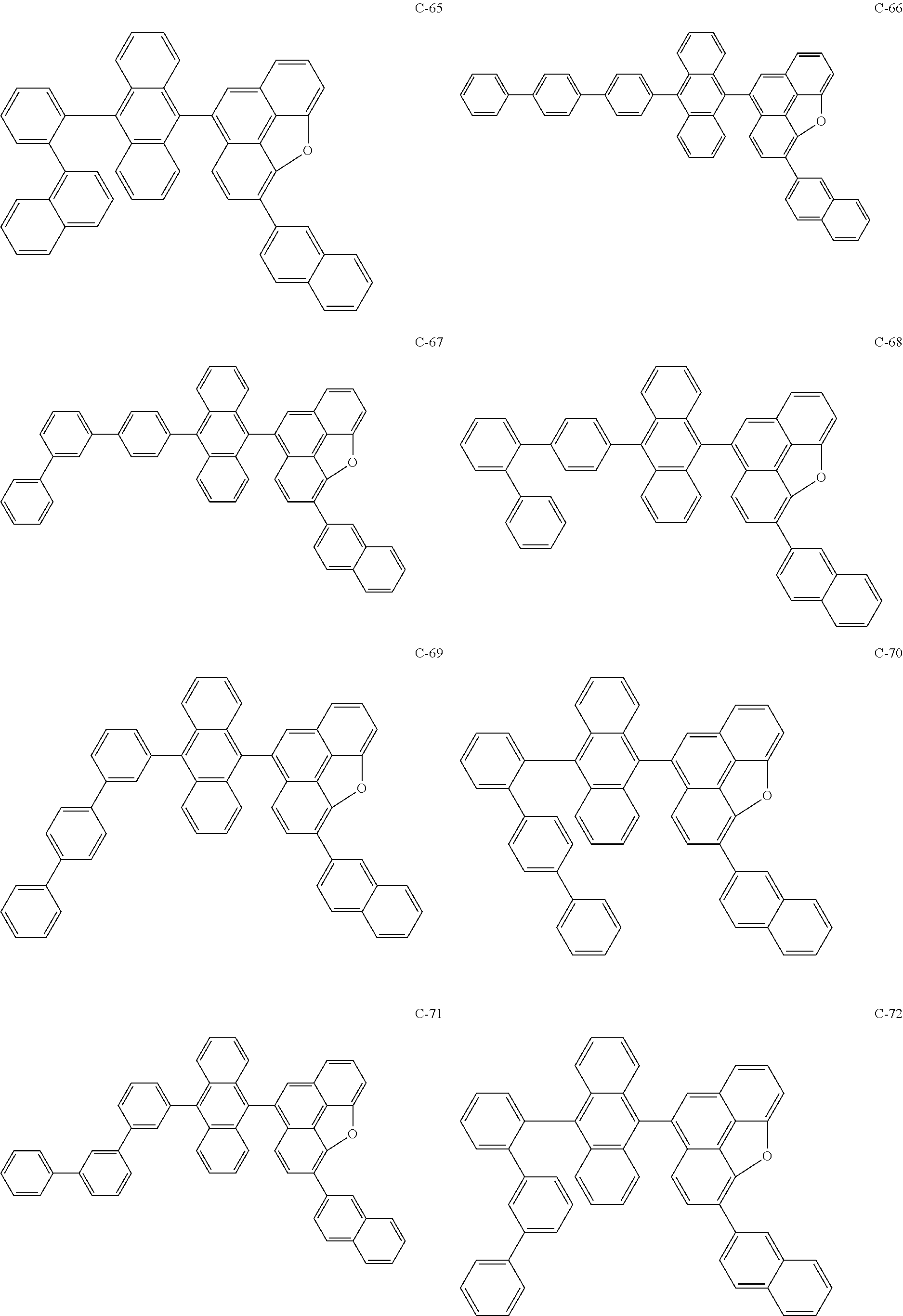

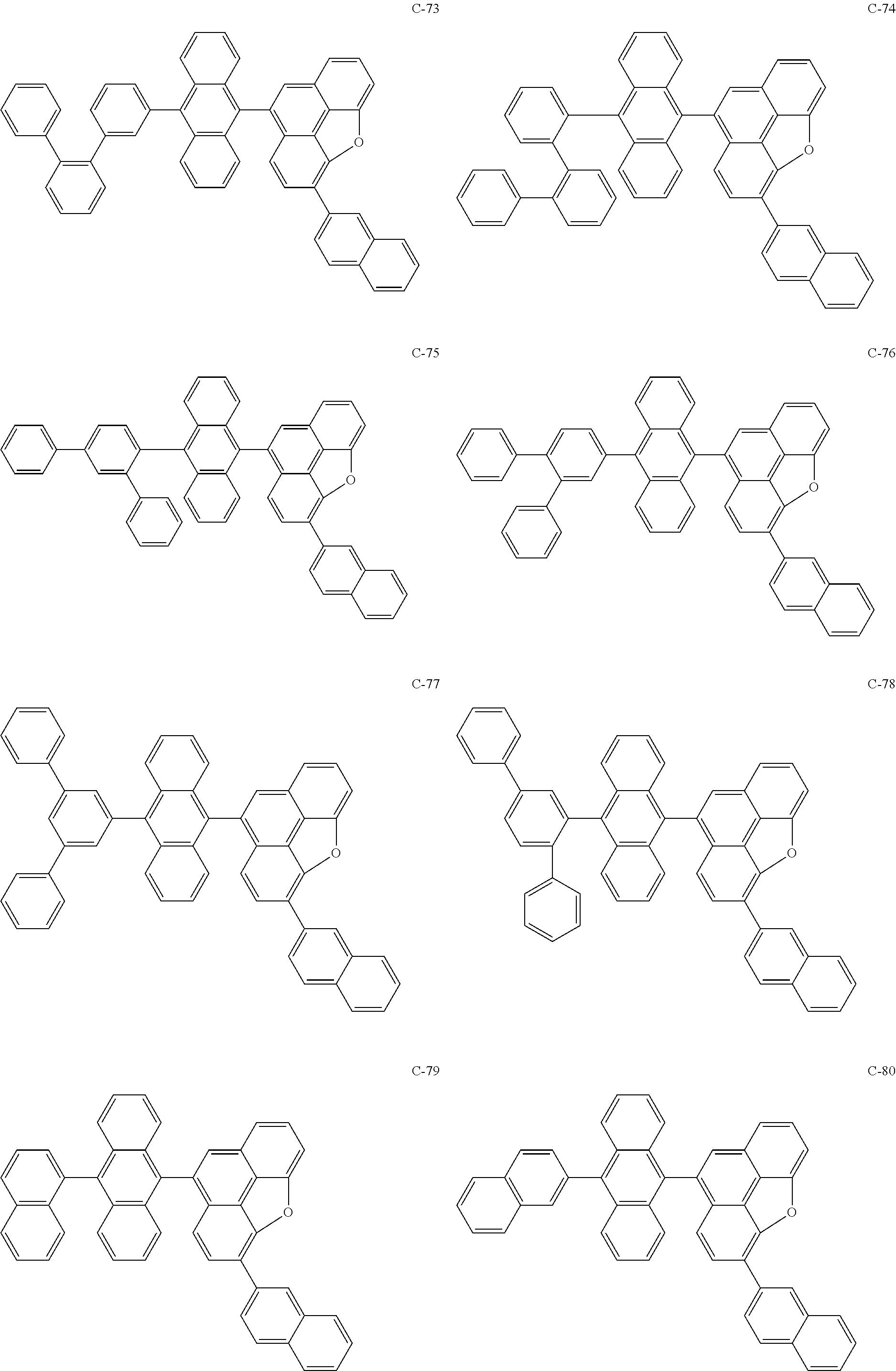

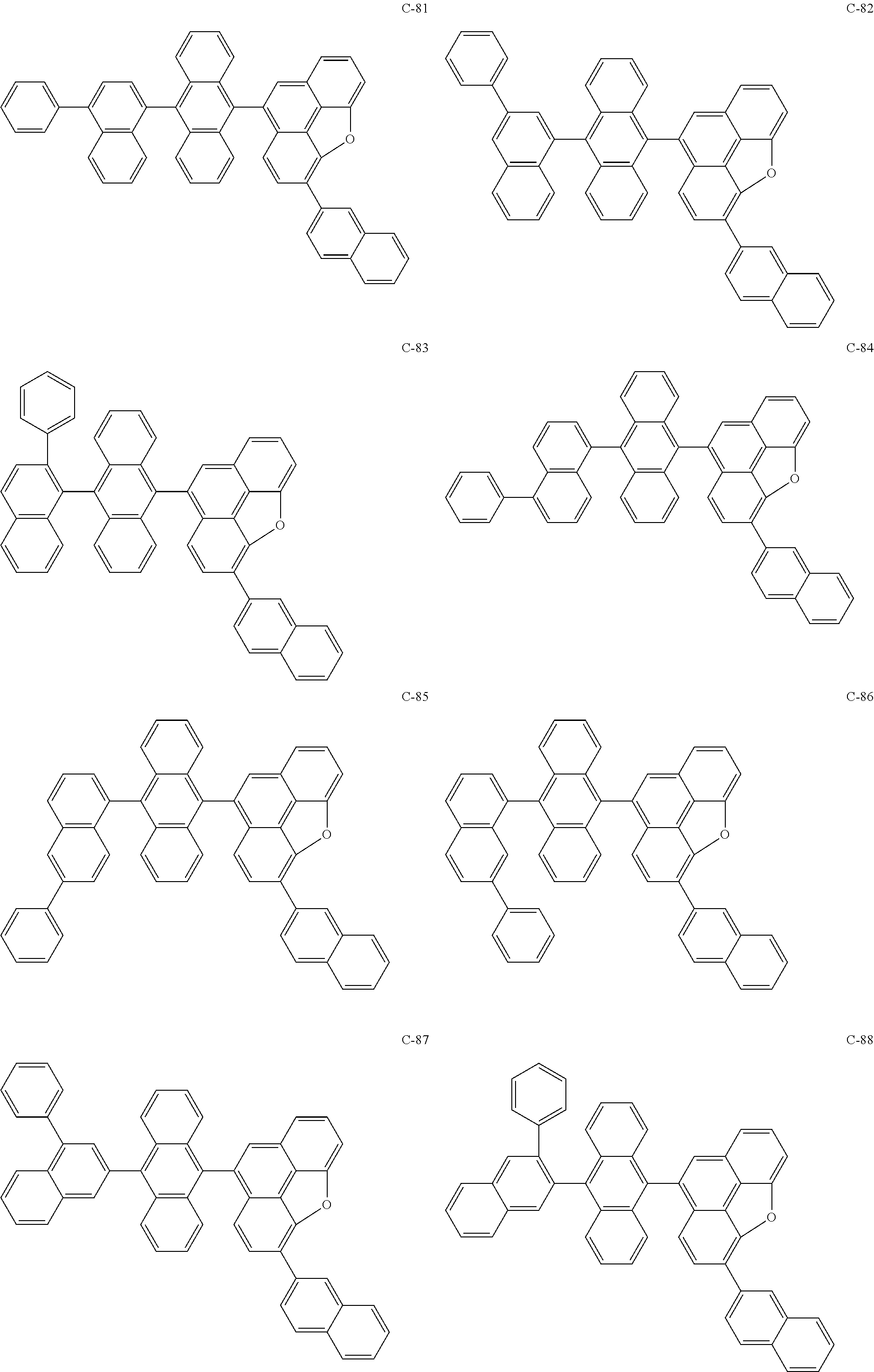

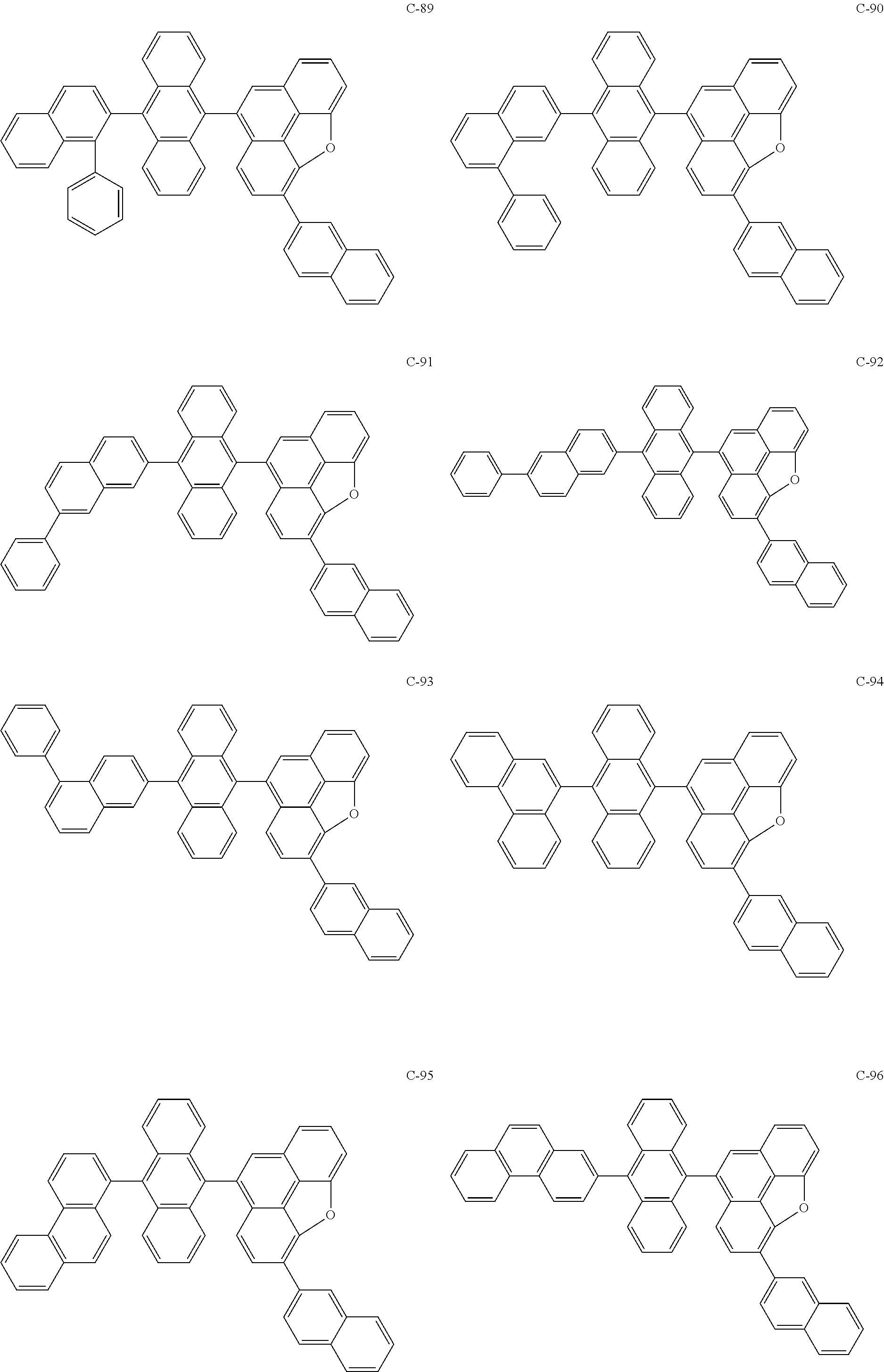

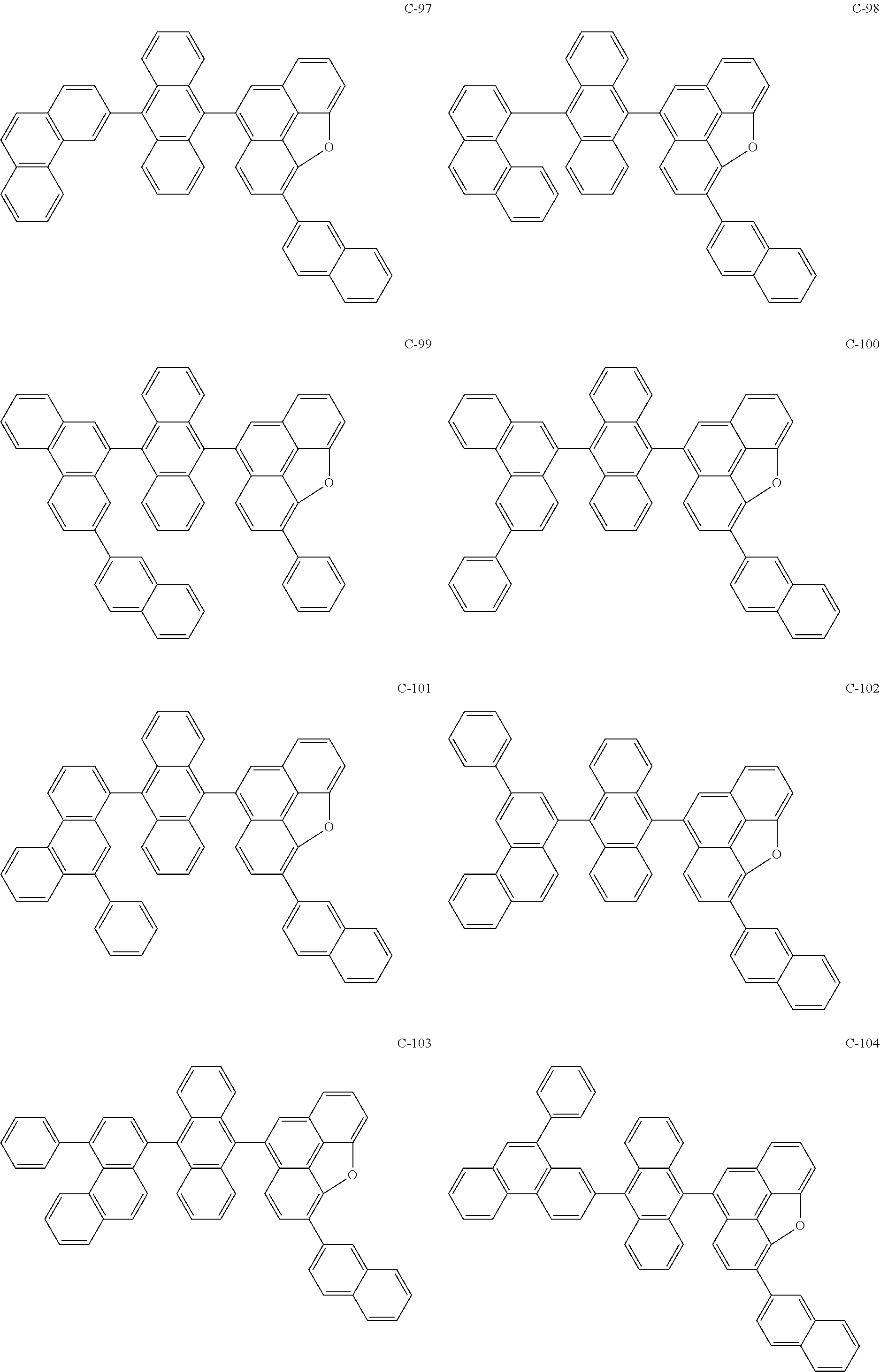

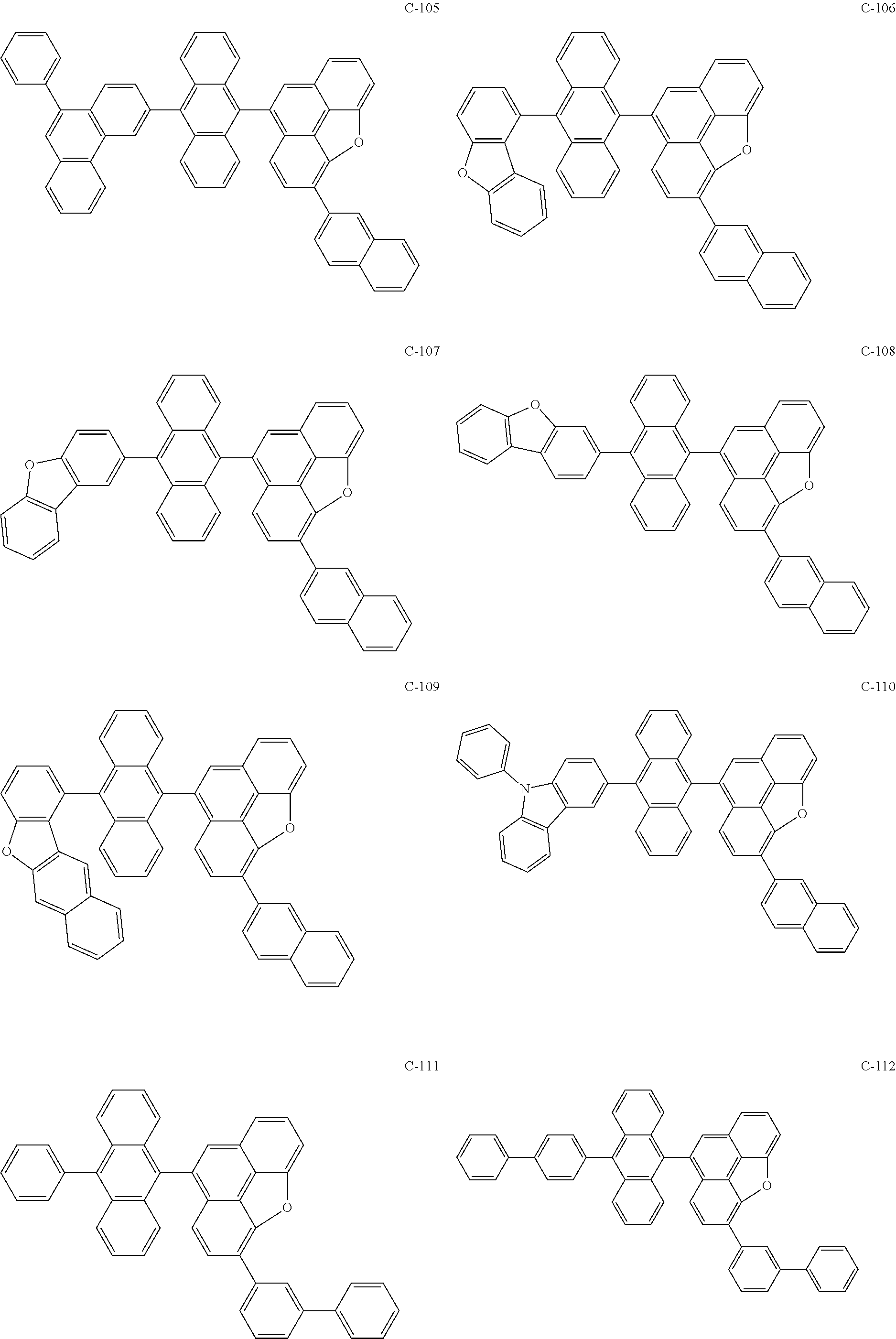

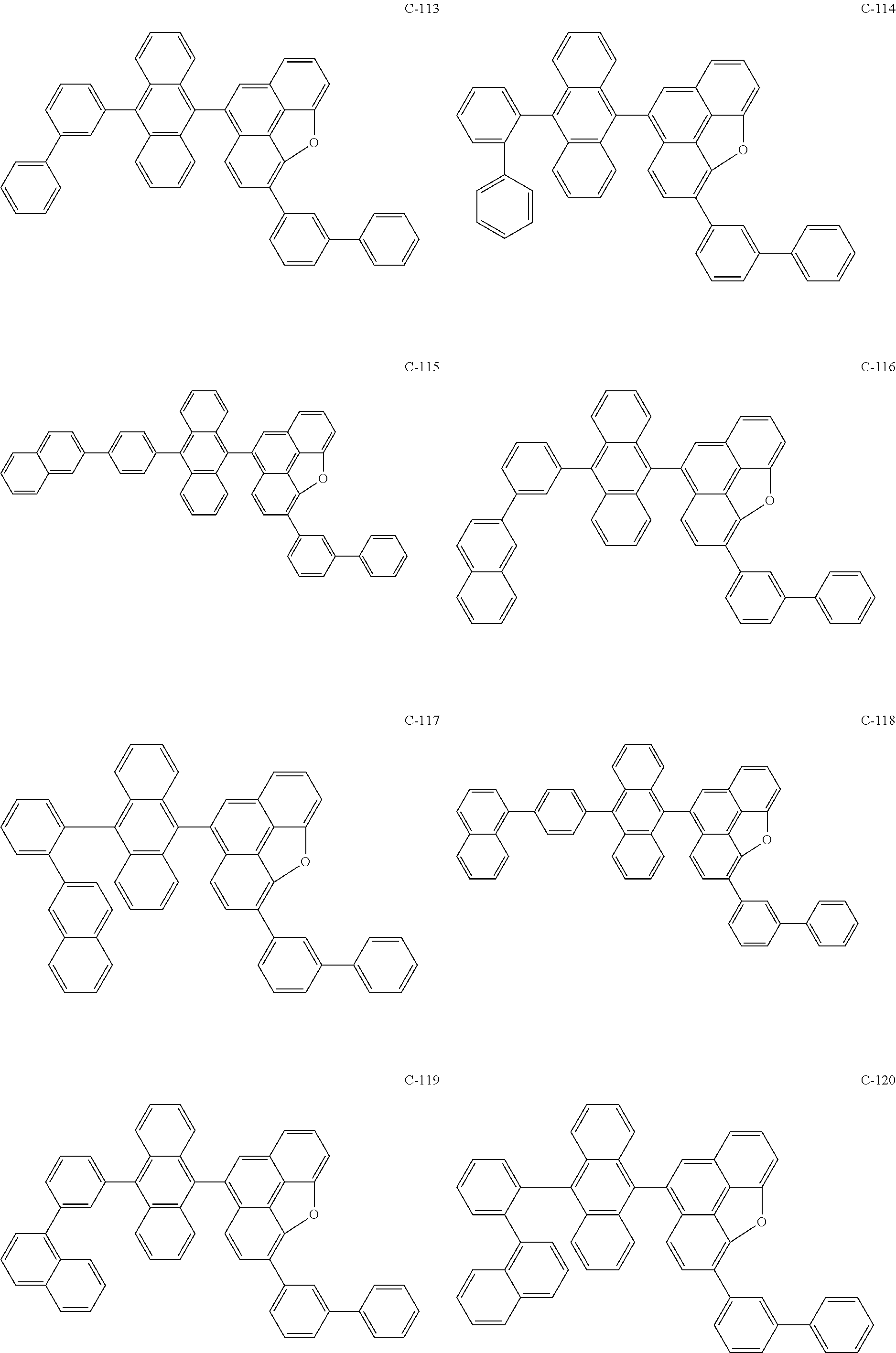

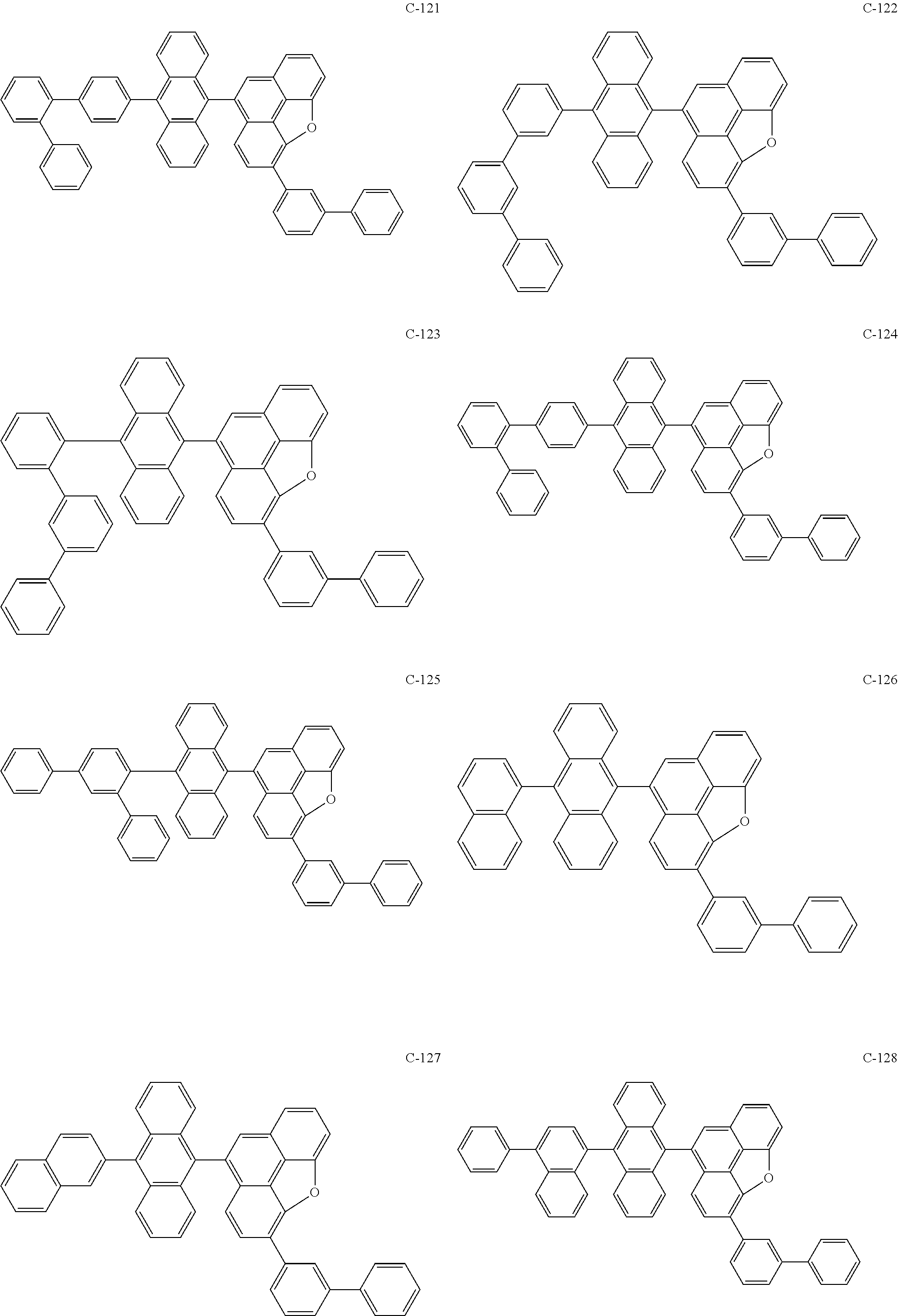

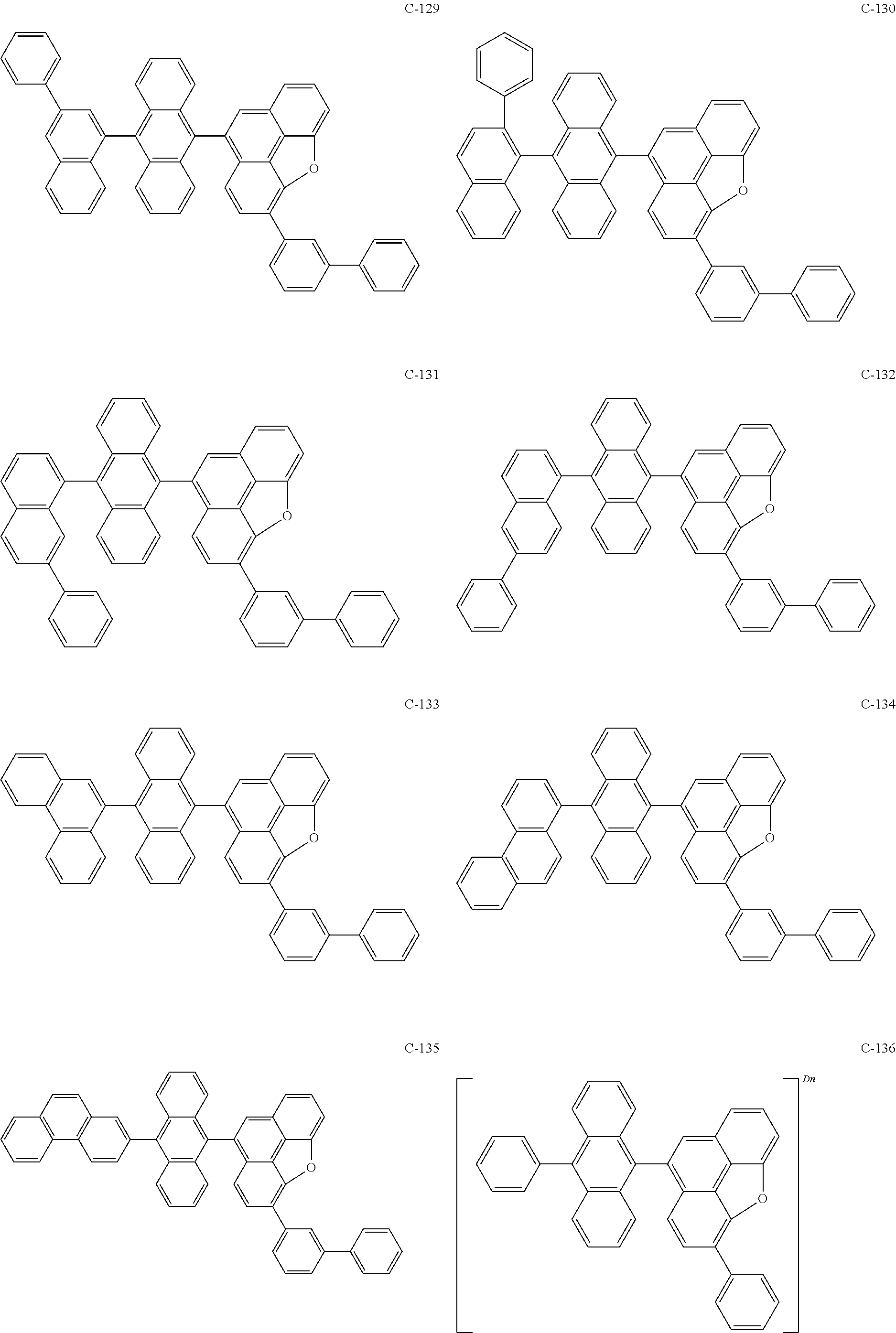

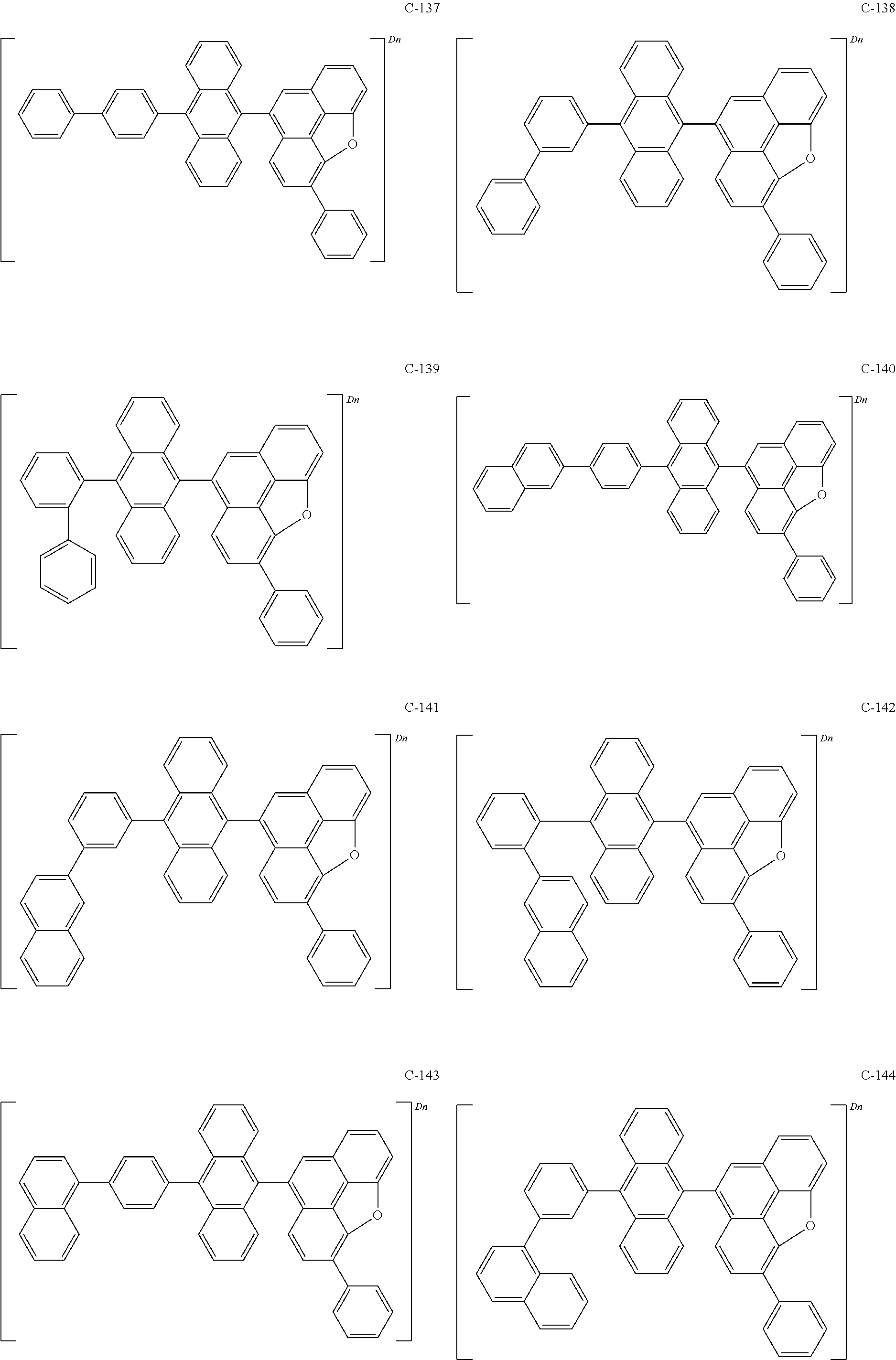

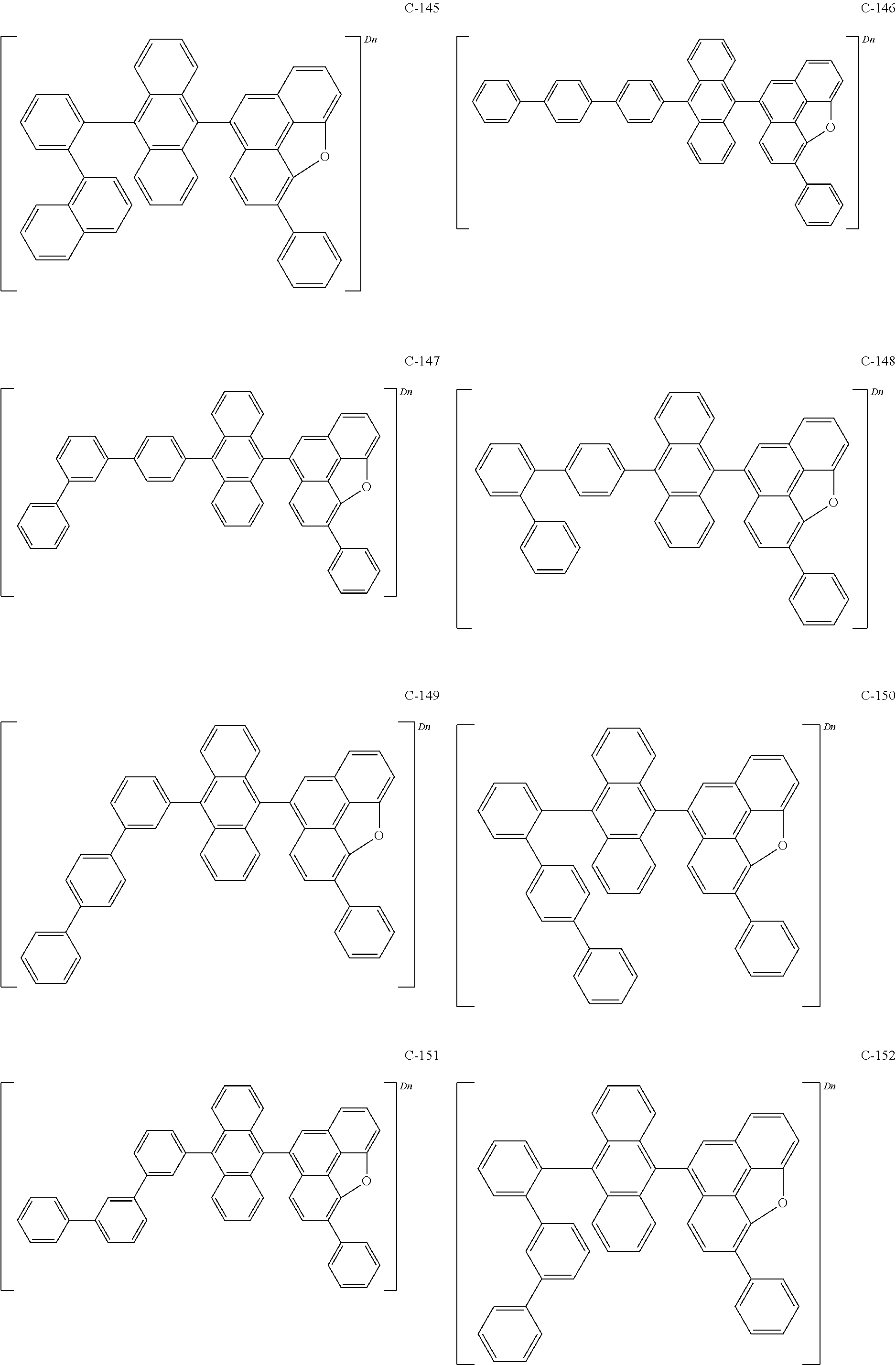

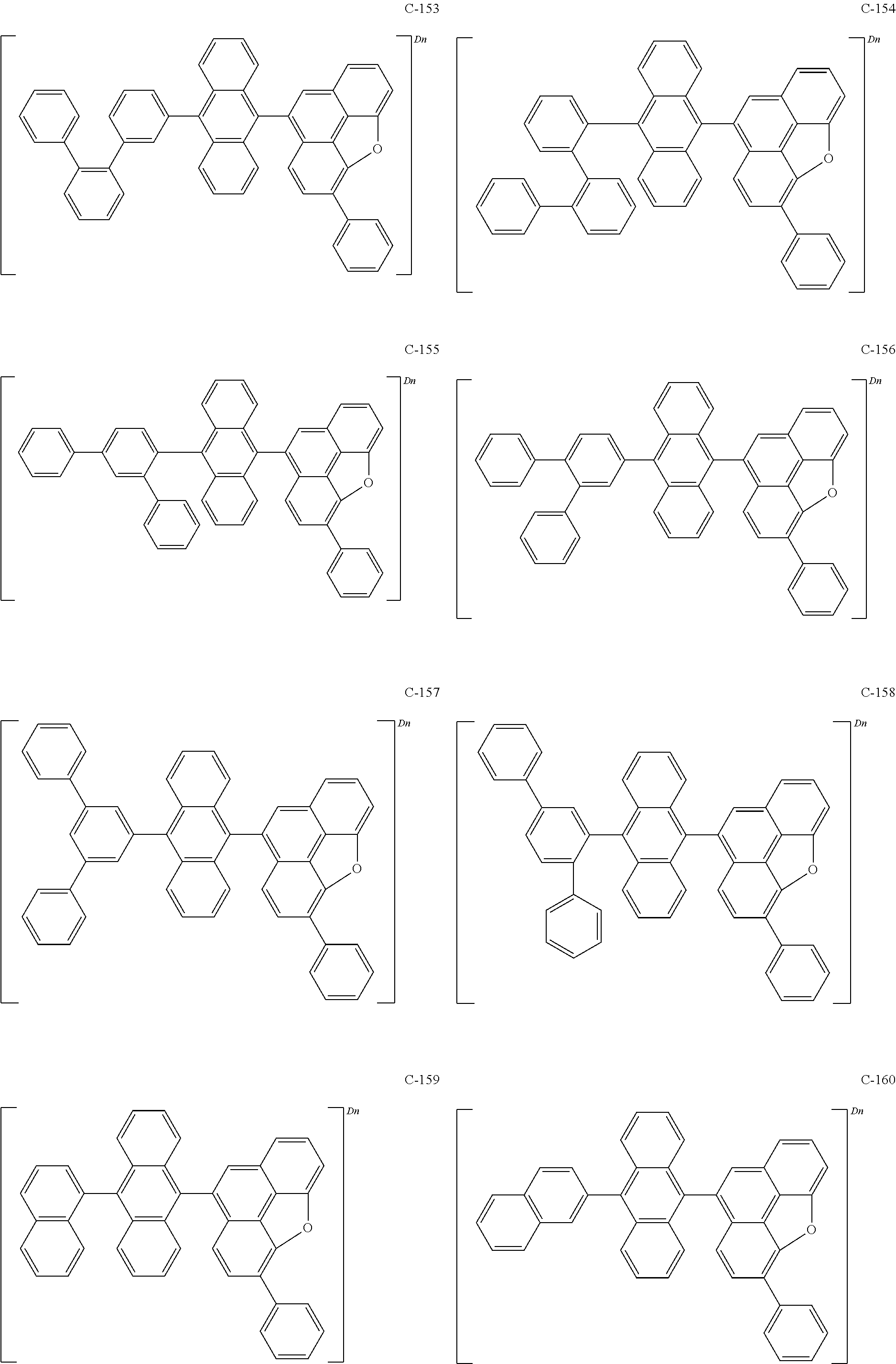

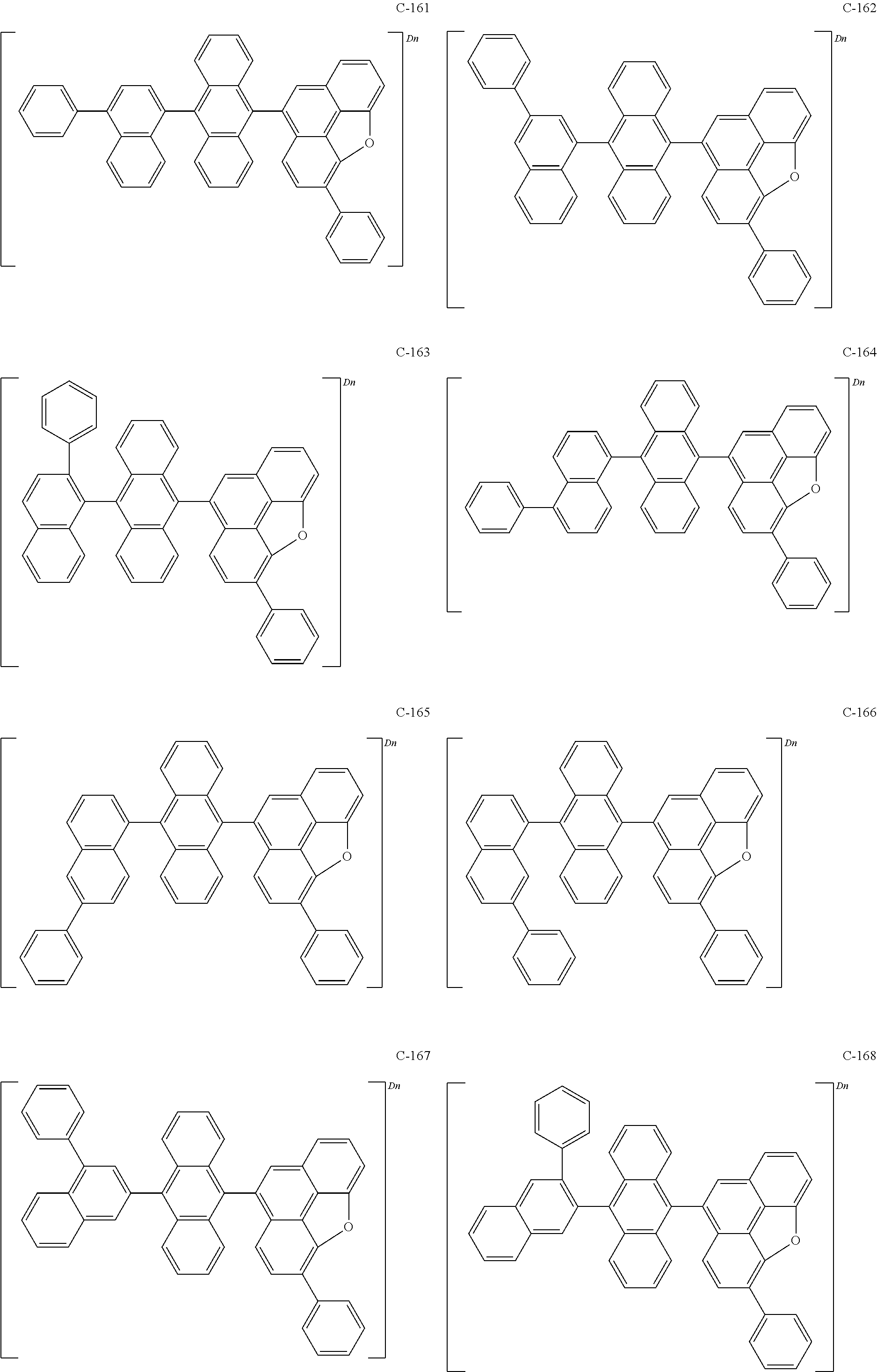

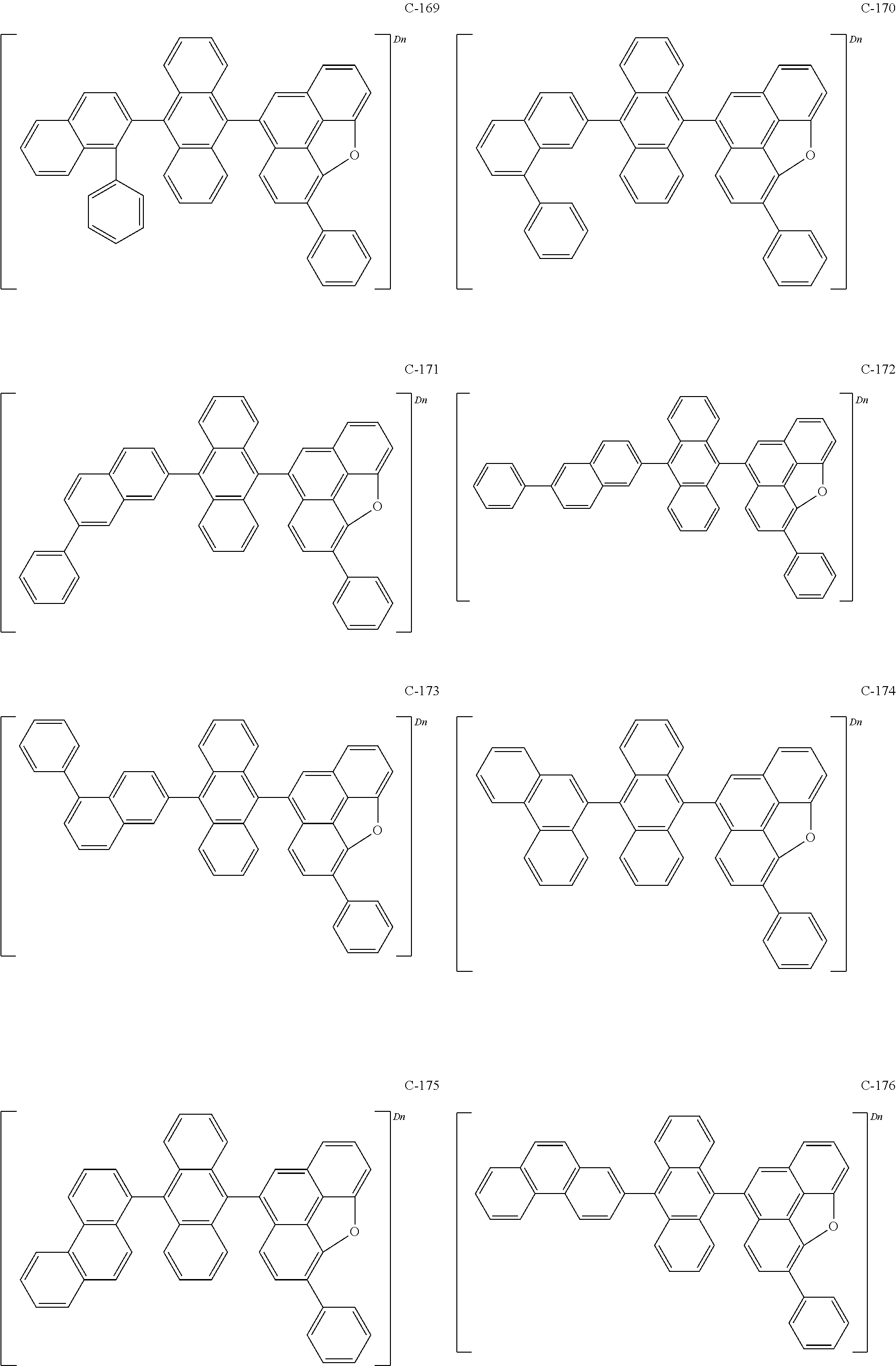

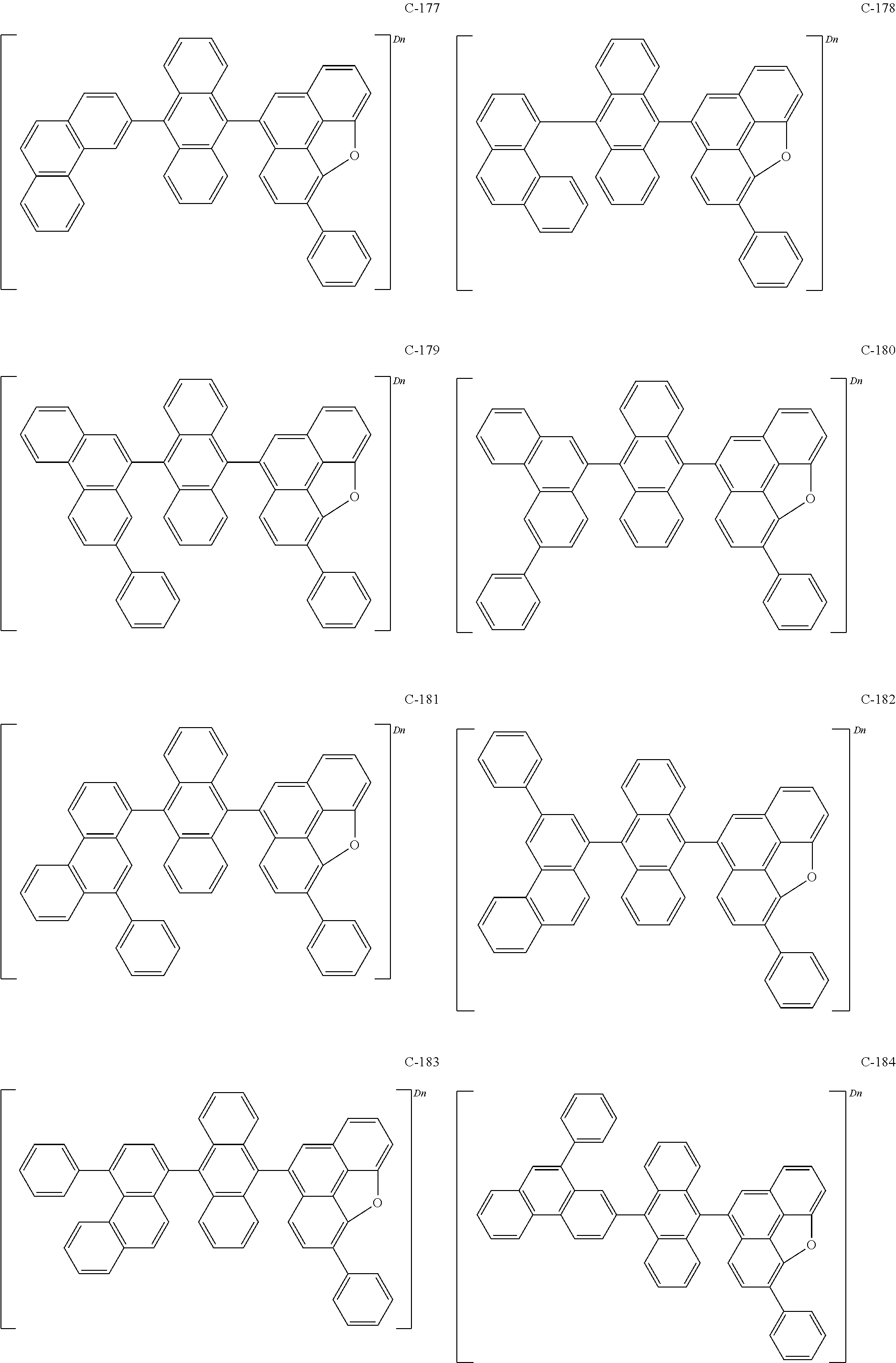

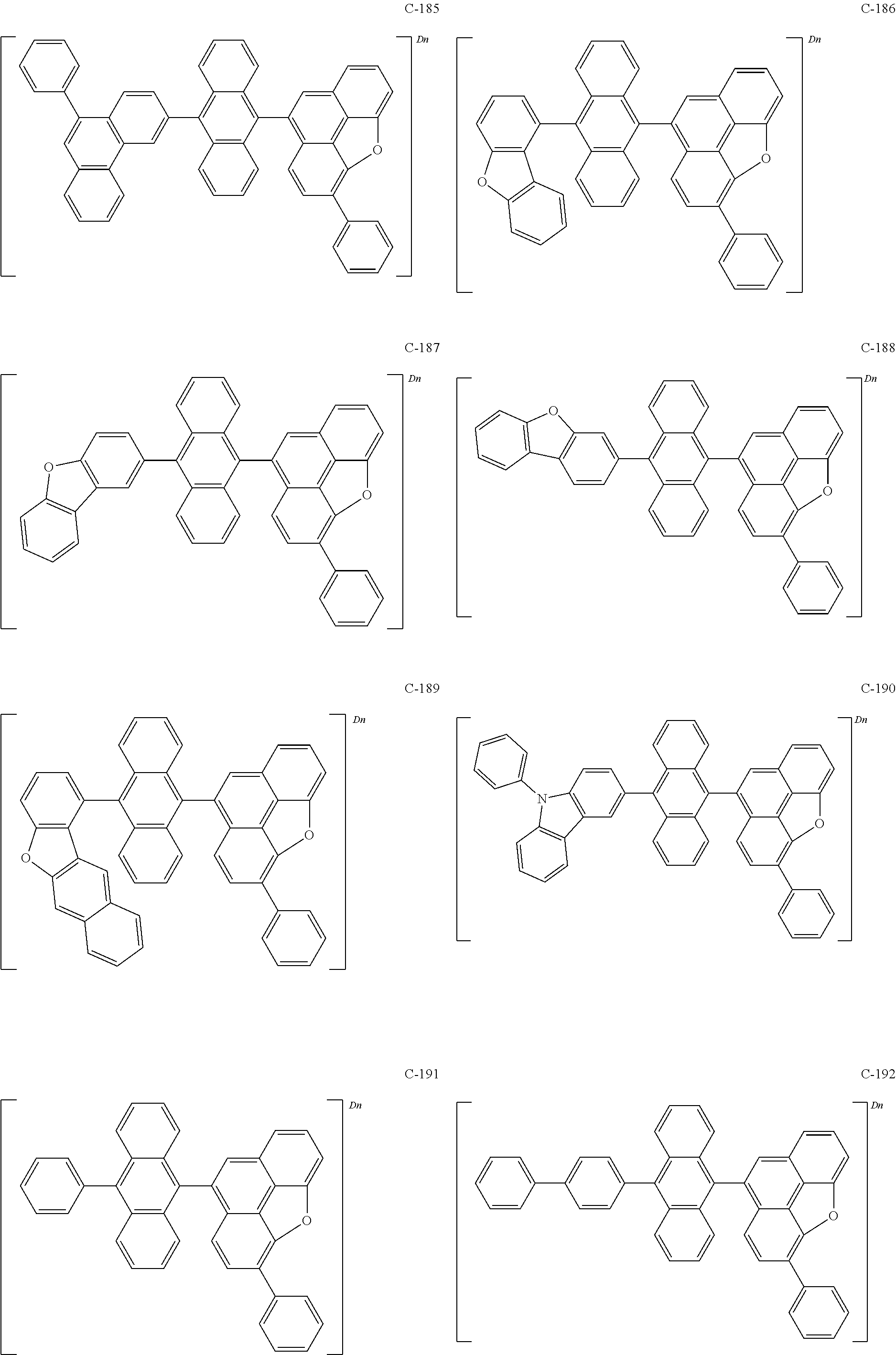

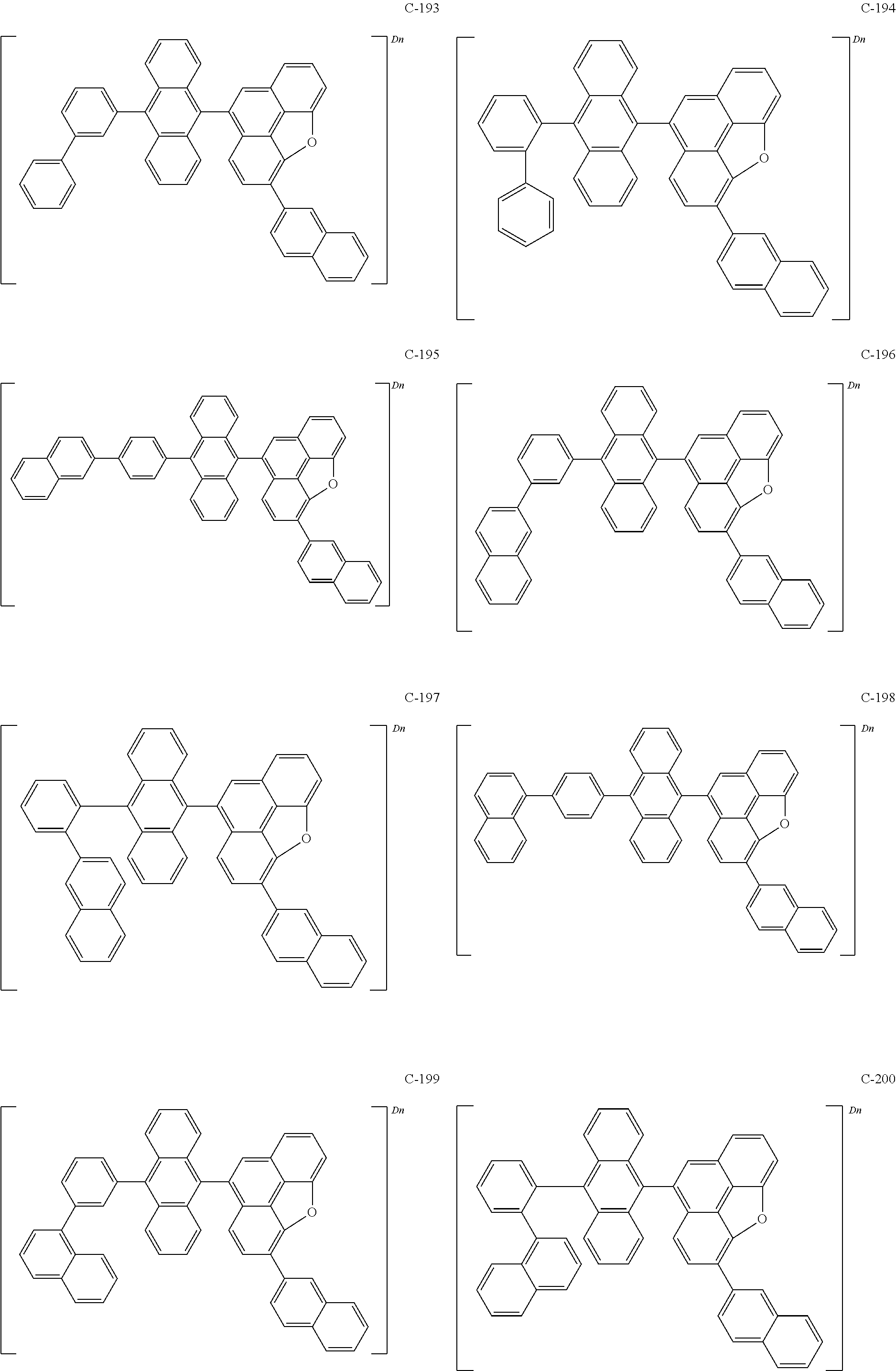

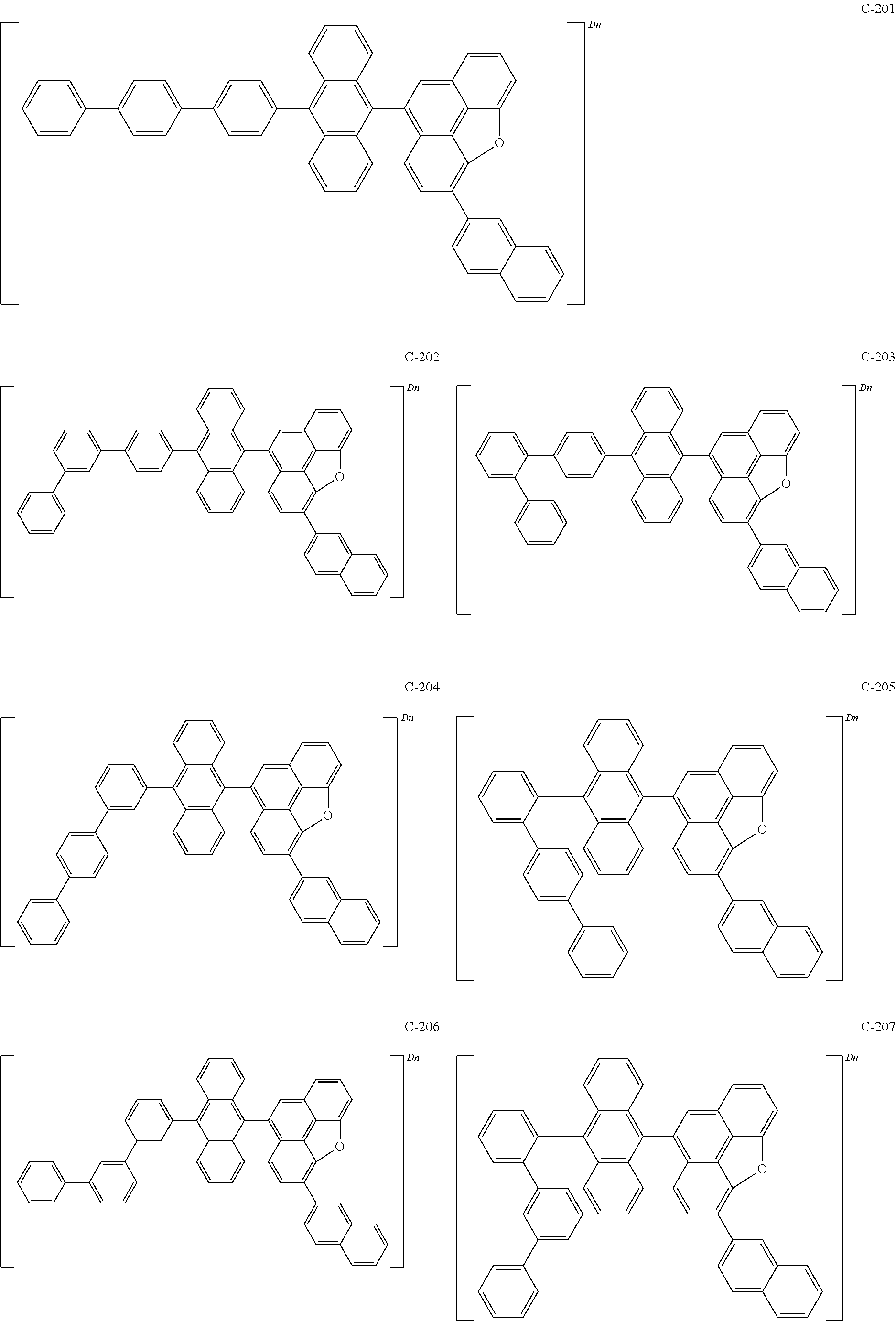

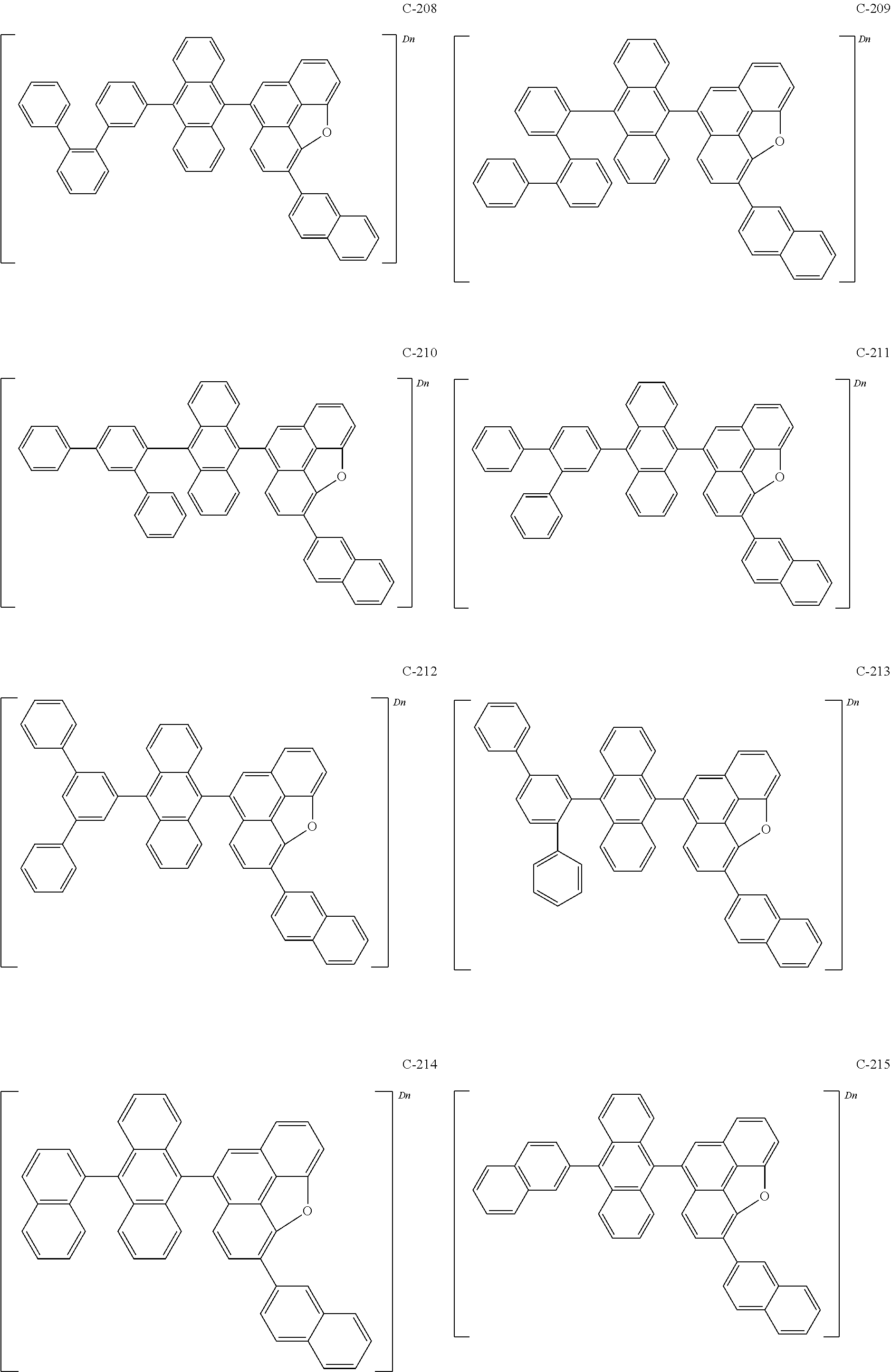

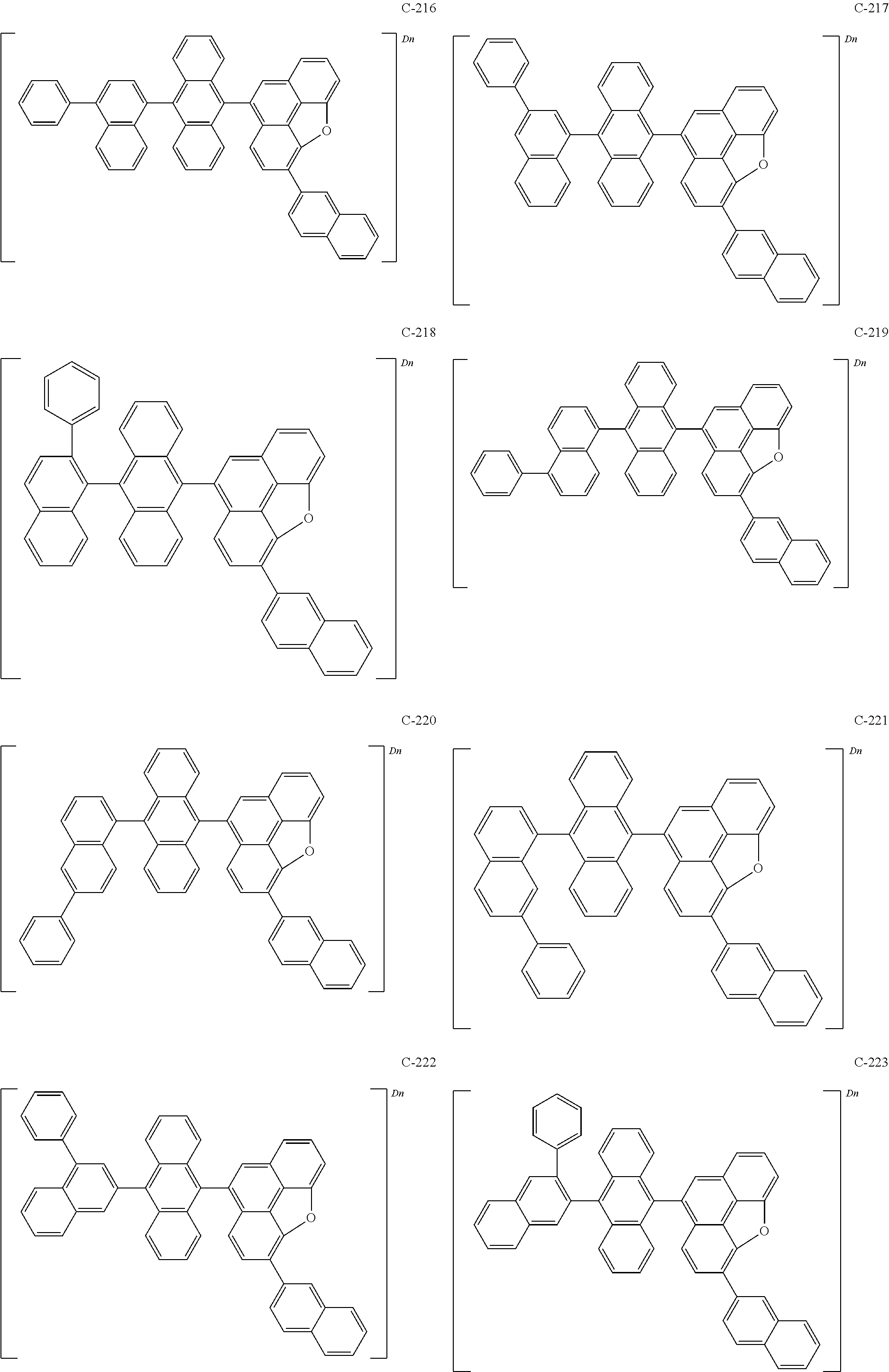

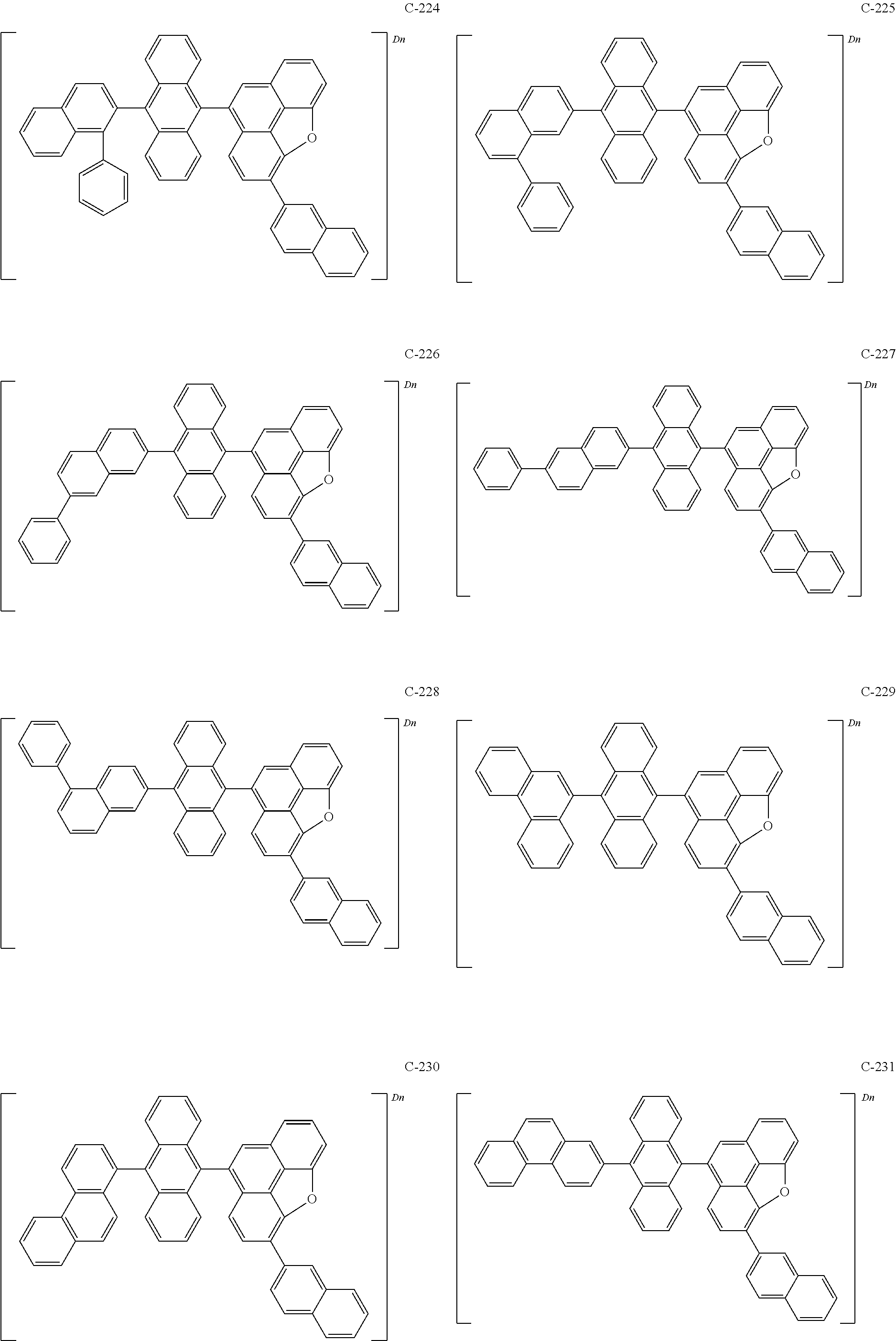

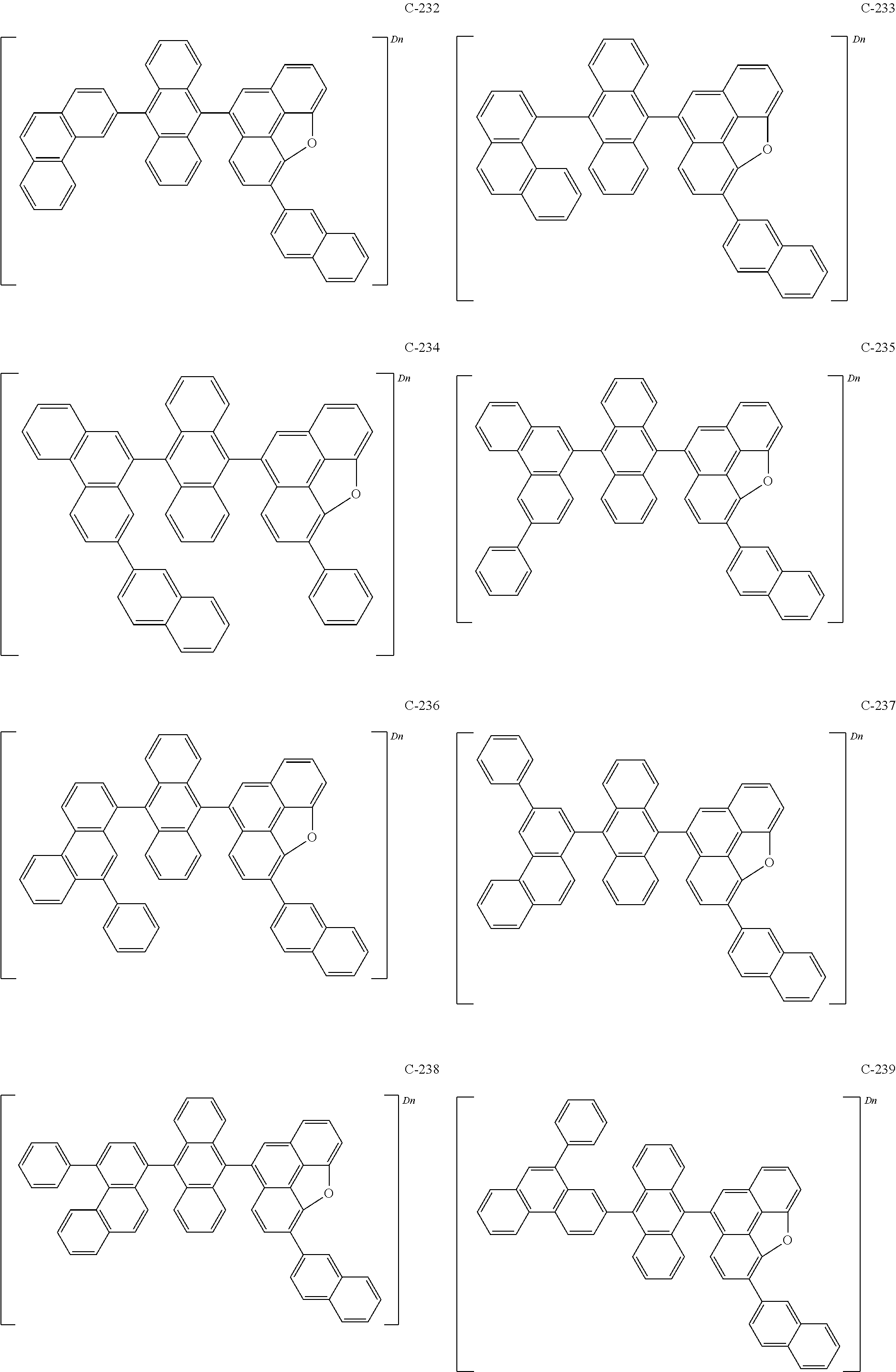

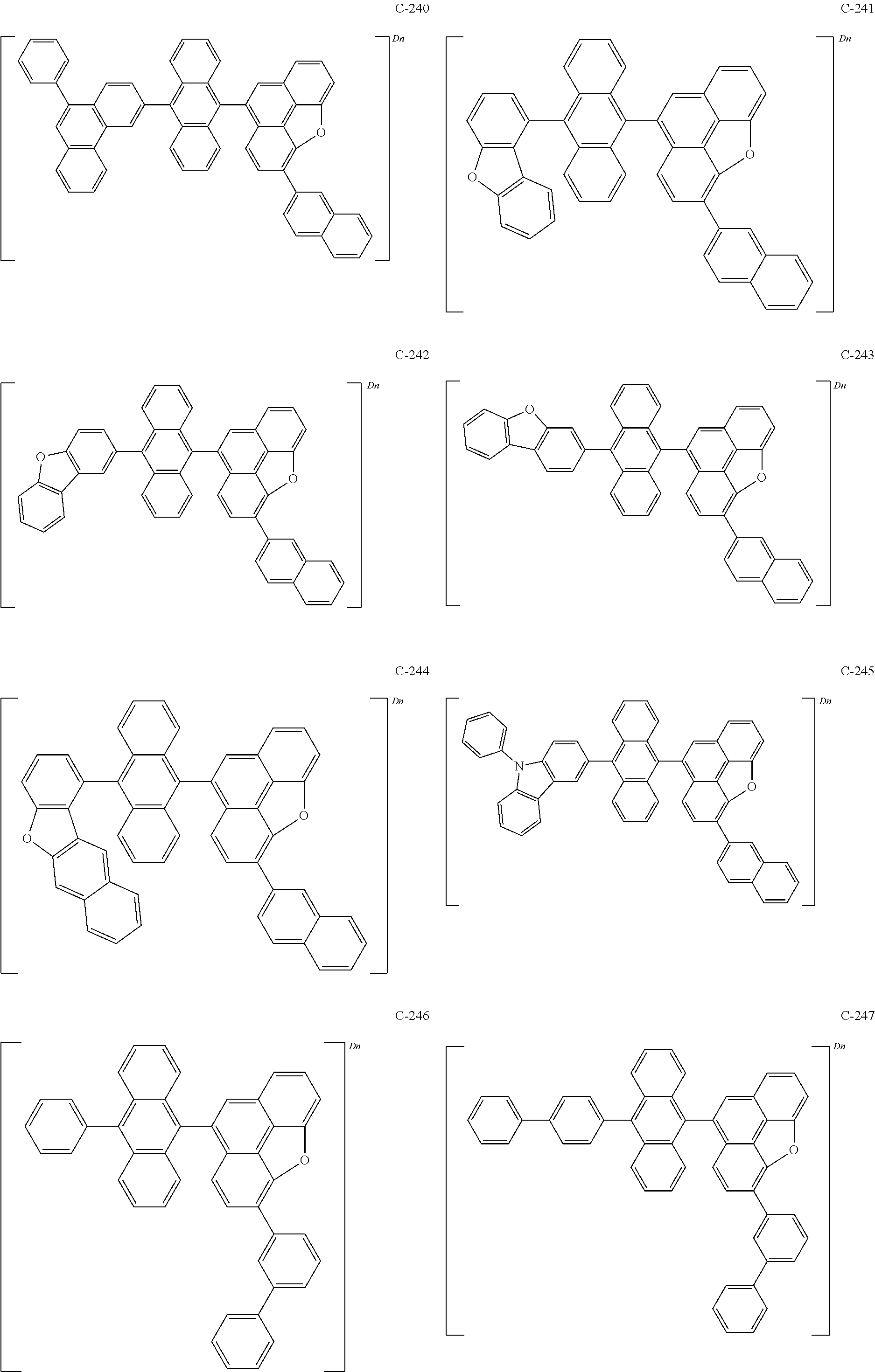

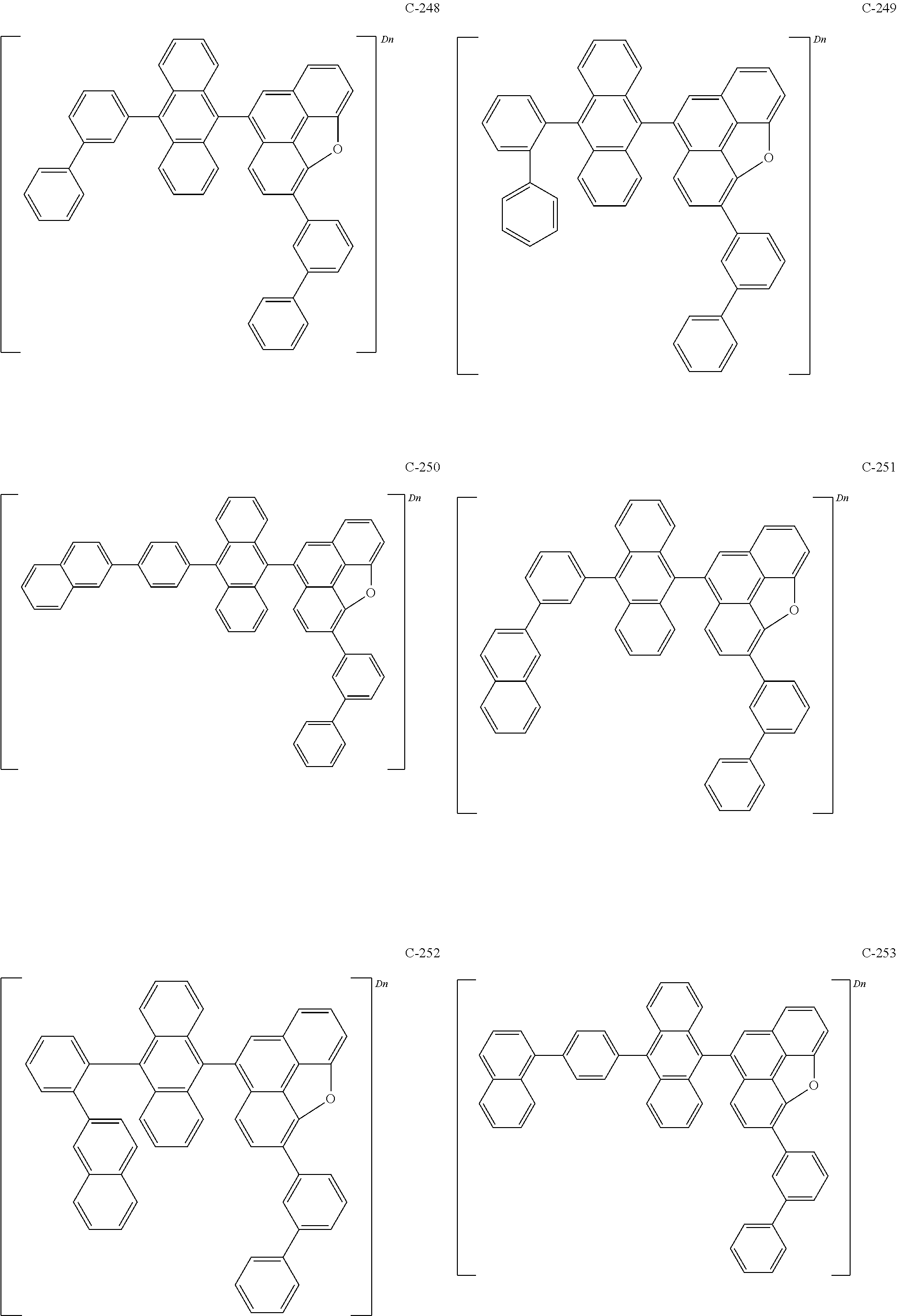

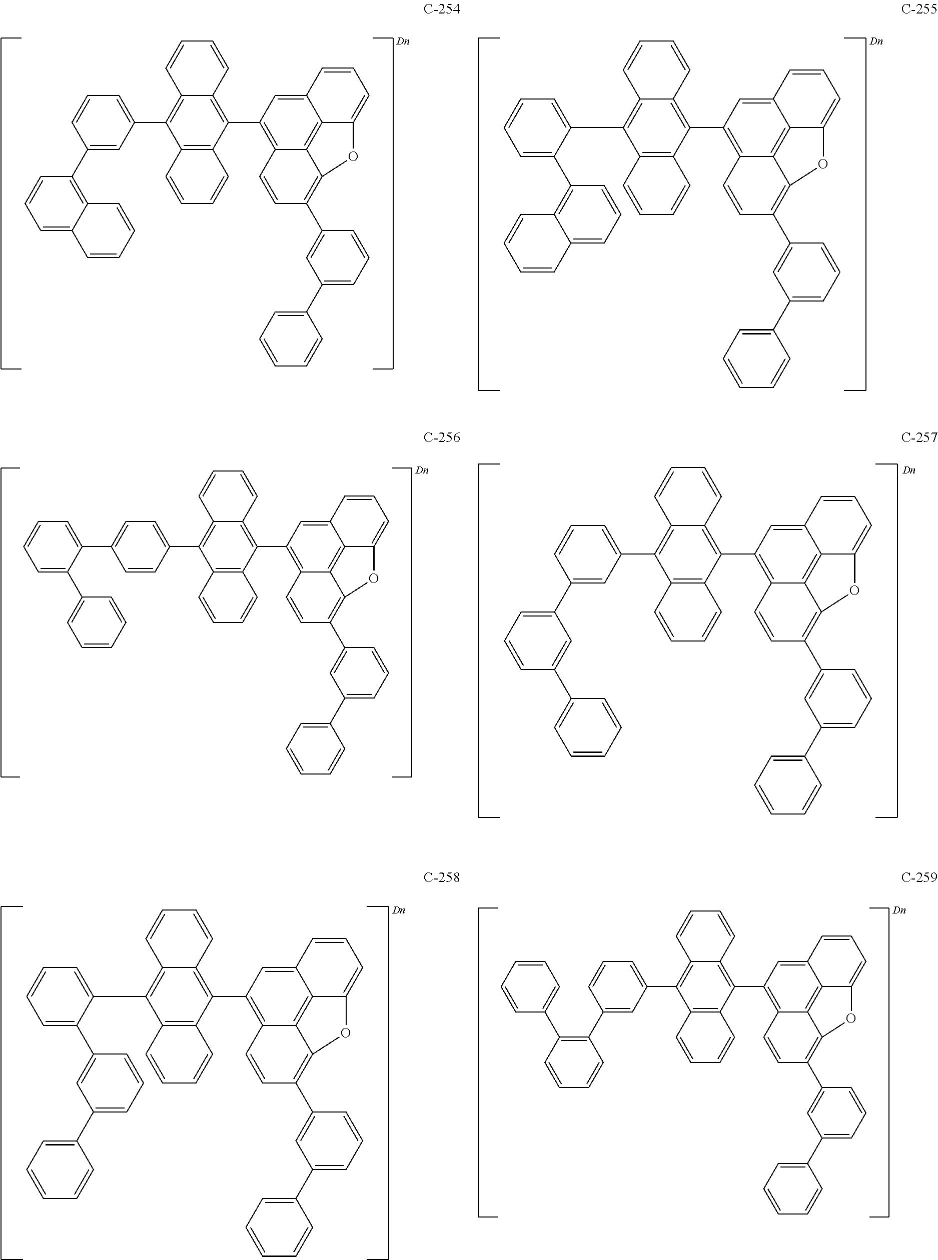

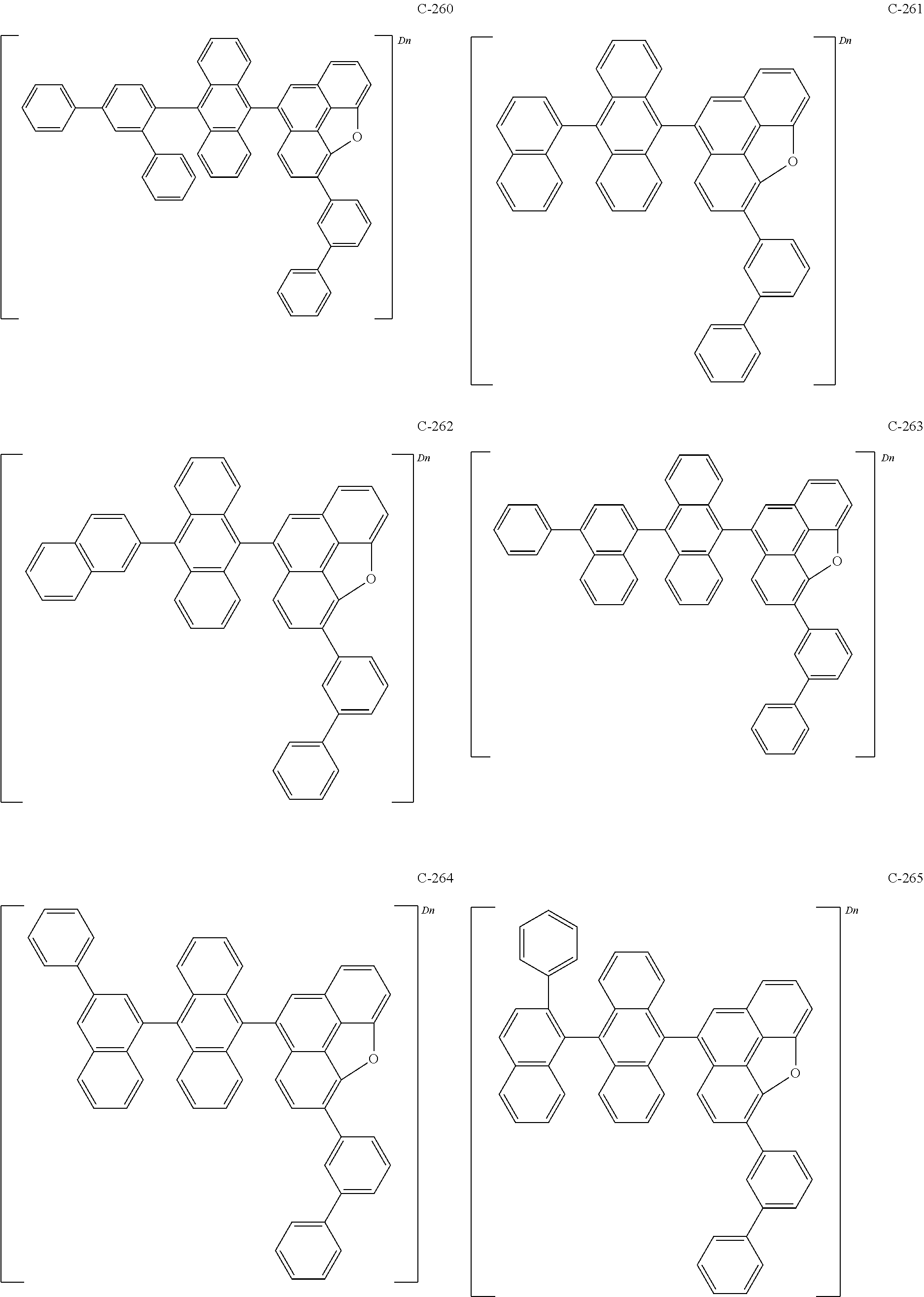

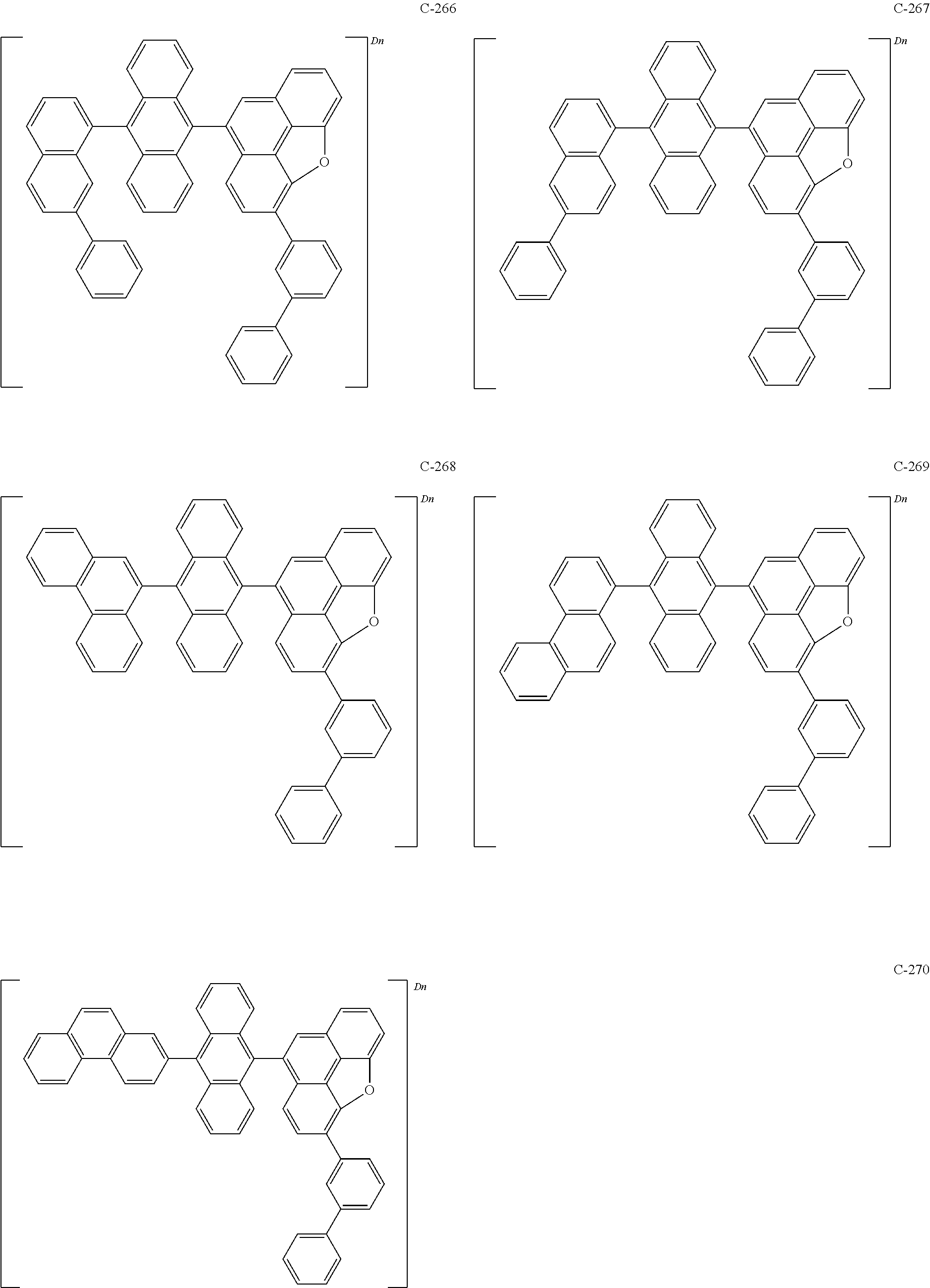

Dn of compounds C-136 to C-270 means that n hydrogens is replaced by deuteriums, and n is between 1 and the maximum number of hydrogens in each compound.

The compounds represented by formula 4 may be described in more detail as follows.

In formula 4, Ar₅ may be a substituted or unsubstituted (C6-C30)aryl, or a substituted or unsubstituted (3- to 30-membered)heteroaryl, preferably a substituted or unsubstituted (C6-C25)aryl, or a substituted or unsubstituted (3- to 25-membered)heteroaryl, more preferably a substituted or unsubstituted (C6-C20)aryl, or a substituted or unsubstituted (3- to 20-membered)heteroaryl. In one embodiment of the present disclosure, Ar₅ of formula 4 may be a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted triphenylenyl, a substituted or unsubstituted fluorenyl, a substituted or unsubstituted dibenzofluorenyl, a substituted or unsubstituted dibenzofuranyl, a substituted or unsubstituted dibenzothiophenyl, a substituted or unsubstituted carbazolyl, or a substituted or unsubstituted dibenzoselenophenyl. For example, in formula 4, Ar₅ may be a substituted or unsubstituted phenyl, a substituted or unsubstituted biphenyl, a substituted or unsubstituted naphthyl, a substituted or unsubstituted phenylnaphthyl, or a substituted or unsubstituted phenanthrenyl, and the hydrogen in each substituent may be substituted with deuterium.

In formula 4, L₅ may be a single bond, a substituted or unsubstituted (C6-C30)arylene, or a substituted or unsubstituted (3- to 30-membered)heteroarylene, preferably a single bond, a substituted or unsubstituted (C6-C25)arylene, or a substituted or unsubstituted (3- to 25-membered)heteroarylene, more preferably a single bond, a substituted or unsubstituted (C6-C25)arylene, or a substituted or unsubstituted (3- to 25-membered)heteroarylene. For example, in formula 4, L₅ may be a single bond.

In formula 4, R₁₃ may be hydrogen or deuterium.

In formula 4, R₁₄ to R₁₈ may each independently be, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(30-membered)heteroarylamino, or a substituted or unsubstituted (C6-C30)aryl(3- to 30-membered)heteroarylamino. For example, in formula 4, R₁₄ to R₁₈ may each independently be hydrogen, deuterium, or a substituted or unsubstituted phenyl, and the hydrogen in each substituent may be substituted with deuterium.

Provided that, in formula 4, at least one of R₁₄, R₁₅ and R₁₆ represents a substituted or unsubstituted (C6-C30)aryl, or a substituted or unsubstituted (3- to 30-membered)heteroaryl; and at least one of R₁₃ to R₁₈ may be deuterium.

In formula 4, k may be an integer of 1 or 2, and may be, for example, 1 or 2.

In formula 4, I may be an integer of 1 to 3, for example, 1, 2, or 3.

In formula 4, m and n may each independently be an integer of 1 to 4, and for example, may be each independently 1, 2, 3, or 4.

According to one embodiment of the present disclosure, formula 4 may be represented by the following formula 4-1 or 4-2.

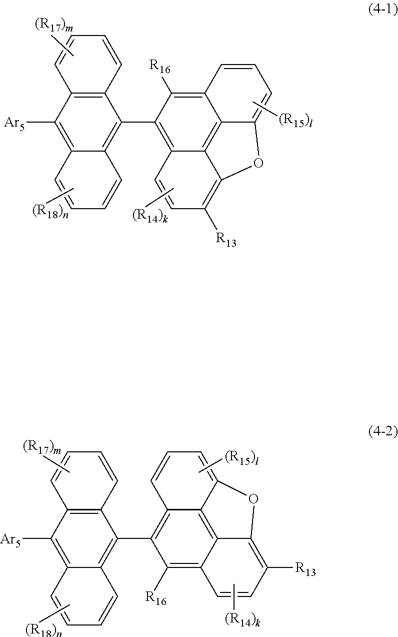

In formulas 4-1 and 4-2,

Ar₅, R₁₃ to R₁₈, and k to n are the same as defined in formula 4.

The compound represented by formula 4 may be selected from the following compounds, but are not limited thereto.

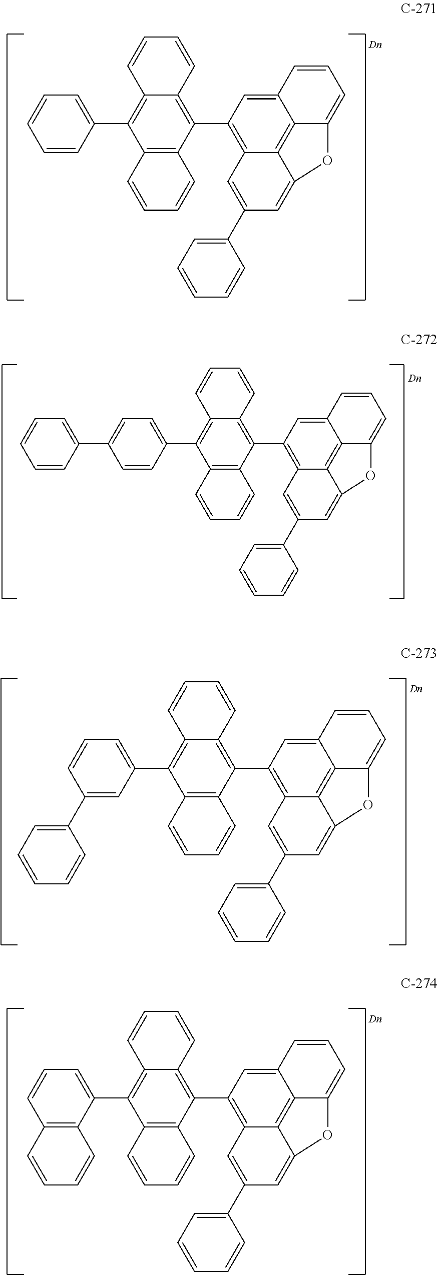

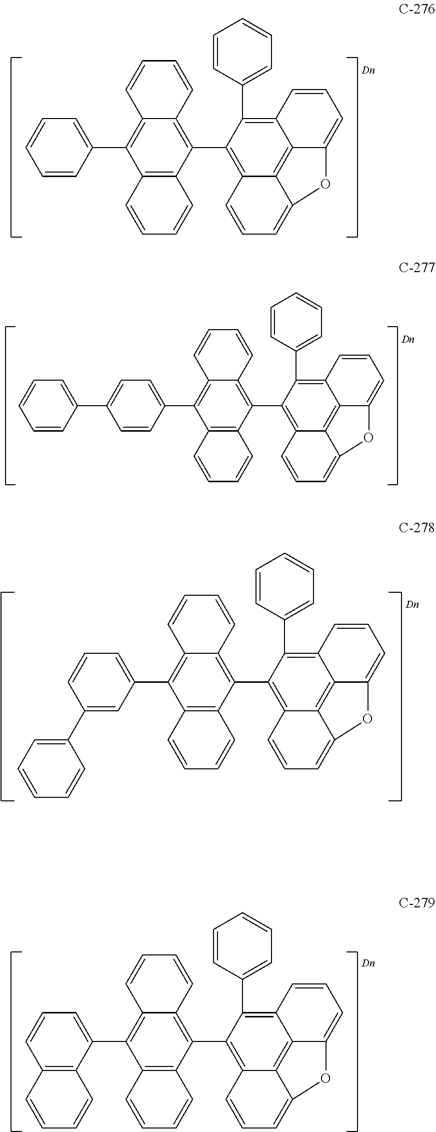

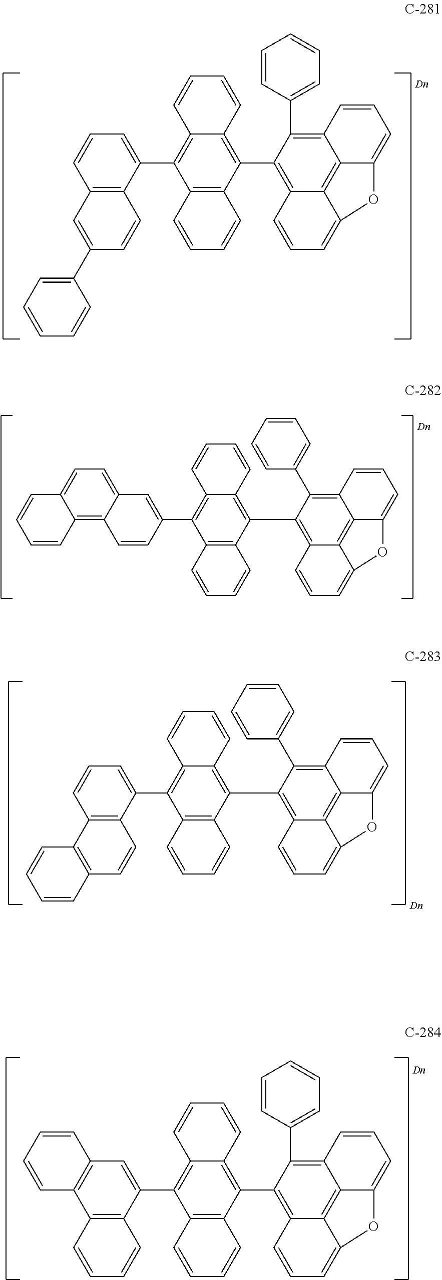

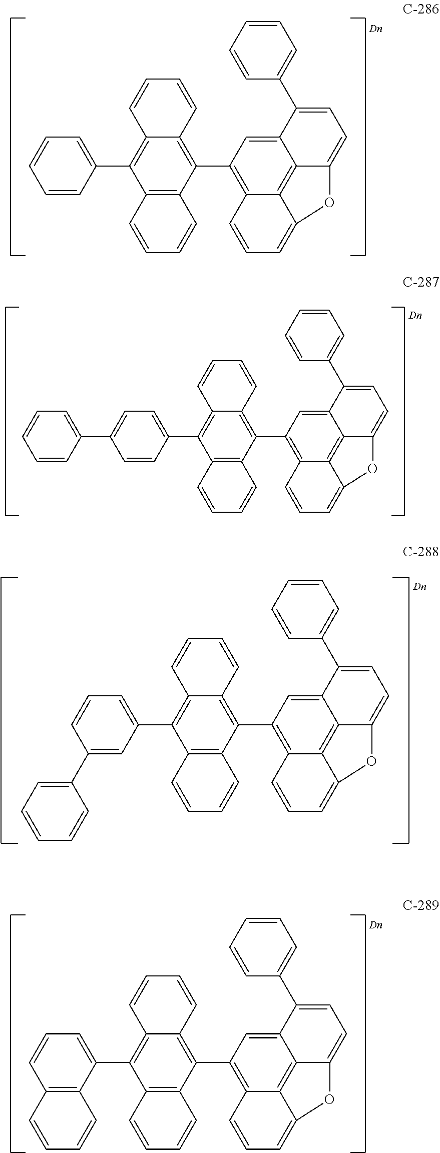

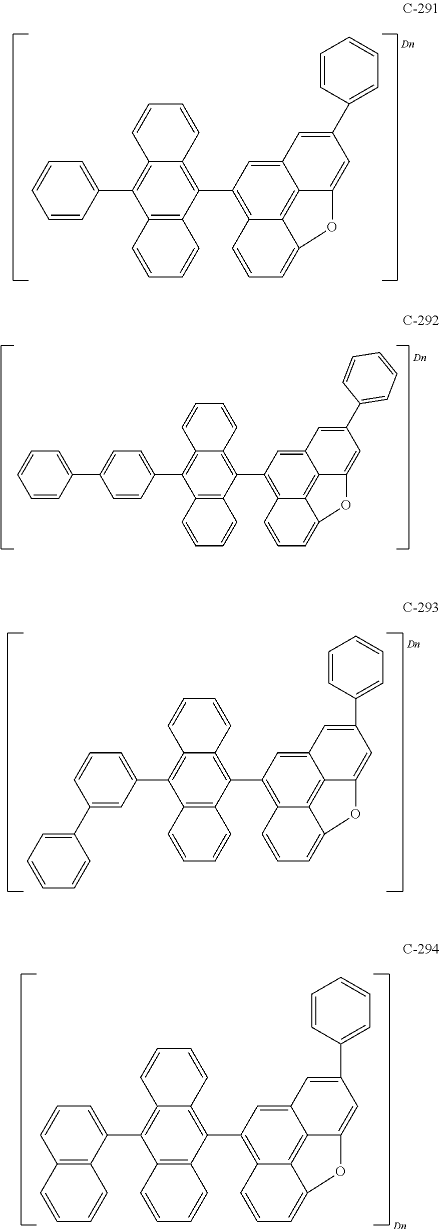

Dn of compounds C-271 to C-295 means that n hydrogens is replaced by deuterium, and n is between 1 and the maximum number of hydrogens in each compound.

The compounds represented by formula 1 according to the present disclosure may be prepared by a synthetic method known to a person skilled in the art, and for example, may be prepared as shown in Reaction Scheme below, but is not limited thereto.

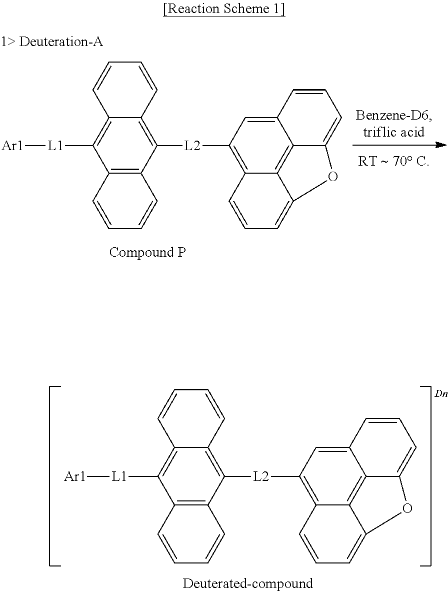

In Reaction Scheme 1, Ar₁, L₁, and L₂ are as defined in formula 1, Dn means that n hydrogens is replaced by deuteriums, and n is between 1 and the maximum number of hydrogens in the compound.

A compound represented by the following formula 5 may be used as the dopant included in the organic electroluminescent device of the present disclosure, but the present disclosure is not limited thereto.

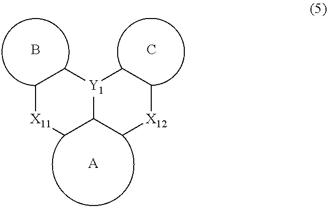

In formula 5,
Ring A, ring B, and ring C each independently represent a substituted or unsubstituted (C6-C30)aryl or a substituted or unsubstituted (3- to 50-membered)heteroaryl;
Y₁ represents B;
X₁₁ and X₁₂ each independently represent NRₐ, O, or S;
Rₐ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₆-N—(Ar₁₅)(Ar₁₆); or Rₐ may be linked to at least one of the ring A, the ring B, and the ring C to form a ring;
L₆ represents a single bond, a substituted or unsubstituted (C6-C30)arylene, a substituted or unsubstituted (3- to 30-membered)heteroarylene, or a substituted or unsubstituted divalent (C2-C30) aliphatic hydrocarbon group, or a substituted or unsubstituted divalent fused ring of a (C3-C30) aliphatic ring and a (C6-C30) aromatic ring;
Ar₁₅ and Ar₁₆ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

According to one embodiment, the luminescent compound may be more specifically exemplified by the following compounds, but is not limited thereto.

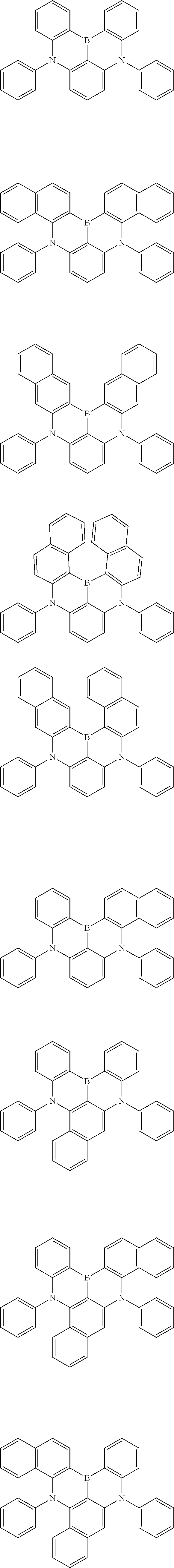

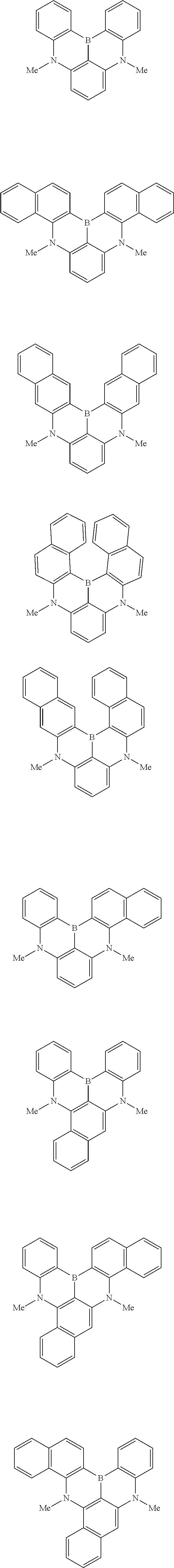

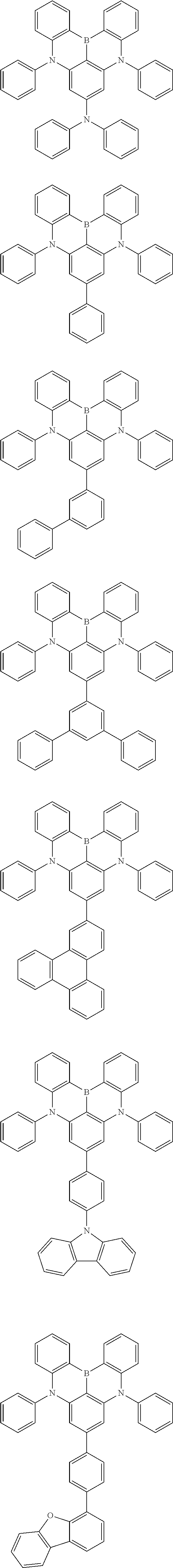

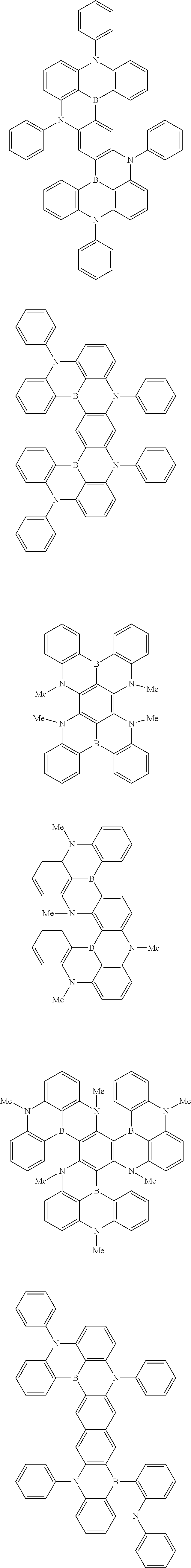

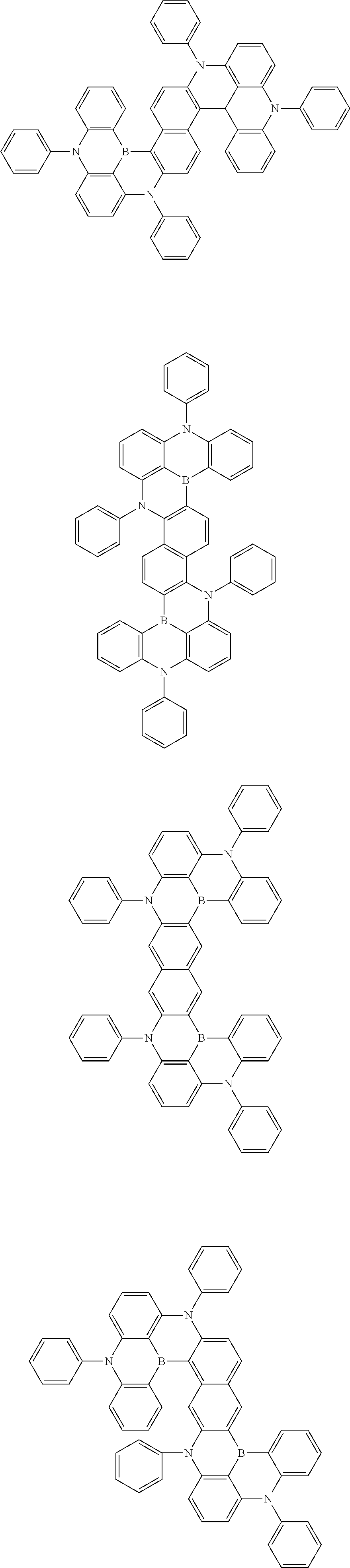 [SMILES: c1ccc(N2c3ccccc3B3c4c2cccc4N(c2ccccc2)c2ccc4c5c(ccc4c23)N(c2ccccc2)c2cccc3c2C5c2ccccc2N3c2ccccc2)cc1 ; c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1 ; c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1]

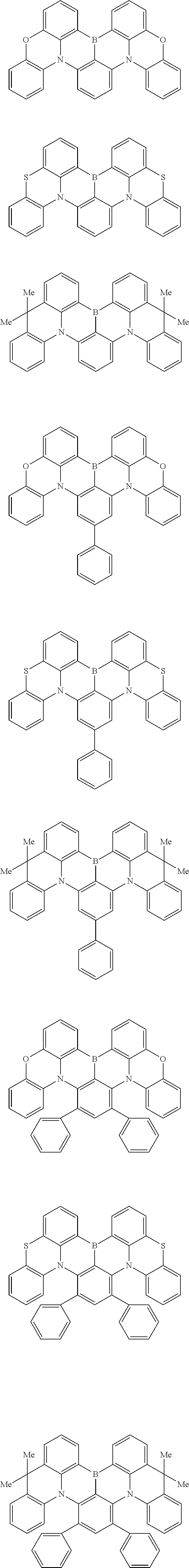

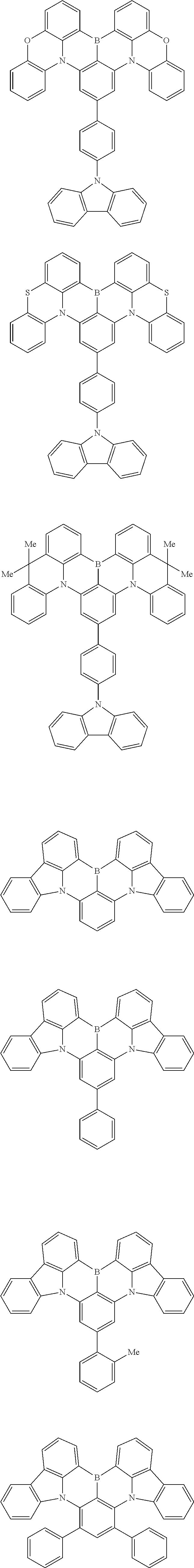

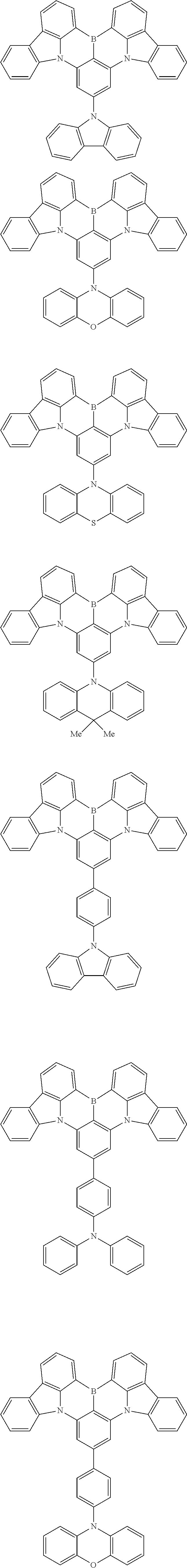

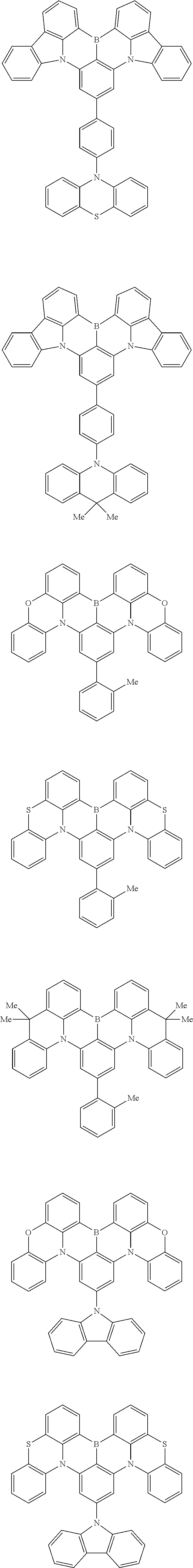

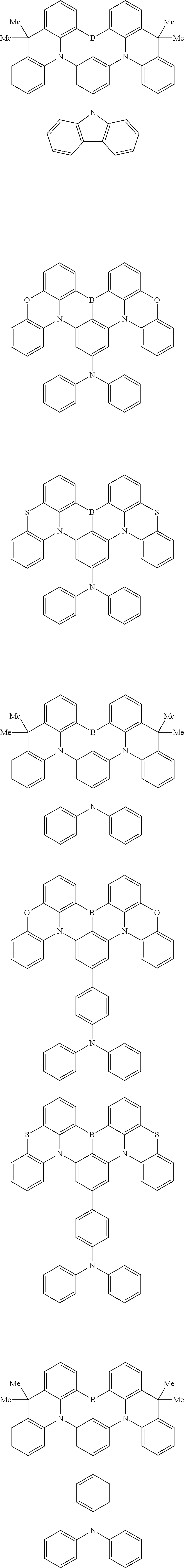

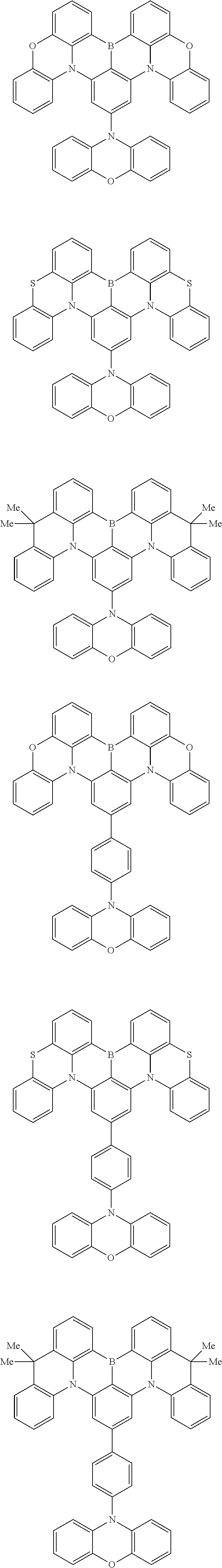

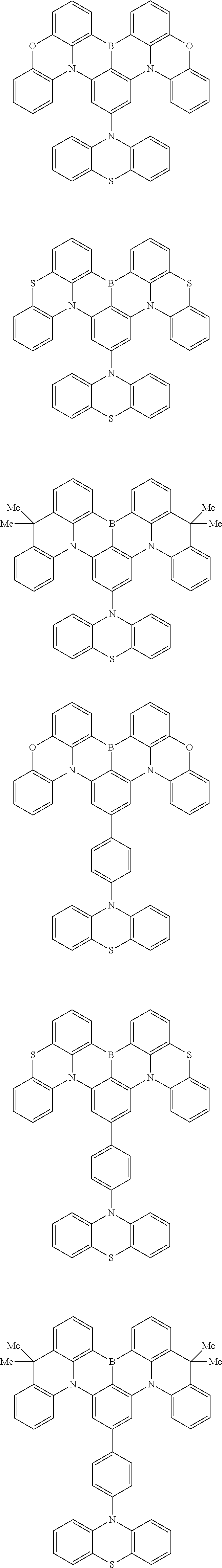

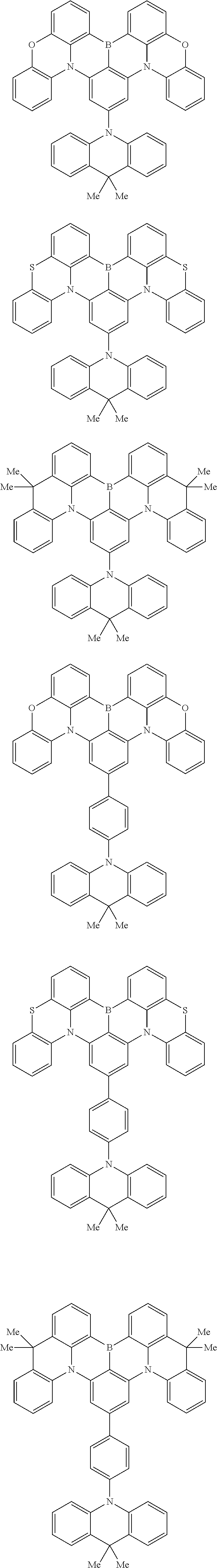

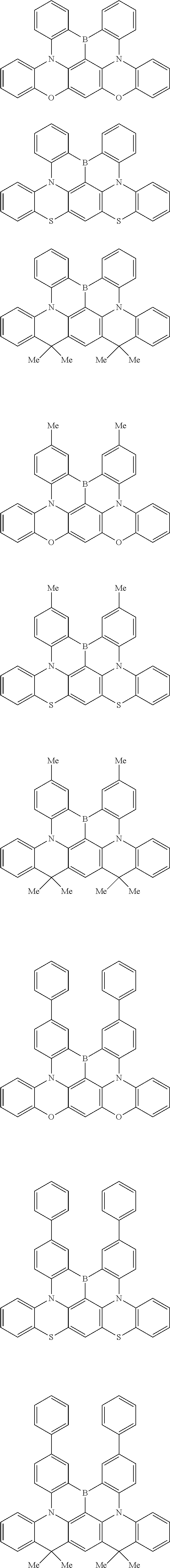

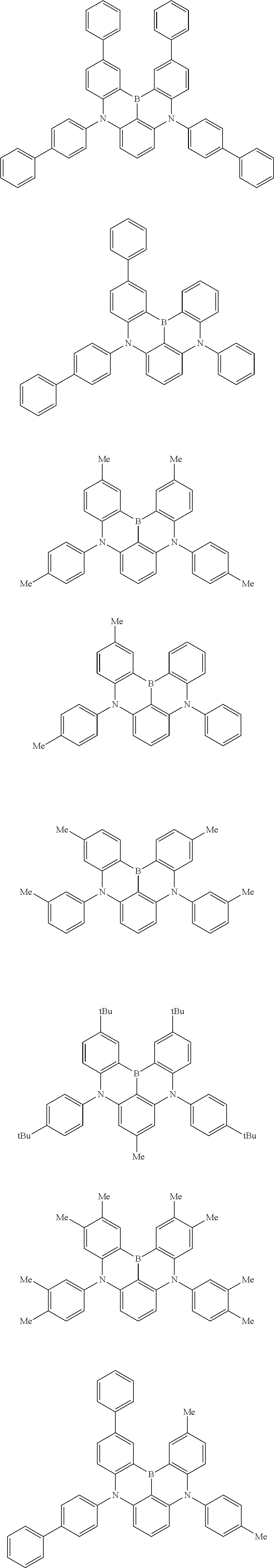

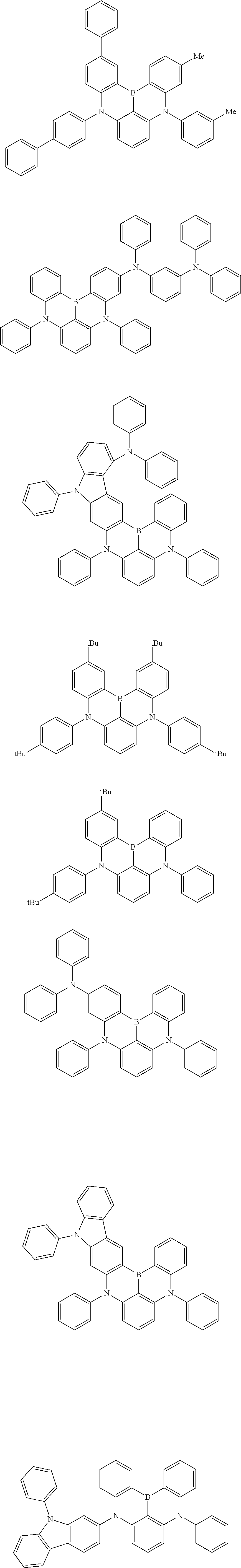

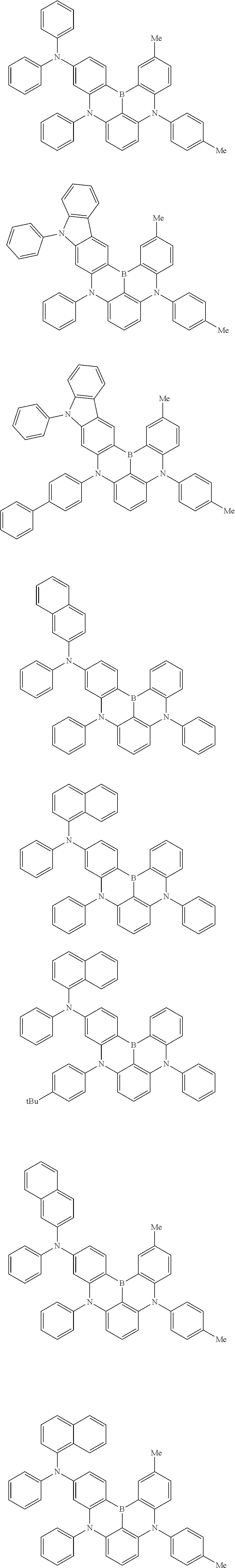

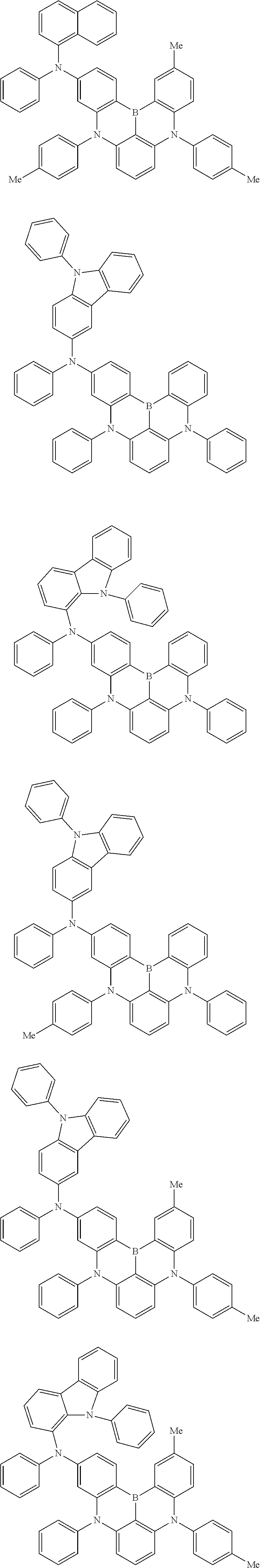

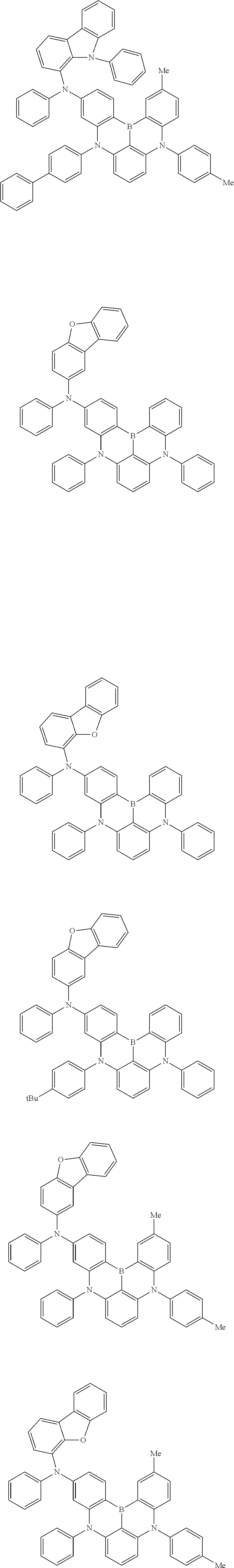

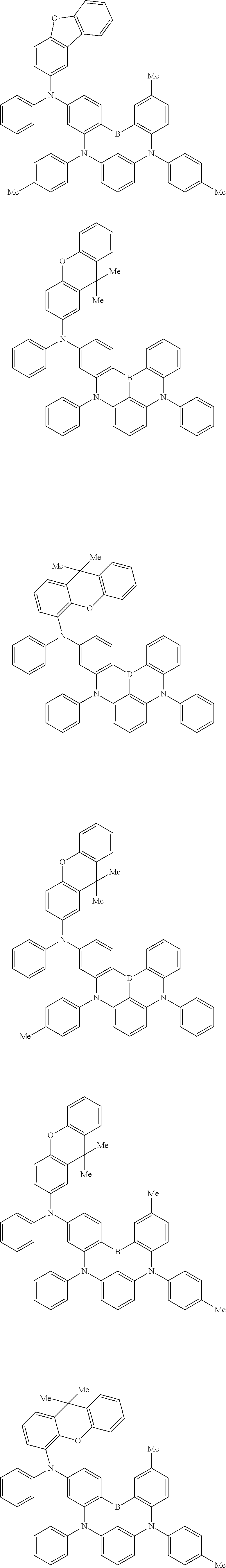

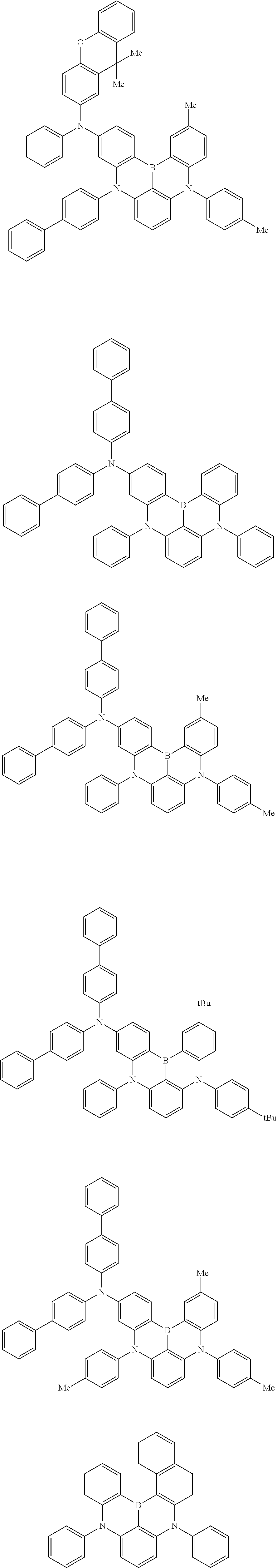

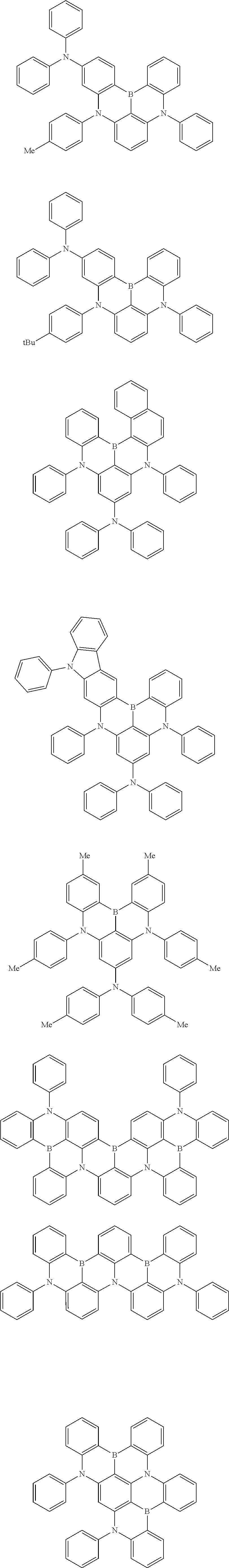

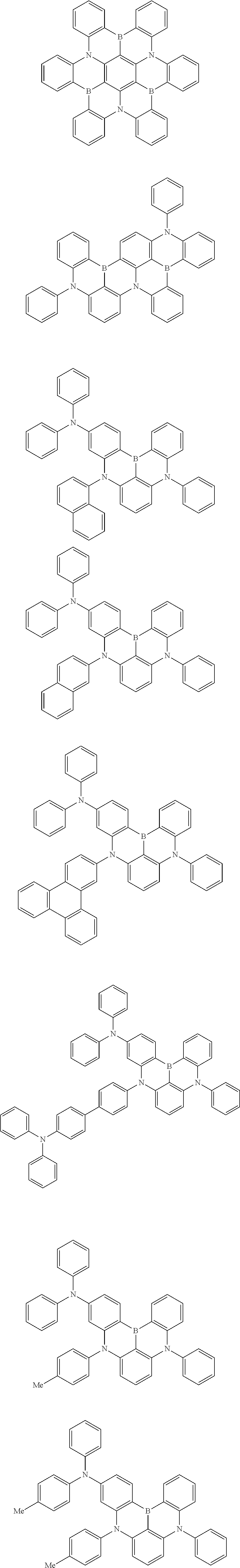

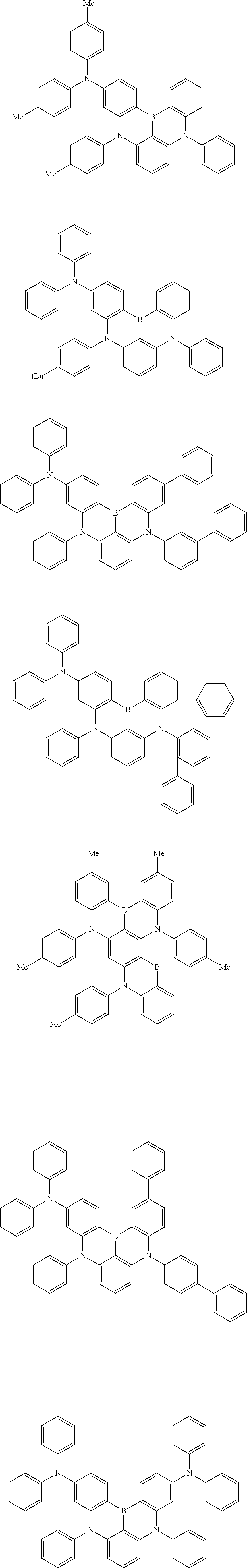

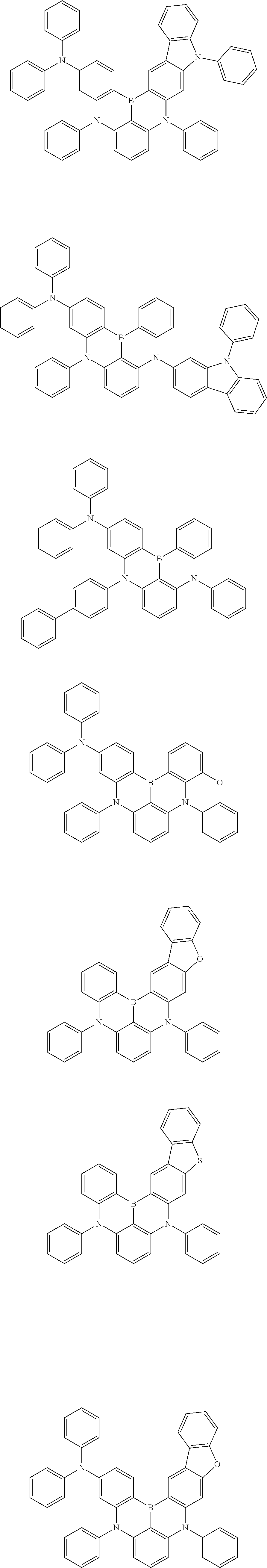

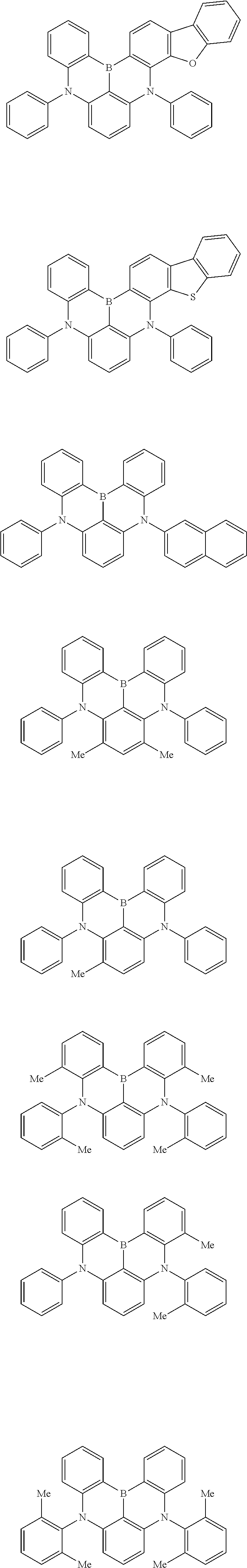

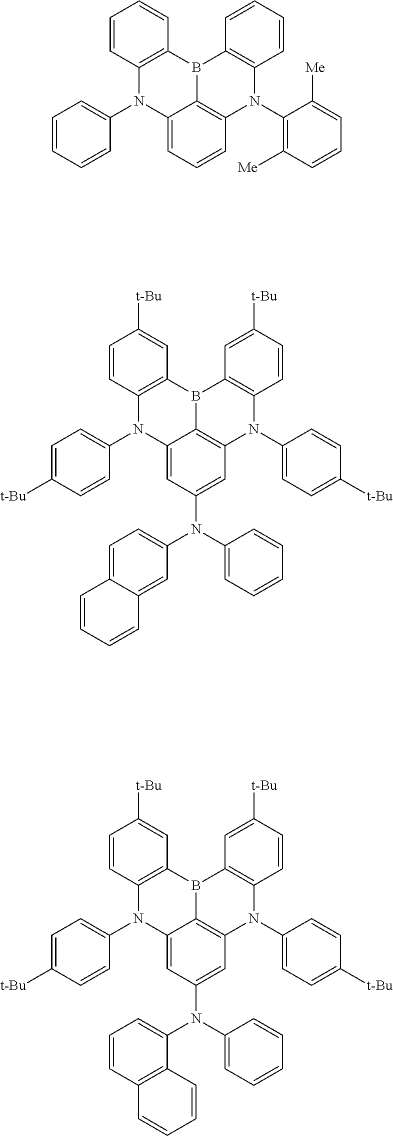 [SMILES: Cc1cccc(C)c1N1c2ccccc2B2c3ccccc3N(c3ccccc3)c3cccc1c32 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1]

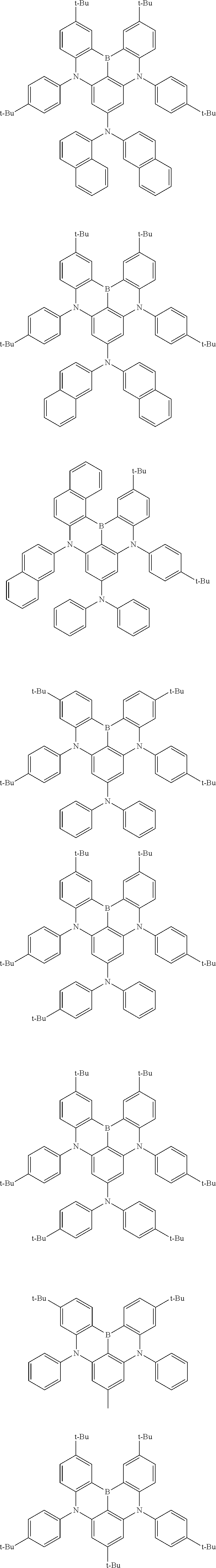

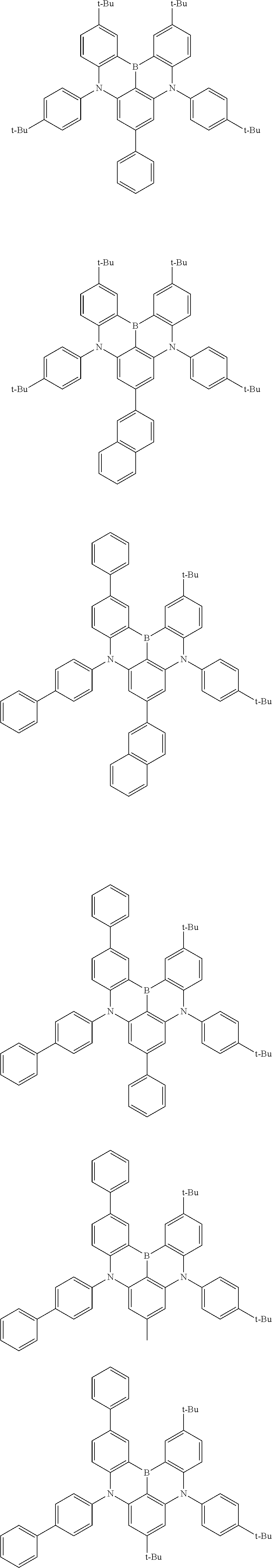

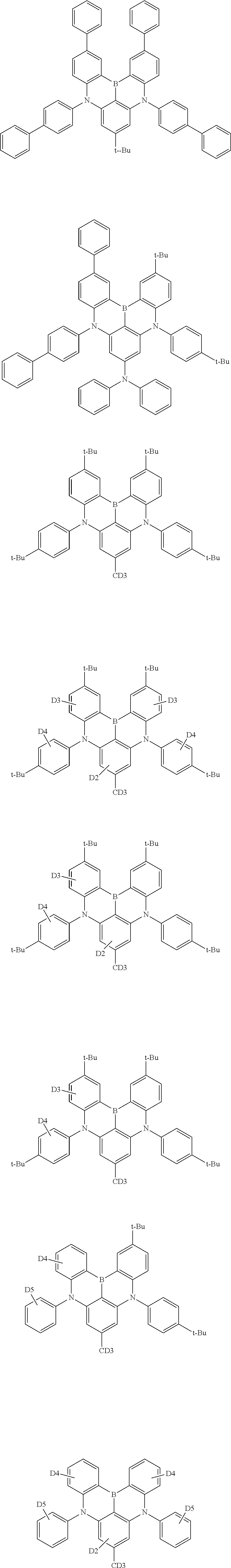

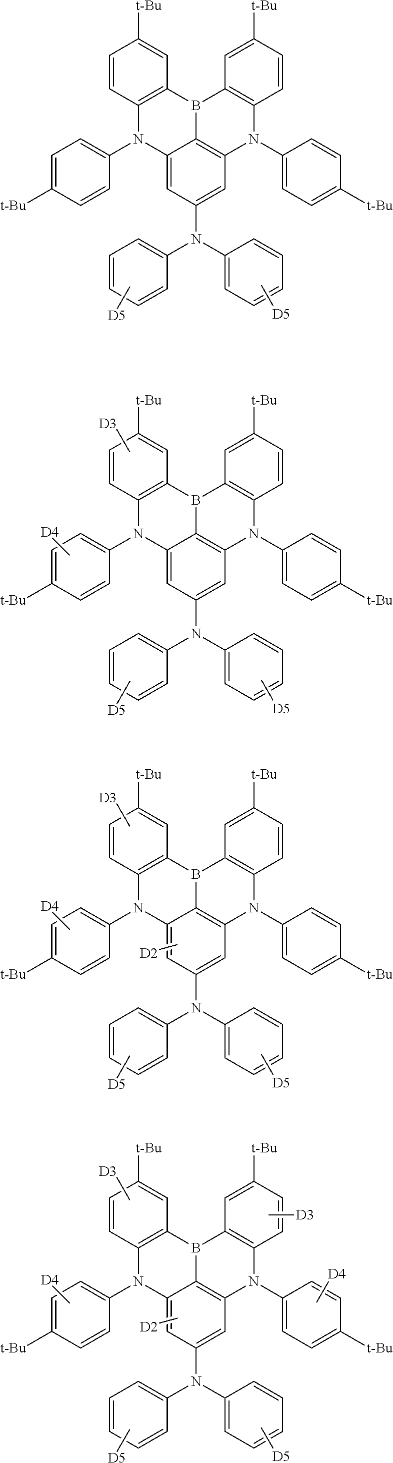

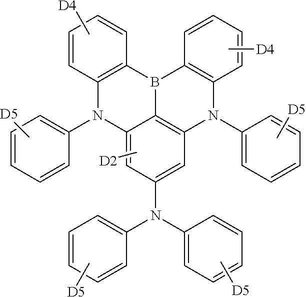

In said compounds, D2 to D5 mean that 2 to 5 hydrogens are replaced with deuteriums, respectively.

The present disclosure provides an organic electroluminescent material for manufacturing an organic electroluminescent device as a further embodiment. The organic electroluminescent material is preferably a composition for preparing a light-emitting layer, a hole transport layer, a hole auxiliary layer or a light-emitting auxiliary layer of the organic electroluminescent device, and includes the compound of the present disclosure. When there are two or more hole transport layers, the compound of the present disclosure may be included in the composition for preparing a hole transport layer (hole auxiliary layer) adjacent to the light-emitting layer.

The organic electroluminescent device according to the present disclosure comprises an anode; a cathode; and one or more organic layers interposed between the anode and the cathode. The organic layer may comprise a light-emitting layer and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further composed of several layers.

Each of the anode and the cathode may be formed of a transparent conductive material, or may be formed of a transflective or reflective conductive material. The organic electroluminescent device may be a top emission type, a bottom emission type, or a dual emission type according to the kind of material forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

In addition, in the organic electroluminescent device of the present disclosure, the organic material layer may further comprise at least one metal selected from the group consisting of organic metals of Group 1, Group 2, Period 4 transition metals, Period 5 transition metals, lanthanide metals, and d-transition elements, or at least one complex compound comprising such a metal.

In addition, the organic electroluminescent device of the present disclosure may further comprise at least one light-emitting layer including a blue, red or green light emitting compound known in the art in addition to the compound of the present disclosure, thereby emitting white light. In addition, if necessary, a yellow or orange light-emitting layer may be further included.

In the organic electroluminescent device of the present disclosure, preferably, at least one inner surface layer selected from a chalcogenide layer, a metal halide layer, and a metal oxide layer (hereinafter referred to as “surface layers”) may be placed on an inner surface(s) of at least one of the pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of a light-emitting medium layer. The operation stability for the organic electroluminescent device may be obtained by the surface layer. Preferred examples of the chalcogenide include SiOₓ (1≤X≤2), AlOₓ (1≤X≤1.5), SiON, SiAlON, etc; preferred examples of the metal halide include LiF, MgF₂, CaF₂, rare earth fluoride metal, etc; and preferred examples of the metal oxide include Cs₂O, Li₂O, MgO, SrO, BaO, CaO, etc.

The light-emitting auxiliary layer is a layer positioned between the anode and the light-emitting layer or between the cathode and the light-emitting layer, and when positioned between the anode and the light-emitting layer, the light-emitting auxiliary layer may be used for facilitating injection and/or transfer of holes or blocking overflow of electrons, or when positioned between the cathode and the light-emitting layer, the light-emitting auxiliary layer may be used for facilitating injection and/or transfer of electrons or blocking overflow of holes. In addition, the hole auxiliary layer is positioned between the hole transport layer (or hole injection layer) and the light-emitting layer, and may show an effect of smoothing or blocking the transfer speed (or injection speed) of holes, thereby adjusting the charge balance. In addition, the electron blocking layer is a layer that is disposed between the hole transport layer (or the hole injection layer) and the light-emitting layer, blocks overflow of electrons from the light-emitting layer, confines excitons in the light-emitting layer, and prevents light emission leakage. When the hole transport layer comprises two or more layers, an additionally included layer may be used as the hole auxiliary layer or the electron blocking layer. The light-emitting auxiliary layer, the hole auxiliary layer, or the electron blocking layer has an effect of improving the efficiency and/or the lifespan of the organic electroluminescent device.

In addition, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. In addition, a reductive dopant layer may be employed as a charge generating layer to prepare an organic electroluminescent device having two or more light-emitting layers and emitting white light.

The organic electroluminescent material according to an embodiment of the present disclosure may be used as a light-emitting material for a white organic light-emitting device. Various structures such as a side-by-side scheme, a stacking scheme, or a color conversion material (CCM) scheme have been proposed according to an arrangement type of R (red), G (green), YG (yellowish green), and B (blue) light emitting units in the white organic electroluminescent device. In addition, the organic electroluminescent material according to an embodiment of the present disclosure may be used in an organic electroluminescent device including a quantum dot QD.

The respective layers of the organic electroluminescent device of the present disclosure may be formed by any one of dry film formation methods such as vacuum deposition, sputtering, plasma, ion plating, and the like, and wet film formation methods such as inkjet printing, nozzle printing, slot coating, spin coating, dip coating, flow coating, etc.

In the wet film-forming method, a material for forming each layer is dissolved or dispersed in an appropriate solvent such as ethanol, chloroform, tetrahydrofuran, dioxane, or the like to form a thin film, and the solvent may be any solvent as long as the material for forming each layer can be dissolved or dispersed and there is no problem in film-forming properties.

In addition, it is possible to manufacture a display device, for example, a display device for smartphones, tablets, laptops, PCs, TVs or vehicles, or lighting devices, for example, an outdoor or indoor lighting device, using the organic electroluminescent device of the present disclosure.

In the organic electroluminescent device of the present disclosure, a layer selected from a hole injection layer, a hole transport layer, or an electron blocking layer or a combination thereof may be used between the anode and the light-emitting layer. As the hole injection layer, a plurality of layers may be used for the purpose of lowering a hole injection barrier (or a hole injection voltage) from the anode to the hole transport layer or the electron blocking layer, and two compounds may be simultaneously used for each layer. A plurality of layers may be used as the hole transport layer or the electron blocking layer.

In addition, a layer selected from an electron buffer layer, a hole blocking layer, an electron transport layer, or an electron injection layer or a combination thereof may be used between the light-emitting layer and the cathode. A plurality of layers may be used as the electron buffer layer in order to control electron injection and improve interfacial properties between the light-emitting layer and the electron injection layer, and two compounds may be simultaneously used as each layer. A plurality of layers may be used for the hole blocking layer or the electron transport layer, and a plurality of compounds may be used for each layer.

In addition, the organic electroluminescent compound or the plurality of host materials according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as inkjet printing, nozzle printing, slot coating, spin coating, dip coating, flow coating methods, etc., may be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, the first and second host compounds of the present disclosure may be formed by the above-described method, and may be often formed by a co-deposition or mixed deposition process. The co-deposition is a method of putting two or more materials in each crucible source and simultaneously evaporating and mixing the materials by applying currents to two cells, and the mixed deposition is a method of mixing and depositing two or more materials in one crucible source before deposition and then evaporating and mixing the materials by applying currents to one cell. In addition, when the first and second host compounds are present in the same layer or different layers in the organic electroluminescent device, the two host compounds may be individually formed. For example, after depositing the first host compound, the second host compound may be deposited.

The present disclosure may provide a display device using a plurality of host materials comprising the compound represented by formula 1 and the compound represented by formula 2. That is, it is possible to manufacture a display device or a lighting device using a plurality of host materials of the present disclosure. Specifically, it is possible to manufacture a display system, for example, a display device for a smartphone, tablet, laptop, PCs, TVs, or vehicle, or a lighting system, such as an outdoor or indoor lighting system, using the plurality of host materials of the present application.

Hereinafter, for a detailed understanding of the present disclosure, the method for preparing the organic electroluminescent compound according to the present disclosure, the physical properties thereof, and the luminescent properties of the organic electroluminescent device comprising the same will be described by using the representative compound of the present disclosure. However, the present disclosure is not limited to the following examples.

### Example 1: Synthesis of Compound C-1

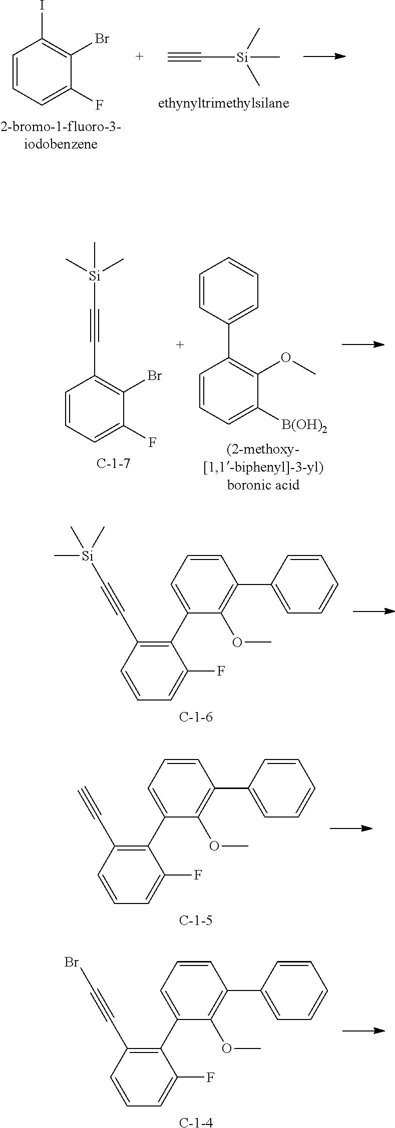

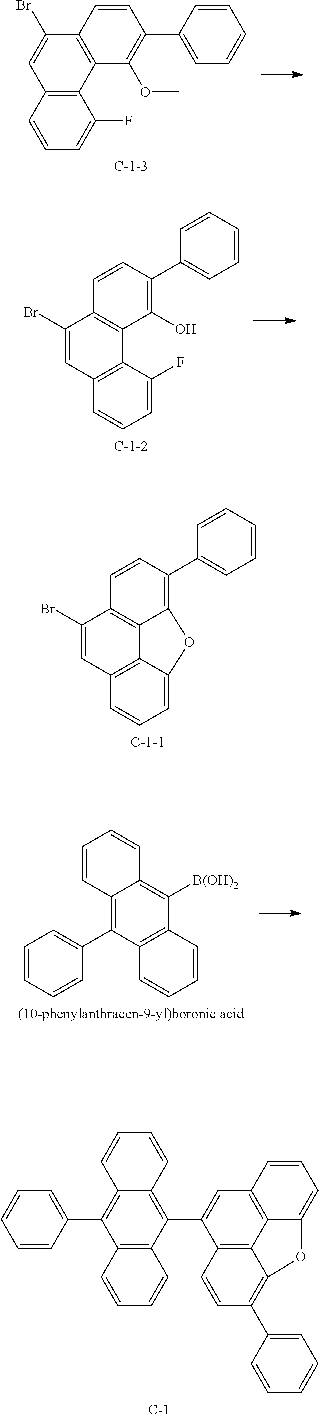

### (1) Synthesis of Compound C-1-7

2-bromo-1-fluoro-3-iodobenzene (60 g, 199.40 mmol), ethynyltrimethylsilane (23.50 g, 239.28 mmol), copper iodide (“CuI”) (1.89 g, 9.97 mmol), PdCl₂(PPh₃)₂(6.99 g, 9.97 mmol), and 420 ml of triethylamine were mixed and stirred at room temperature. After 2 hours, methylene chloride was added thereto, and the resultant was filtered under reduced pressure with celite. The residue was distilled under reduced pressure, and separated by column chromatography to obtain compound C-1-7 (52.2 g, yield: 96.67%).

### (2) Synthesis of Compound C-1-6

Compound C-1-7 (29 g, 106.9 mmol), 2-methoxy-[1,1′-biphenyl]-3-yl)boronic acid (26.82 g, 117.6 mmol), PdCl₂(Amphos)₂ (2.27 g, 3.20 mmol), 800 ml of toluene, Aliquat336 (2.4 ml, 5.34 mmol), Na₂CO₃ (22.66 g, 213.8 mmol), and 200 ml of distilled water were mixed and stirred at 135° C. After 2 hours, the mixture was cooled to room temperature, distilled water was added, and the organic layer was extracted with ethyl acetate. The organic layer was then dried with MgSO₄ and filtered under reduced pressure. The residue was distilled under reduced pressure, and separated by column chromatography to obtain compound C-1-6 (31.0 g, yield: 77.42%).

### (3) Synthesis of Compound C-1-5

Compound C-1-6 (31.0 g, 82.77 mmol) was dissolved in 93 ml of methylene chloride, then 310 ml of methanol, K₂CO₃ (14.87 g, 107.60 mmol), and 16 ml of distilled water were added, and the mixture was stirred at room temperature. After 1 hour, distilled water was added, and the organic layer was extracted with methylene chloride. The organic layer was then dried with MgSO₄ and filtered under reduced pressure. The residue was distilled under reduced pressure, and separated by column chromatography to obtain compound C-1-5 (24 g, yield: 95.92%).

### (4) Synthesis of Compound C-1-4

Compound C-1-5 (24 g, 79.37 mmol) and 1000 ml of acetone were mixed, NBS (17.65 g, 99.22 mmol) and AgNO₃ (1.4 g, 8.26 mmol) were added, and then the mixture was stirred at room temperature. After 1 hour, an aqueous sodium thiosulfate solution was added, followed by stirring. After 30 minutes, the organic layer was extracted with methylene chloride. An aqueous solution of Na₂CO₃ was added to neutralize, and was then dried over MgSO₄ and filtered under reduced pressure. The residue was distilled under reduced pressure, and separated by column chromatography to obtain compound C-1-4 (24 g, yield: 79.31%).

### (5) Synthesis of Compound C-1-3

Compound C-1-4 (24 g, 62.95 mmol) and 800 ml of toluene were mixed, PtCl₂ (1.0 g, 3.77 mmol) was added, and the mixture was stirred under reflux. After 5 hours, the mixture was cooled to room temperature, distilled water was added, and the organic layer was extracted with ethyl acetate. It was then dried with MgSO₄ and filtered under reduced pressure. The residue was distilled under reduced pressure, and separated by column chromatography to obtain compound C-1-3 (18.0 g, yield: 75.00%).

### (6) Synthesis of Compound C-1-2

Compound C-1-3 (18.0 g, 47.21 mmol) was dissolved in methylene chloride, 70.82 ml of BBr₃ (1M in methylene chloride) was added at 0° C., and after 30 minutes, the mixture was stirred at room temperature for 12 hours. Then, the reaction solution was added to ice water and stirred. After 30 minutes, the organic layer was extracted with methylene chloride. An aqueous solution of Na₂CO₃ was added to neutralize, was dried over MgSO₄, and then filtered under reduced pressure. The residue was distilled under reduced pressure, and separated by column chromatography to obtain compound C-1-2 (8.0 g, yield: 46.16%).

### (7) Synthesis of Compound C-1-1

Compound C-1-2 (8.0 g, 21.78 mmol) was mixed with N-methyl-2-pyrrolidone (NMP), and K₂CO₃ (15 g, 108.93 mmol) was added. After stirring at 120° C. for 1 hour, the mixture was cooled to room temperature. Distilled water was added, and the resulting solid was filtered under reduced pressure. The solid was dissolved in methylene chloride, dried over MgSO₄, and filtered under reduced pressure. After distillation under reduced pressure, the solid was separated by column chromatography to obtain compound C-1-1 (7.5 g, yield: 99.20%) was obtained.

### (8) Synthesis of Compound C-1

Compound C-1-1 (7.0 g, 20.16 mmol), (10-phenylanthracen-9-yl)boronic acid (6.61 g, 22.17 mmol), Pd(OAc)2 (0.18 g, 0.80 mmol), S-phos (0.99 g, 2.42 mmol), K₃PO₄ (10.69 g, 50.40 mmol), 150 ml of toluene, 50 ml of distilled water, and 25 ml of ethanol were added, and the mixture was stirred under reflux. After 2 hours and 40 minutes, the mixture was cooled to room temperature, distilled water was added, and the organic layer was extracted with methylene chloride. It was then dried with MgSO₄ and filtered under reduced pressure. The residue was distilled under reduced pressure, and separated by column chromatography to obtain compound C-1 (7.2 g, yield: 68.63%).

| | MW | MP |
|---|---|---|
| C-1 | 520.63 | 282.4° C. |

### Example 2: Synthesis of Compound C-136

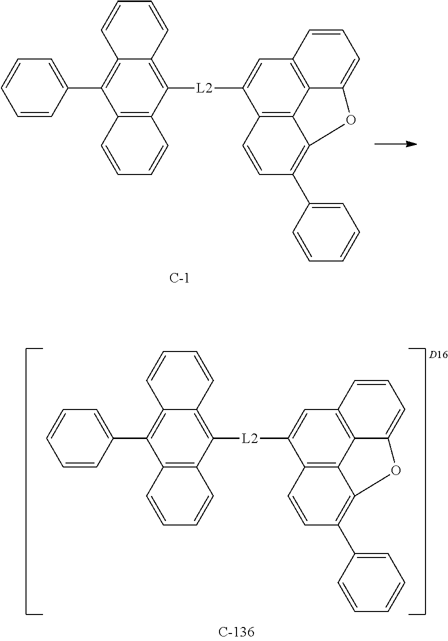

Compound C-1 was synthesized by a method selected from the deuteration methods disclosed in Korean Patent Nos. 10-2283849 and 10-1427457 to obtain compound C-136-D16 (4.4 g yield: 73,66%, MS: [M+H]+=537.2).

| | MW | MP |
|---|---|---|
| C-136 | 536.2 | 298° C. |

### Device Example 1: Preparation of OLED Comprising the Compound According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI was introduced into a cell of the vacuum vapor deposition apparatus, and compound HT-1 was introduced into another cell. The two materials were evaporated at different rates, and compound HI was deposited in a doping amount of 5 wt % based to the total amount of compound HI and compound HT-1 to form a hole injection layer with a thickness of 10 nm. Subsequently, compound HT-1 was deposited on the hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 15 nm on the first hole transport layer. After forming the hole injection layer and the hole transport layers, a light-emitting layer was deposited thereon as follows: compound C-1 was added as a host into a cell of the vacuum vapor deposition equipment, compound BD was added as a dopant into another cell, and the two materials were evaporated at different rates to dope 2 wt % of the dopant with respect to the total amount of the host and the dopant, thereby depositing a light-emitting layer having a thickness of 22.5 nm on the second hole transport layer. Compound ET-1 was deposited to a thickness of 5 nm as an electron buffer layer, and then Compound EI-1 and Compound El-2 were added to two other cells and evaporated at a rate of 2:1, thereby forming an electron transport layer having a thickness of 25 nm on the light-emitting layer. Compound Yb:LiF was added to two other cells as an electron injection layer and evaporated at a rate of 2:1, an electron injection layer was deposited to a thickness of 1 nm on the electron transport layer, and then an Al cathode was deposited to a thickness of 80 nm using other vacuum deposition equipment to prepare OLED. All of the compounds used for the materials were purified by vacuum sublimation under 10⁻⁶ torr.

### Device Example 2: Preparation of OLED Comprising the Compound According to the Present Disclosure

OLED was prepared in the same manner as in Device Example 1, except that only compound C-136 was used, alone, as the host material of the light-emitting layer.

### Device Comparative Example 1: Preparation of OLED Comprising Conventional Compound

OLED was prepared in the same manner as in Device Example 1, except that only compound F-1 was used, alone, as the host material of the light-emitting layer.

The driving voltage, current efficiency (cd/A), and CIE color coordinates at a luminance of 1,000 nits of the organic electroluminescence devices of Device Examples 1 and 2 and Device Comparative Example 1, prepared as described above, were measured; the results thereof are shown in Table 1, as follows:

TABLE 1

| | | |
|---|---|---|
| Driving | Current | CIE color |
| Voltage | Efficiency | coordinates |
| (V) | (cd/A) | (x, y) |

| | |
|---|---|
| Host | 1,000 nit |

| | | | | |
|---|---|---|---|---|
| Device | C-1 | 3.6 | 6.1 | 0.137, 0.069 |
| Example 1 | | | | |
| Device | C-136 | 3.6 | 6.1 | 0.137, 0.068 |
| Example 2 | | | | |
| Device | F-1 | 3.5 | 5.0 | 0.138, 0.064 |
| Comparative | | | | |
| Example 1 | | | | |

### Device Examples 3 to 7: Preparation of OLEDs Comprising the Compound According to the Present Disclosure

OLEDs were prepared in the same manner as in Device Example 1, except that the first host compound and the second host compound were each added in a weight ratio of 1:1 as the host material of the light-emitting layer, and deposited.

### Device Comparative Examples 2 and 3: Preparation of OLEDs Comprising Conventional Compound

OLEDs were prepared in the same manner as in Device Example 1, except that only compounds H1-42 and F-1, respectively, were used alone as the host material of the light-emitting layer.

The minimum time (lifespan: T95) taken for the intensity of light to fall from 100% to 95% at the luminance according to the driving voltage, and power efficiency (cd/A), CIE color coordinate, and CIE color coordinate at a luminance of 1,000 nits of the organic electroluminescent device of Device Examples 3 to 7 and Device Comparative Examples 2 and 3, prepared as described above, were measured; the results thereof are shown in Table 2, as follows:

TABLE 2

| | | | Driving | Current | CIE color | |
|---|---|---|---|---|---|---|
| | | | Voltage | Efficiency | coordinates | |
| | Host 1 | Host 2 | (V) | (cd/A) | (x, y) | Lifespan |
| | Material | Material | 1,000 nit | | | (T95, hr) |
| Device | H1-82 | C-136 | 3.7 | 5.9 | 0.138, 0.064 | 87 |
| Example 3 | | | | | | |
| Device | H1-66 | C-136 | 3.7 | 6.0 | 0.138, 0.064 | 69 |
| Example 4 | | | | | | |
| Device | H1-105 | C-1 | 3.8 | 5.9 | 0.139, 0.064 | 47 |
| Example 5 | | | | | | |
| Device | H1-106 | C-1 | 3.8 | 6.1 | 0.138, 0.065 | 39 |
| Example 6 | | | | | | |
| Device | H1-42 | C-1 | 3.7 | 6.0 | 0.137, 0.067 | 27 |
| Example 7 | | | | | | |
| Device | H1-42 | — | 3.8 | 5.7 | 0.137, 0.069 | 23 |
| Comparative | | | | | | |
| Example 2 | | | | | | |
| Device | F-1 | — | 3.5 | 5.0 | 0.138, 0.064 | 42 |
| Comparative | | | | | | |
| Example 3 | | | | | | |

From Table 1, it can be confirmed that the organic electroluminescent device comprising the specific compound according to the present disclosure showed significantly improved driving voltage and/or current efficiency compared to the conventional organic electroluminescent device. In particular, it can be confirmed that the blue OLED shows excellent power efficiency of 6 cd/A or more. The inventors of the present disclosure have predicted that the excellent effect of the specific compound according to the present disclosure is based on an increase in the resonance of molecules, and thus the intermolecular interaction as the phenyl substituent of the phenyl-substituted phenanthrofuran bound to anthracene is bound at a specific position in the compound.

From Table 2, it can be confirmed that the organic electroluminescent device comprising the plurality of host materials according to the present disclosure has significantly improved driving voltage and/or current efficiency compared to the conventional organic electroluminescent device. In particular, it can be confirmed that the blue OLED exhibits excellent power efficiency of more than 6 cd/A.

The compounds used in the Device Examples and the Device Comparative Examples are specifically shown in Table 3, as follows:

TABLE 3

| | |
|---|---|
| Hole Injection Layer/ Hole Transport Layer | 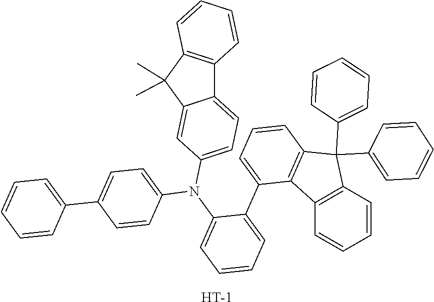 HI 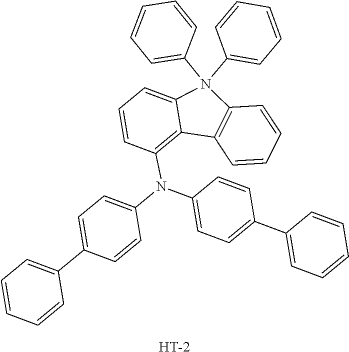 |
| Light-emitting Layer | C-1 |
| | C-136-D16 |
| | F-1 |
| | H1-82-D20 |
| | H1-66-D22 |
| | H1-105 |
| | H1-106 |
| | H1-42 |
| Electron Buffer | |
| Layer | |
| Electron Transport | |
| Layer/ | |
| Electron Injection | |
| Layer | |

1. A plurality of host materials, comprising one or more first host compounds and one or more second host compounds different from the first host compounds, wherein the first host compound is represented by the following formula 1: 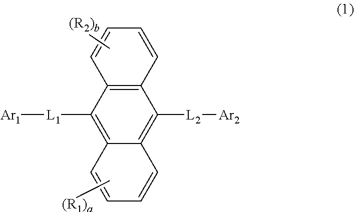
wherein,
Ar₁ and Ar₂ each independently represent a substituted or unsubstituted (C6-C30)aryl or a substituted or unsubstituted (3- to 30-membered)heteroaryl;
L₁ and L₂ each independently represent a single bond, a substituted or unsubstituted (C6-C30)arylene, or a substituted or unsubstituted (3- to 30-membered)heteroarylene;
R₁ and R₂ each independently represent: hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —N—(Ar₇)(Ar₈);
Ar₇ and Ar₈ each independently represent: hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
a and b each independently represent an integer of 1 to 4, where if a and b each independently represent an integer of 2 or more, each of R₁ and R₂ may be the same or different, and
wherein the second host compound is represented by the following formula 2: 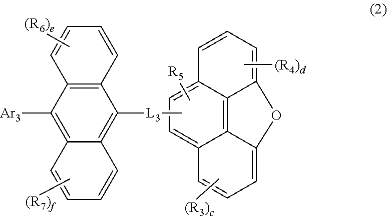
wherein,
Ar₃ represents a substituted or unsubstituted (C6-C30)aryl or a substituted or unsubstituted (3- to 30-membered)heteroaryl;
L₃ represents a single bond, a substituted or unsubstituted (C6-C30)arylene, or a substituted or unsubstituted (3- to 30-membered)heteroarylene;
R₃ to R₇ each independently represent: hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —N—(Ar₉)(Ar₁₀);
Ar₉ and Ar₁₀ each independently represent: hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
c and d each independently represent an integer of 1 to 3; and e and f each independently represent an integer of 1 to 4, where if c to f are an integer of 2 or more, each of R₃, R₄, R₆, and R₇ may be the same or different.

2. The plurality of host materials according to claim 1, wherein Ar₁ and Ar₂ of formula 1 and Ar₃ of formula 2 each independently represent: a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted triphenylenyl, a substituted or unsubstituted fluorenyl, a substituted or unsubstituted dibenzofluorenyl, a substituted or unsubstituted dibenzofuranyl, a substituted or unsubstituted dibenzothiophenyl, a substituted or unsubstituted carbazolyl, or a substituted or unsubstituted dibenzoselenophenyl.

3. The plurality of host materials according to claim 1, wherein the substituted (C1-C30)alkyl, the substituted (C3-C30)cycloalkyl, the substituted (C1-C30)alkoxy, the substituted (C6-C30)aryl, the substituted (C6-C30)arylene, the substituted (3- to 30-membered)heteroaryl, and the substituted (3- to 30-membered)heteroarylene are each independently substituted with one or more selected from the group consisting of: deuterium, halogen, cyano, carboxyl, nitro, hydroxy, phosphine oxide, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (3- to 30-membered)heteroaryl substituted or unsubstituted with at least one of deuterium and (C6-C30)aryl, (C6-C30)aryl substituted or unsubstituted with at least one of deuterium and (3- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, mono- or di-(C2-C30)alkenylamino; mono- or di-(C6-C30)arylamino, mono- or di-(3- to 30-membered)heteroarylamino, (C1-C30)alkyl(C2-C30)alkenylamino, (C1-C30)alkyl(C6-C30)arylamino, (C1-C30)alkyl(3- to 30-membered)heteroarylamino, (C2-C30)alkenyl(C6-C30)arylamino, (C2-C30)alkenyl(3- to 30-membered)heteroarylamino, (C6-C30)aryl(3- to 30-membered)heteroarylamino, (C1-C30)alkylcarbonyl, (C1-C30)alkoxycarbonyl, (C6-C30)arylcarbonyl, (C6-C30)arylphosphine, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl.

4. The plurality of host materials according to claim 1, wherein the compound represented by formula 1 is selected from the following compounds: 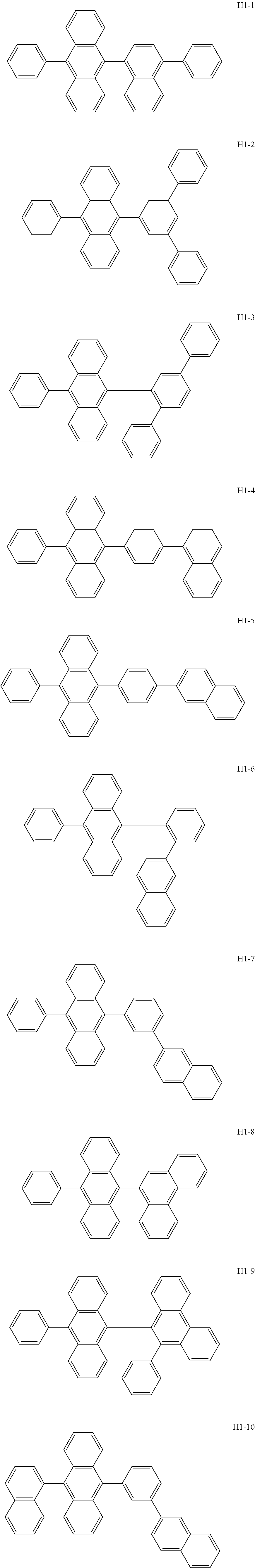 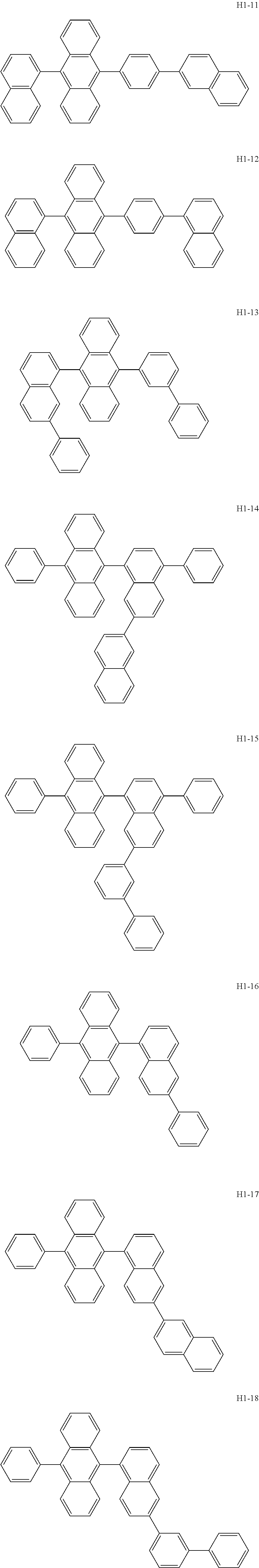 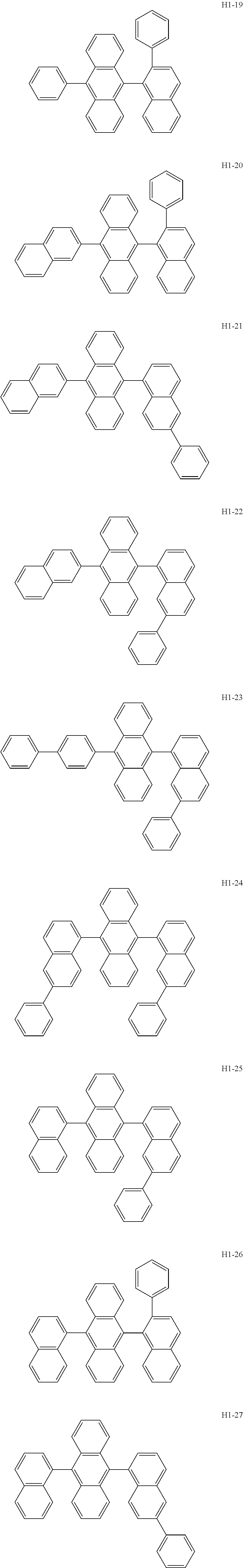 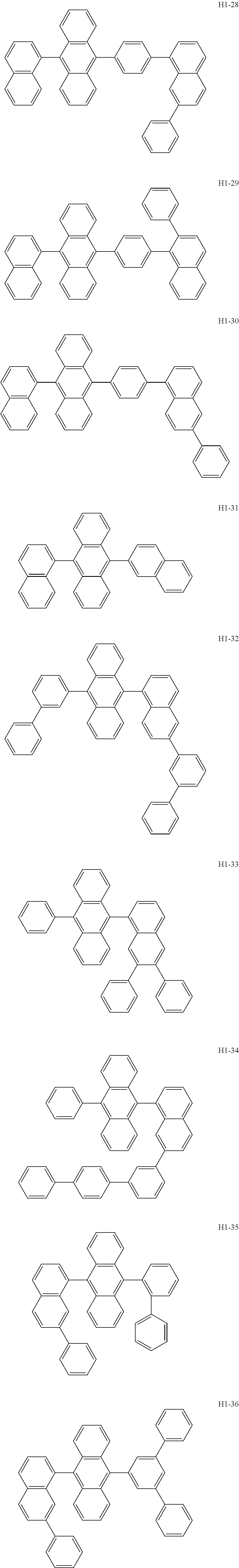 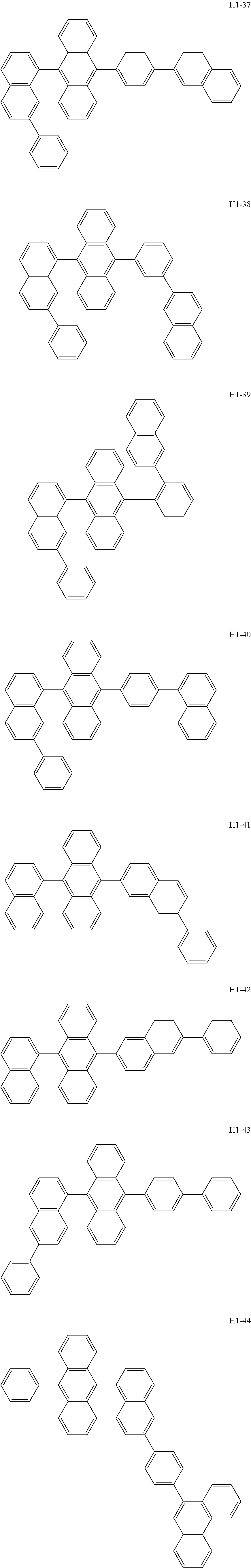 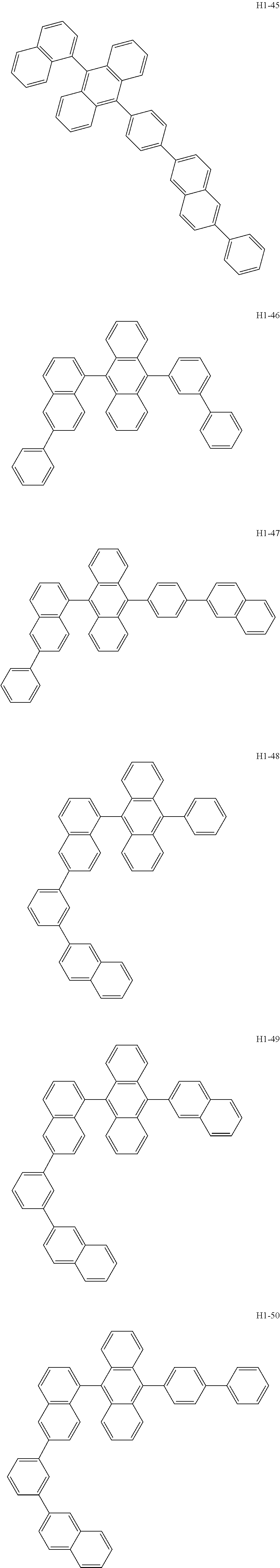 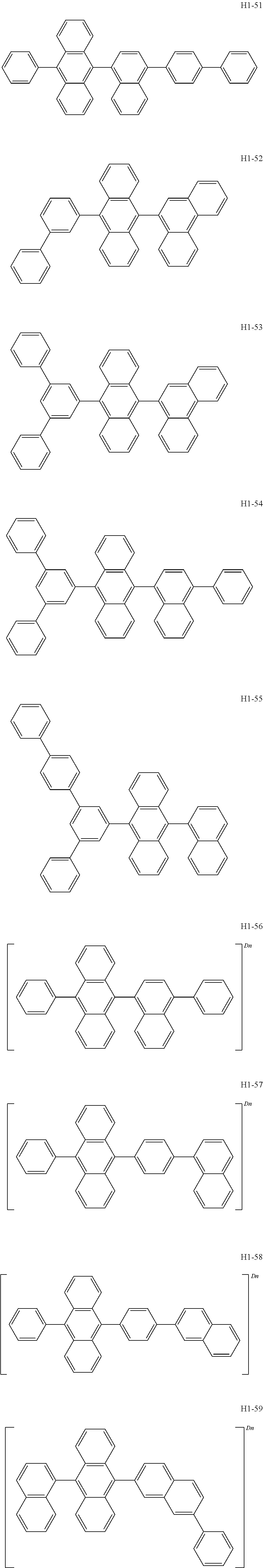 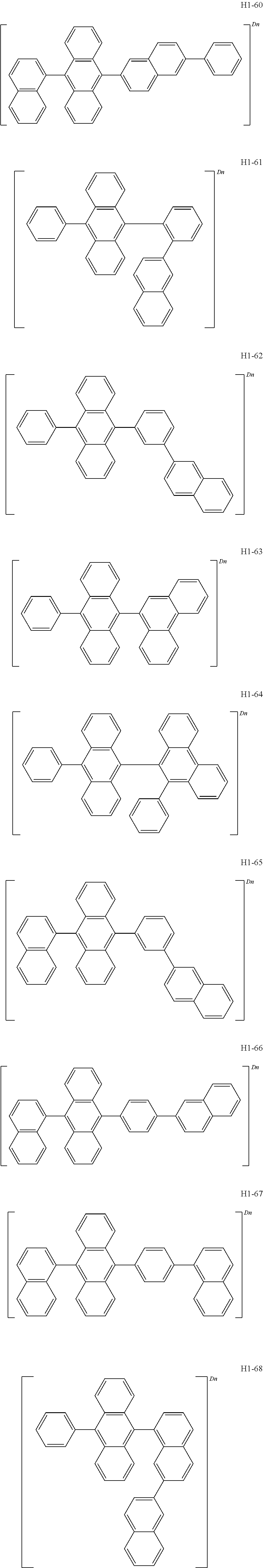 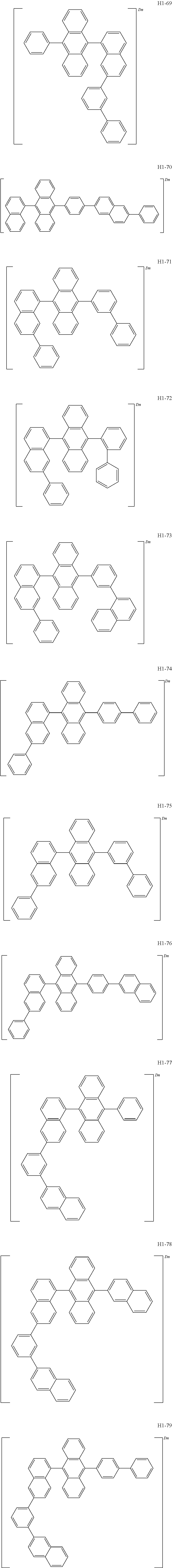 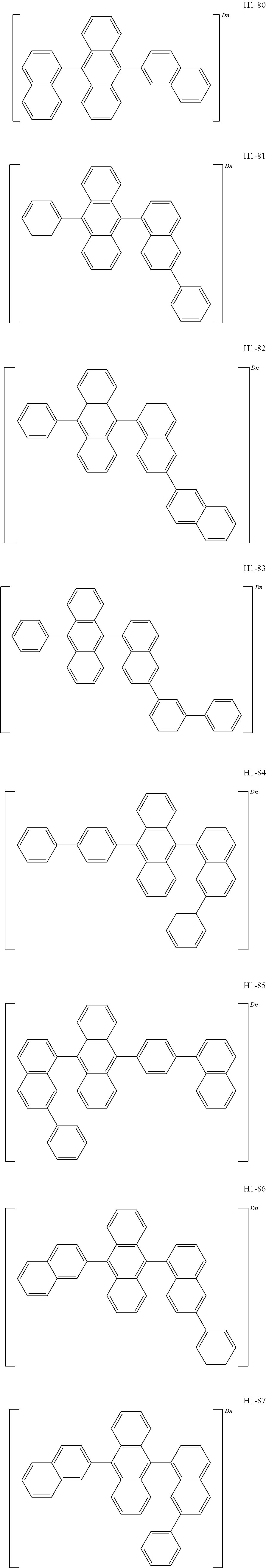 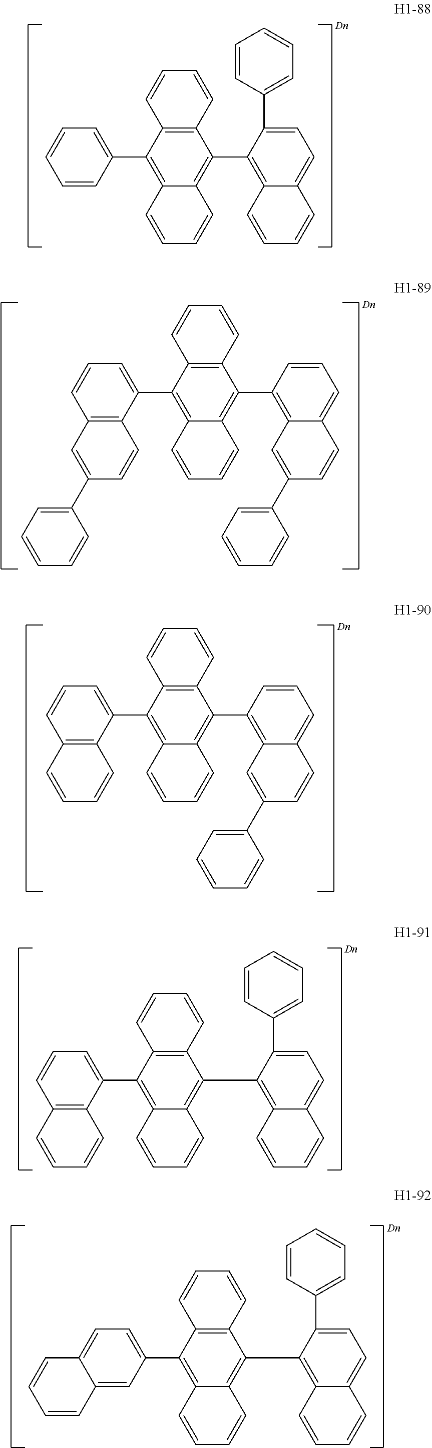 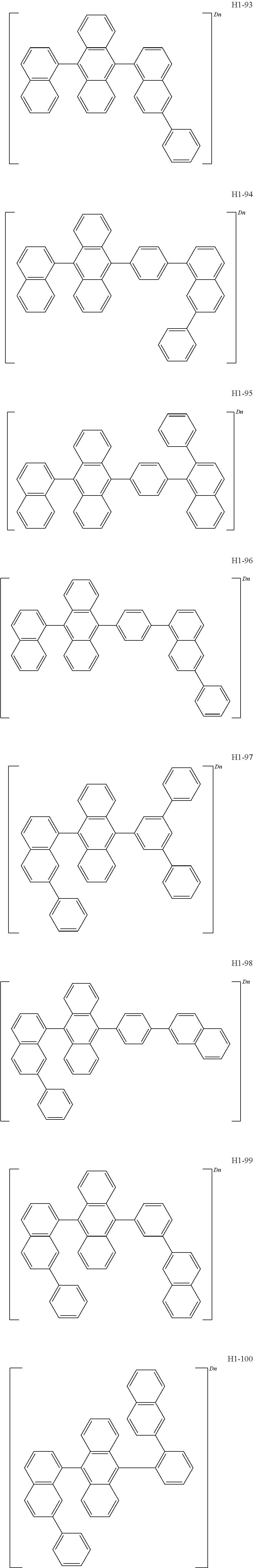 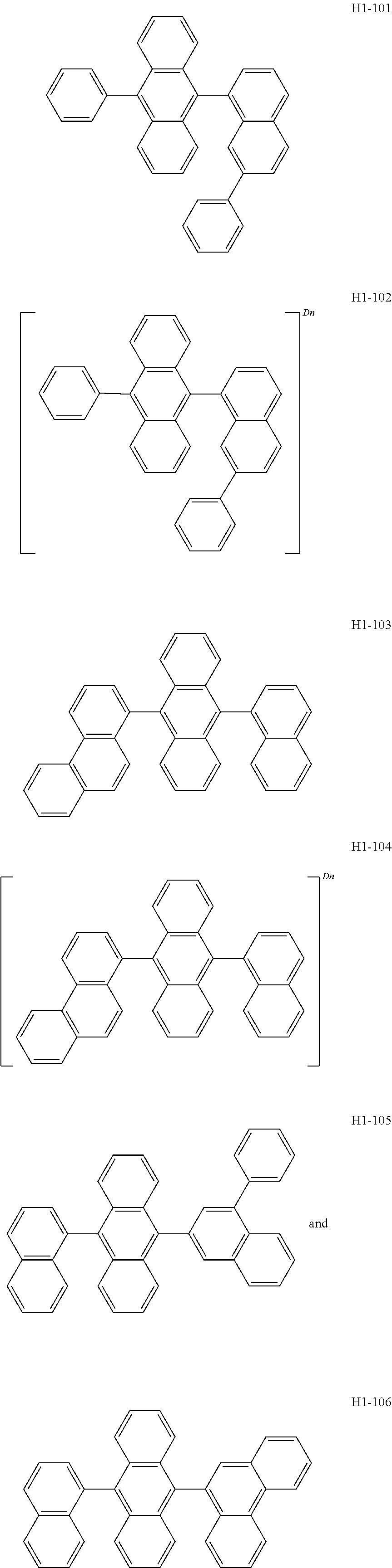
wherein Dn of compounds H1-56 to H1-100, H1-102, and H1-104 means that n hydrogens are replaced by deuteriums, and n is between 1 and the maximum number of hydrogens in each compound.

5. The plurality of host materials according to claim 1, wherein the compound represented by formula 2 is selected from the following compounds: 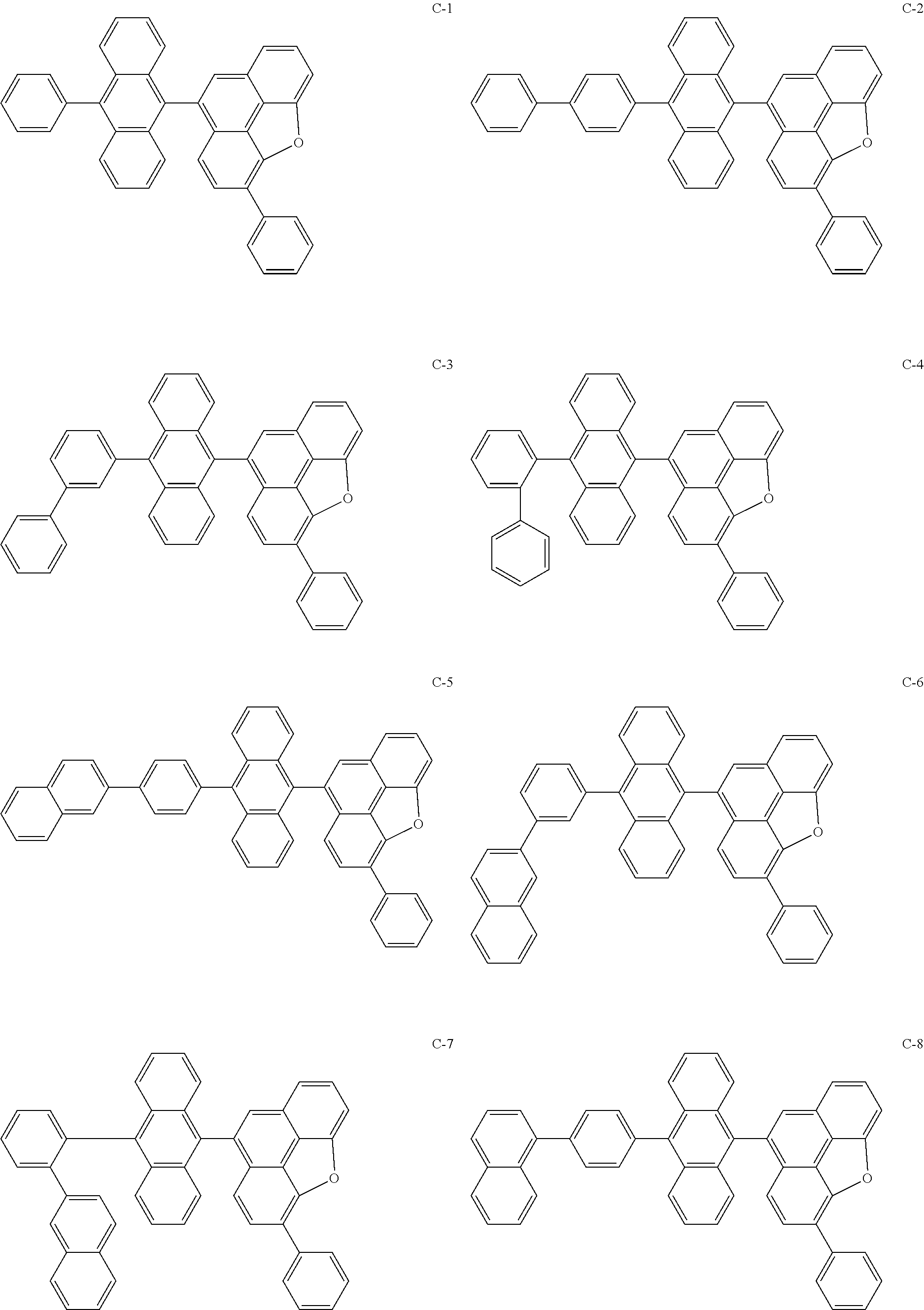 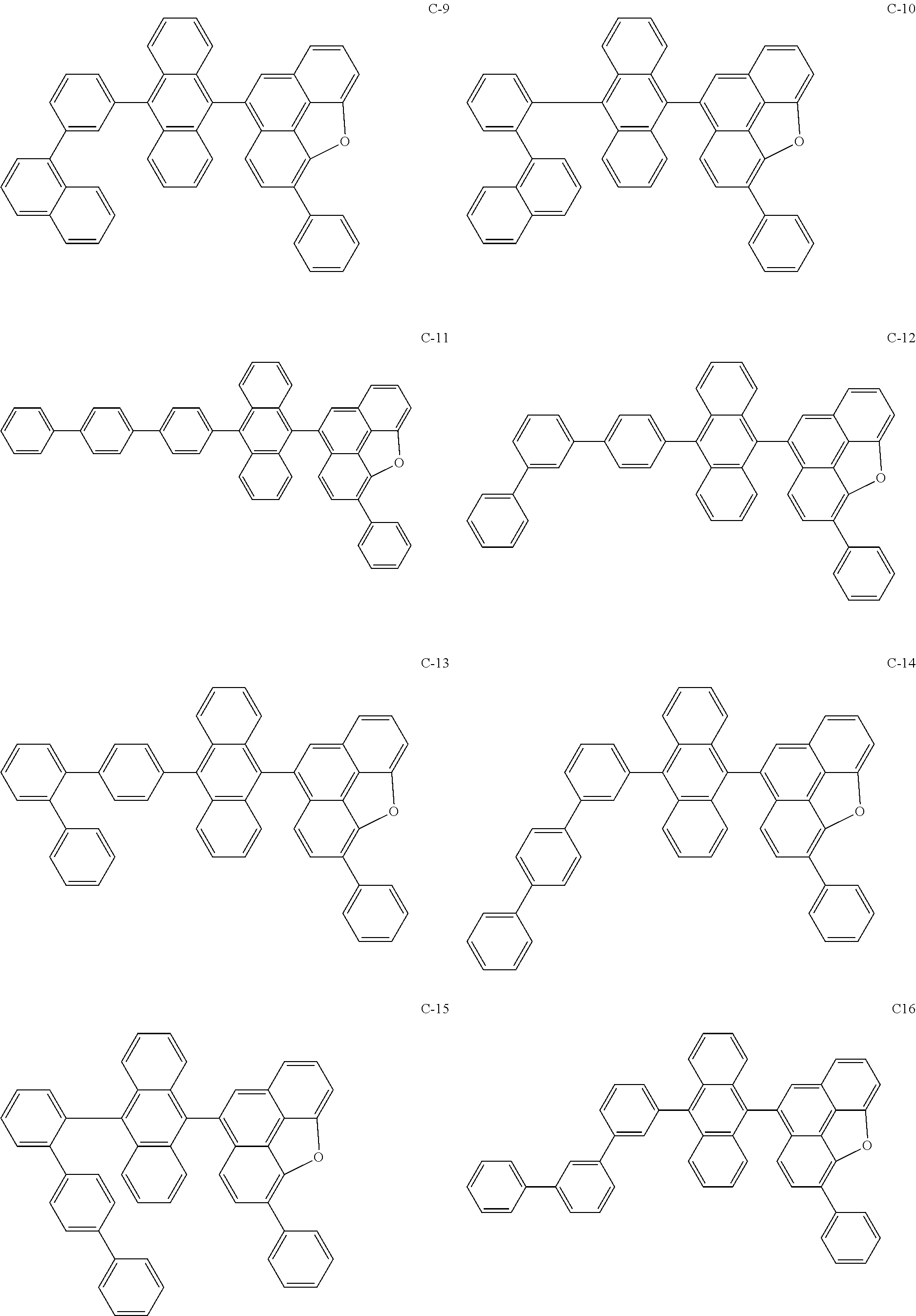 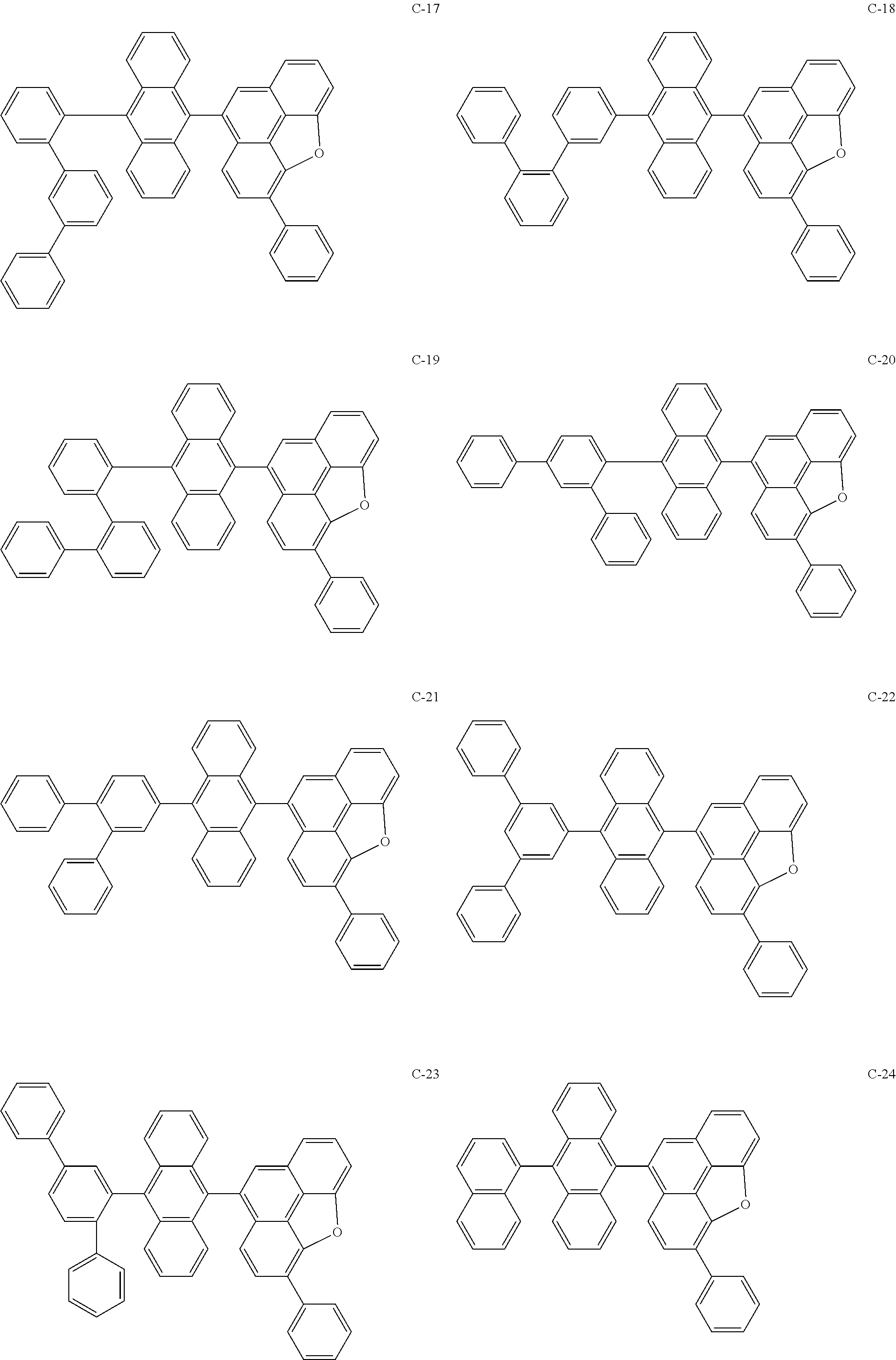 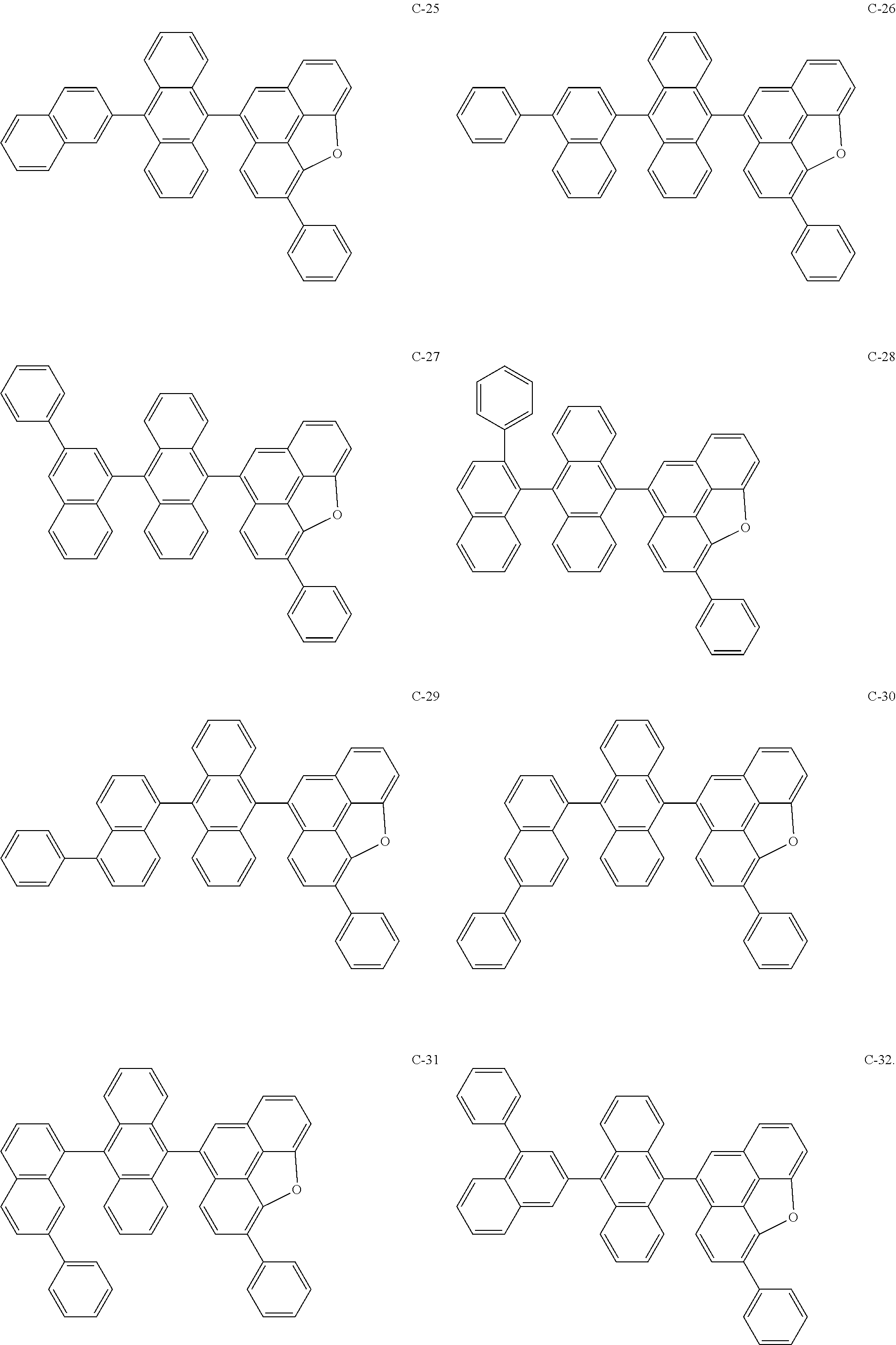 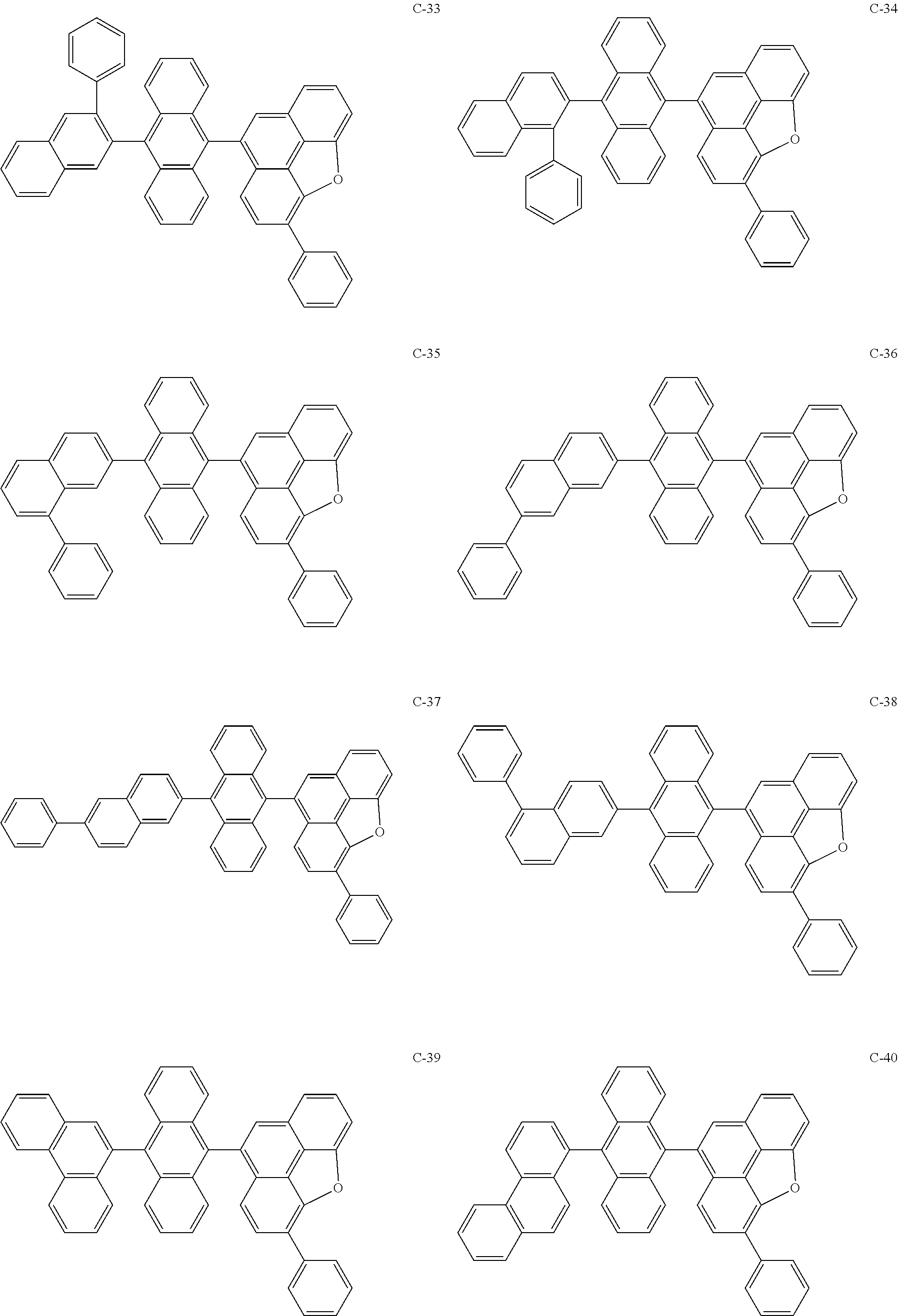 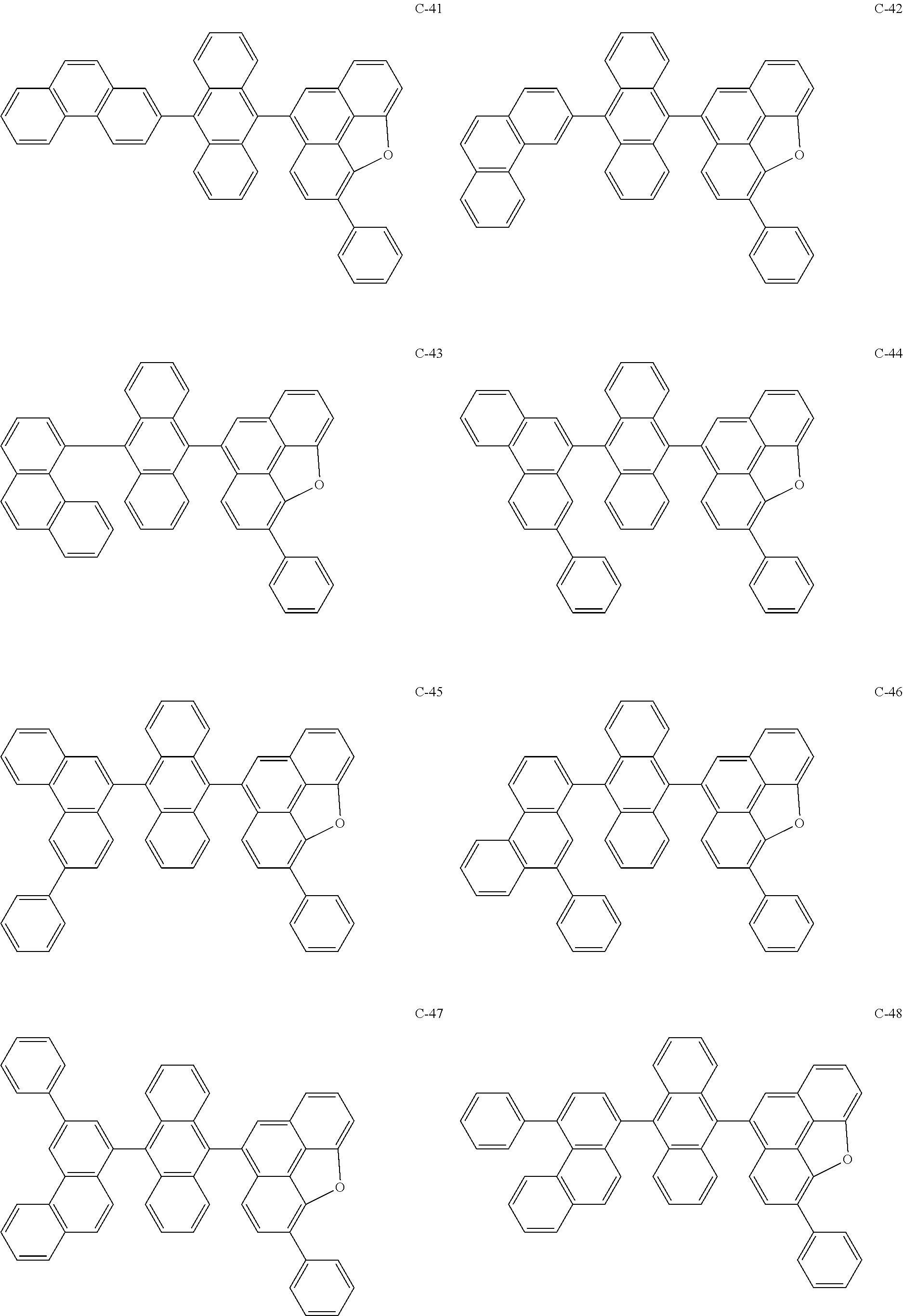 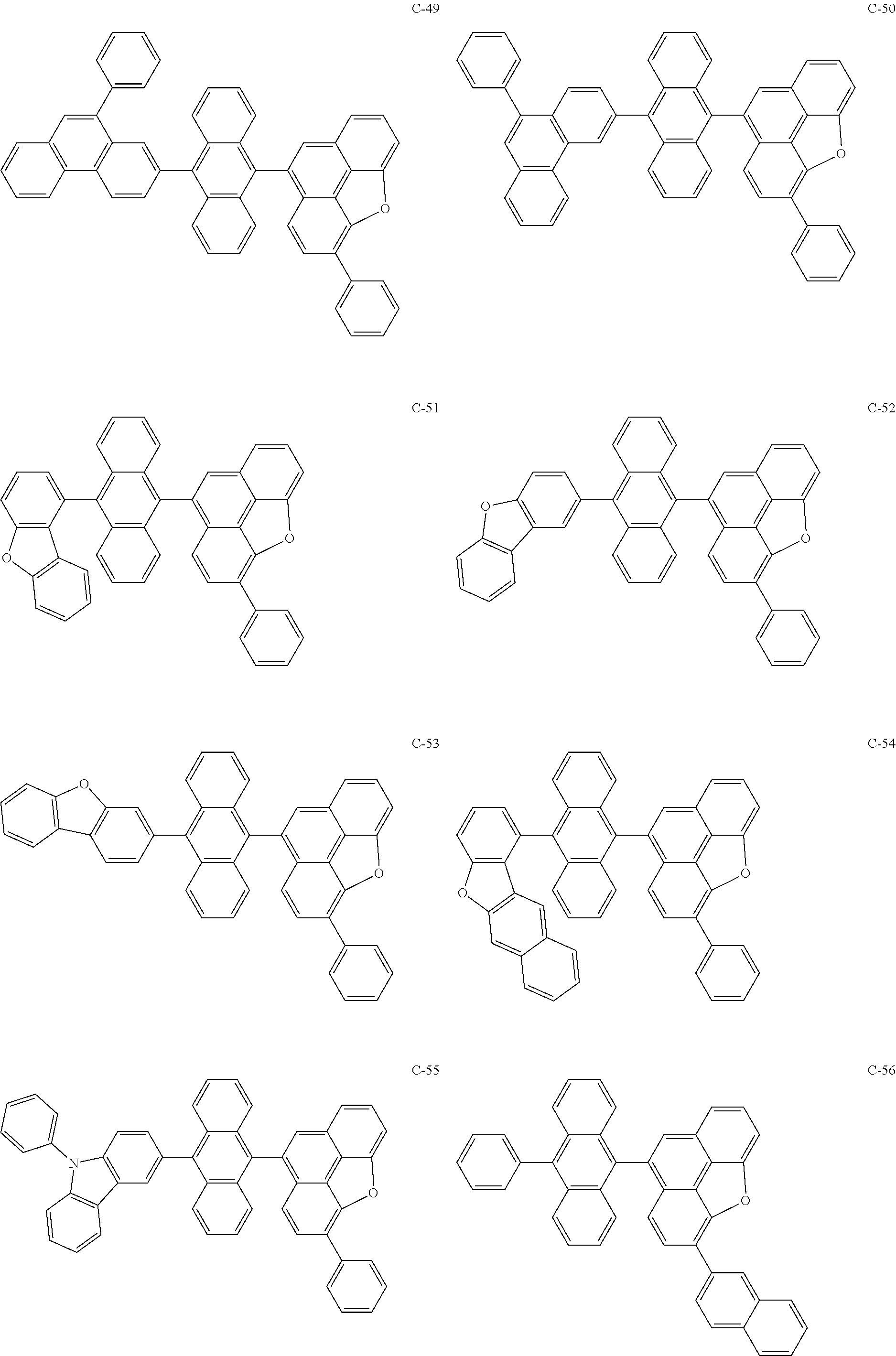 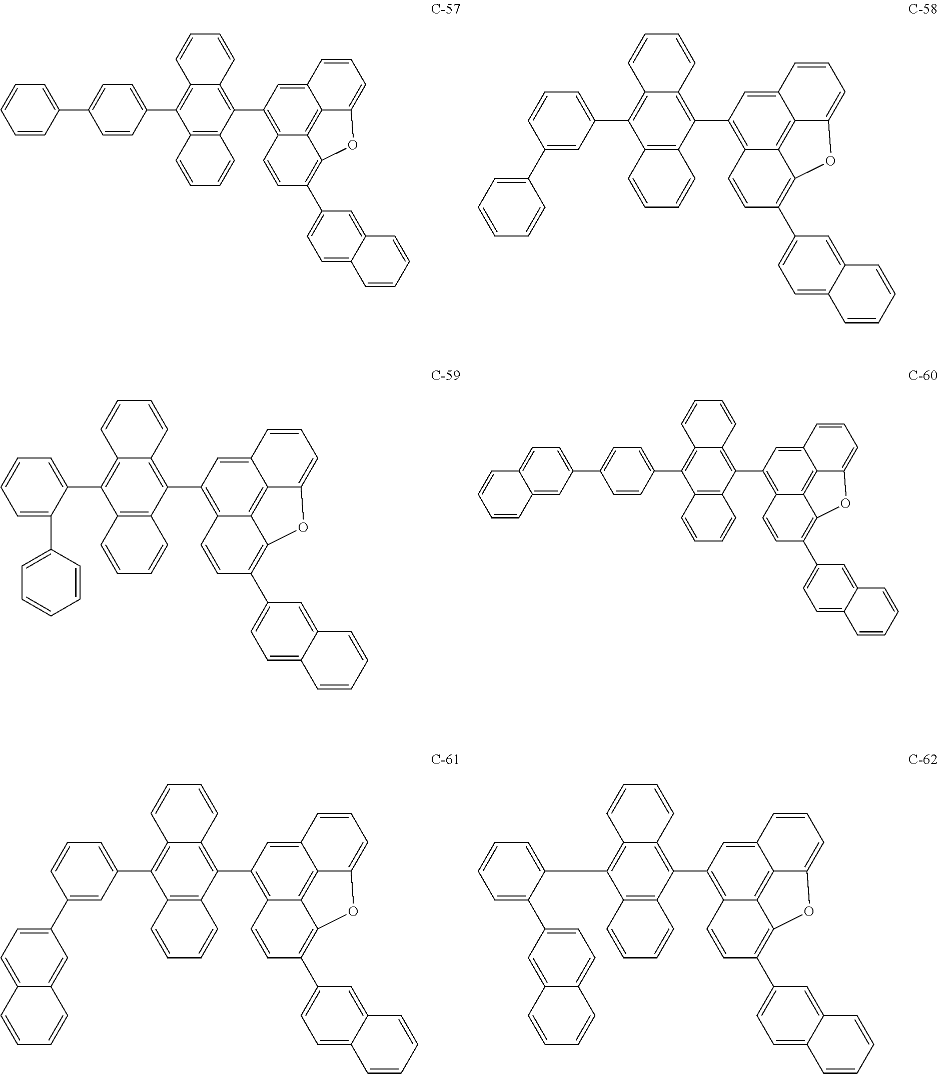 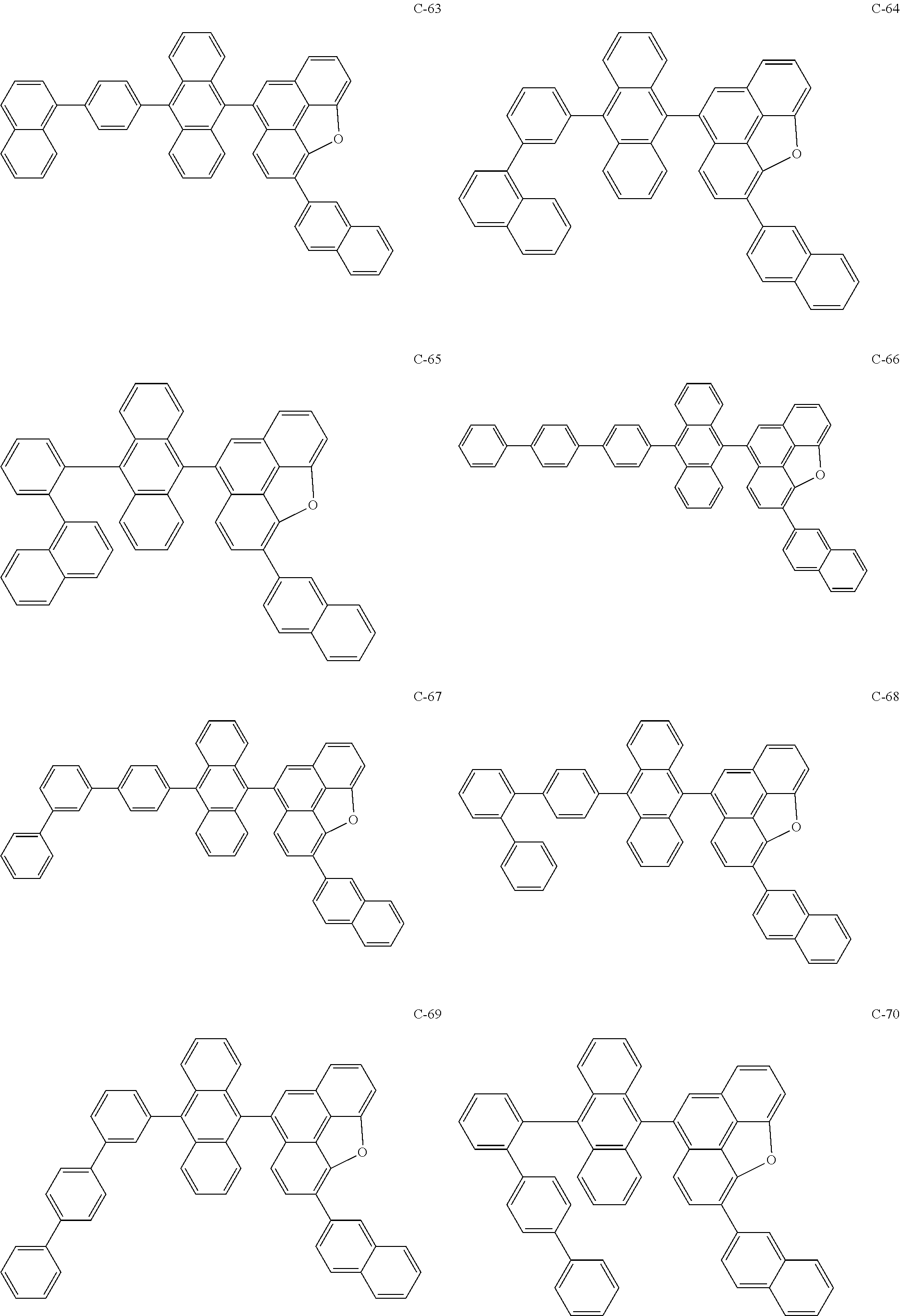 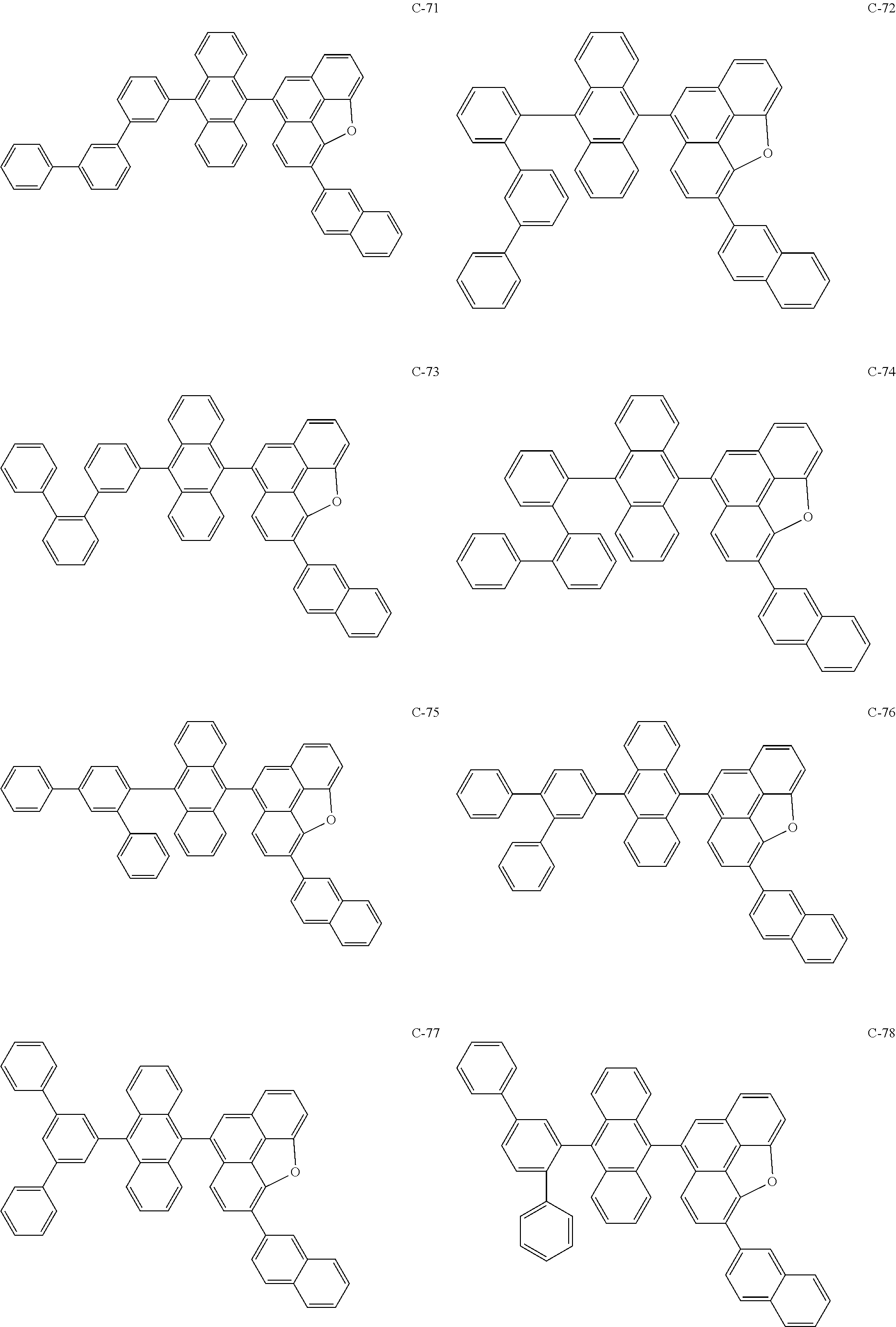 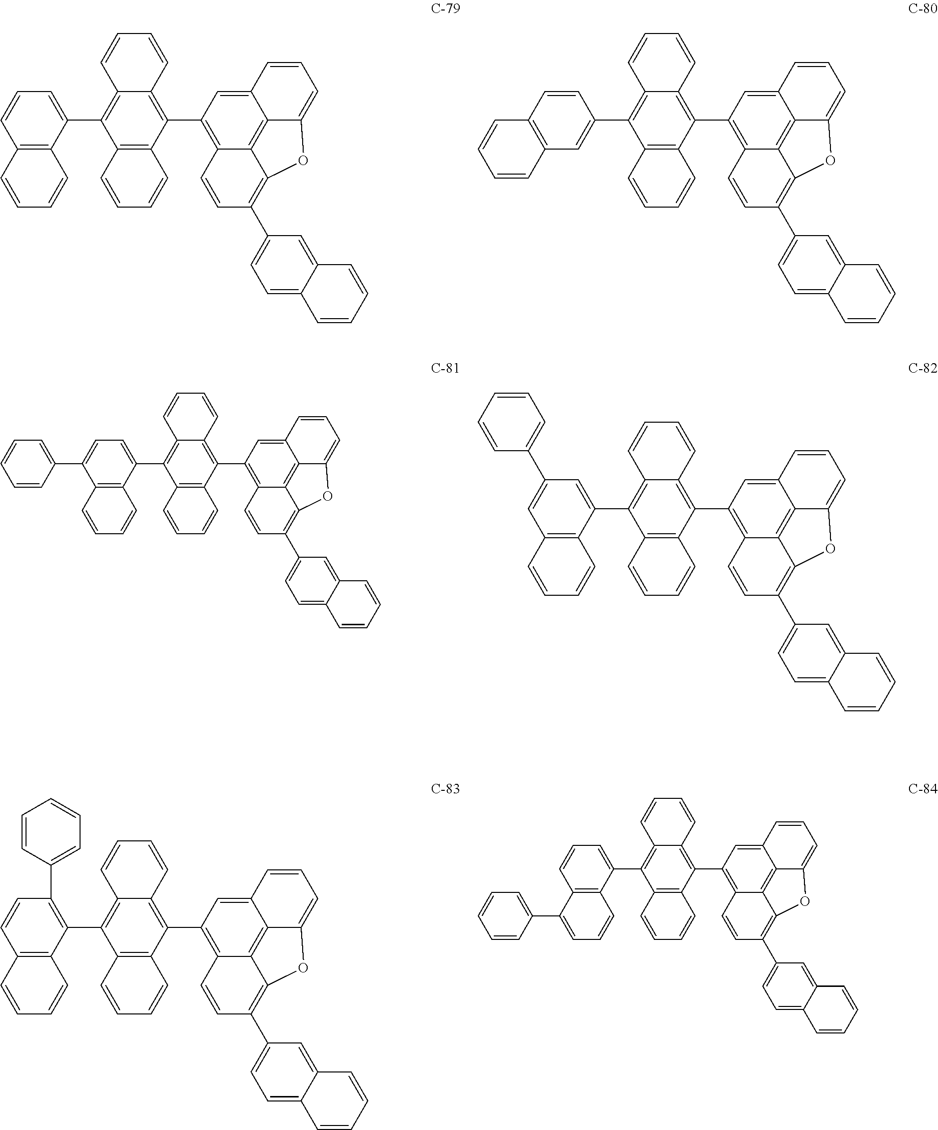 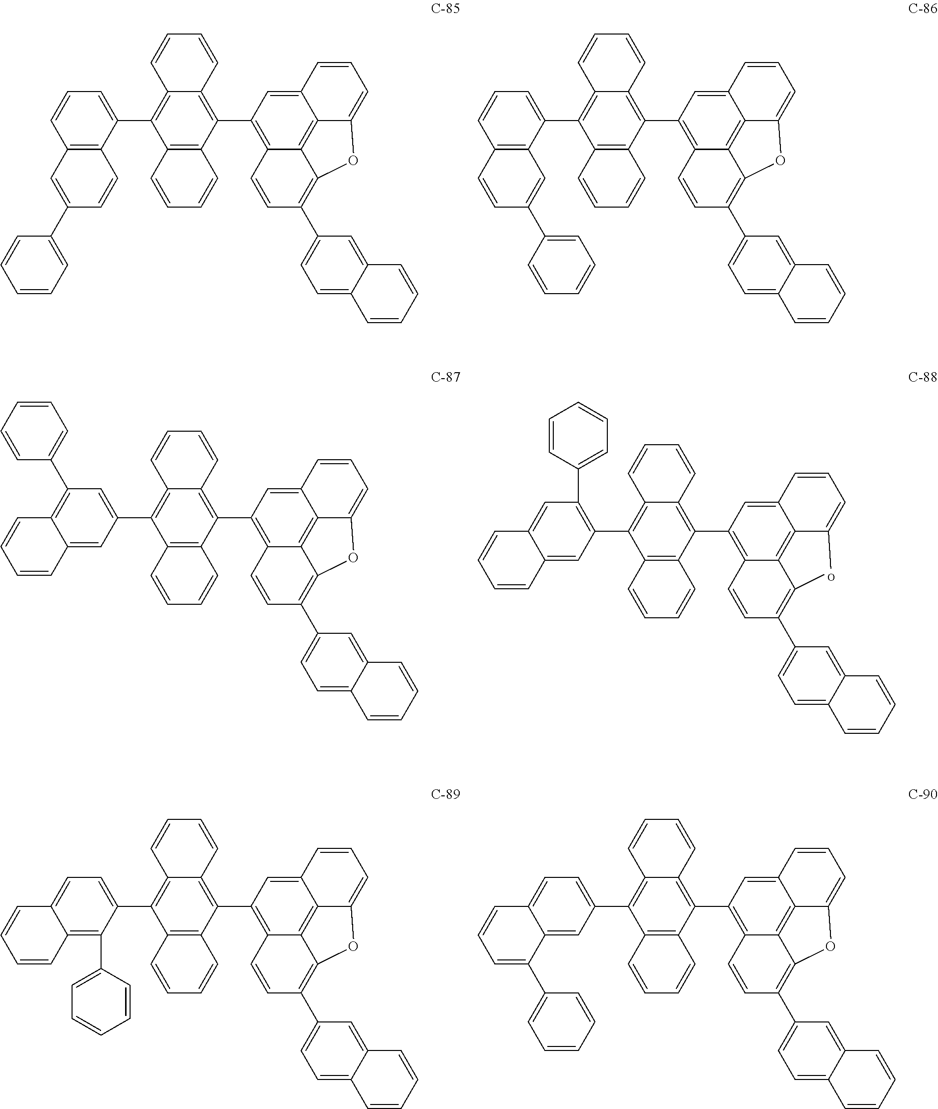 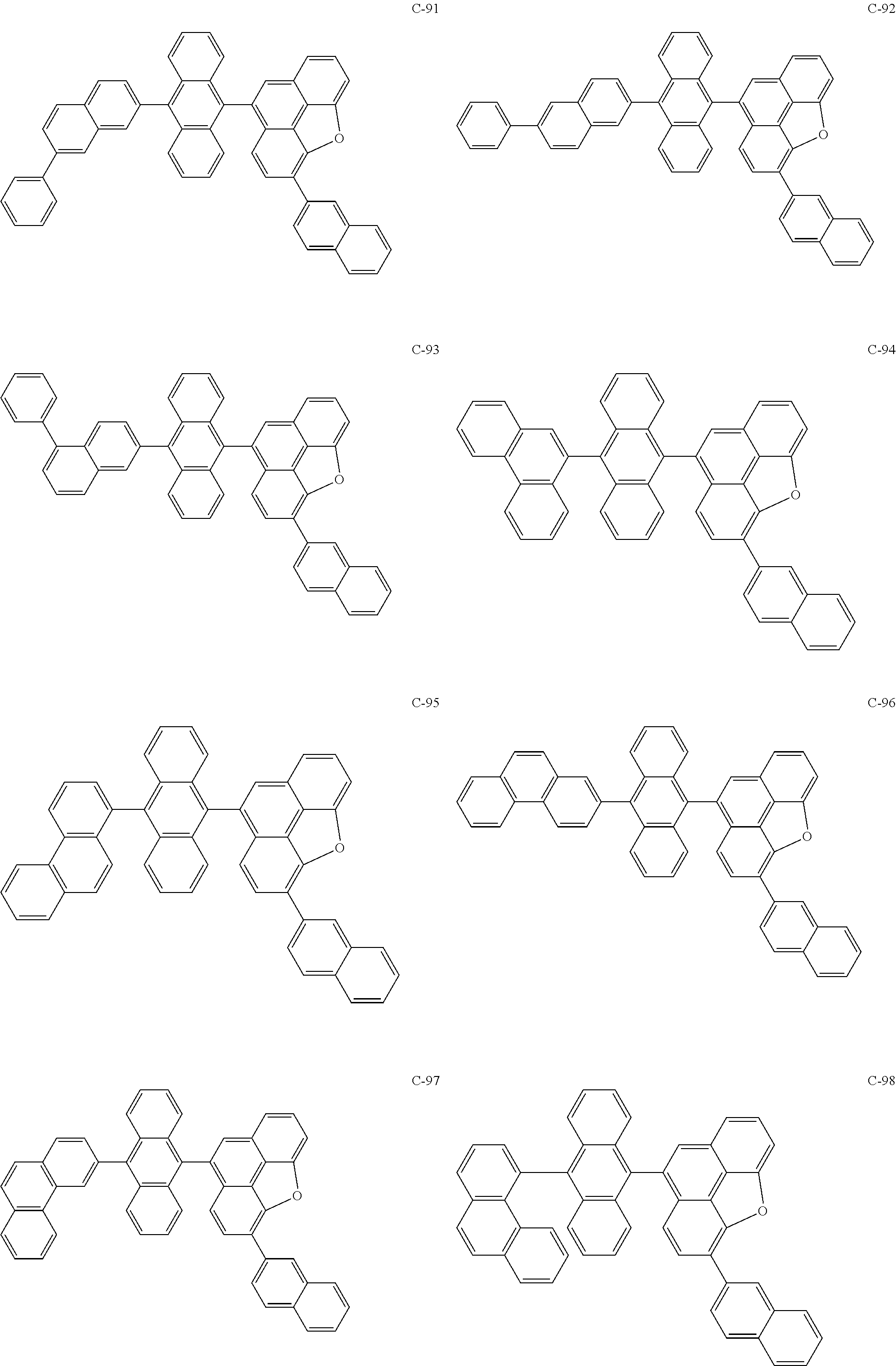 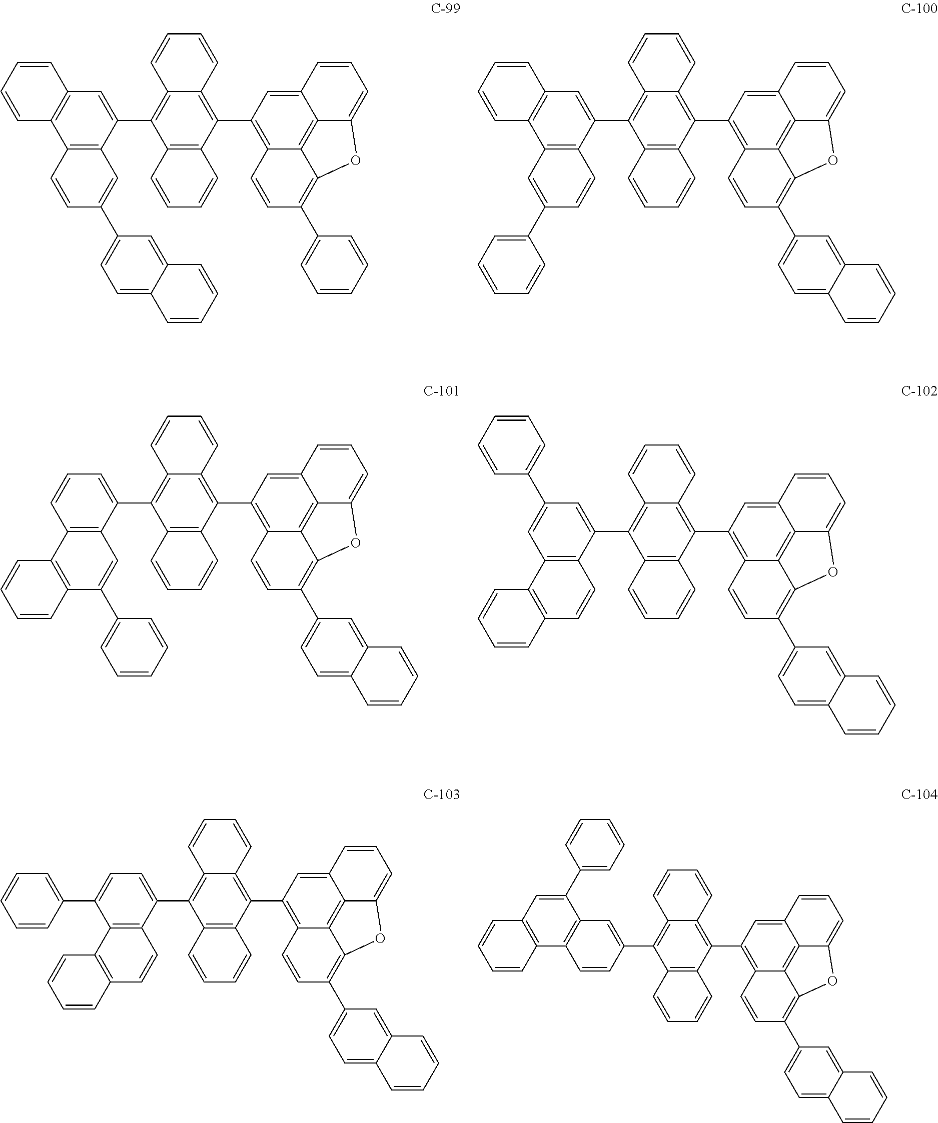 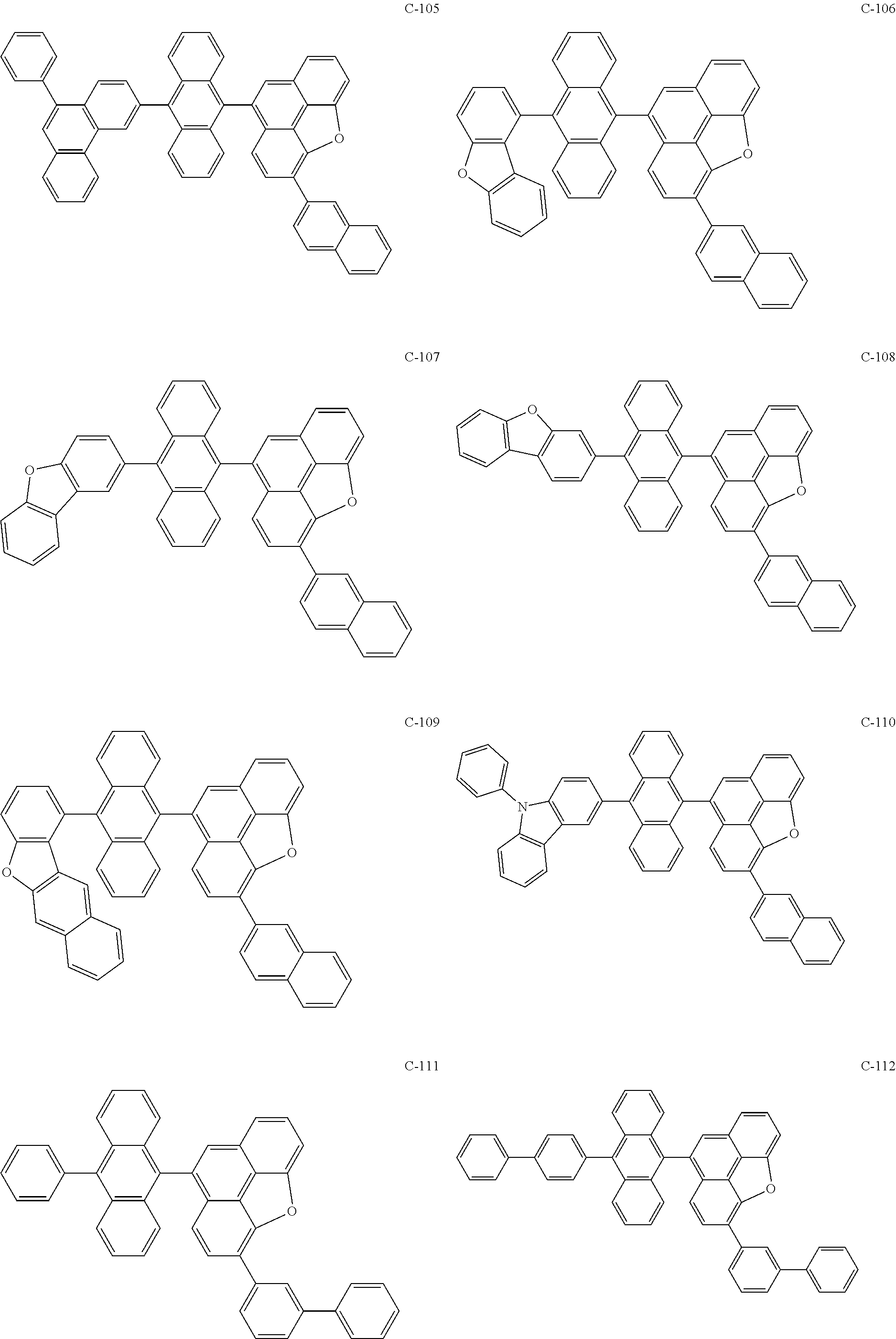 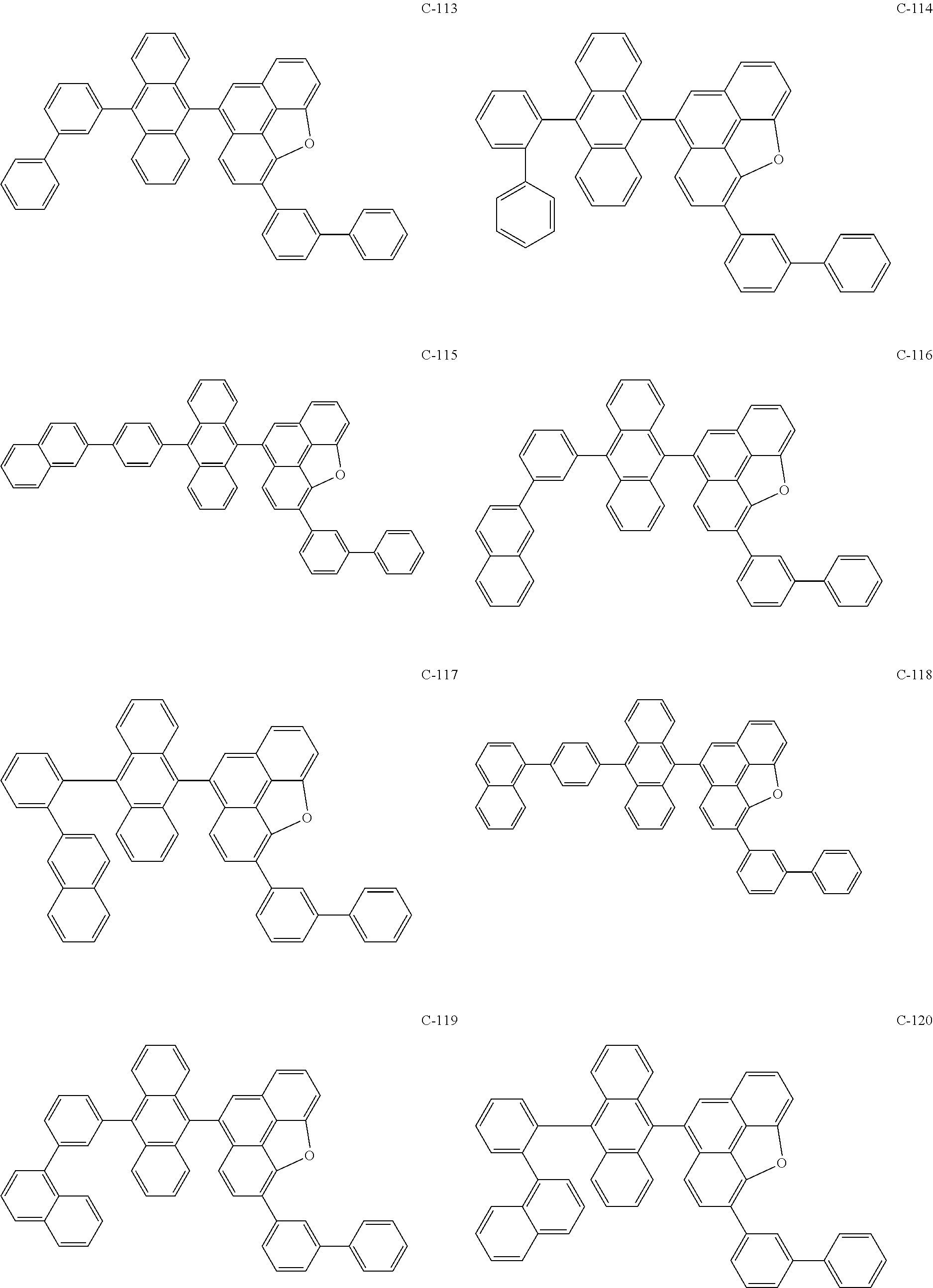 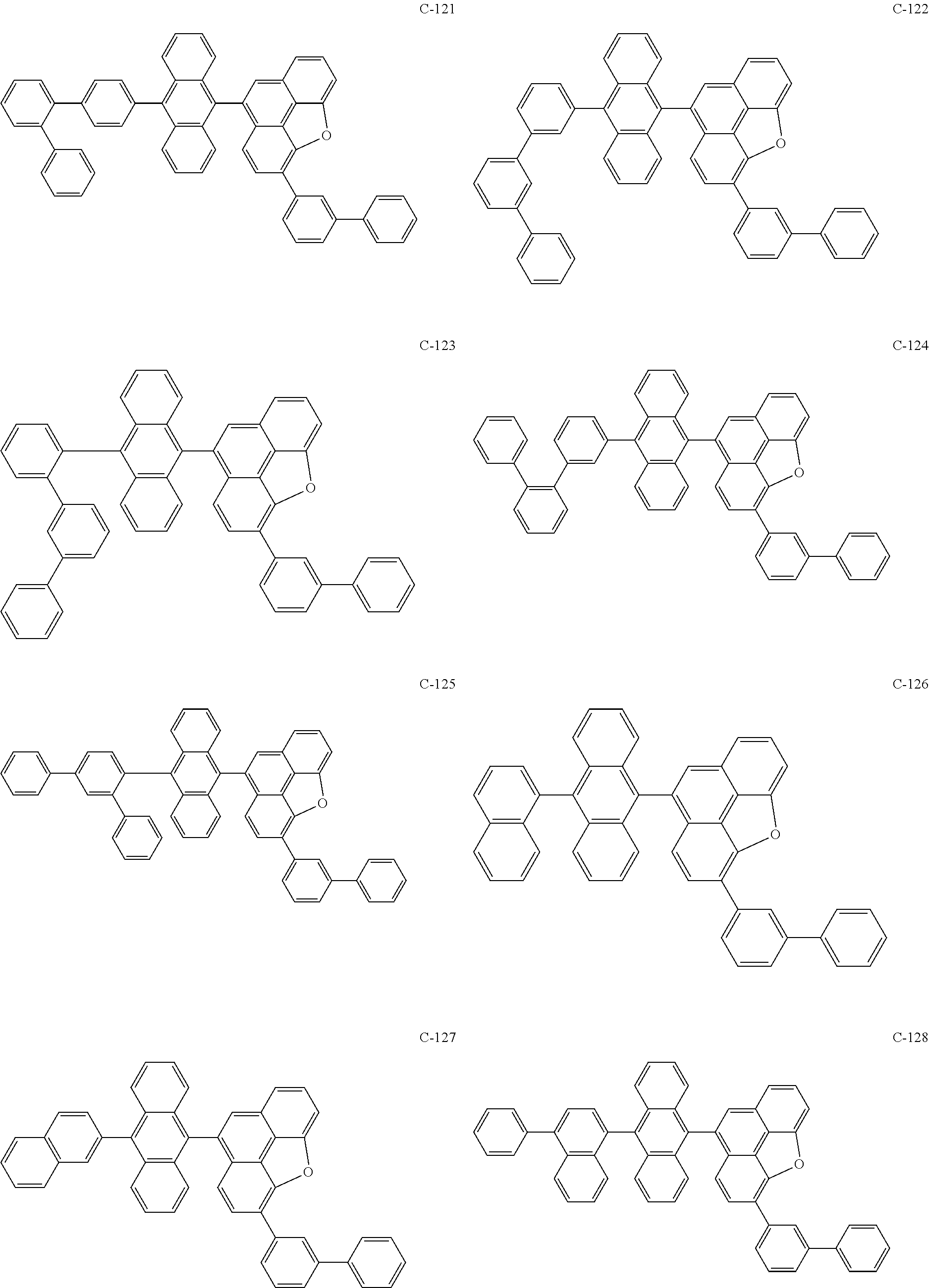 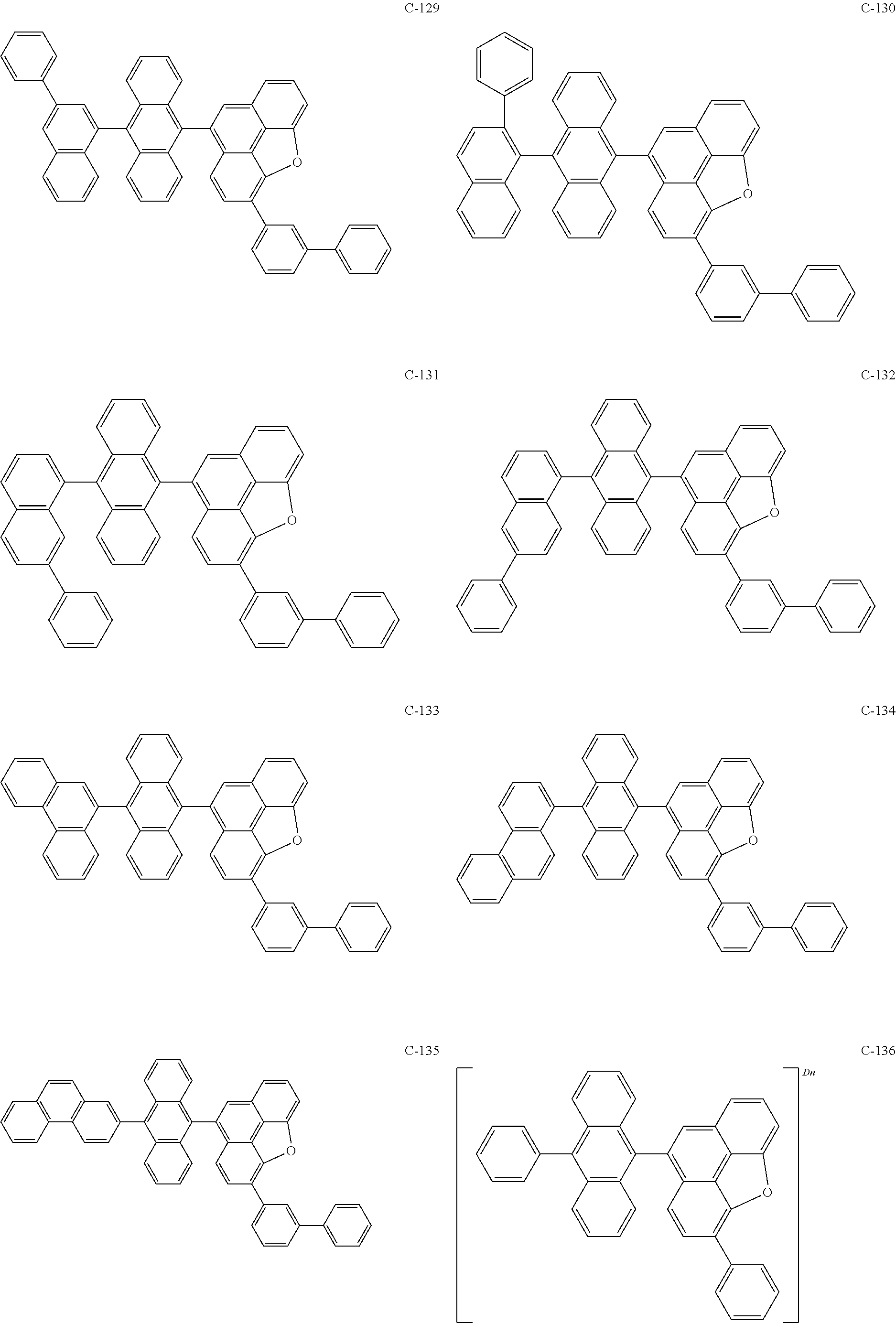 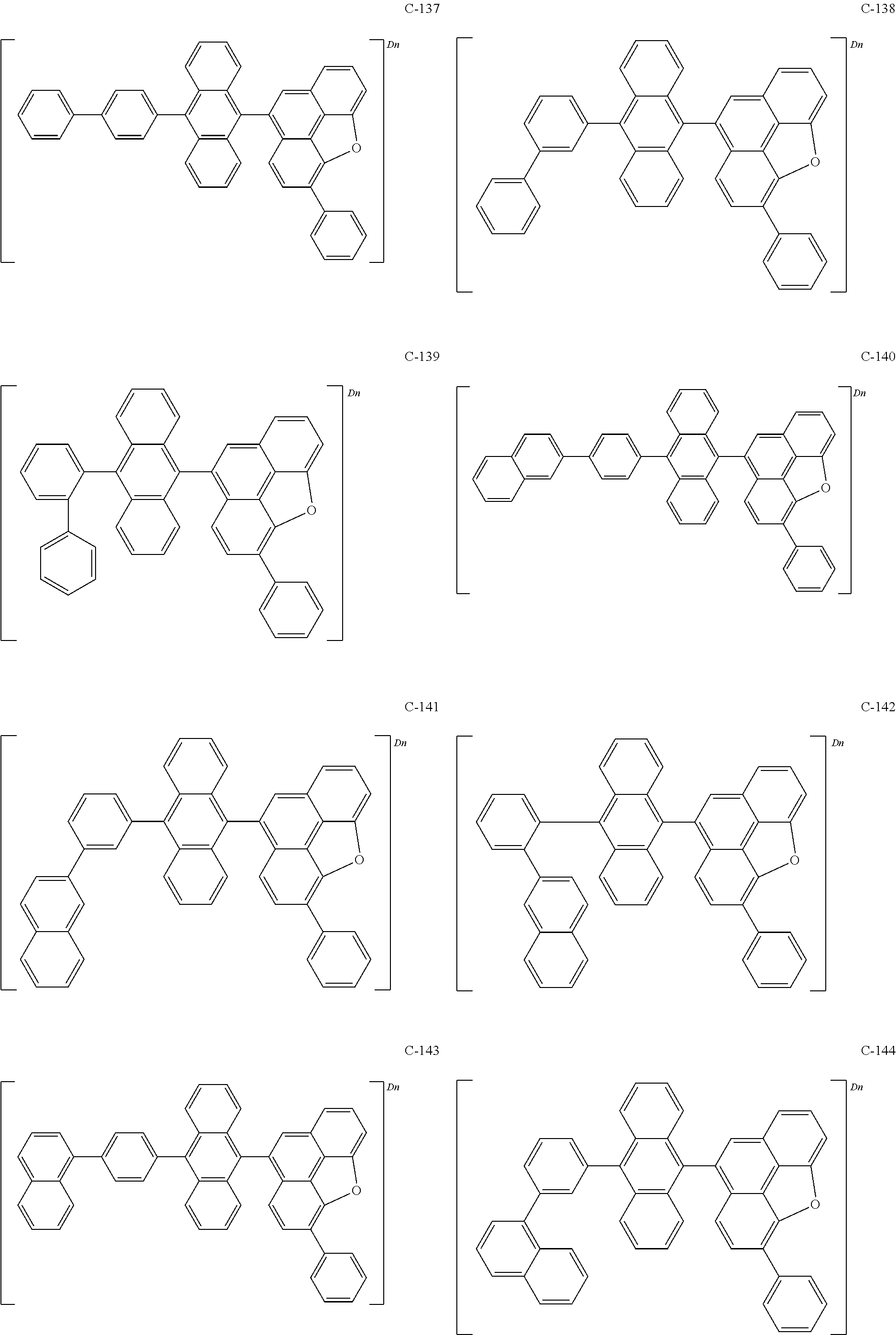 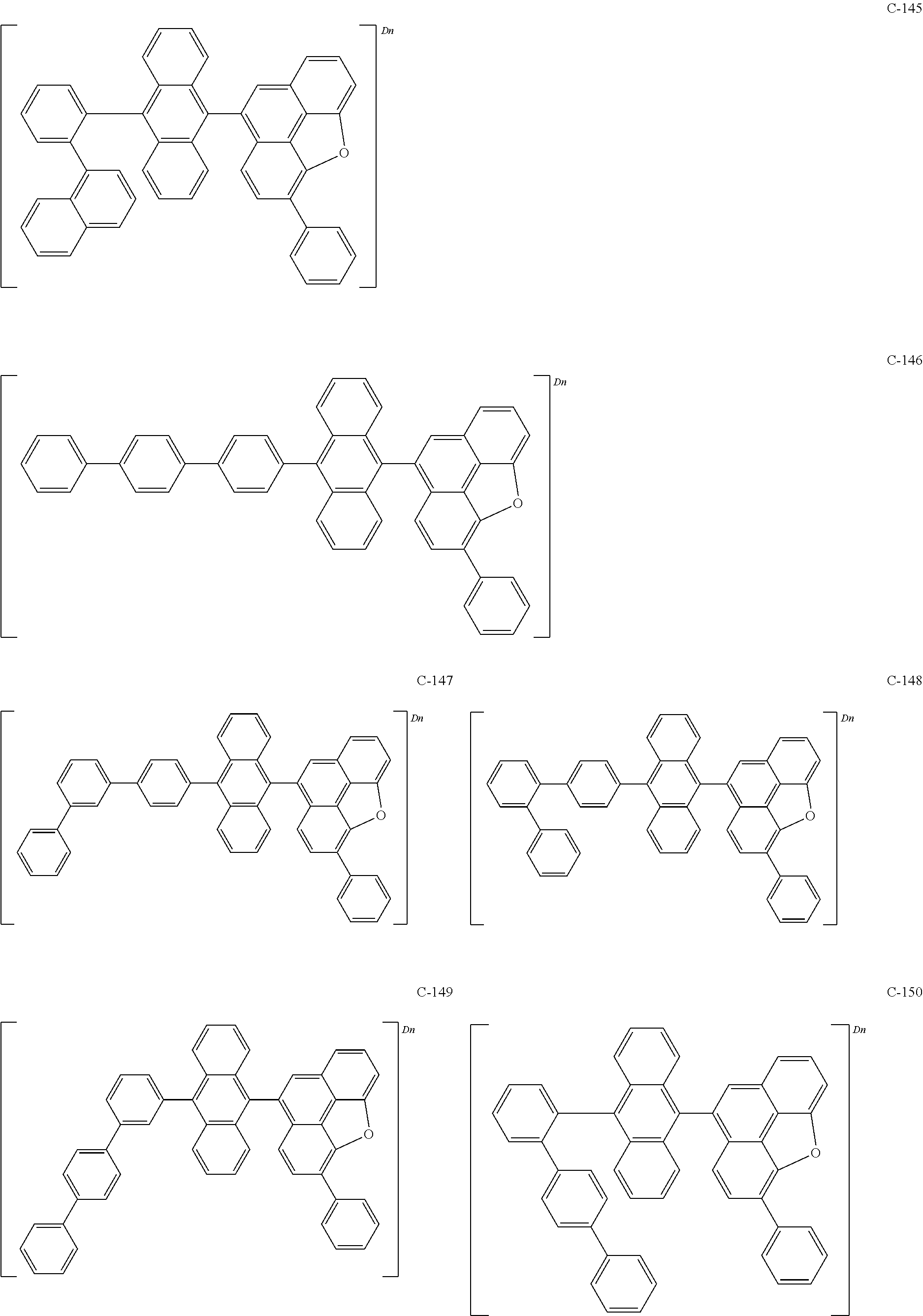 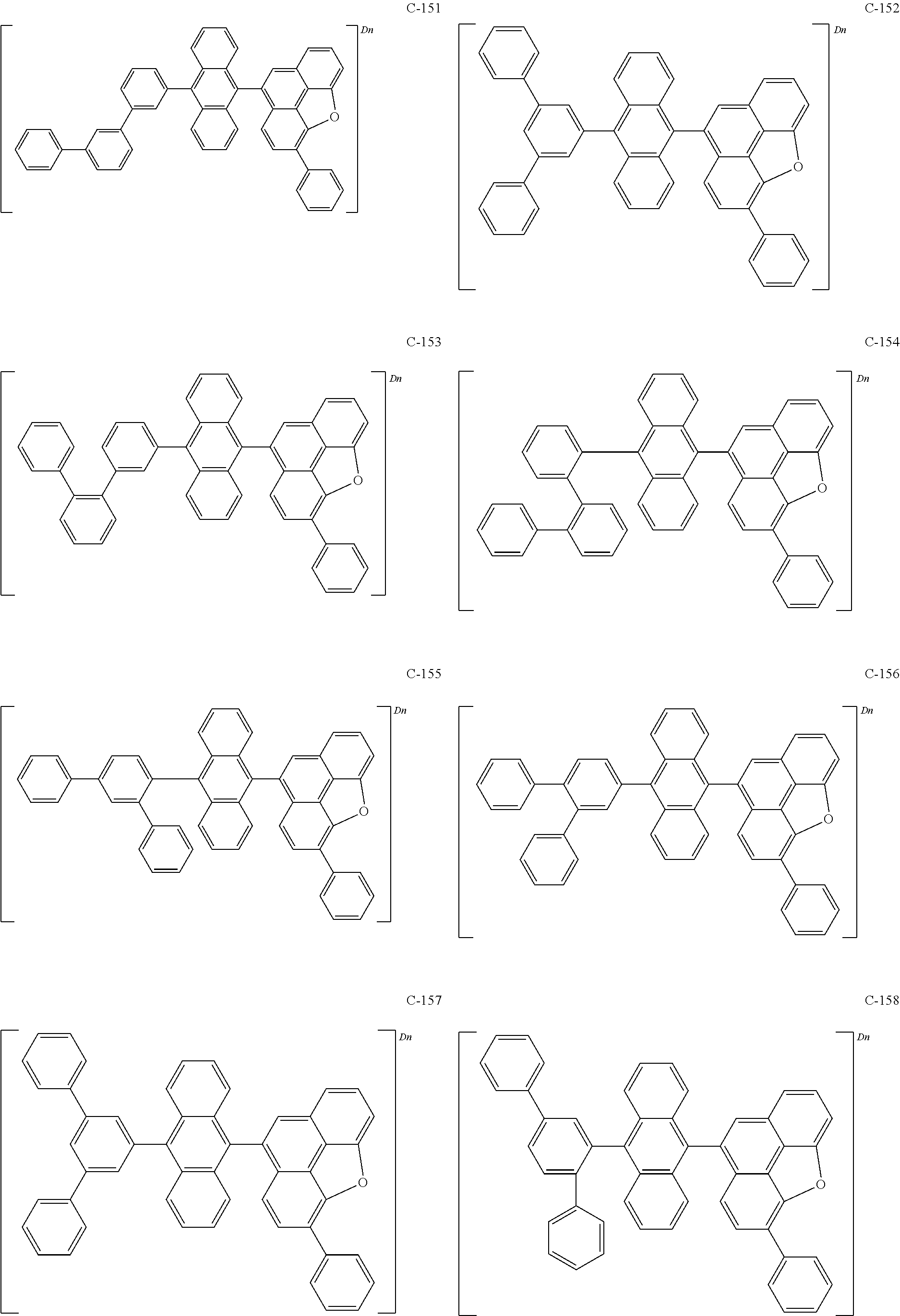 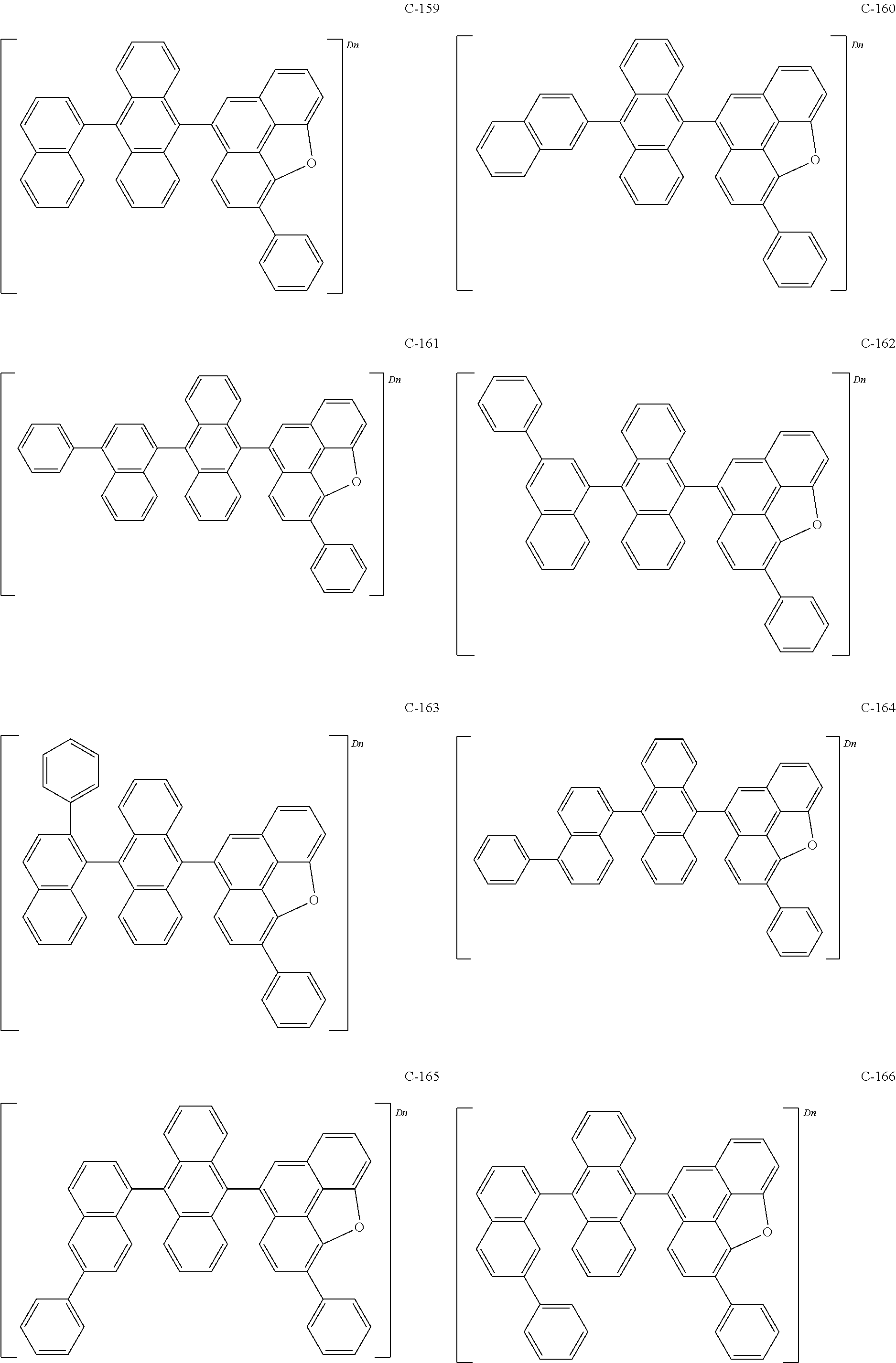 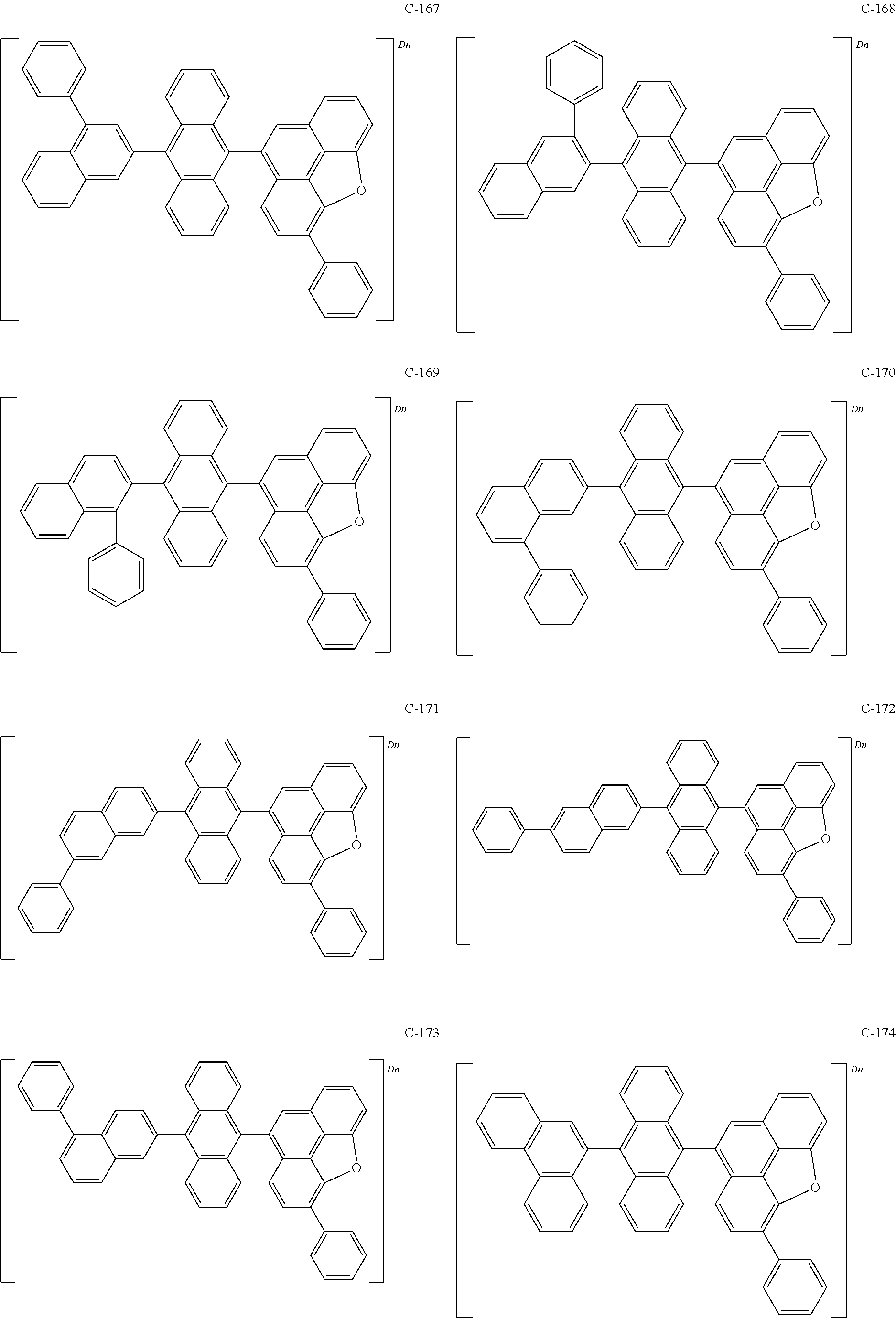 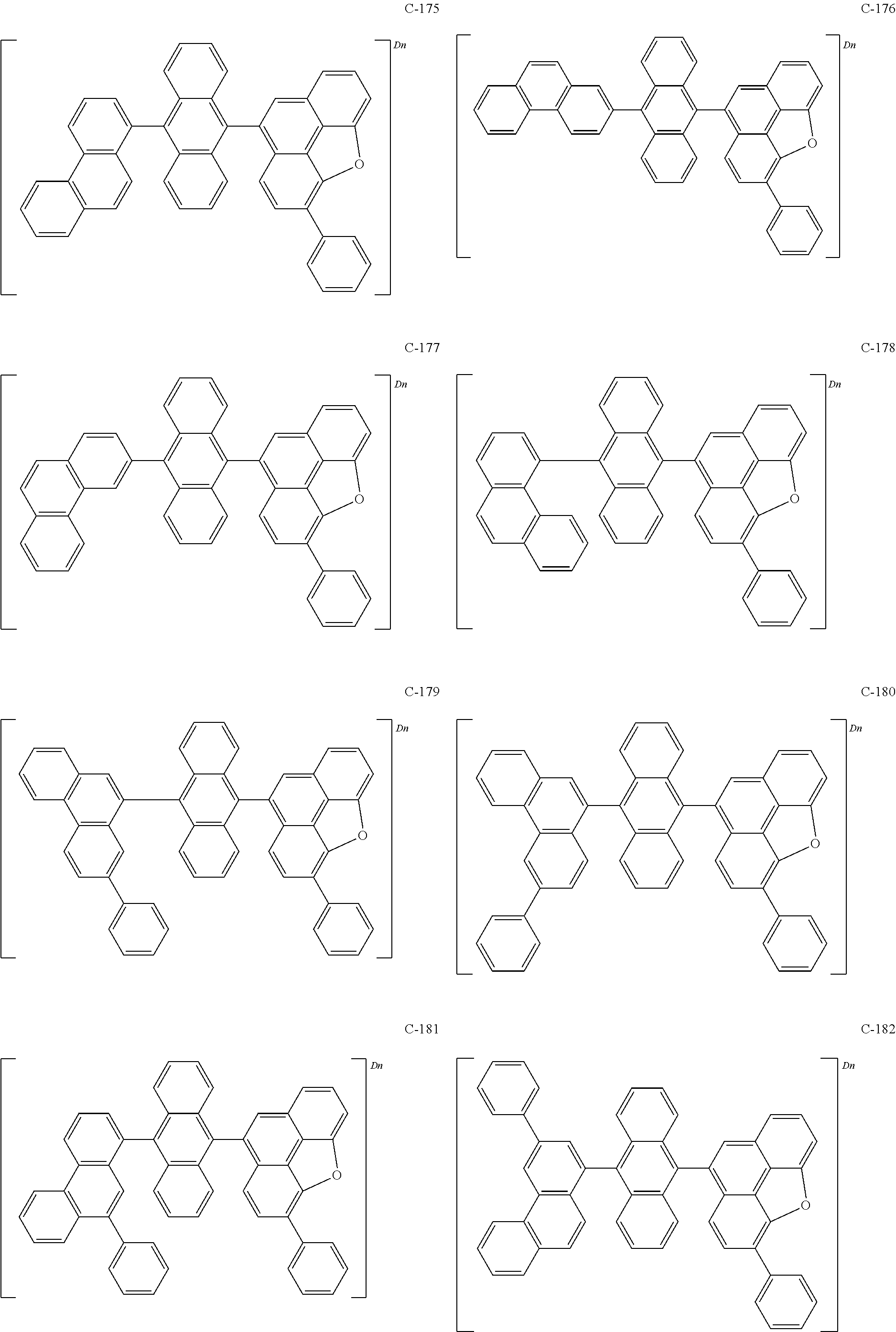 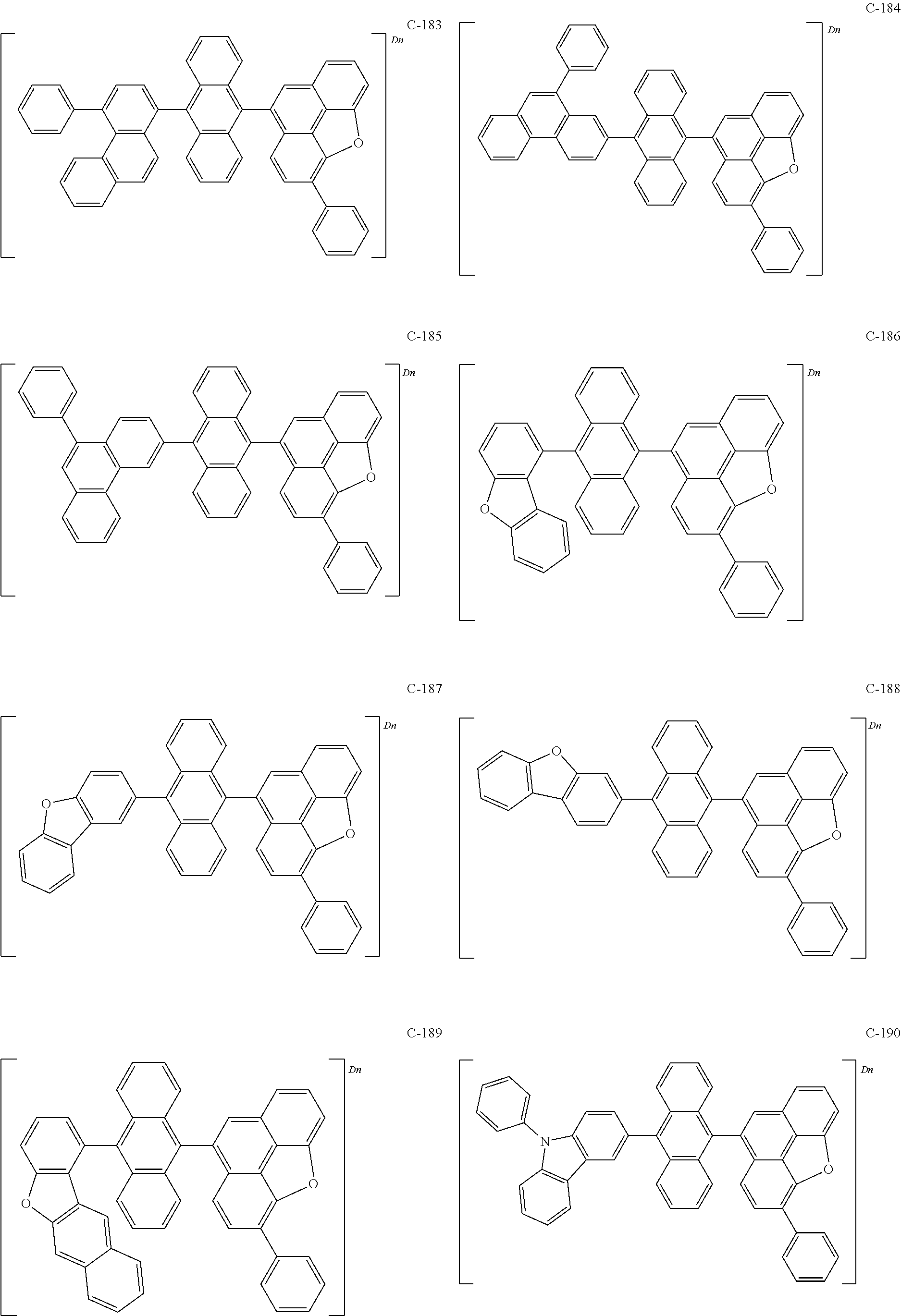 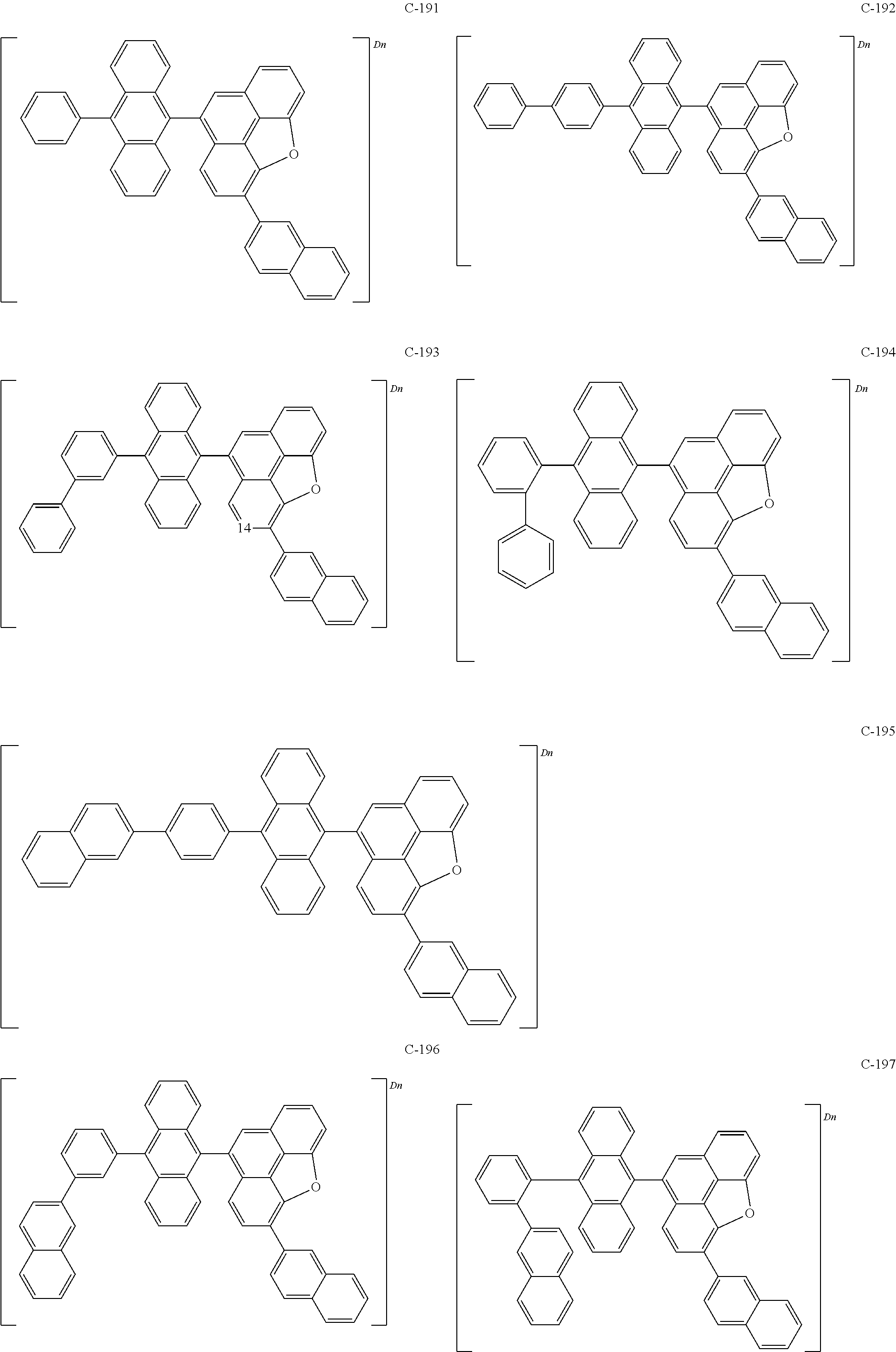 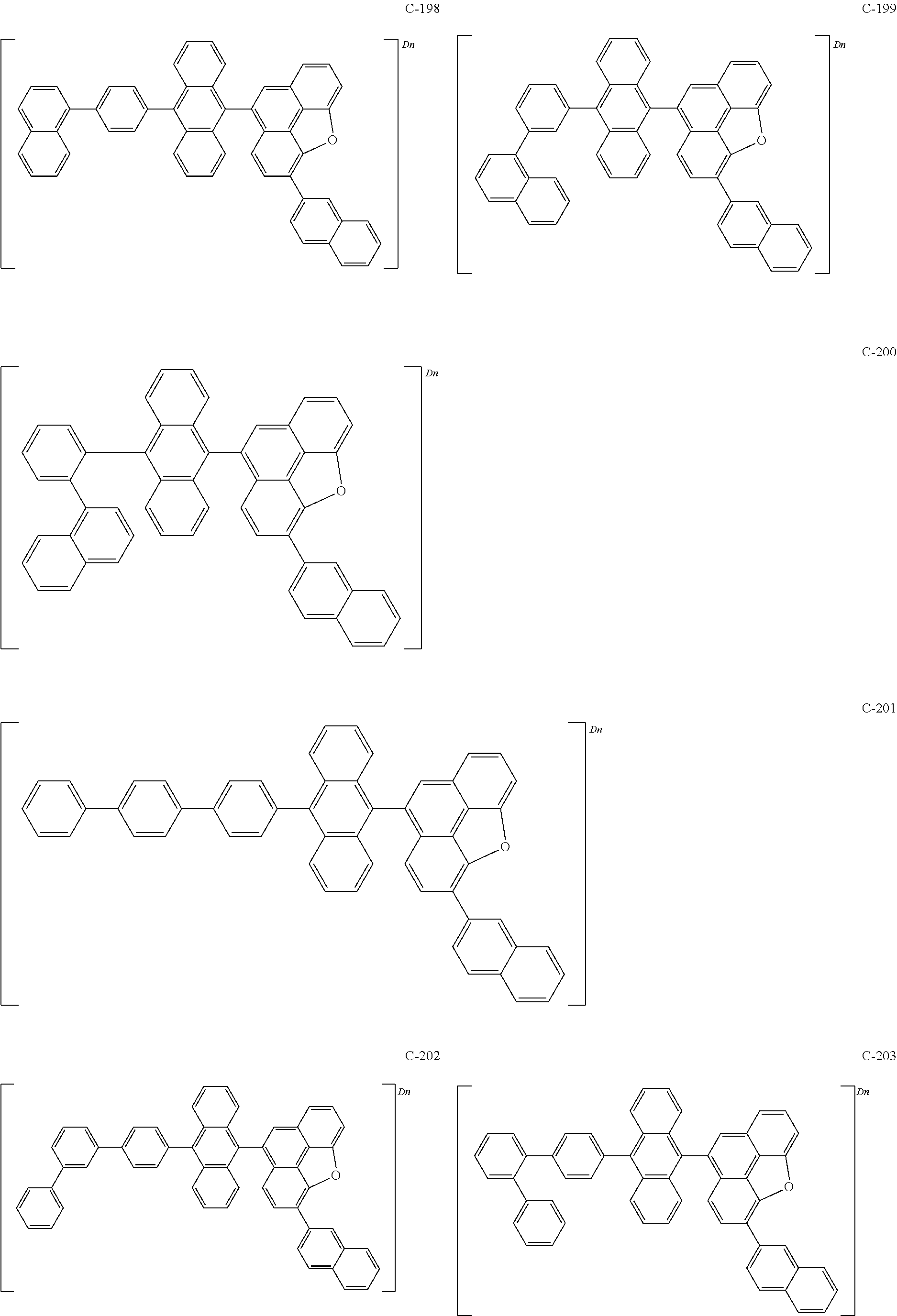 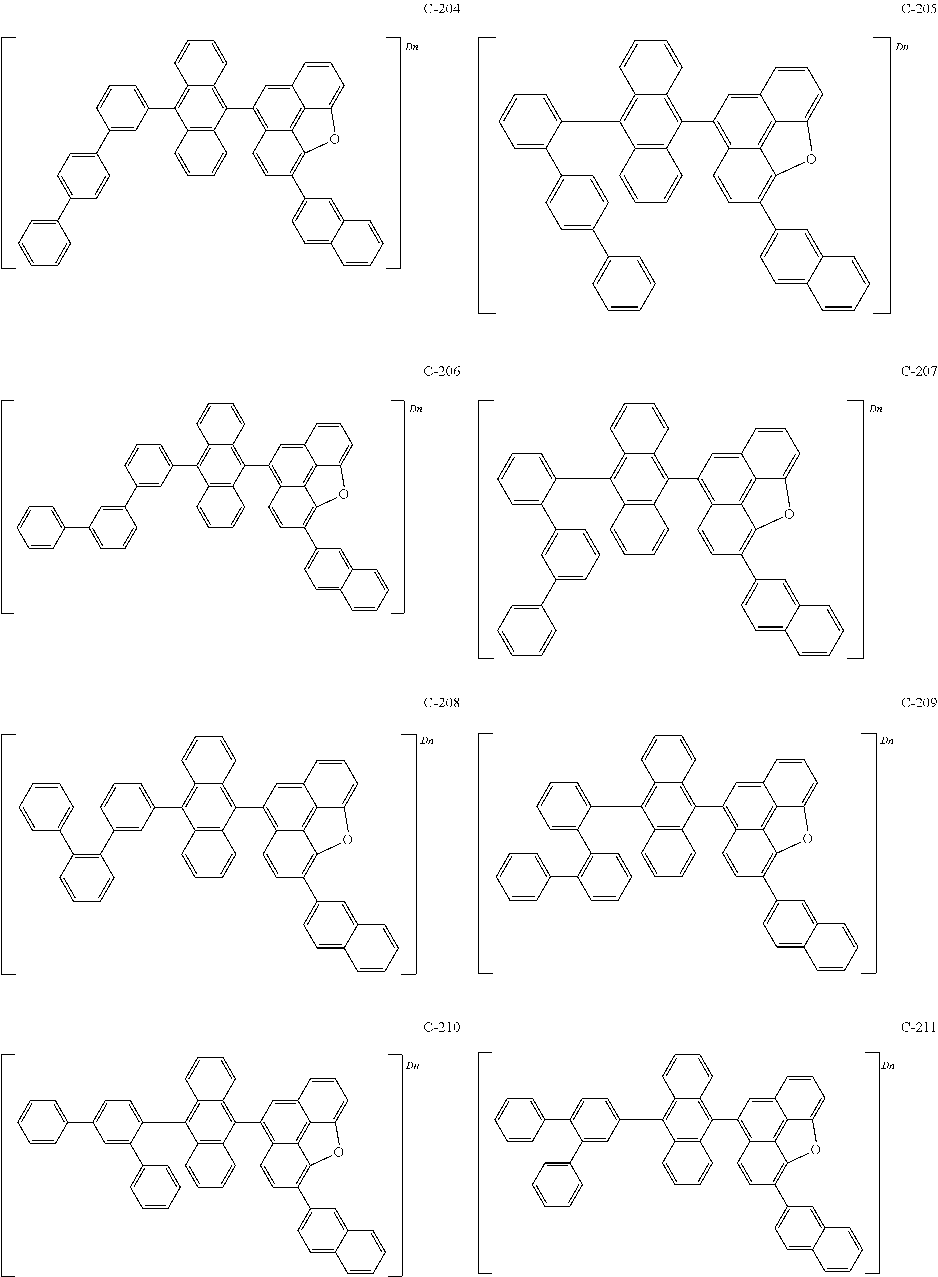 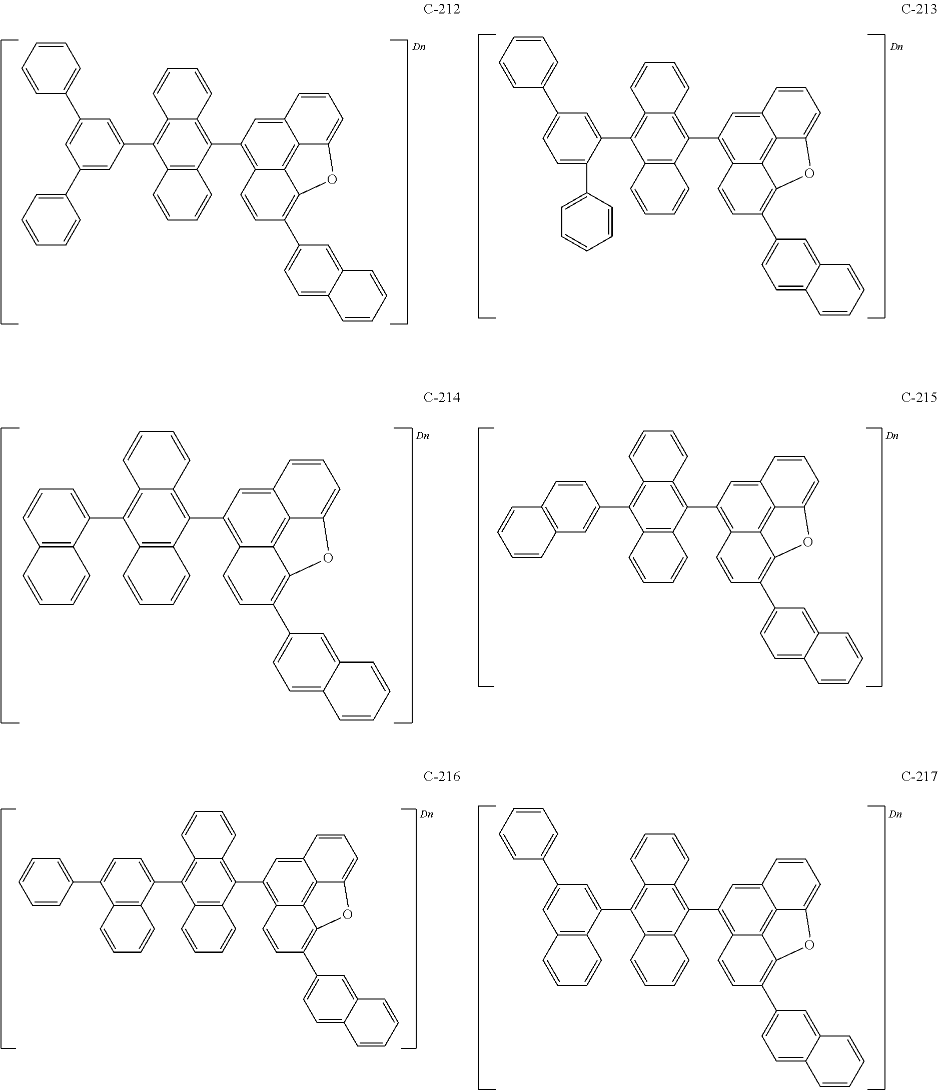 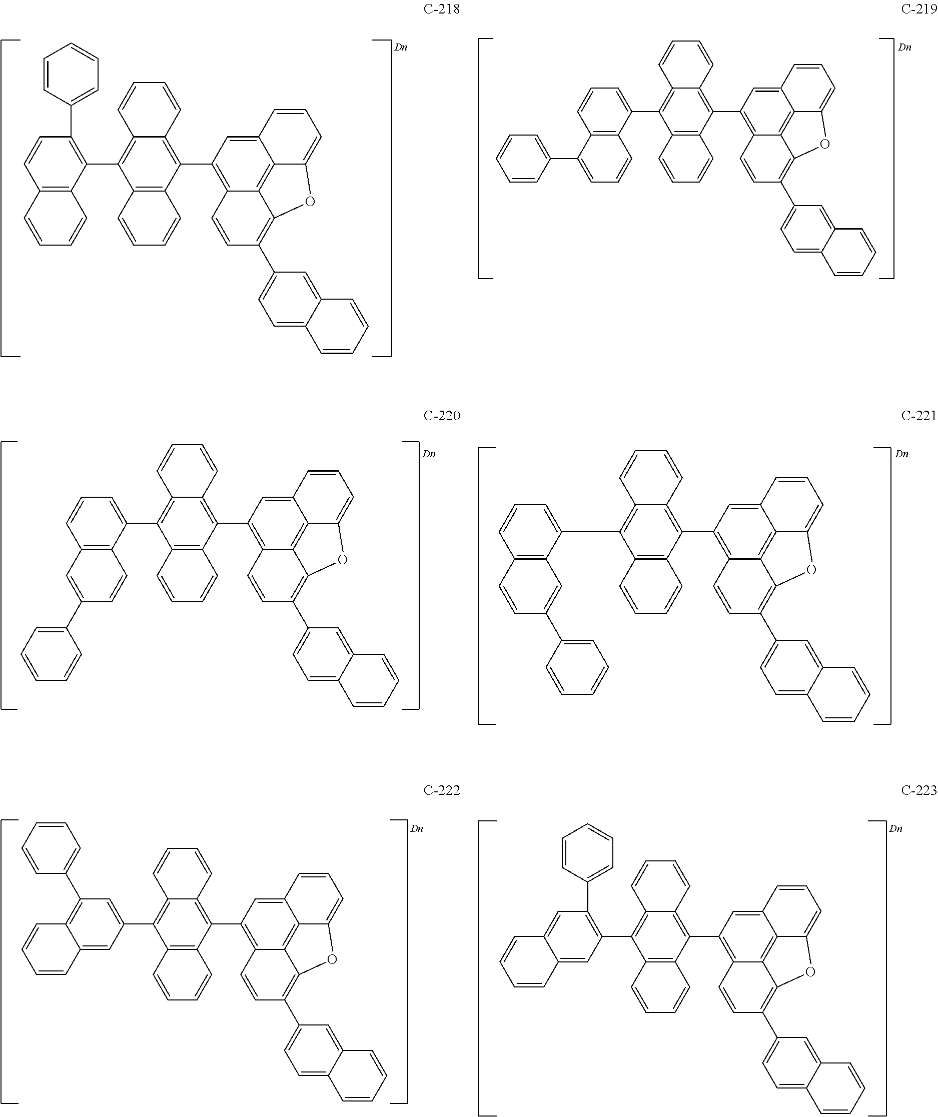 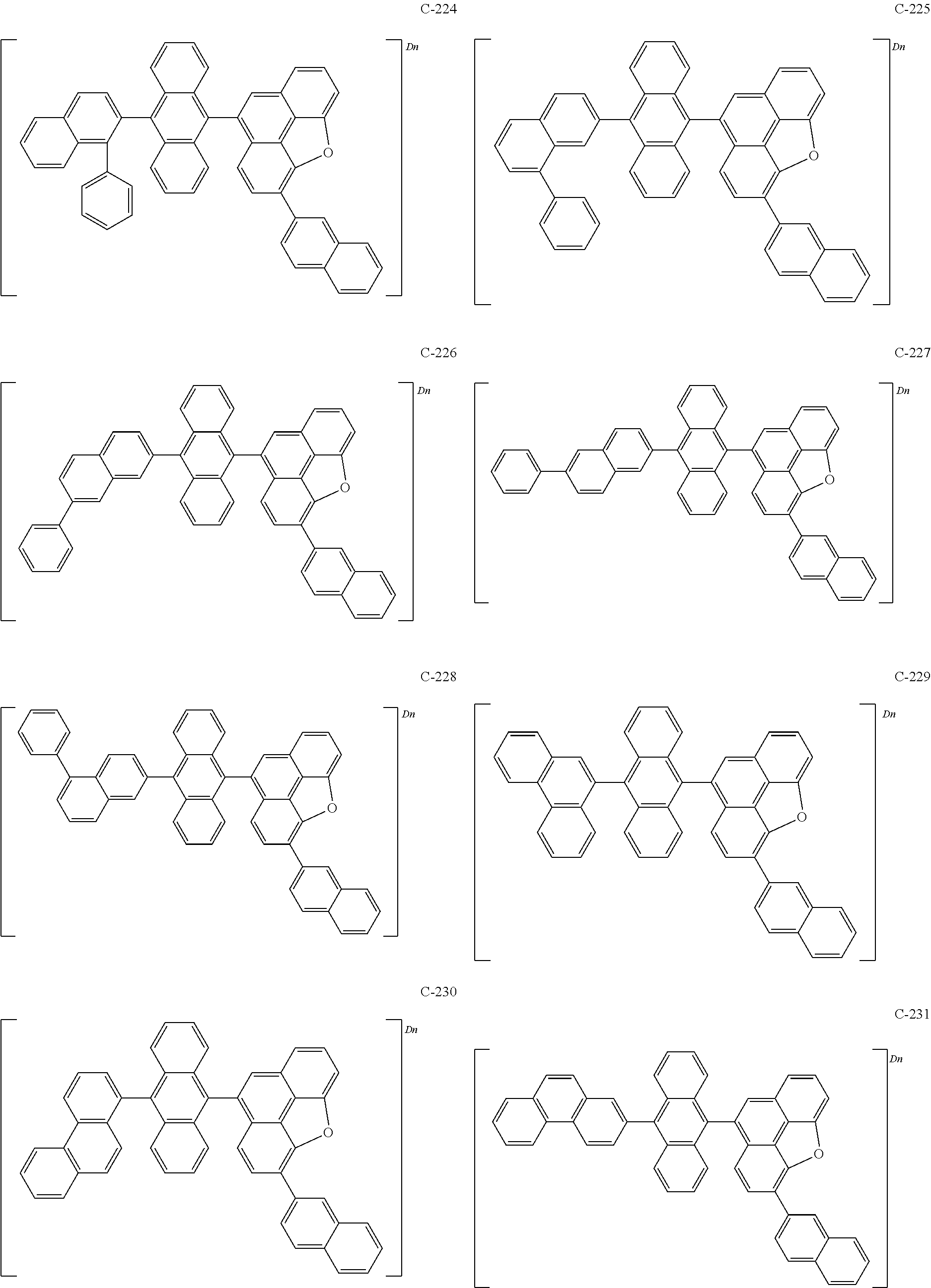 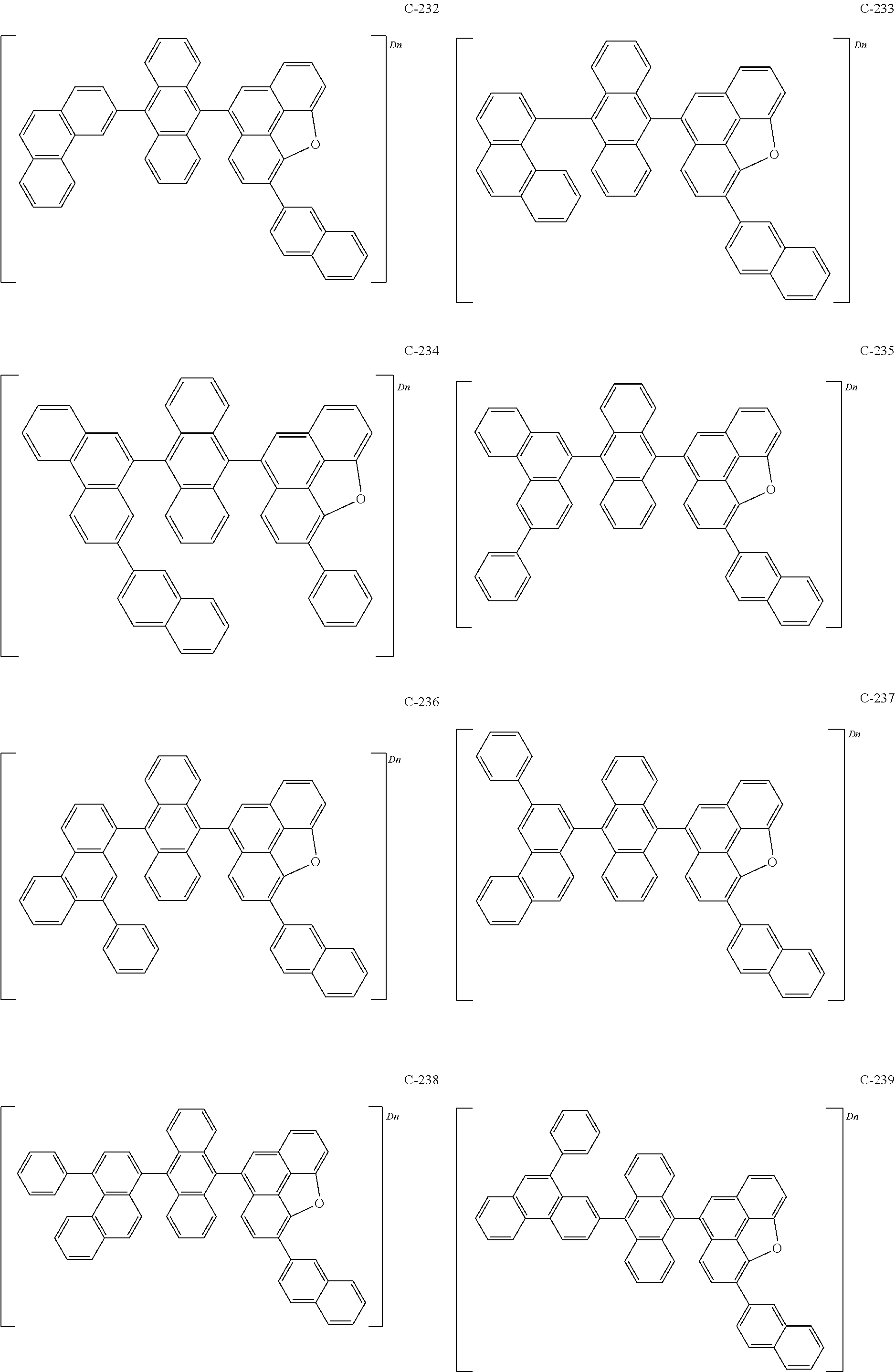 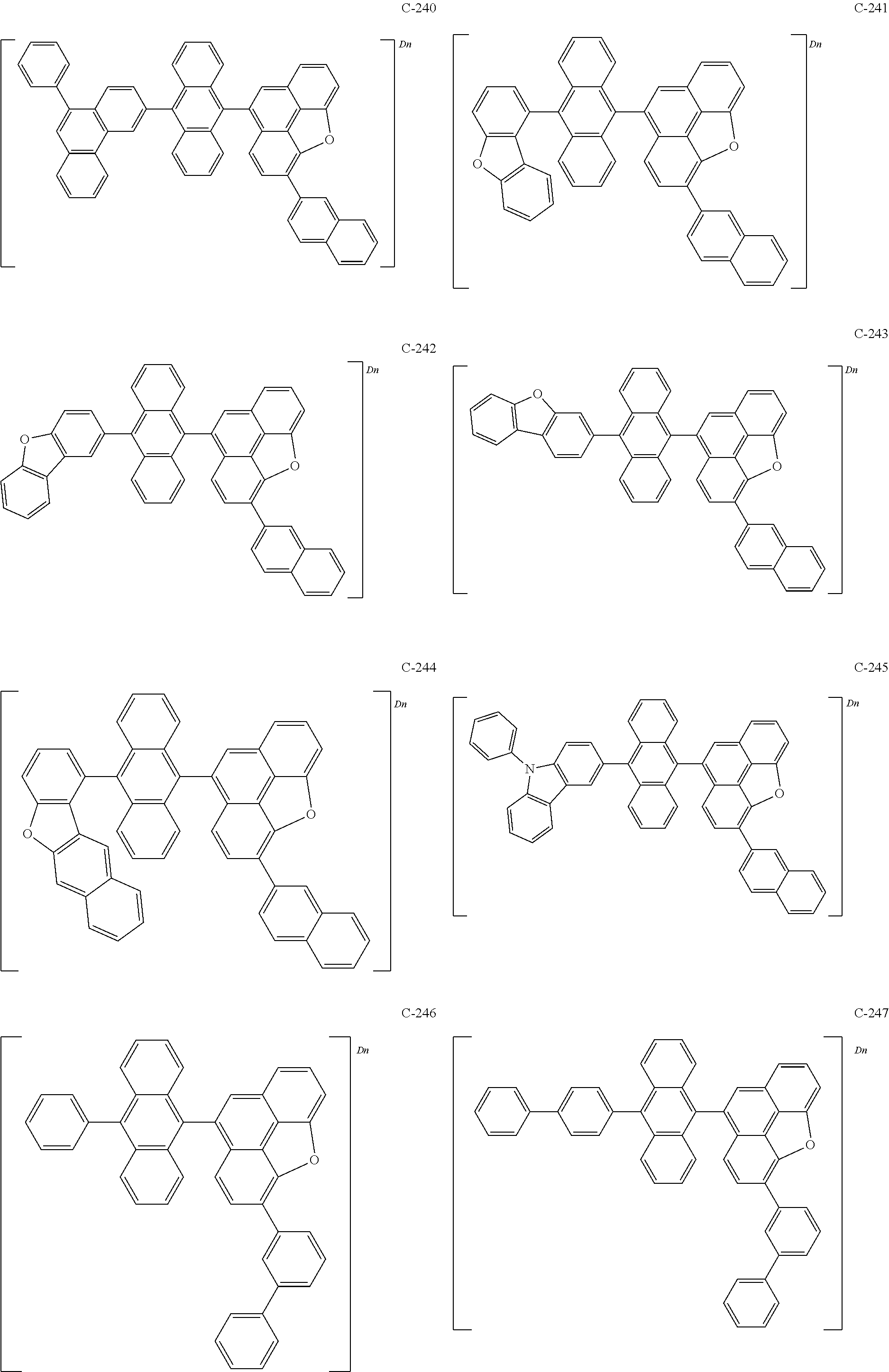 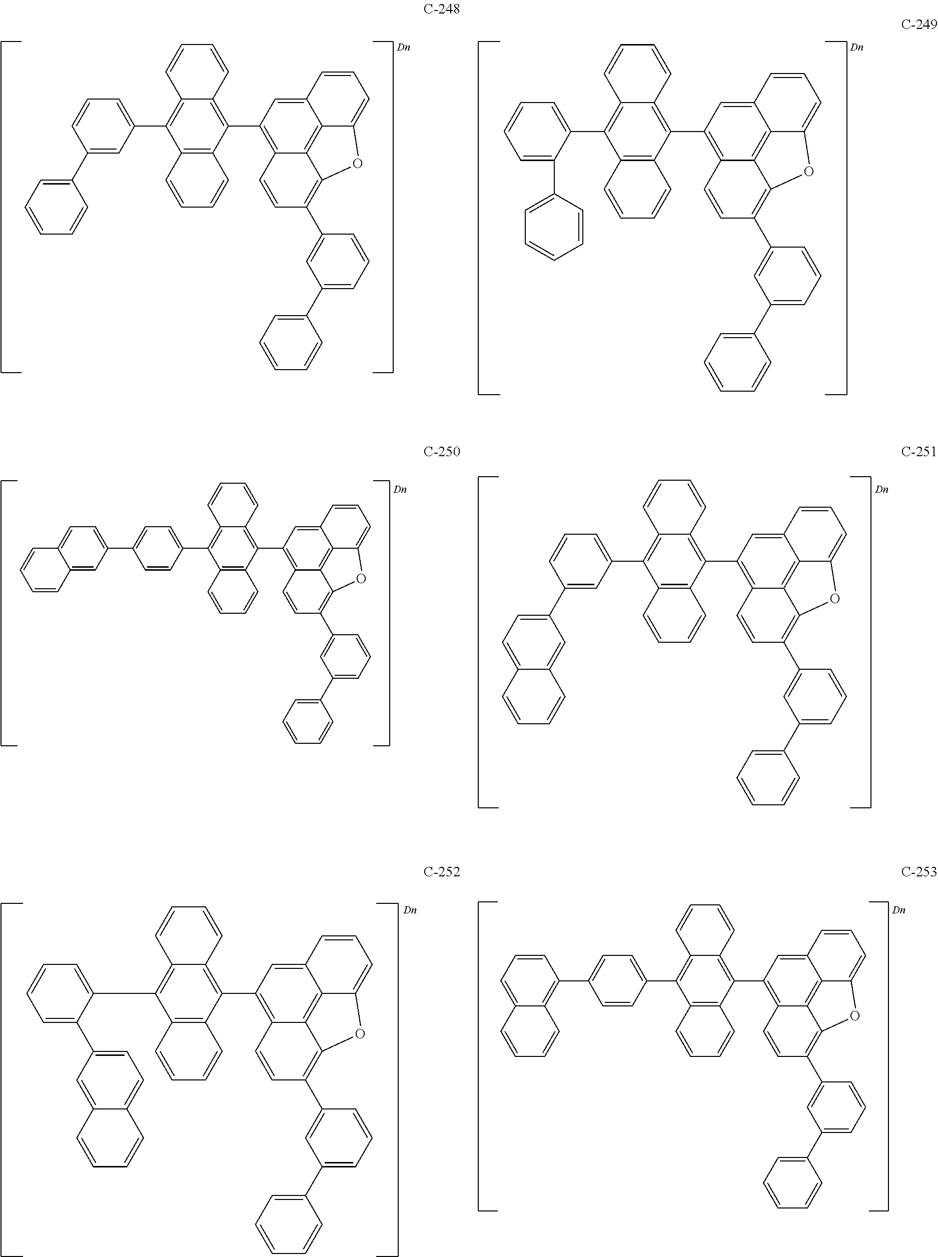 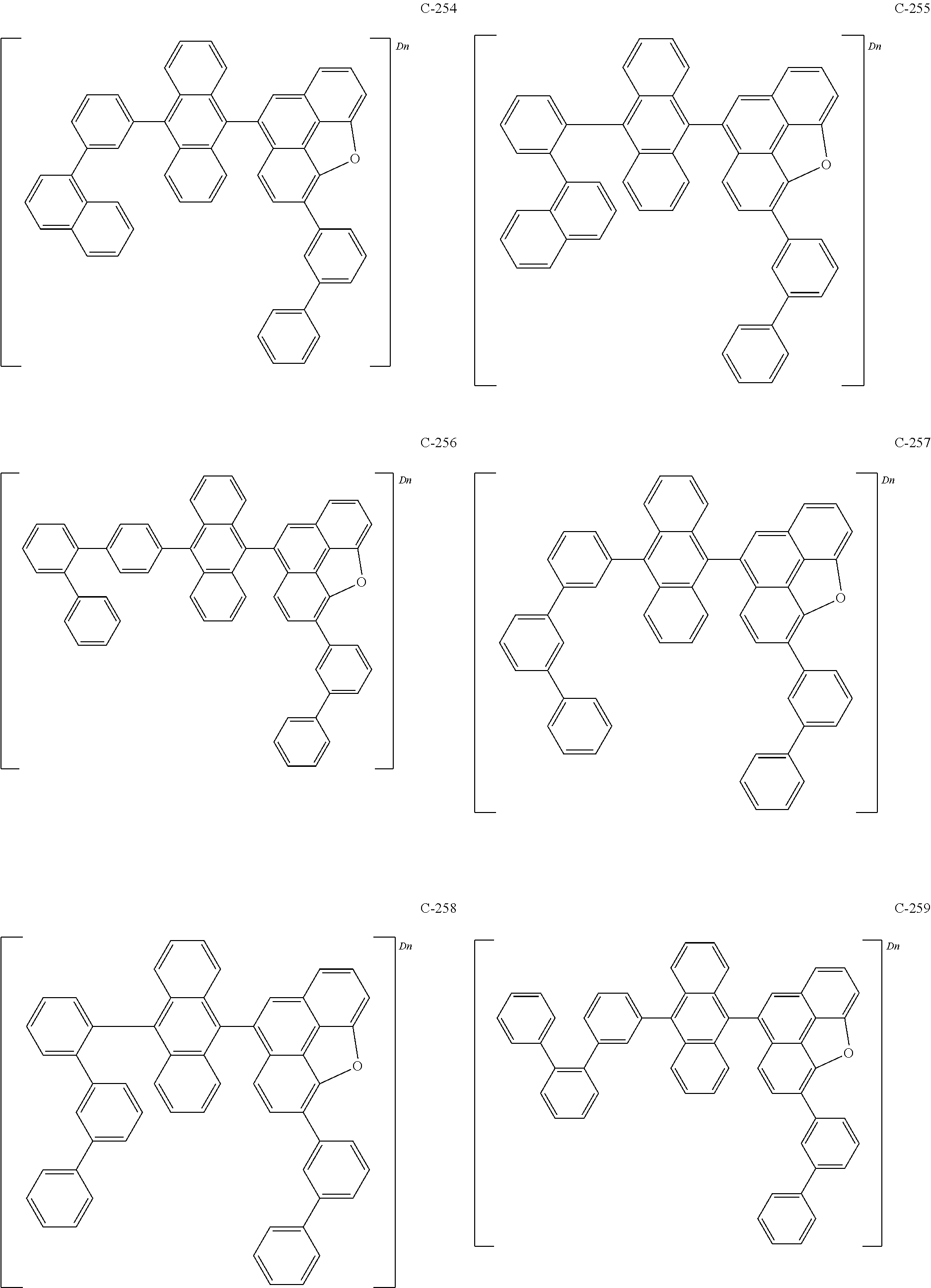 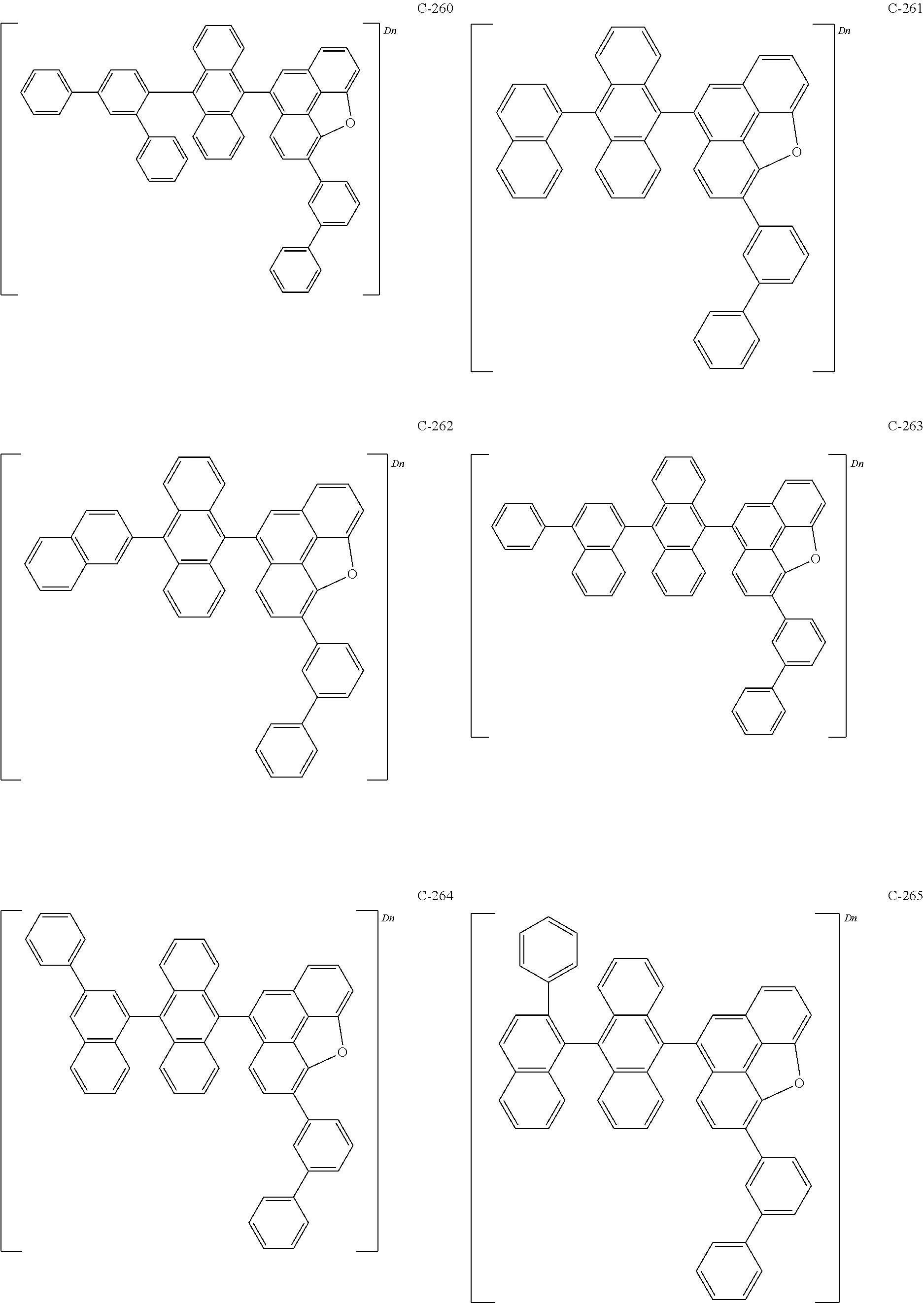 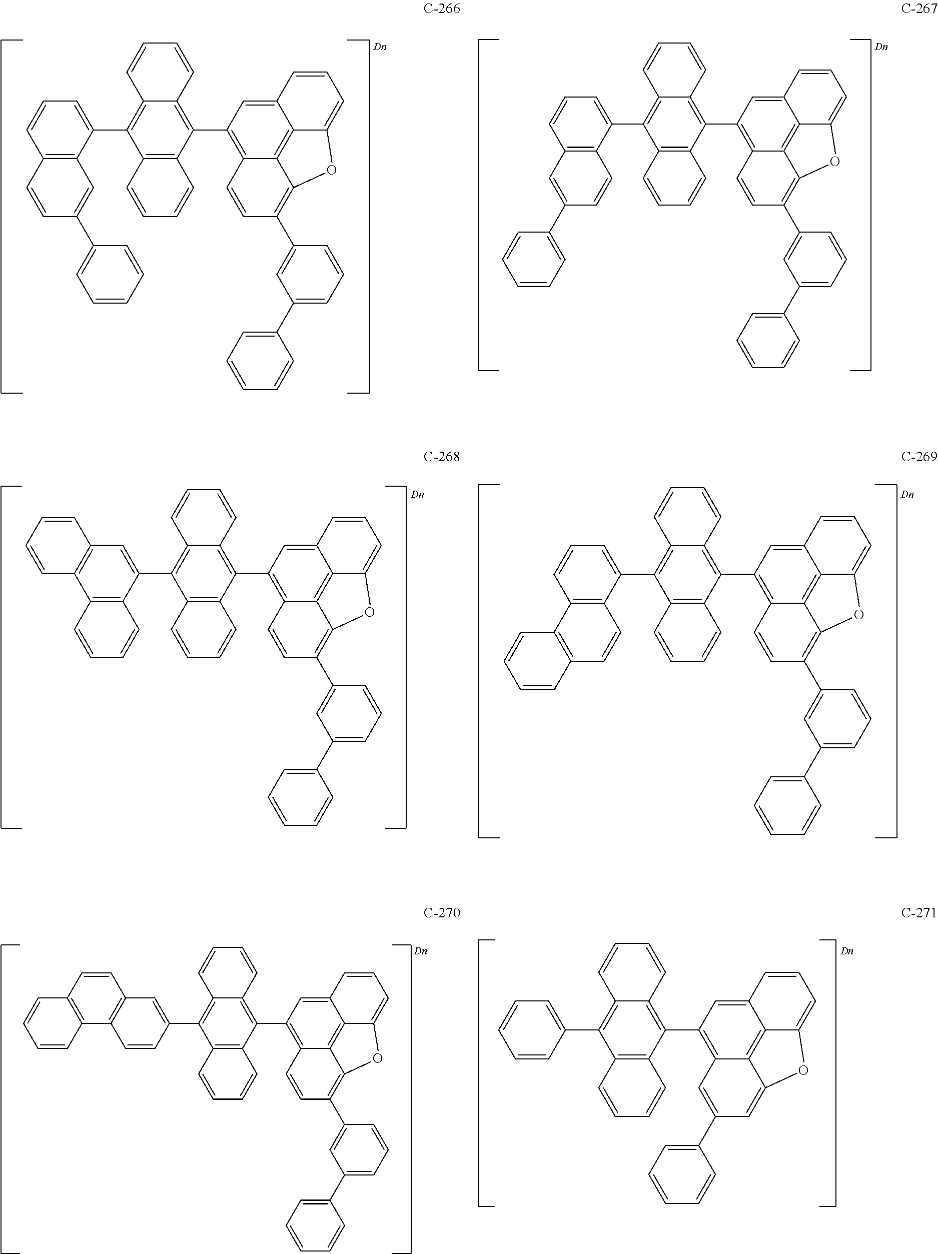 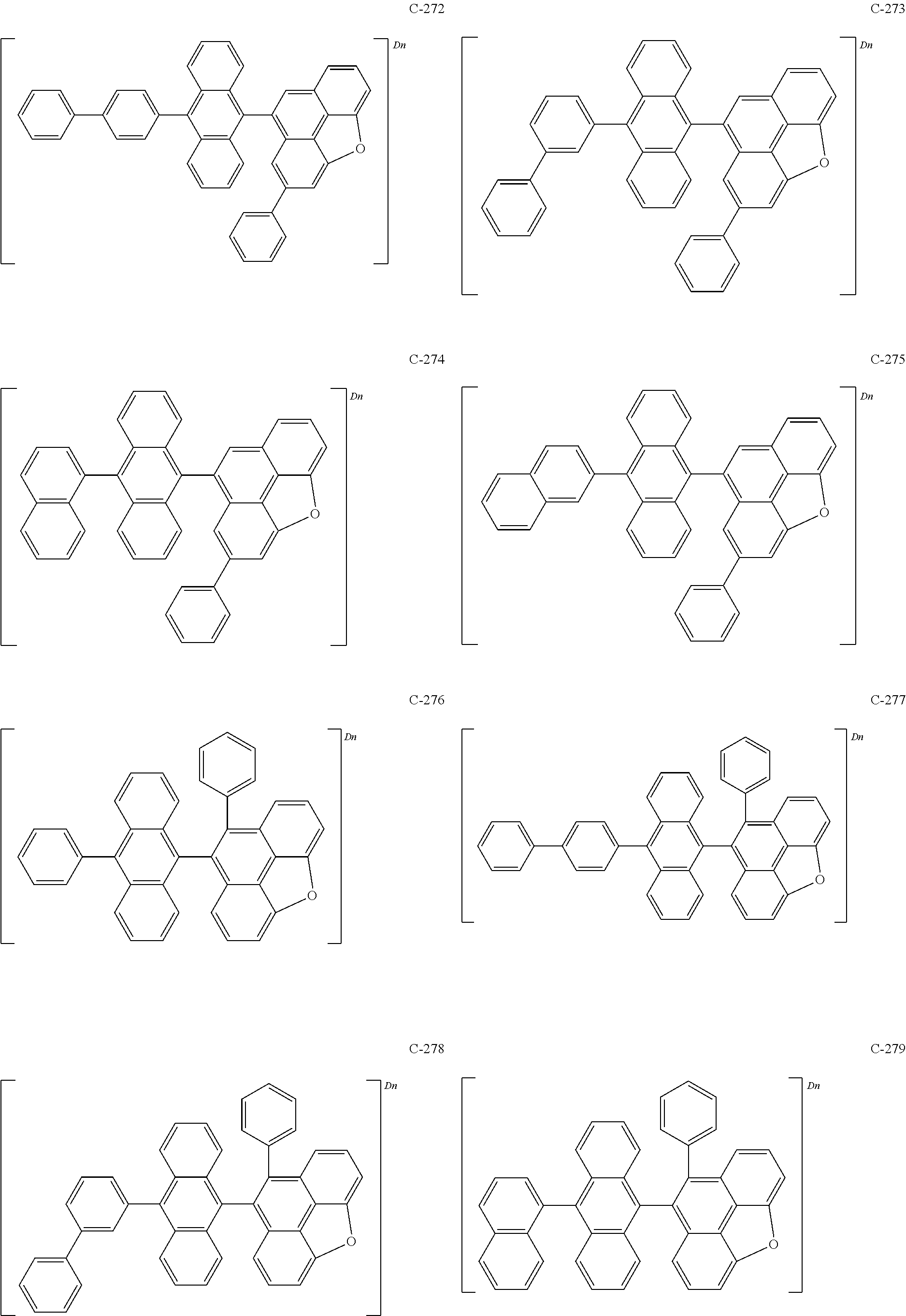 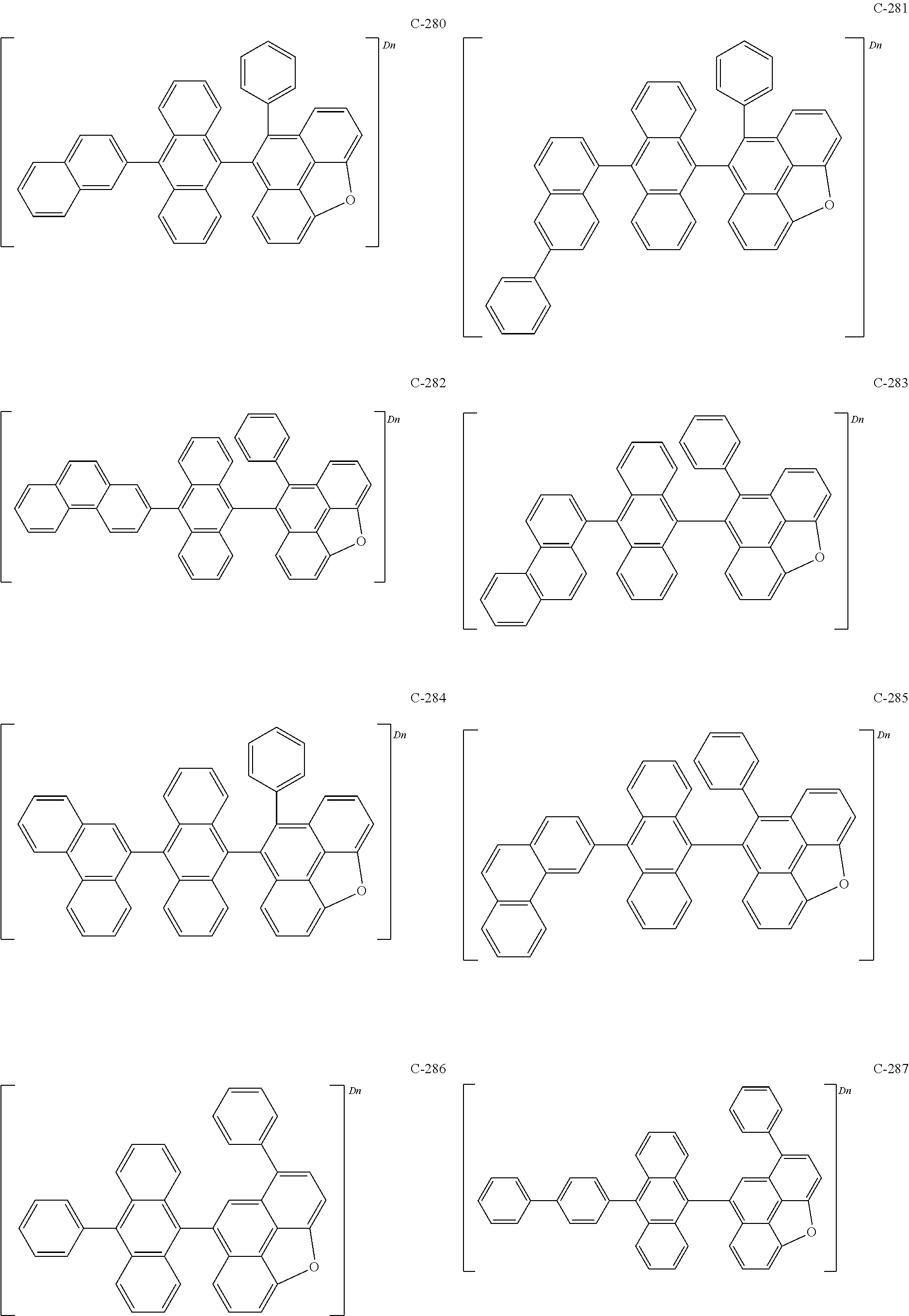 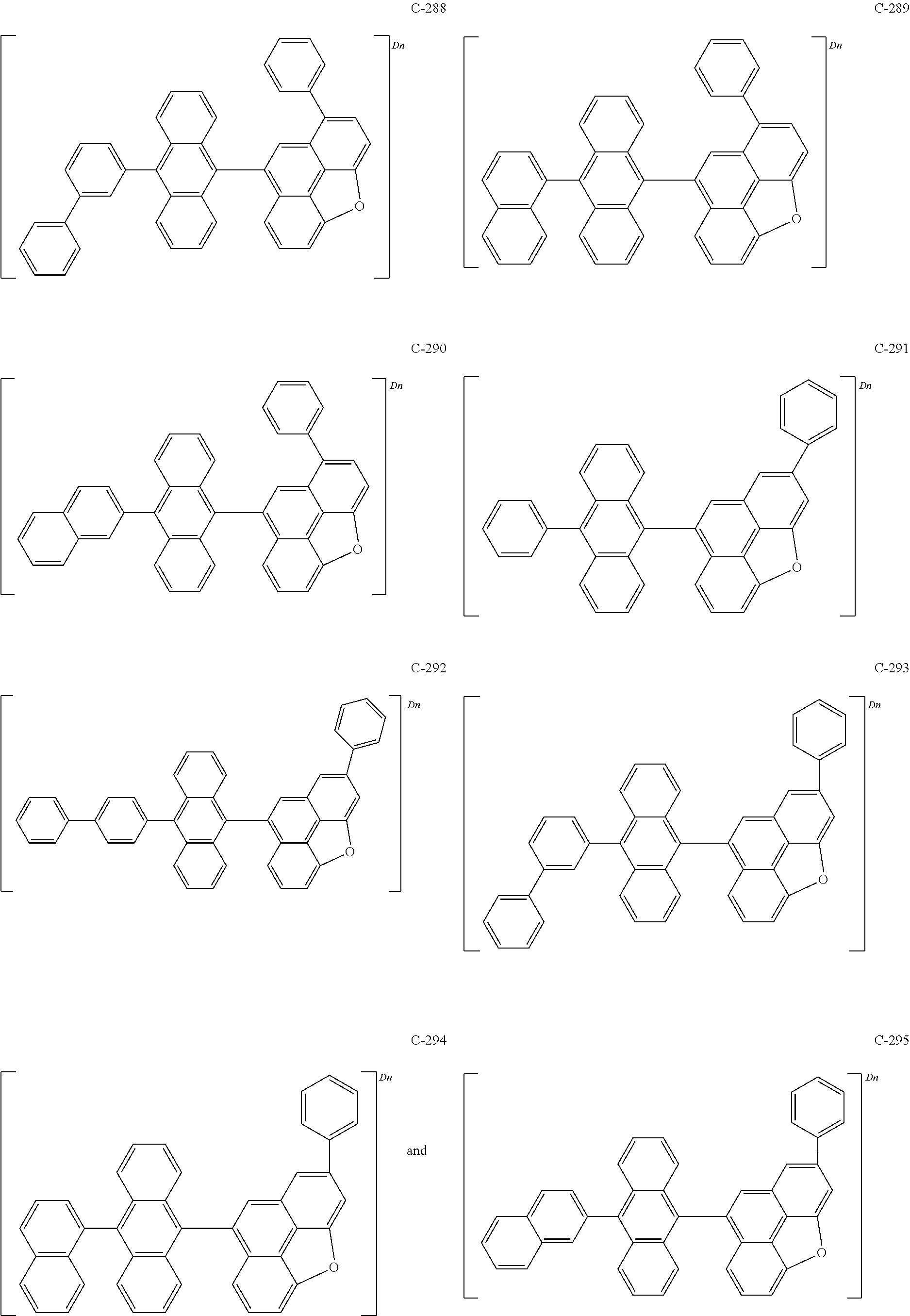
wherein Dn of compounds C-136 to C-295 means that n hydrogens are replaced by deuteriums, and n is between 1 and the maximum number of hydrogens in each compound.

6. A compound represented by the following formula 3: 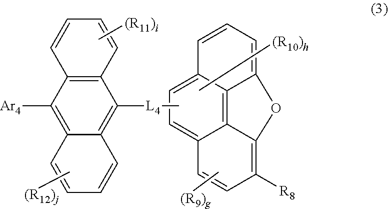
wherein,
Ar₄ represents a substituted or unsubstituted (C6-C30)aryl or a substituted or unsubstituted (3- to 30-membered)heteroaryl;
L₄ represents a single bond, a substituted or unsubstituted (C6-C30)arylene, or a substituted or unsubstituted (3- to 30-membered)heteroarylene;
R₈ represents a substituted or unsubstituted (C6-C30)aryl or a substituted or unsubstituted (3- to 30-membered)heteroaryl;
R₉ to R₁₂ each independently represent: hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or—N—(Ar₁₁)(Ar₁₂);
Ar₁₁ and Ar₁₂ each independently represent: hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
g represents an integer of 1 or 2; and h, i and j each independently represent an integer of 1 to 4, where if g to j are an integer of 2 or more, each of R₉ to R₁₂ may be the same or different.

7. The compound of claim 6, wherein formula 3 is represented by the following formula 3-1 or 3-2: 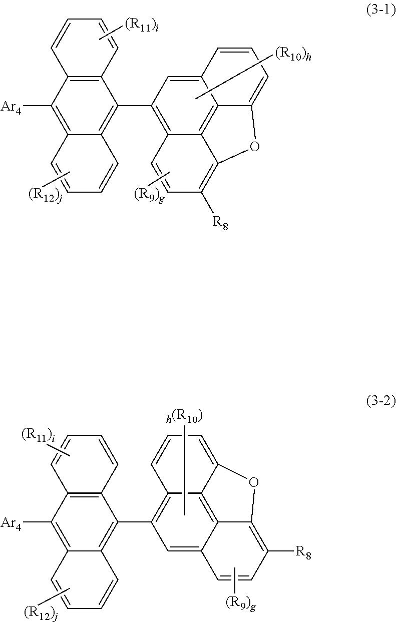
wherein Ar₄, R₈ to R₁₂, and g to j are the same as defined in claim 6.

8. The compound of claim 6, wherein the compound represented by formula 3 is selected from the following compounds: 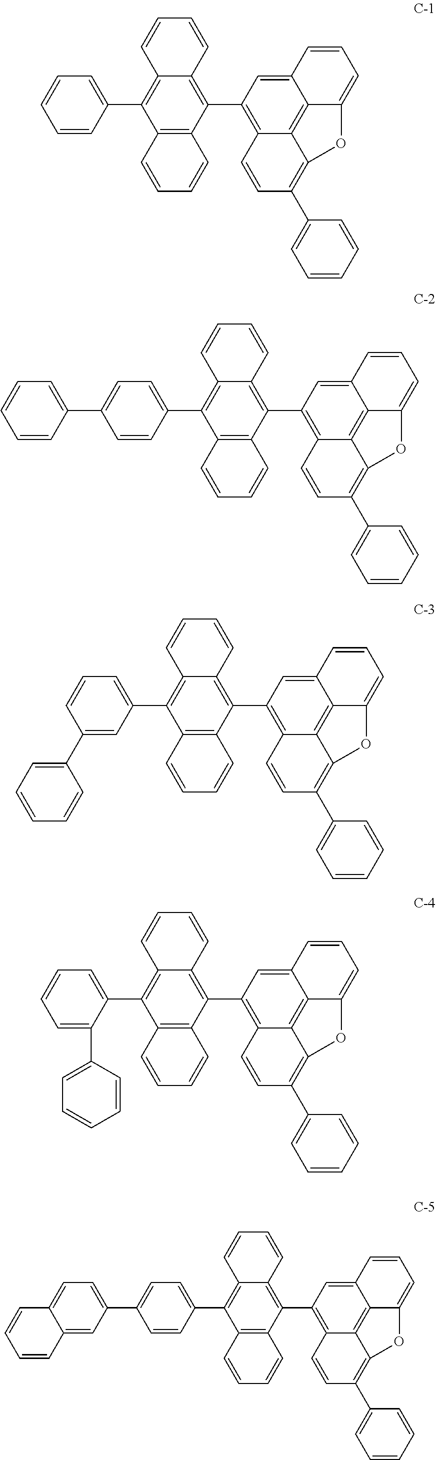 [SMILES: c1ccc(-c2c3ccccc3c(-c3cc4cccc5oc6c(-c7ccccc7)ccc3c6c45)c3ccccc23)cc1 ; c1ccc(-c2ccc(-c3c4ccccc4c(-c4cc5cccc6oc7c(-c8ccccc8)ccc4c7c56)c4ccccc34)cc2)cc1 ; c1ccc(-c2cccc(-c3c4ccccc4c(-c4cc5cccc6oc7c(-c8ccccc8)ccc4c7c56)c4ccccc34)c2)cc1 ; c1ccc(-c2ccccc2-c2c3ccccc3c(-c3cc4cccc5oc6c(-c7ccccc7)ccc3c6c45)c3ccccc23)cc1 ; c1ccc(-c2ccc3c(-c4c5ccccc5c(-c5ccc(-c6ccc7ccccc7c6)cc5)c5ccccc45)cc4cccc5oc2c3c45)cc1] 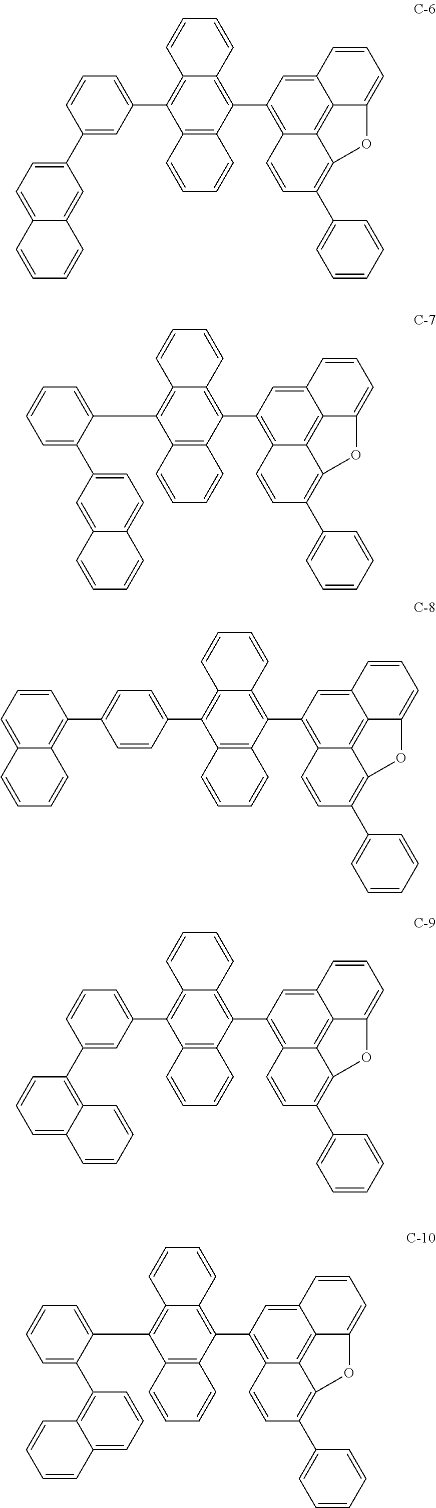 [SMILES: c1ccc(-c2ccc3c(-c4c5ccccc5c(-c5cccc(-c6ccc7ccccc7c6)c5)c5ccccc45)cc4cccc5oc2c3c45)cc1 ; c1ccc(-c2ccc3c(-c4c5ccccc5c(-c5ccccc5-c5ccc6ccccc6c5)c5ccccc45)cc4cccc5oc2c3c45)cc1 ; c1ccc(-c2ccc3c(-c4c5ccccc5c(-c5ccc(-c6cccc7ccccc67)cc5)c5ccccc45)cc4cccc5oc2c3c45)cc1 ; c1ccc(-c2ccc3c(-c4c5ccccc5c(-c5cccc(-c6cccc7ccccc67)c5)c5ccccc45)cc4cccc5oc2c3c45)cc1 ; c1ccc(-c2ccc3c(-c4c5ccccc5c(-c5ccccc5-c5cccc6ccccc56)c5ccccc45)cc4cccc5oc2c3c45)cc1] 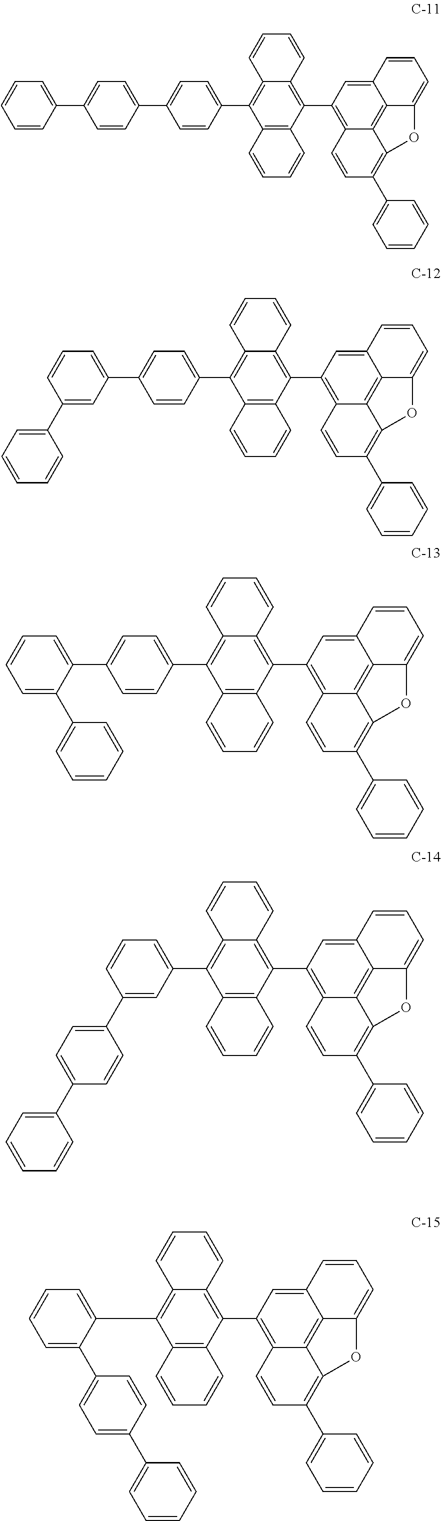 [SMILES: c1ccc(-c2ccc(-c3ccc(-c4c5ccccc5c(-c5cc6cccc7oc8c(-c9ccccc9)ccc5c8c67)c5ccccc45)cc3)cc2)cc1 ; c1ccc(-c2cccc(-c3ccc(-c4c5ccccc5c(-c5cc6cccc7oc8c(-c9ccccc9)ccc5c8c67)c5ccccc45)cc3)c2)cc1 ; c1ccc(-c2ccccc2-c2ccc(-c3c4ccccc4c(-c4cc5cccc6oc7c(-c8ccccc8)ccc4c7c56)c4ccccc34)cc2)cc1 ; c1ccc(-c2ccc(-c3cccc(-c4c5ccccc5c(-c5cc6cccc7oc8c(-c9ccccc9)ccc5c8c67)c5ccccc45)c3)cc2)cc1 ; c1ccc(-c2ccc(-c3ccccc3-c3c4ccccc4c(-c4cc5cccc6oc7c(-c8ccccc8)ccc4c7c56)c4ccccc34)cc2)cc1] 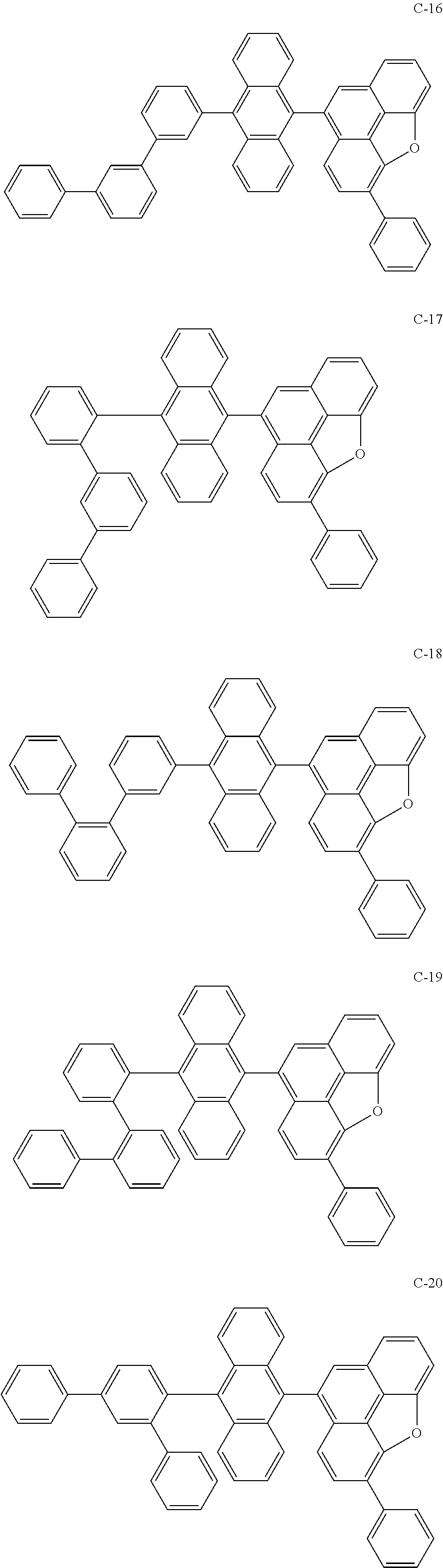 [SMILES: c1ccc(-c2cccc(-c3cccc(-c4c5ccccc5c(-c5cc6cccc7oc8c(-c9ccccc9)ccc5c8c67)c5ccccc45)c3)c2)cc1 ; c1ccc(-c2cccc(-c3ccccc3-c3c4ccccc4c(-c4cc5cccc6oc7c(-c8ccccc8)ccc4c7c56)c4ccccc34)c2)cc1 ; c1ccc(-c2ccccc2-c2cccc(-c3c4ccccc4c(-c4cc5cccc6oc7c(-c8ccccc8)ccc4c7c56)c4ccccc34)c2)cc1 ; c1ccc(-c2ccccc2-c2ccccc2-c2c3ccccc3c(-c3cc4cccc5oc6c(-c7ccccc7)ccc3c6c45)c3ccccc23)cc1 ; c1ccc(-c2ccc(-c3c4ccccc4c(-c4cc5cccc6oc7c(-c8ccccc8)ccc4c7c56)c4ccccc34)c(-c3ccccc3)c2)cc1] 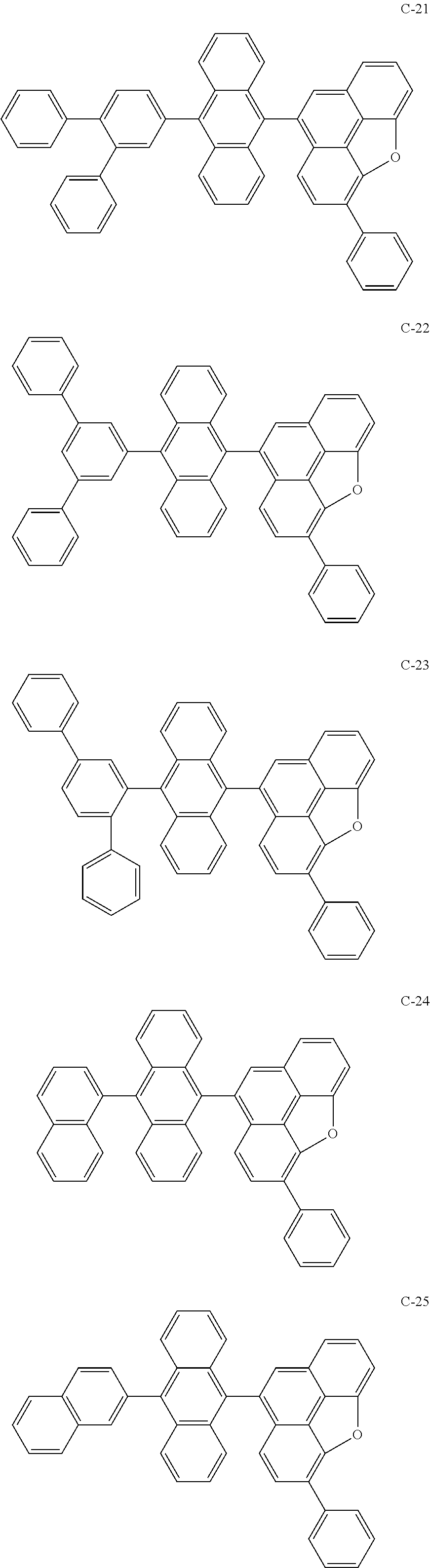 [SMILES: c1ccc(-c2ccc(-c3c4ccccc4c(-c4cc5cccc6oc7c(-c8ccccc8)ccc4c7c56)c4ccccc34)cc2-c2ccccc2)cc1 ; c1ccc(-c2cc(-c3ccccc3)cc(-c3c4ccccc4c(-c4cc5cccc6oc7c(-c8ccccc8)ccc4c7c56)c4ccccc34)c2)cc1 ; c1ccc(-c2ccc(-c3ccccc3)c(-c3c4ccccc4c(-c4cc5cccc6oc7c(-c8ccccc8)ccc4c7c56)c4ccccc34)c2)cc1 ; c1ccc(-c2ccc3c(-c4c5ccccc5c(-c5cccc6ccccc56)c5ccccc45)cc4cccc5oc2c3c45)cc1 ; c1ccc(-c2ccc3c(-c4c5ccccc5c(-c5ccc6ccccc6c5)c5ccccc45)cc4cccc5oc2c3c45)cc1] 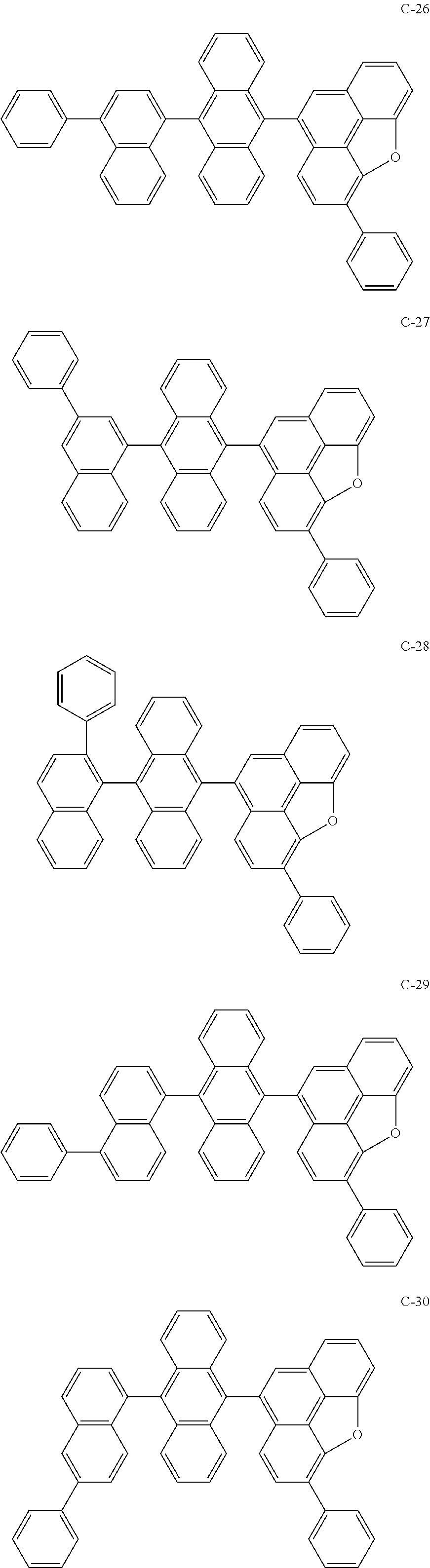 [SMILES: c1ccc(-c2ccc(-c3c4ccccc4c(-c4cc5cccc6oc7c(-c8ccccc8)ccc4c7c56)c4ccccc34)c3ccccc23)cc1 ; c1ccc(-c2cc(-c3c4ccccc4c(-c4cc5cccc6oc7c(-c8ccccc8)ccc4c7c56)c4ccccc34)c3ccccc3c2)cc1 ; c1ccc(-c2ccc3ccccc3c2-c2c3ccccc3c(-c3cc4cccc5oc6c(-c7ccccc7)ccc3c6c45)c3ccccc23)cc1 ; c1ccc(-c2cccc3c(-c4c5ccccc5c(-c5cc6cccc7oc8c(-c9ccccc9)ccc5c8c67)c5ccccc45)cccc23)cc1 ; c1ccc(-c2ccc3c(-c4c5ccccc5c(-c5cc6cccc7oc8c(-c9ccccc9)ccc5c8c67)c5ccccc45)cccc3c2)cc1] 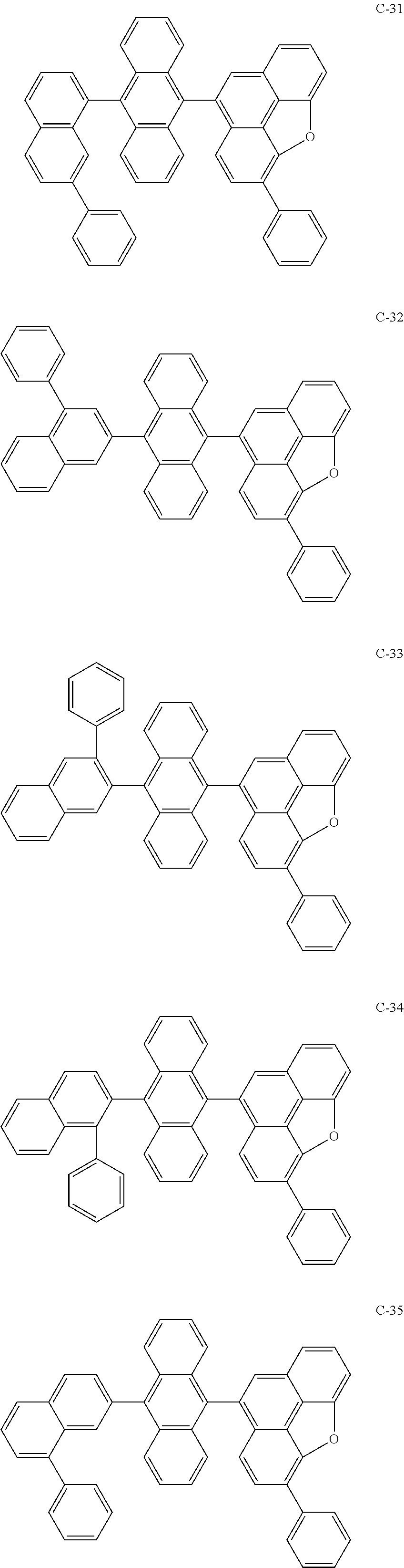 [SMILES: c1ccc(-c2ccc3cccc(-c4c5ccccc5c(-c5cc6cccc7oc8c(-c9ccccc9)ccc5c8c67)c5ccccc45)c3c2)cc1 ; c1ccc(-c2cc(-c3c4ccccc4c(-c4cc5cccc6oc7c(-c8ccccc8)ccc4c7c56)c4ccccc34)cc3ccccc23)cc1 ; c1ccc(-c2cc3ccccc3cc2-c2c3ccccc3c(-c3cc4cccc5oc6c(-c7ccccc7)ccc3c6c45)c3ccccc23)cc1 ; c1ccc(-c2c(-c3c4ccccc4c(-c4cc5cccc6oc7c(-c8ccccc8)ccc4c7c56)c4ccccc34)ccc3ccccc23)cc1 ; c1ccc(-c2cccc3ccc(-c4c5ccccc5c(-c5cc6cccc7oc8c(-c9ccccc9)ccc5c8c67)c5ccccc45)cc23)cc1] 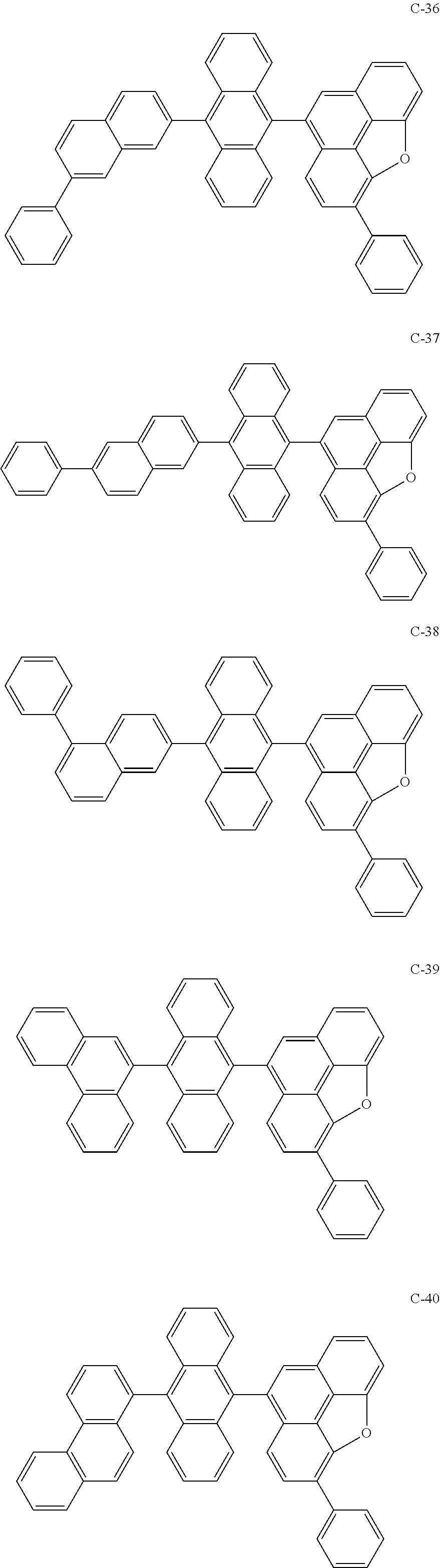 [SMILES: c1ccc(-c2ccc3ccc(-c4c5ccccc5c(-c5cc6cccc7oc8c(-c9ccccc9)ccc5c8c67)c5ccccc45)cc3c2)cc1 ; c1ccc(-c2ccc3cc(-c4c5ccccc5c(-c5cc6cccc7oc8c(-c9ccccc9)ccc5c8c67)c5ccccc45)ccc3c2)cc1 ; c1ccc(-c2cccc3cc(-c4c5ccccc5c(-c5cc6cccc7oc8c(-c9ccccc9)ccc5c8c67)c5ccccc45)ccc23)cc1 ; c1ccc(-c2ccc3c(-c4c5ccccc5c(-c5cc6ccccc6c6ccccc56)c5ccccc45)cc4cccc5oc2c3c45)cc1 ; c1ccc(-c2ccc3c(-c4c5ccccc5c(-c5cccc6c5ccc5ccccc56)c5ccccc45)cc4cccc5oc2c3c45)cc1] 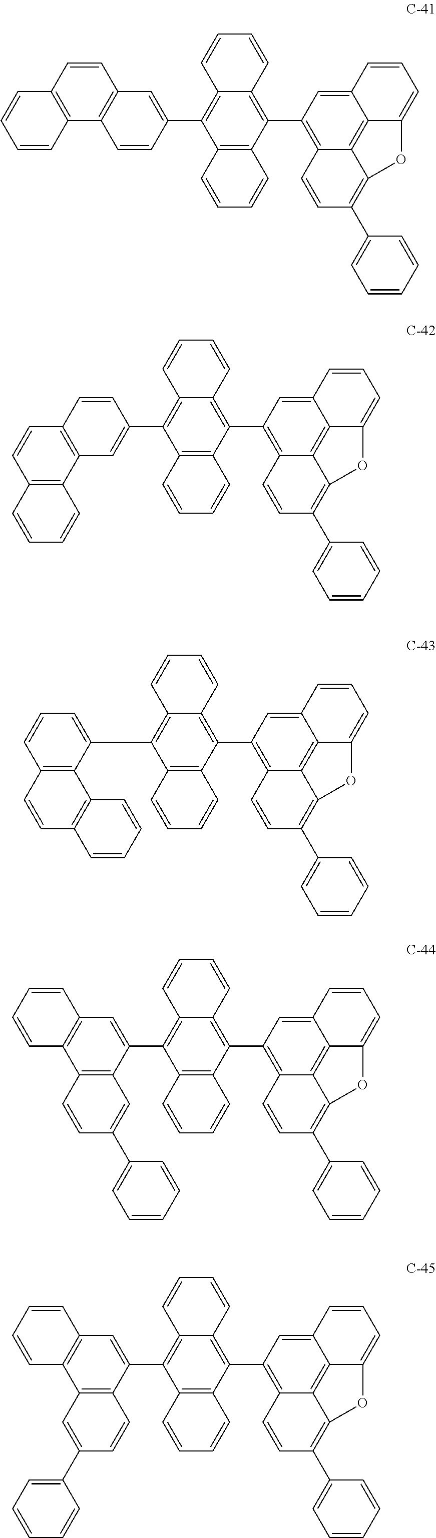 [SMILES: c1ccc(-c2ccc3c(-c4c5ccccc5c(-c5ccc6c(ccc7ccccc76)c5)c5ccccc45)cc4cccc5oc2c3c45)cc1 ; c1ccc(-c2ccc3c(-c4c5ccccc5c(-c5ccc6ccc7ccccc7c6c5)c5ccccc45)cc4cccc5oc2c3c45)cc1 ; c1ccc(-c2ccc3c(-c4c5ccccc5c(-c5cccc6ccc7ccccc7c56)c5ccccc45)cc4cccc5oc2c3c45)cc1 ; c1ccc(-c2ccc3c(c2)c(-c2c4ccccc4c(-c4cc5cccc6oc7c(-c8ccccc8)ccc4c7c56)c4ccccc24)cc2ccccc23)cc1 ; c1ccc(-c2ccc3c(-c4c5ccccc5c(-c5cc6cccc7oc8c(-c9ccccc9)ccc5c8c67)c5ccccc45)cc4ccccc4c3c2)cc1] 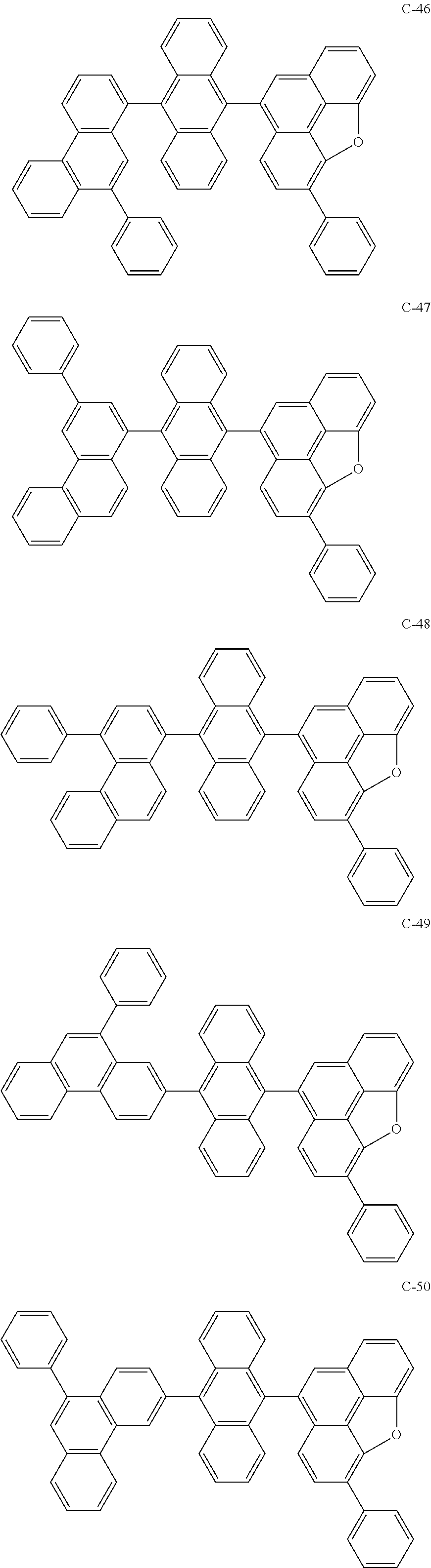 [SMILES: c1ccc(-c2cc3c(-c4c5ccccc5c(-c5cc6cccc7oc8c(-c9ccccc9)ccc5c8c67)c5ccccc45)cccc3c3ccccc23)cc1 ; c1ccc(-c2cc(-c3c4ccccc4c(-c4cc5cccc6oc7c(-c8ccccc8)ccc4c7c56)c4ccccc34)c3ccc4ccccc4c3c2)cc1 ; c1ccc(-c2ccc3c(-c4c5ccccc5c(-c5ccc(-c6ccccc6)c6c5ccc5ccccc56)c5ccccc45)cc4cccc5oc2c3c45)cc1 ; c1ccc(-c2cc3ccccc3c3ccc(-c4c5ccccc5c(-c5cc6cccc7oc8c(-c9ccccc9)ccc5c8c67)c5ccccc45)cc23)cc1 ; c1ccc(-c2cc3ccccc3c3cc(-c4c5ccccc5c(-c5cc6cccc7oc8c(-c9ccccc9)ccc5c8c67)c5ccccc45)ccc23)cc1] 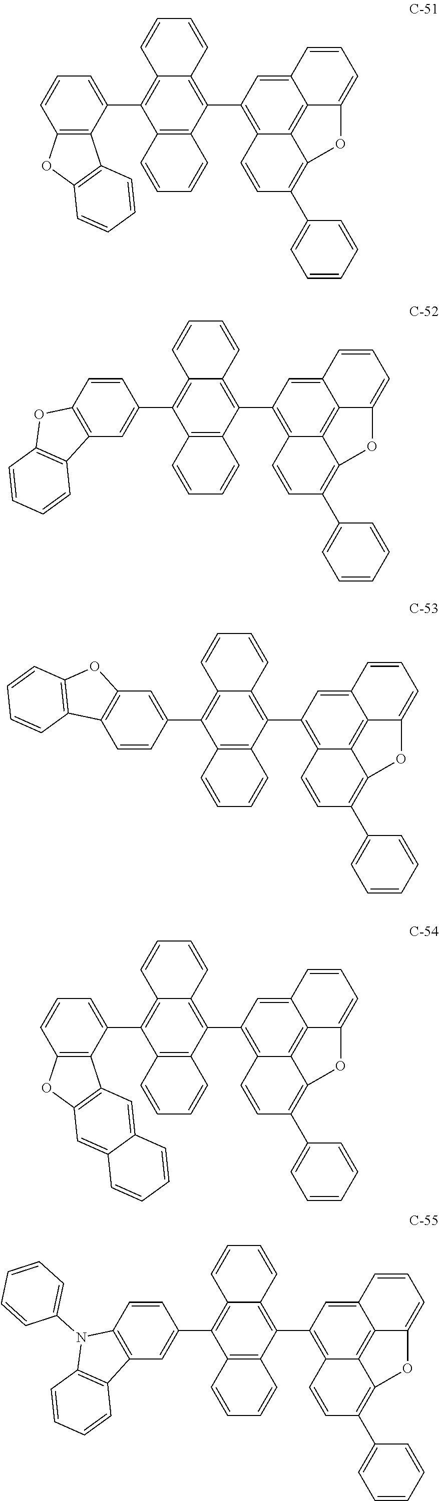 [SMILES: c1ccc(-c2ccc3c(-c4c5ccccc5c(-c5cccc6oc7ccccc7c56)c5ccccc45)cc4cccc5oc2c3c45)cc1 ; c1ccc(-c2ccc3c(-c4c5ccccc5c(-c5ccc6oc7ccccc7c6c5)c5ccccc45)cc4cccc5oc2c3c45)cc1 ; c1ccc(-c2ccc3c(-c4c5ccccc5c(-c5ccc6c(c5)oc5ccccc56)c5ccccc45)cc4cccc5oc2c3c45)cc1 ; c1ccc(-c2ccc3c(-c4c5ccccc5c(-c5cccc6oc7cc8ccccc8cc7c56)c5ccccc45)cc4cccc5oc2c3c45)cc1 ; c1ccc(-c2ccc3c(-c4c5ccccc5c(-c5ccc6c(c5)c5ccccc5n6-c5ccccc5)c5ccccc45)cc4cccc5oc2c3c45)cc1] 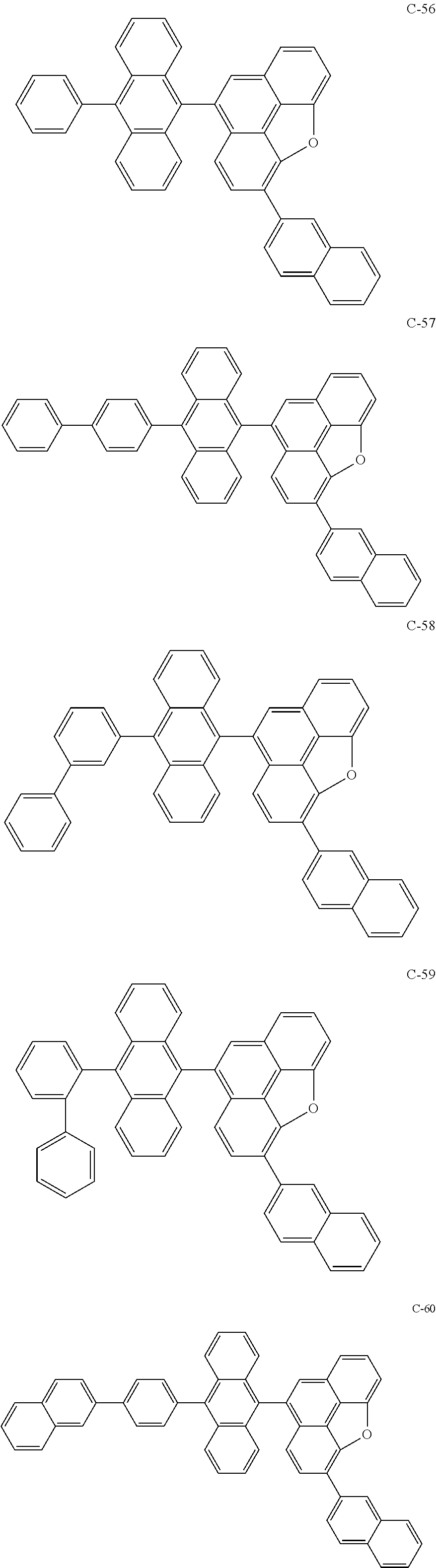 [SMILES: c1ccc(-c2c3ccccc3c(-c3cc4cccc5oc6c(-c7ccc8ccccc8c7)ccc3c6c45)c3ccccc23)cc1 ; c1ccc(-c2ccc(-c3c4ccccc4c(-c4cc5cccc6oc7c(-c8ccc9ccccc9c8)ccc4c7c56)c4ccccc34)cc2)cc1 ; c1ccc(-c2cccc(-c3c4ccccc4c(-c4cc5cccc6oc7c(-c8ccc9ccccc9c8)ccc4c7c56)c4ccccc34)c2)cc1 ; c1ccc(-c2ccccc2-c2c3ccccc3c(-c3cc4cccc5oc6c(-c7ccc8ccccc8c7)ccc3c6c45)c3ccccc23)cc1 ; c1ccc2cc(-c3ccc(-c4c5ccccc5c(-c5cc6cccc7oc8c(-c9ccc%10ccccc%10c9)ccc5c8c67)c5ccccc45)cc3)ccc2c1] 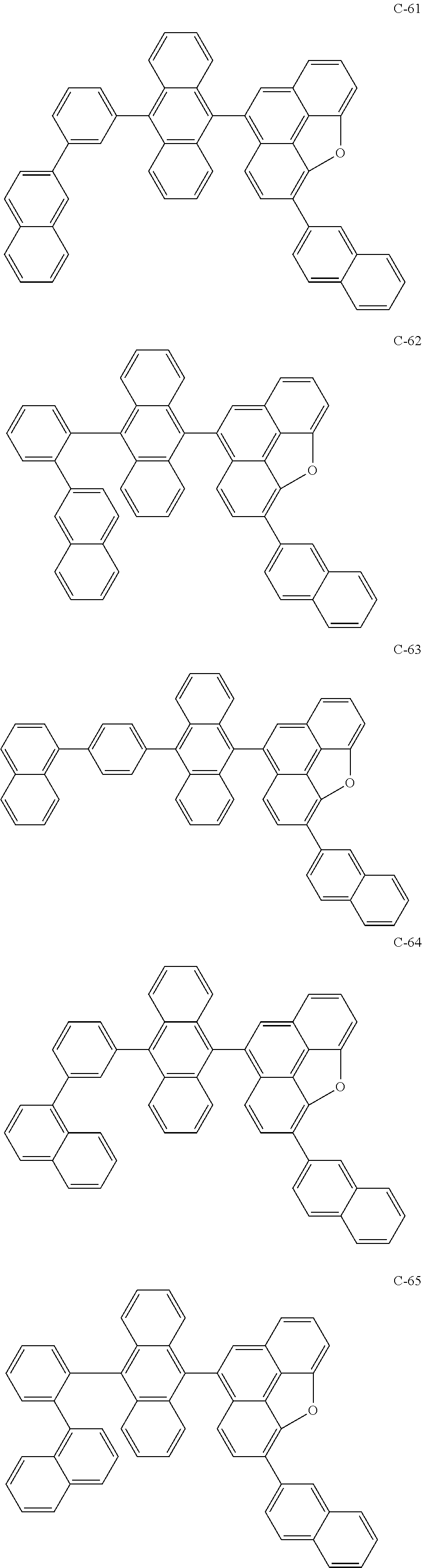 [SMILES: c1cc(-c2ccc3ccccc3c2)cc(-c2c3ccccc3c(-c3cc4cccc5oc6c(-c7ccc8ccccc8c7)ccc3c6c45)c3ccccc23)c1 ; c1ccc(-c2c3ccccc3c(-c3cc4cccc5oc6c(-c7ccc8ccccc8c7)ccc3c6c45)c3ccccc23)c(-c2ccc3ccccc3c2)c1 ; c1ccc2cc(-c3ccc4c(-c5c6ccccc6c(-c6ccc(-c7cccc8ccccc78)cc6)c6ccccc56)cc5cccc6oc3c4c56)ccc2c1 ; c1cc(-c2cccc3ccccc23)cc(-c2c3ccccc3c(-c3cc4cccc5oc6c(-c7ccc8ccccc8c7)ccc3c6c45)c3ccccc23)c1 ; c1ccc(-c2c3ccccc3c(-c3cc4cccc5oc6c(-c7ccc8ccccc8c7)ccc3c6c45)c3ccccc23)c(-c2cccc3ccccc23)c1] 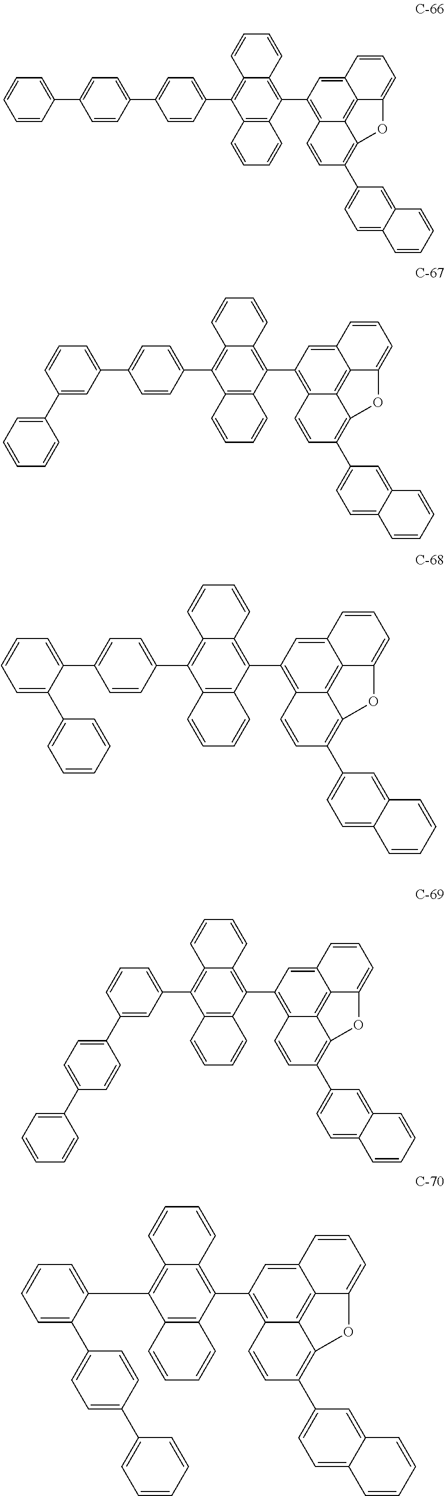 [SMILES: c1ccc(-c2ccc(-c3ccc(-c4c5ccccc5c(-c5cc6cccc7oc8c(-c9ccc%10ccccc%10c9)ccc5c8c67)c5ccccc45)cc3)cc2)cc1 ; c1ccc(-c2cccc(-c3ccc(-c4c5ccccc5c(-c5cc6cccc7oc8c(-c9ccc%10ccccc%10c9)ccc5c8c67)c5ccccc45)cc3)c2)cc1 ; c1ccc(-c2ccccc2-c2ccc(-c3c4ccccc4c(-c4cc5cccc6oc7c(-c8ccc9ccccc9c8)ccc4c7c56)c4ccccc34)cc2)cc1 ; c1ccc(-c2ccc(-c3cccc(-c4c5ccccc5c(-c5cc6cccc7oc8c(-c9ccc%10ccccc%10c9)ccc5c8c67)c5ccccc45)c3)cc2)cc1 ; c1ccc(-c2ccc(-c3ccccc3-c3c4ccccc4c(-c4cc5cccc6oc7c(-c8ccc9ccccc9c8)ccc4c7c56)c4ccccc34)cc2)cc1] 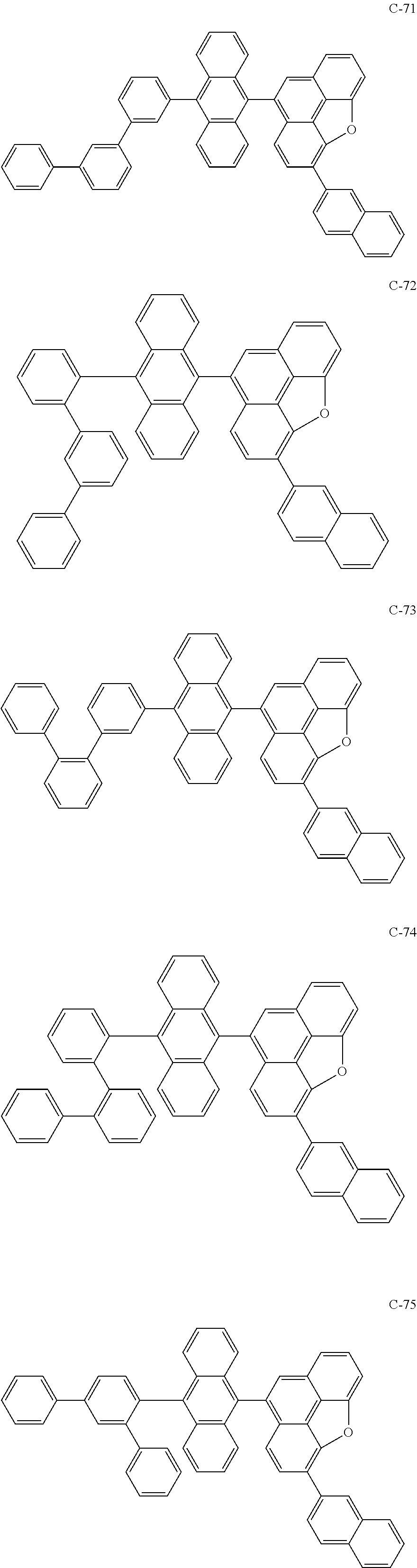 [SMILES: c1ccc(-c2cccc(-c3cccc(-c4c5ccccc5c(-c5cc6cccc7oc8c(-c9ccc%10ccccc%10c9)ccc5c8c67)c5ccccc45)c3)c2)cc1 ; c1ccc(-c2cccc(-c3ccccc3-c3c4ccccc4c(-c4cc5cccc6oc7c(-c8ccc9ccccc9c8)ccc4c7c56)c4ccccc34)c2)cc1 ; c1ccc(-c2ccccc2-c2cccc(-c3c4ccccc4c(-c4cc5cccc6oc7c(-c8ccc9ccccc9c8)ccc4c7c56)c4ccccc34)c2)cc1 ; c1ccc(-c2ccccc2-c2ccccc2-c2c3ccccc3c(-c3cc4cccc5oc6c(-c7ccc8ccccc8c7)ccc3c6c45)c3ccccc23)cc1 ; c1ccc(-c2ccc(-c3c4ccccc4c(-c4cc5cccc6oc7c(-c8ccc9ccccc9c8)ccc4c7c56)c4ccccc34)c(-c3ccccc3)c2)cc1] 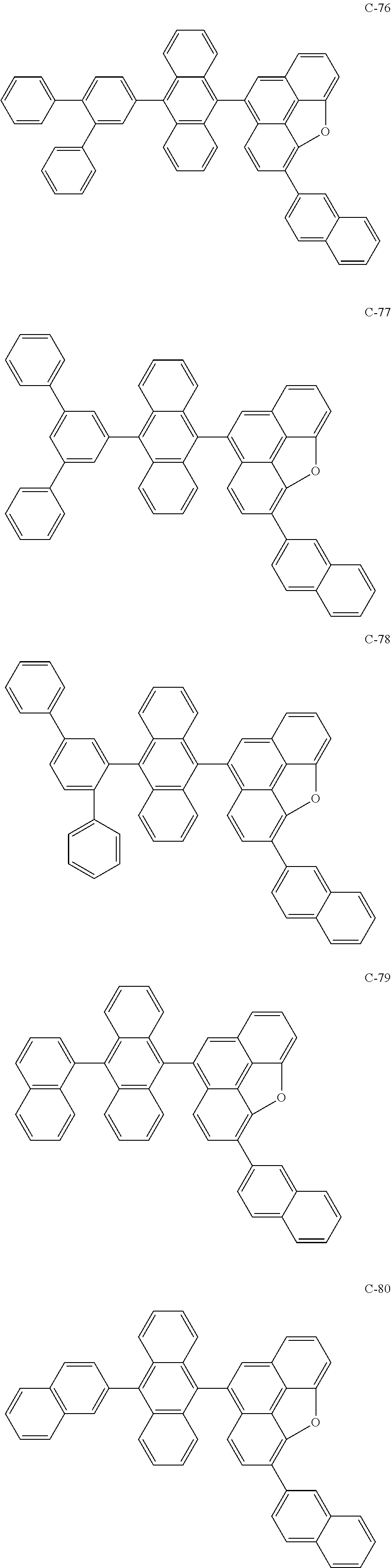 [SMILES: c1ccc(-c2ccc(-c3c4ccccc4c(-c4cc5cccc6oc7c(-c8ccc9ccccc9c8)ccc4c7c56)c4ccccc34)cc2-c2ccccc2)cc1 ; c1ccc(-c2cc(-c3ccccc3)cc(-c3c4ccccc4c(-c4cc5cccc6oc7c(-c8ccc9ccccc9c8)ccc4c7c56)c4ccccc34)c2)cc1 ; c1ccc(-c2ccc(-c3ccccc3)c(-c3c4ccccc4c(-c4cc5cccc6oc7c(-c8ccc9ccccc9c8)ccc4c7c56)c4ccccc34)c2)cc1 ; c1ccc2cc(-c3ccc4c(-c5c6ccccc6c(-c6cccc7ccccc67)c6ccccc56)cc5cccc6oc3c4c56)ccc2c1 ; c1ccc2cc(-c3c4ccccc4c(-c4cc5cccc6oc7c(-c8ccc9ccccc9c8)ccc4c7c56)c4ccccc34)ccc2c1] 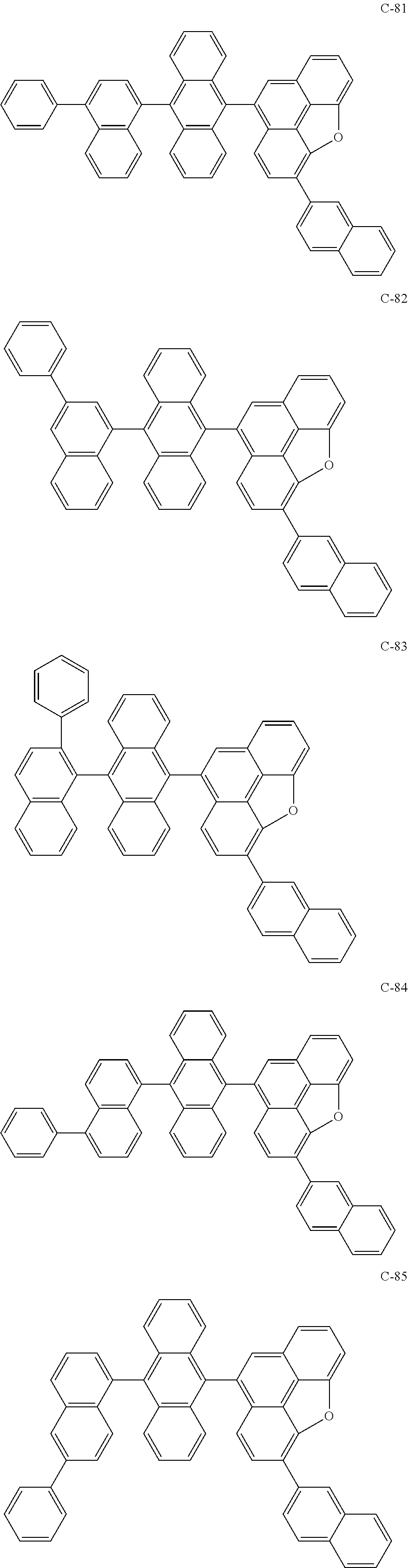 [SMILES: c1ccc(-c2ccc(-c3c4ccccc4c(-c4cc5cccc6oc7c(-c8ccc9ccccc9c8)ccc4c7c56)c4ccccc34)c3ccccc23)cc1 ; c1ccc(-c2cc(-c3c4ccccc4c(-c4cc5cccc6oc7c(-c8ccc9ccccc9c8)ccc4c7c56)c4ccccc34)c3ccccc3c2)cc1 ; c1ccc(-c2ccc3ccccc3c2-c2c3ccccc3c(-c3cc4cccc5oc6c(-c7ccc8ccccc8c7)ccc3c6c45)c3ccccc23)cc1 ; c1ccc(-c2cccc3c(-c4c5ccccc5c(-c5cc6cccc7oc8c(-c9ccc%10ccccc%10c9)ccc5c8c67)c5ccccc45)cccc23)cc1 ; c1ccc(-c2ccc3c(-c4c5ccccc5c(-c5cc6cccc7oc8c(-c9ccc%10ccccc%10c9)ccc5c8c67)c5ccccc45)cccc3c2)cc1] 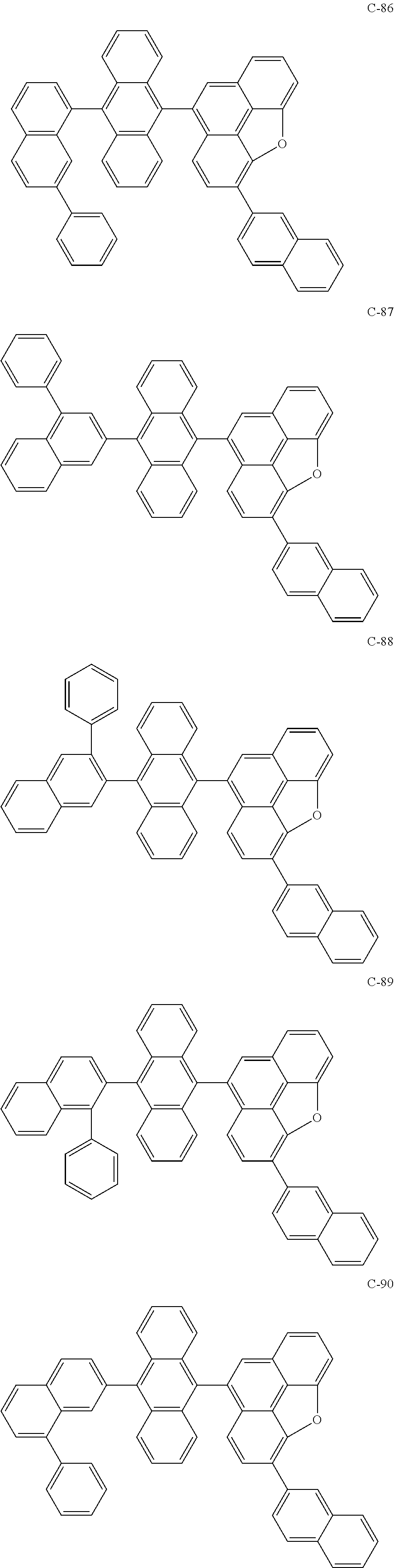 [SMILES: c1ccc(-c2ccc3cccc(-c4c5ccccc5c(-c5cc6cccc7oc8c(-c9ccc%10ccccc%10c9)ccc5c8c67)c5ccccc45)c3c2)cc1 ; c1ccc(-c2cc(-c3c4ccccc4c(-c4cc5cccc6oc7c(-c8ccc9ccccc9c8)ccc4c7c56)c4ccccc34)cc3ccccc23)cc1 ; c1ccc(-c2cc3ccccc3cc2-c2c3ccccc3c(-c3cc4cccc5oc6c(-c7ccc8ccccc8c7)ccc3c6c45)c3ccccc23)cc1 ; c1ccc(-c2c(-c3c4ccccc4c(-c4cc5cccc6oc7c(-c8ccc9ccccc9c8)ccc4c7c56)c4ccccc34)ccc3ccccc23)cc1 ; c1ccc(-c2cccc3ccc(-c4c5ccccc5c(-c5cc6cccc7oc8c(-c9ccc%10ccccc%10c9)ccc5c8c67)c5ccccc45)cc23)cc1] 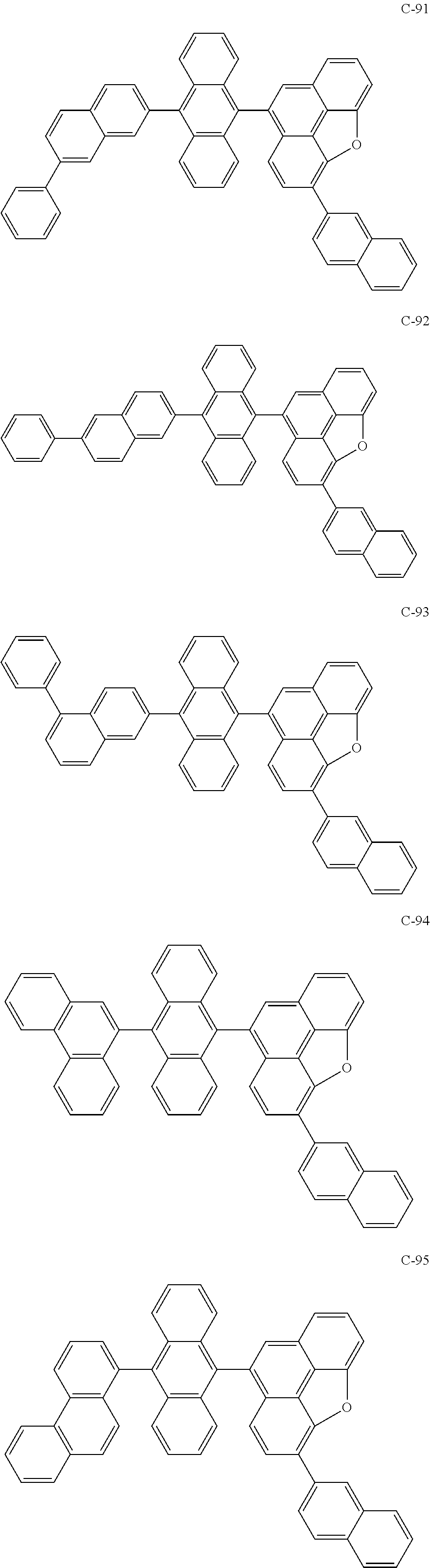 [SMILES: c1ccc(-c2ccc3ccc(-c4c5ccccc5c(-c5cc6cccc7oc8c(-c9ccc%10ccccc%10c9)ccc5c8c67)c5ccccc45)cc3c2)cc1 ; c1ccc(-c2ccc3cc(-c4c5ccccc5c(-c5cc6cccc7oc8c(-c9ccc%10ccccc%10c9)ccc5c8c67)c5ccccc45)ccc3c2)cc1 ; c1ccc(-c2cccc3cc(-c4c5ccccc5c(-c5cc6cccc7oc8c(-c9ccc%10ccccc%10c9)ccc5c8c67)c5ccccc45)ccc23)cc1 ; c1ccc2cc(-c3ccc4c(-c5c6ccccc6c(-c6cc7ccccc7c7ccccc67)c6ccccc56)cc5cccc6oc3c4c56)ccc2c1 ; c1ccc2cc(-c3ccc4c(-c5c6ccccc6c(-c6cccc7c6ccc6ccccc67)c6ccccc56)cc5cccc6oc3c4c56)ccc2c1] 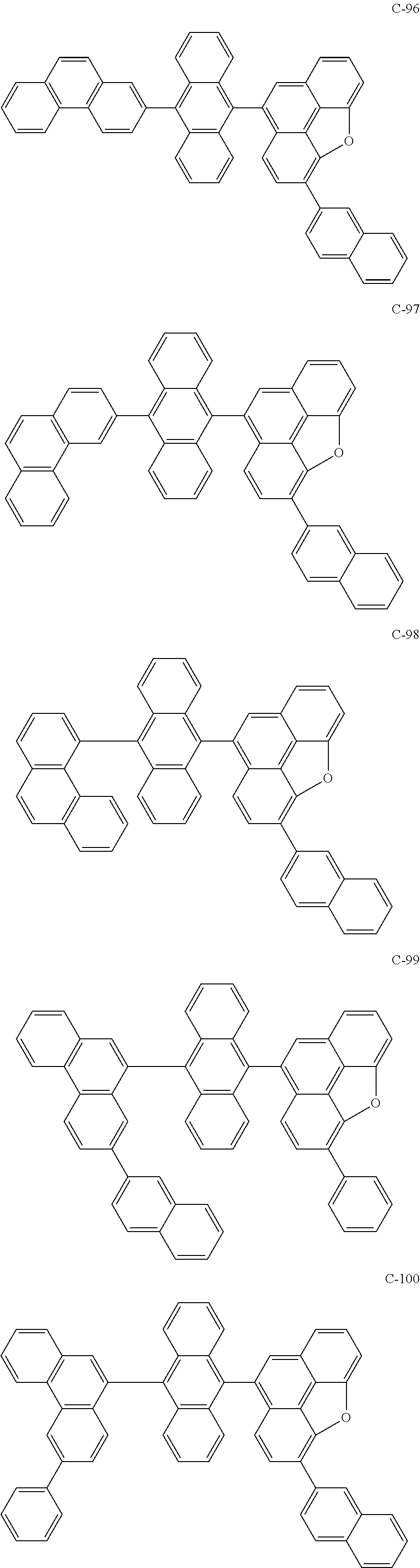 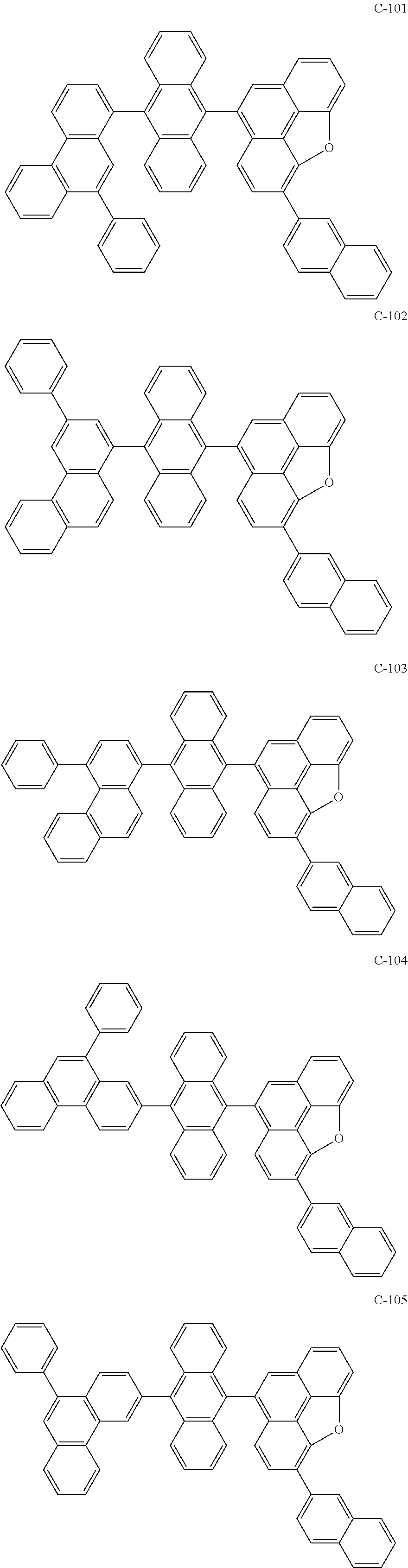 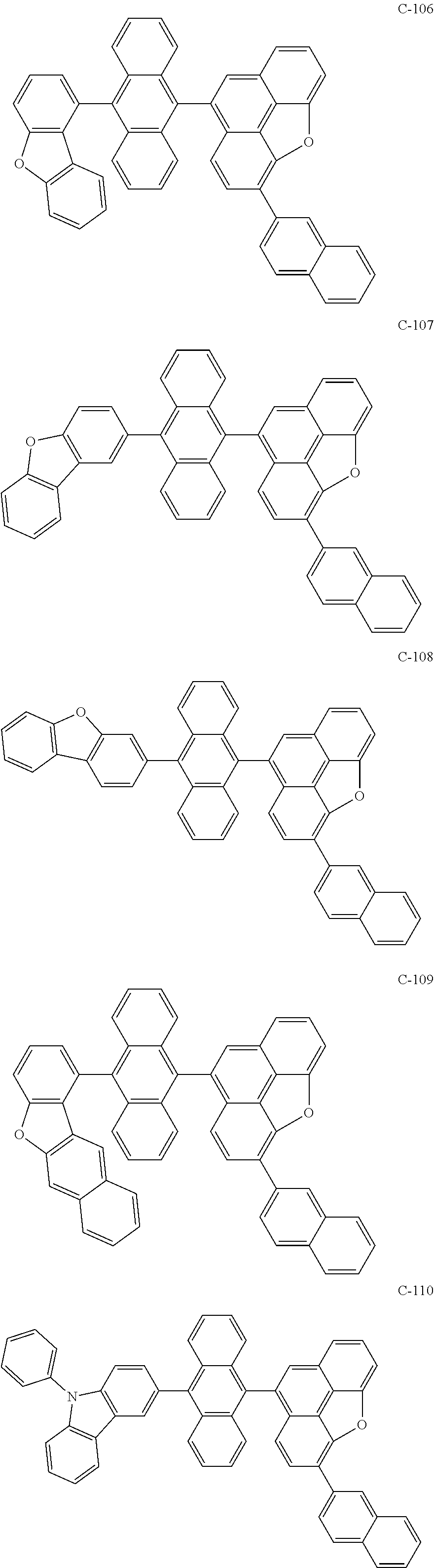 [SMILES: c1ccc2cc(-c3ccc4c(-c5c6ccccc6c(-c6cccc7oc8ccccc8c67)c6ccccc56)cc5cccc6oc3c4c56)ccc2c1 ; c1ccc2cc(-c3ccc4c(-c5c6ccccc6c(-c6ccc7oc8ccccc8c7c6)c6ccccc56)cc5cccc6oc3c4c56)ccc2c1 ; c1ccc2cc(-c3ccc4c(-c5c6ccccc6c(-c6ccc7c(c6)oc6ccccc67)c6ccccc56)cc5cccc6oc3c4c56)ccc2c1 ; c1ccc2cc(-c3ccc4c(-c5c6ccccc6c(-c6cccc7oc8cc9ccccc9cc8c67)c6ccccc56)cc5cccc6oc3c4c56)ccc2c1 ; c1ccc(-n2c3ccccc3c3cc(-c4c5ccccc5c(-c5cc6cccc7oc8c(-c9ccc%10ccccc%10c9)ccc5c8c67)c5ccccc45)ccc32)cc1] 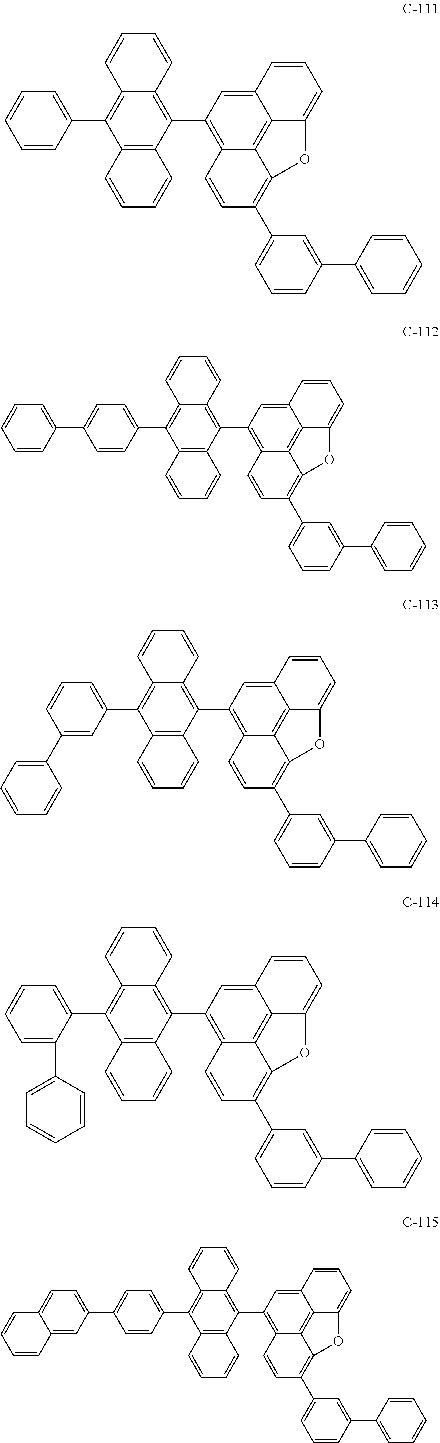 [SMILES: c1ccc(-c2cccc(-c3ccc4c(-c5c6ccccc6c(-c6ccccc6)c6ccccc56)cc5cccc6oc3c4c56)c2)cc1 ; c1ccc(-c2ccc(-c3c4ccccc4c(-c4cc5cccc6oc7c(-c8cccc(-c9ccccc9)c8)ccc4c7c56)c4ccccc34)cc2)cc1 ; c1ccc(-c2cccc(-c3c4ccccc4c(-c4cc5cccc6oc7c(-c8cccc(-c9ccccc9)c8)ccc4c7c56)c4ccccc34)c2)cc1 ; c1ccc(-c2cccc(-c3ccc4c(-c5c6ccccc6c(-c6ccccc6-c6ccccc6)c6ccccc56)cc5cccc6oc3c4c56)c2)cc1 ; c1ccc(-c2cccc(-c3ccc4c(-c5c6ccccc6c(-c6ccc(-c7ccc8ccccc8c7)cc6)c6ccccc56)cc5cccc6oc3c4c56)c2)cc1] 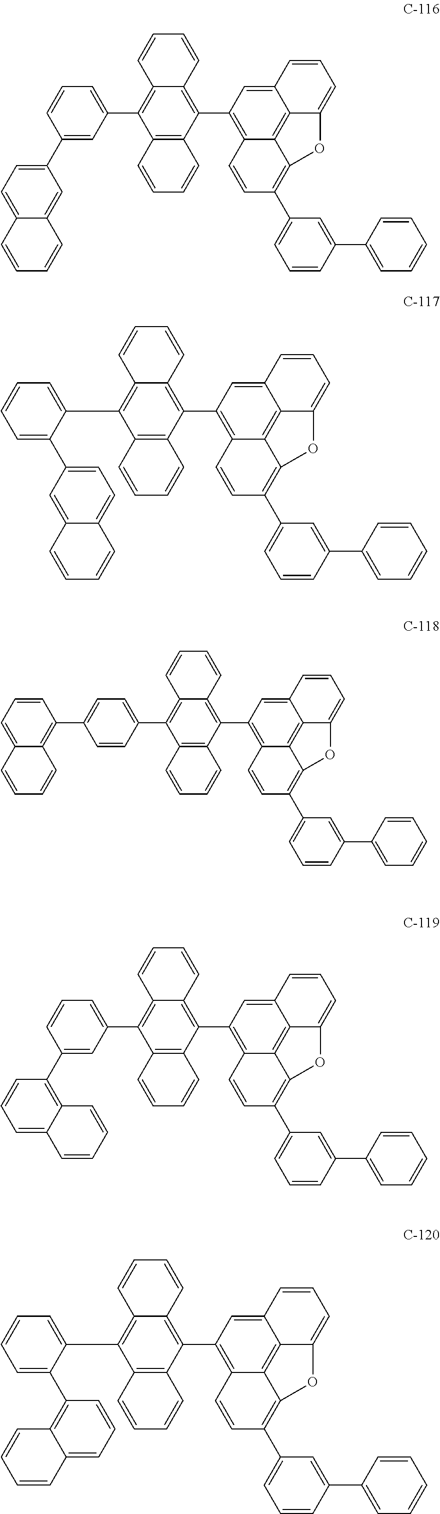 [SMILES: c1ccc(-c2cccc(-c3ccc4c(-c5c6ccccc6c(-c6cccc(-c7ccc8ccccc8c7)c6)c6ccccc56)cc5cccc6oc3c4c56)c2)cc1 ; c1ccc(-c2cccc(-c3ccc4c(-c5c6ccccc6c(-c6ccccc6-c6ccc7ccccc7c6)c6ccccc56)cc5cccc6oc3c4c56)c2)cc1 ; c1ccc(-c2cccc(-c3ccc4c(-c5c6ccccc6c(-c6ccc(-c7cccc8ccccc78)cc6)c6ccccc56)cc5cccc6oc3c4c56)c2)cc1 ; c1ccc(-c2cccc(-c3ccc4c(-c5c6ccccc6c(-c6cccc(-c7cccc8ccccc78)c6)c6ccccc56)cc5cccc6oc3c4c56)c2)cc1 ; c1ccc(-c2cccc(-c3ccc4c(-c5c6ccccc6c(-c6ccccc6-c6cccc7ccccc67)c6ccccc56)cc5cccc6oc3c4c56)c2)cc1] 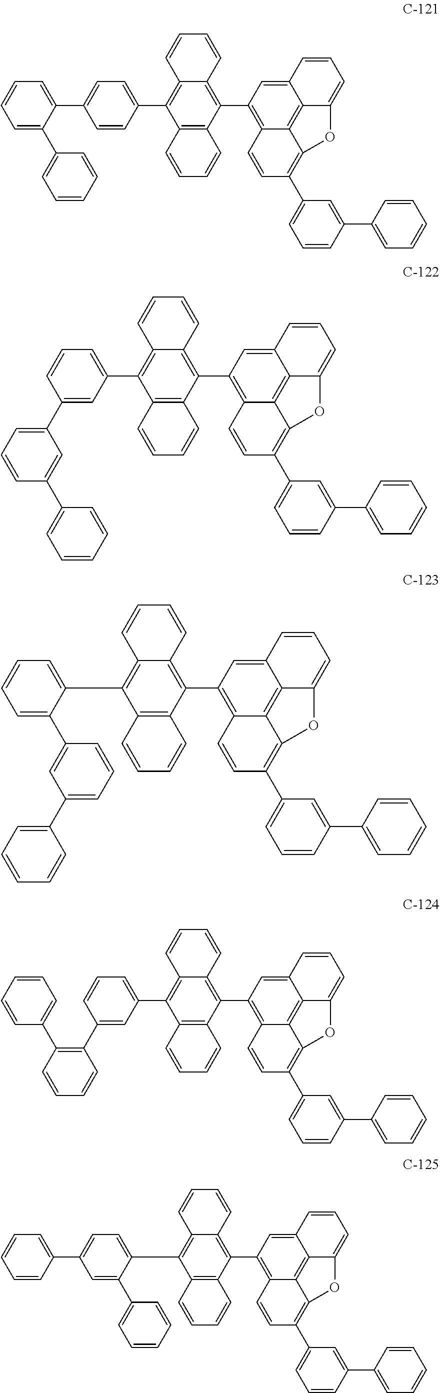 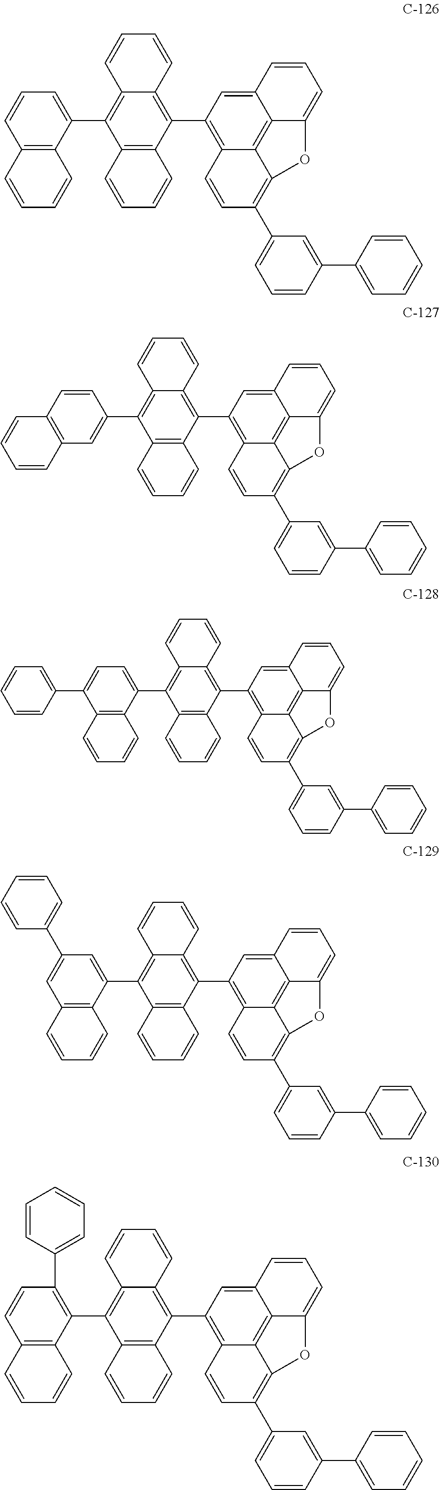 [SMILES: c1ccc(-c2cccc(-c3ccc4c(-c5c6ccccc6c(-c6cccc7ccccc67)c6ccccc56)cc5cccc6oc3c4c56)c2)cc1 ; c1ccc(-c2cccc(-c3ccc4c(-c5c6ccccc6c(-c6ccc7ccccc7c6)c6ccccc56)cc5cccc6oc3c4c56)c2)cc1 ; c1ccc(-c2cccc(-c3ccc4c(-c5c6ccccc6c(-c6ccc(-c7ccccc7)c7ccccc67)c6ccccc56)cc5cccc6oc3c4c56)c2)cc1 ; c1ccc(-c2cccc(-c3ccc4c(-c5c6ccccc6c(-c6cc(-c7ccccc7)cc7ccccc67)c6ccccc56)cc5cccc6oc3c4c56)c2)cc1 ; c1ccc(-c2cccc(-c3ccc4c(-c5c6ccccc6c(-c6c(-c7ccccc7)ccc7ccccc67)c6ccccc56)cc5cccc6oc3c4c56)c2)cc1] 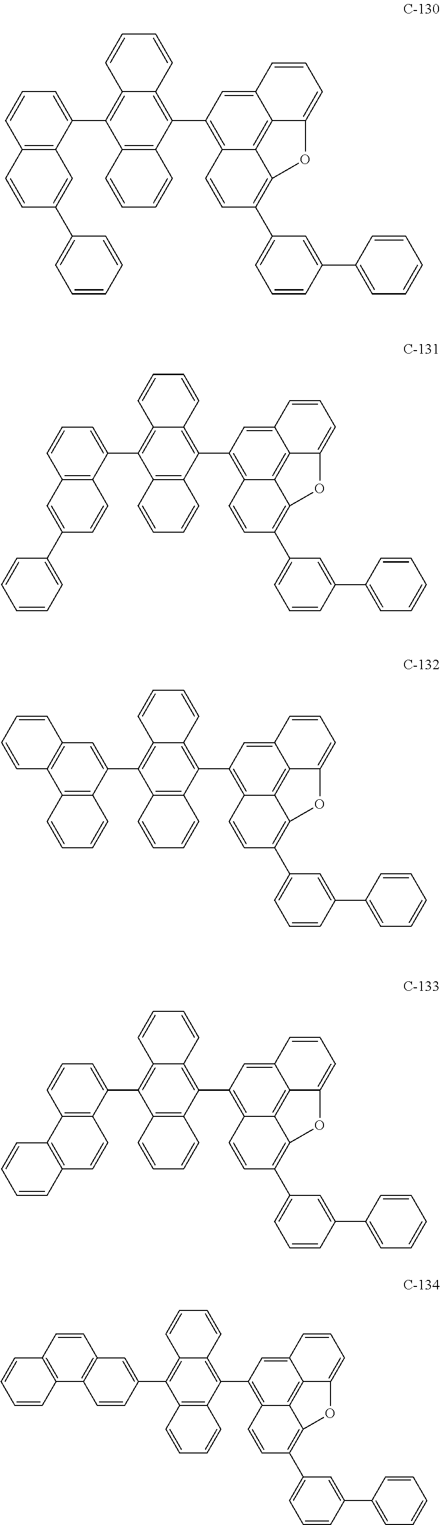 [SMILES: c1ccc(-c2cccc(-c3ccc4c(-c5c6ccccc6c(-c6cccc7ccc(-c8ccccc8)cc67)c6ccccc56)cc5cccc6oc3c4c56)c2)cc1 ; c1ccc(-c2cccc(-c3ccc4c(-c5c6ccccc6c(-c6cccc7cc(-c8ccccc8)ccc67)c6ccccc56)cc5cccc6oc3c4c56)c2)cc1 ; c1ccc(-c2cccc(-c3ccc4c(-c5c6ccccc6c(-c6cc7ccccc7c7ccccc67)c6ccccc56)cc5cccc6oc3c4c56)c2)cc1 ; c1ccc(-c2cccc(-c3ccc4c(-c5c6ccccc6c(-c6cccc7c6ccc6ccccc67)c6ccccc56)cc5cccc6oc3c4c56)c2)cc1 ; c1ccc(-c2cccc(-c3ccc4c(-c5c6ccccc6c(-c6ccc7c(ccc8ccccc87)c6)c6ccccc56)cc5cccc6oc3c4c56)c2)cc1] 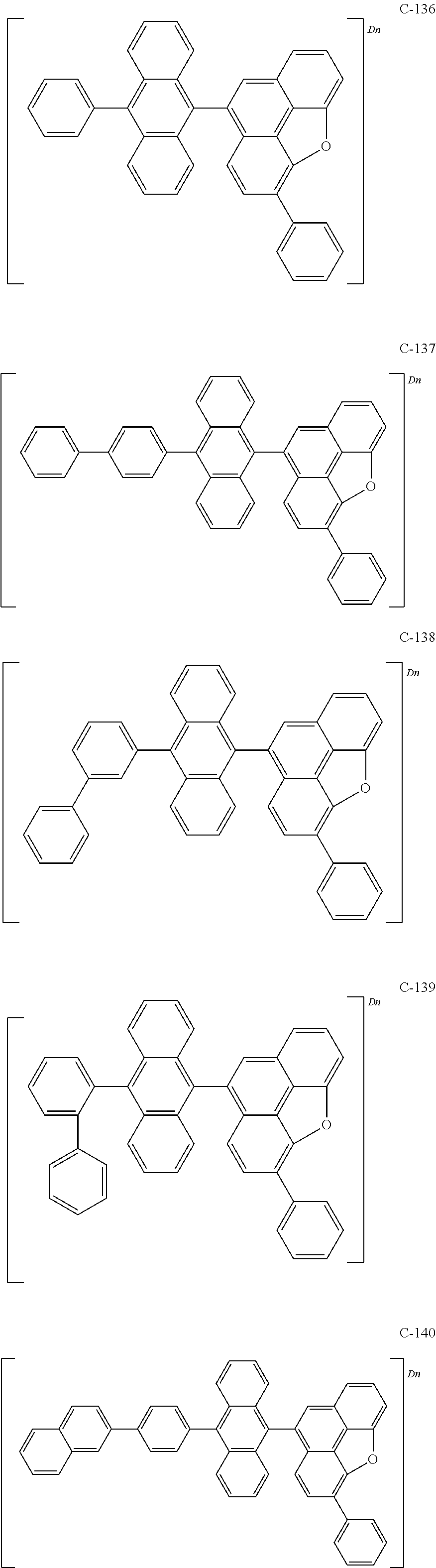 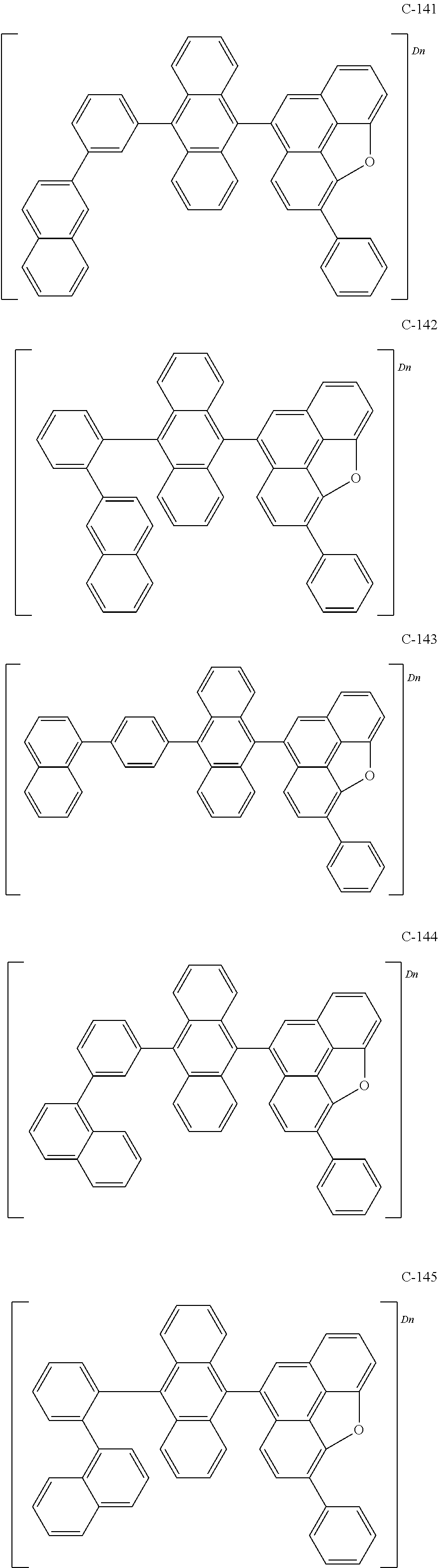 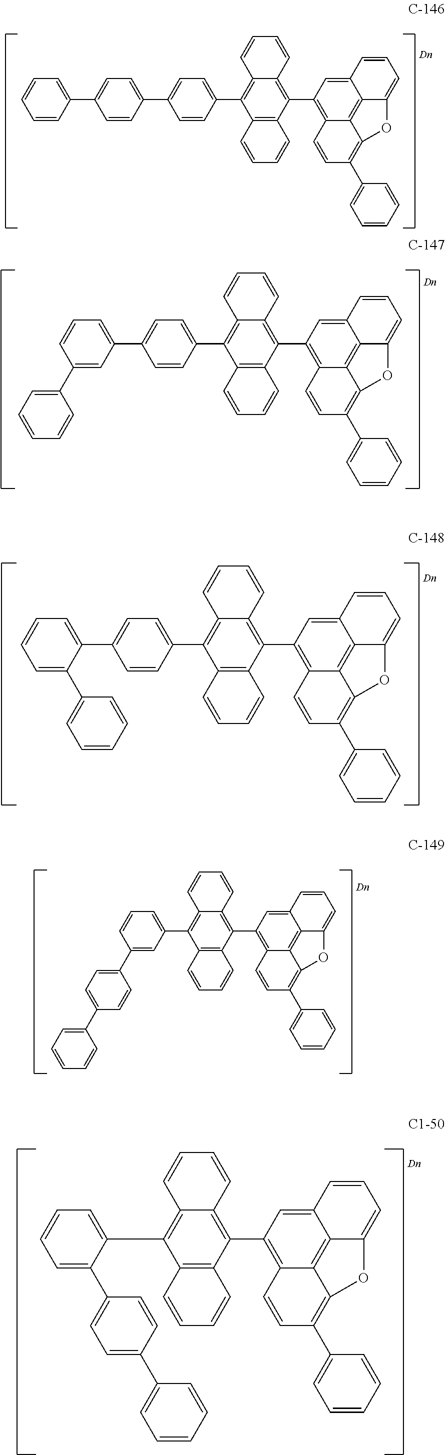 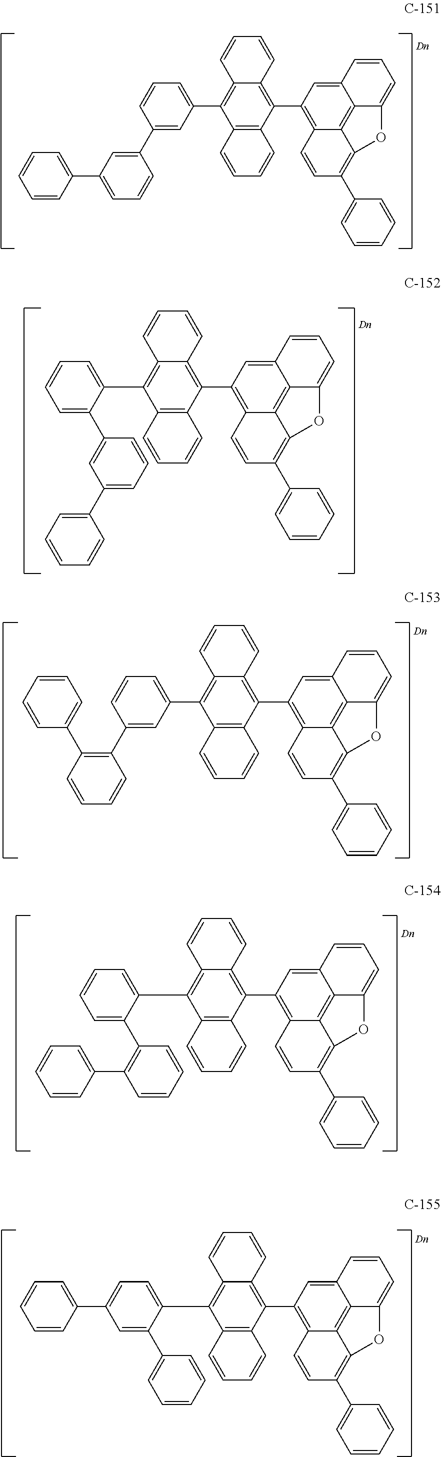 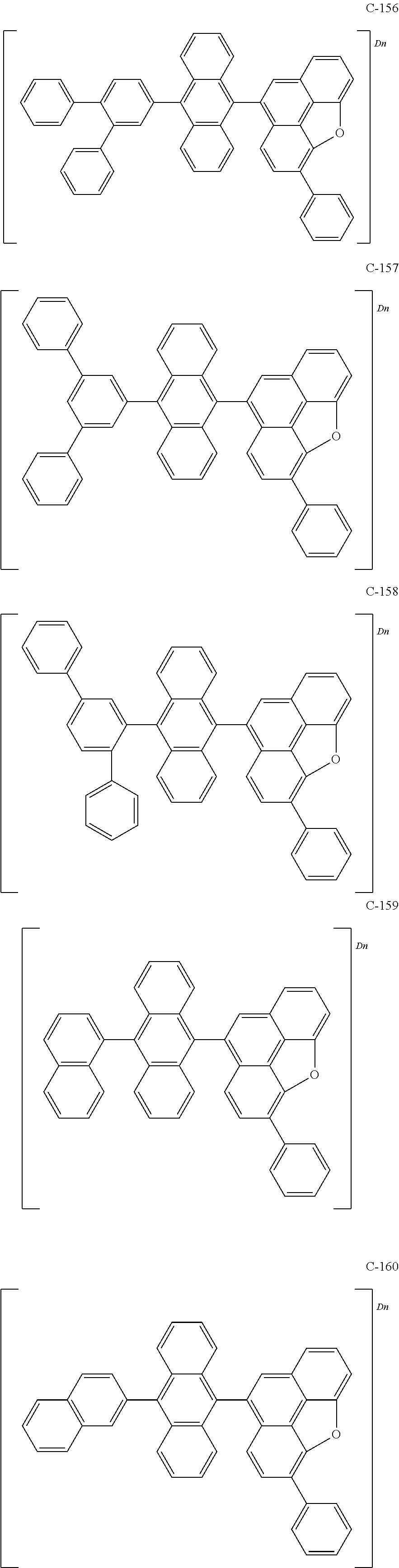 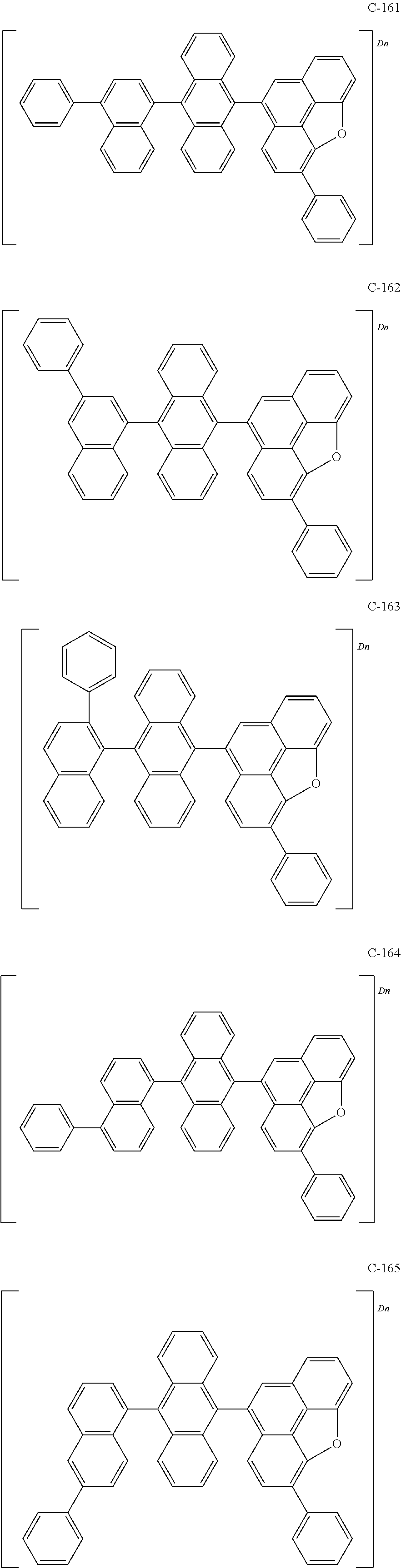 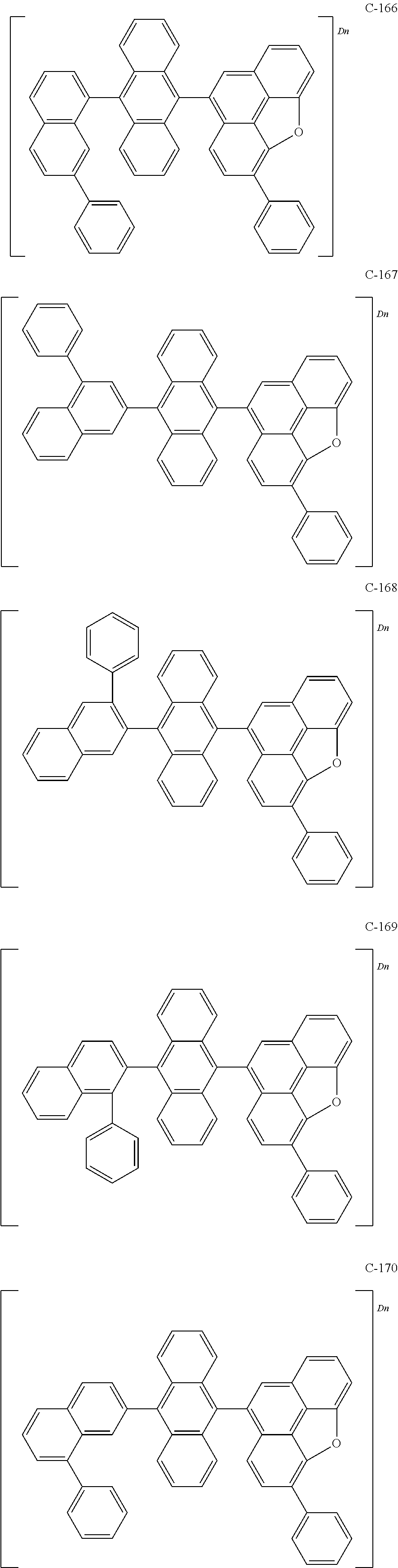 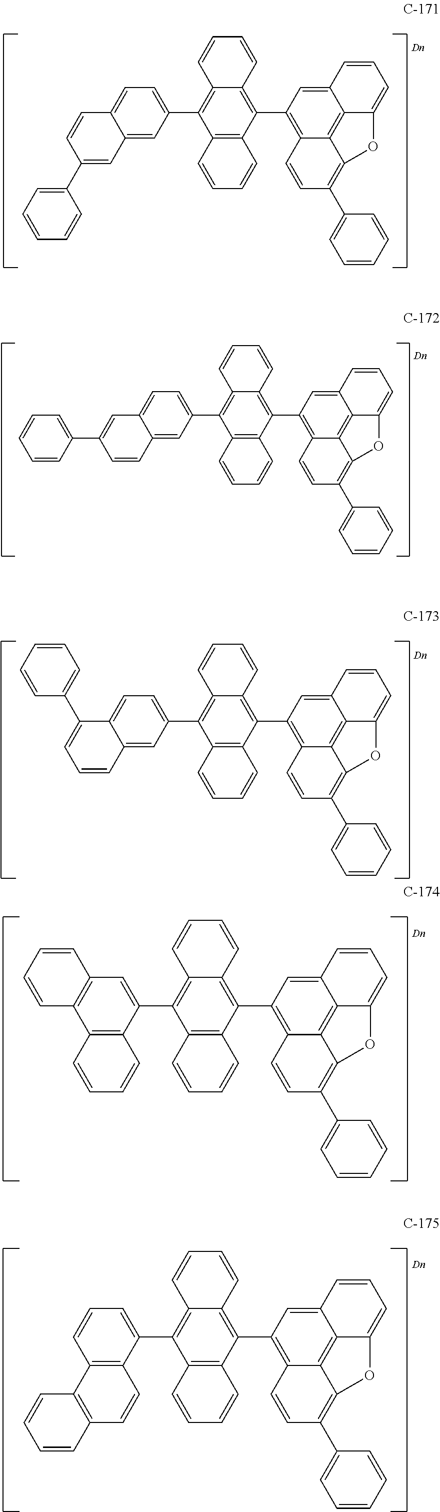 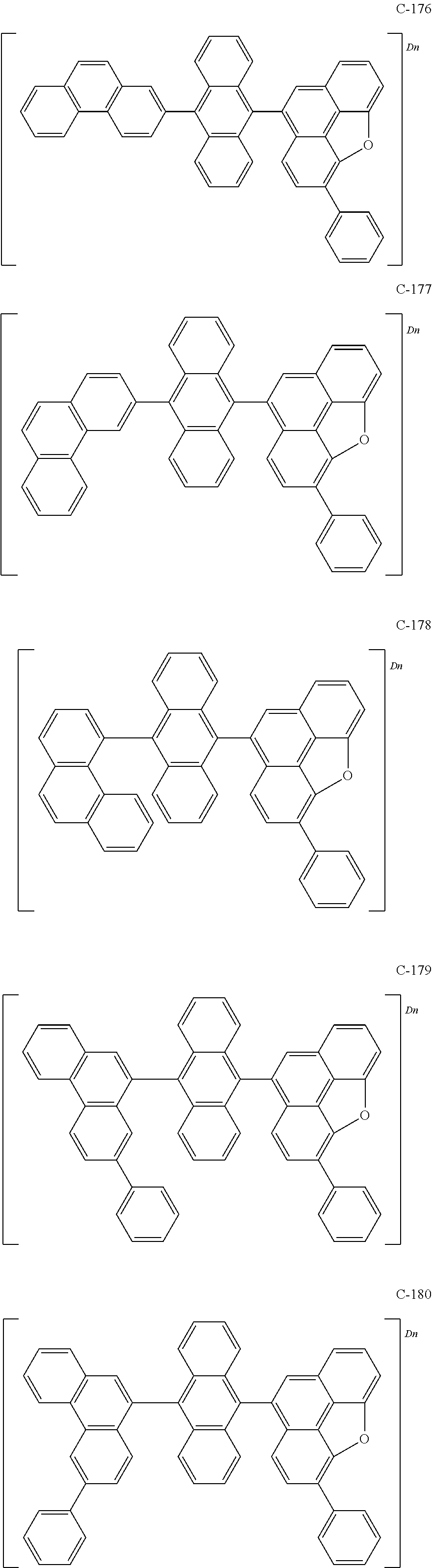 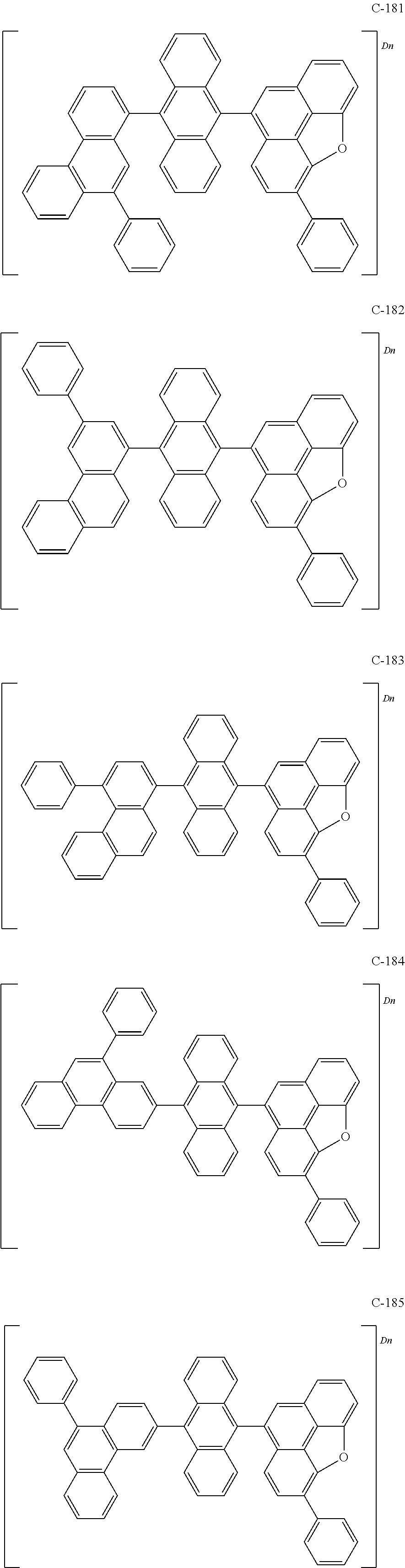 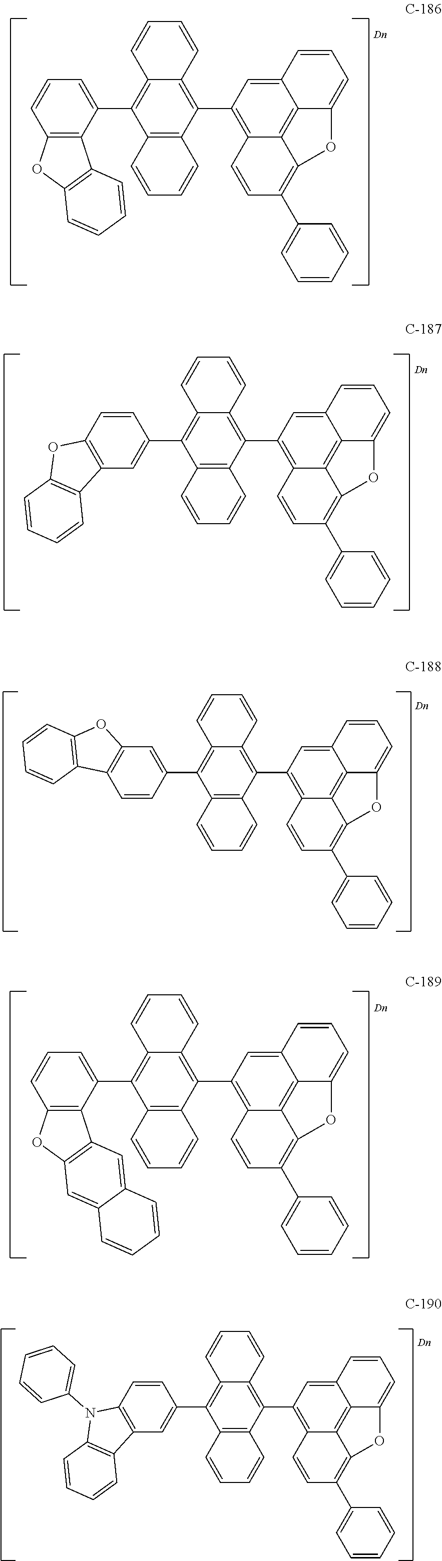 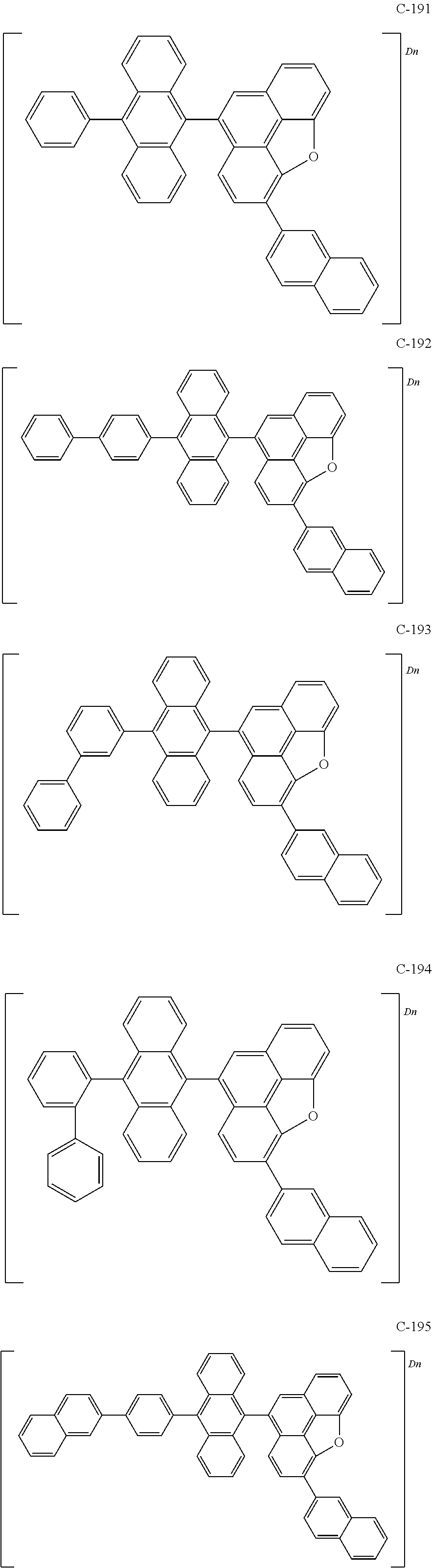 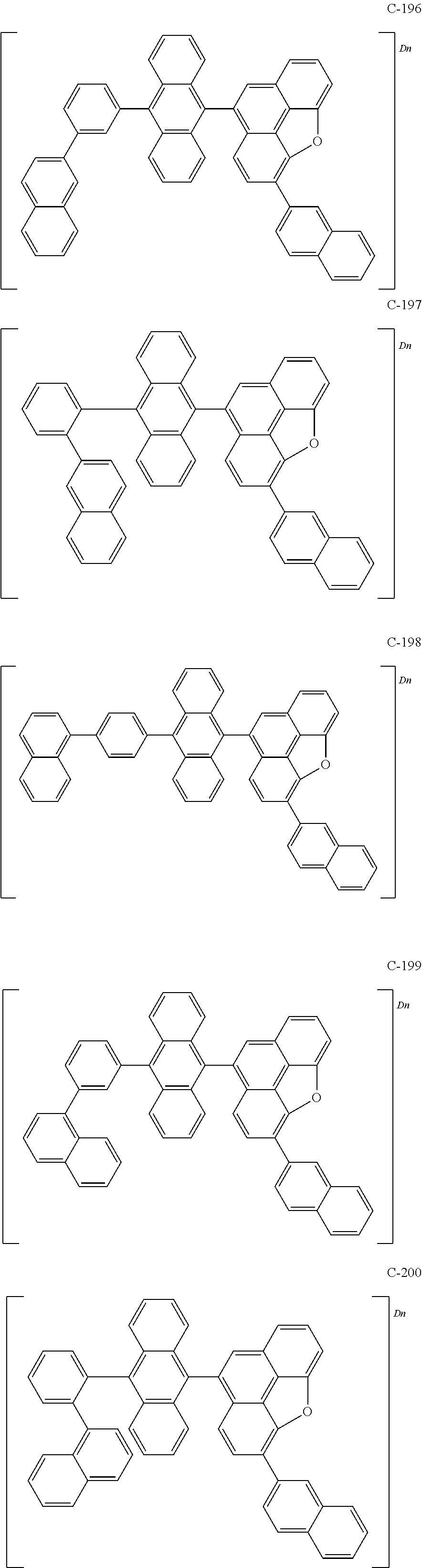 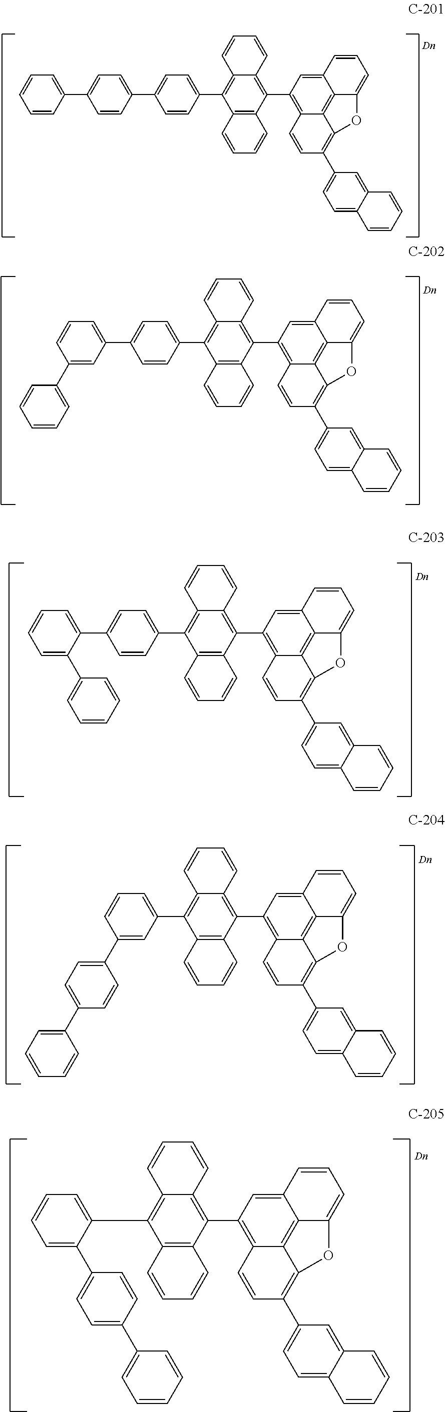 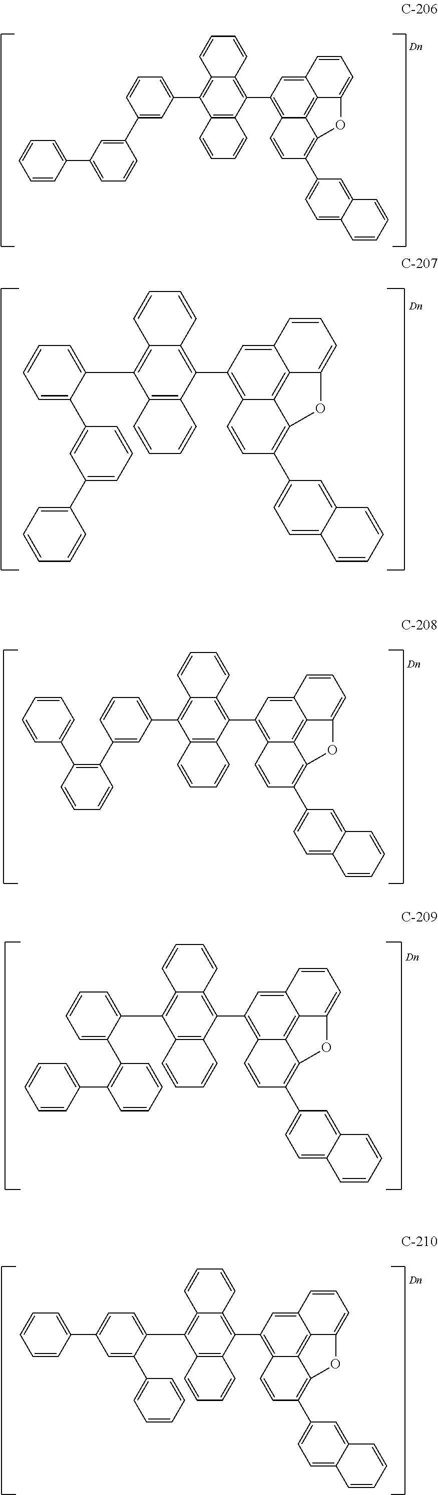 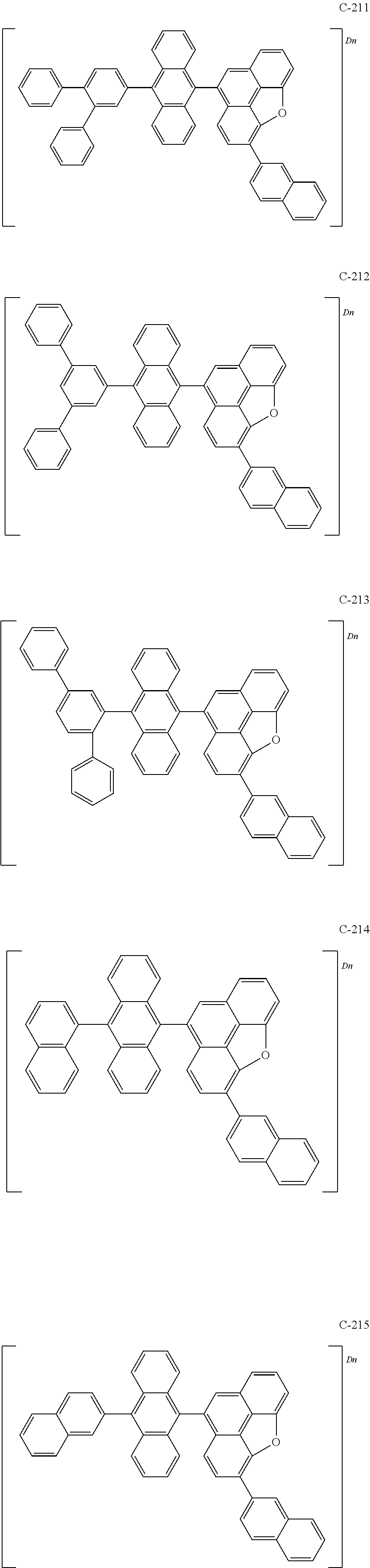 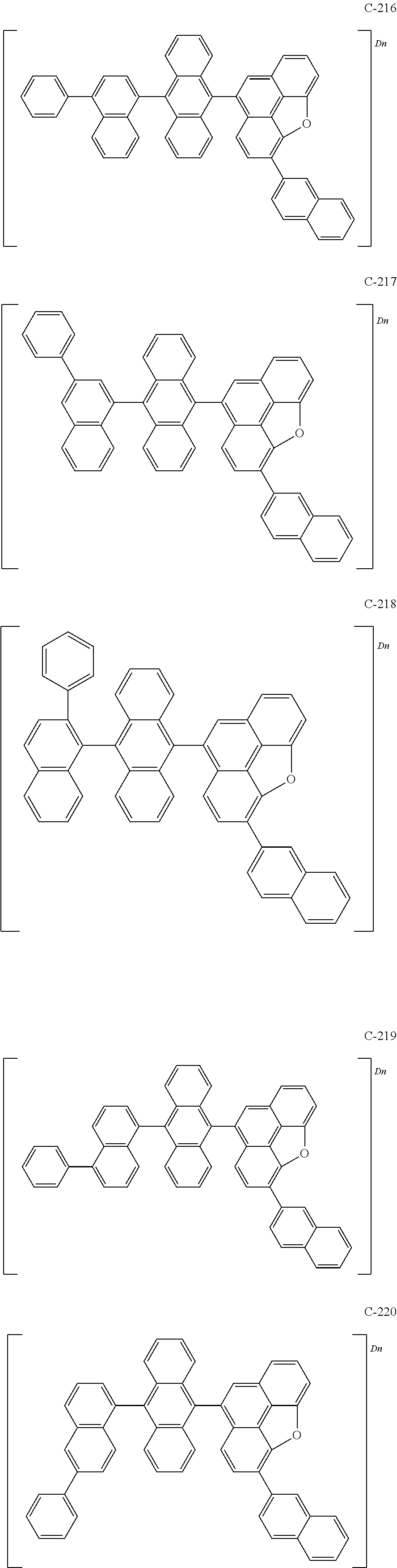 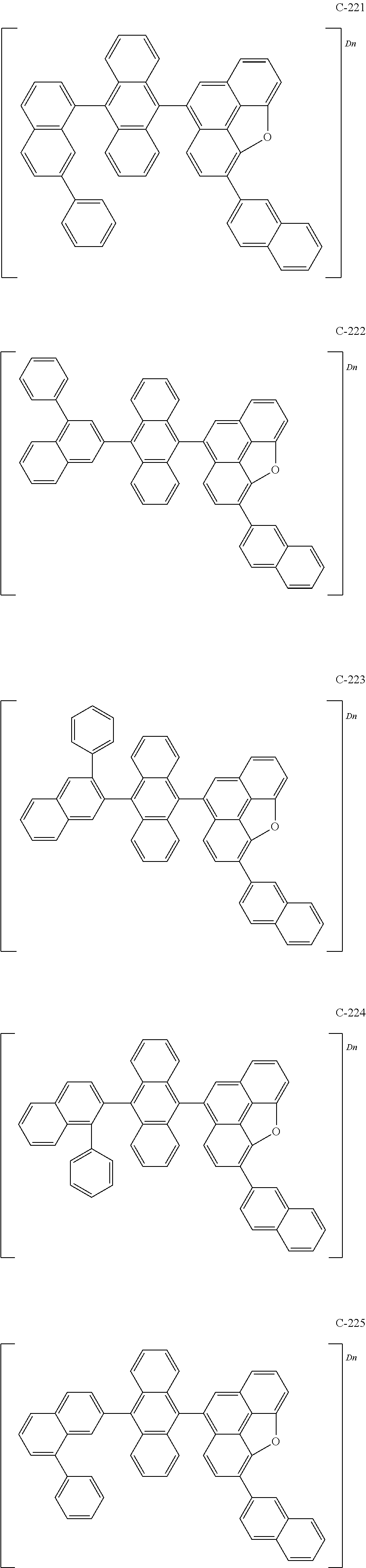 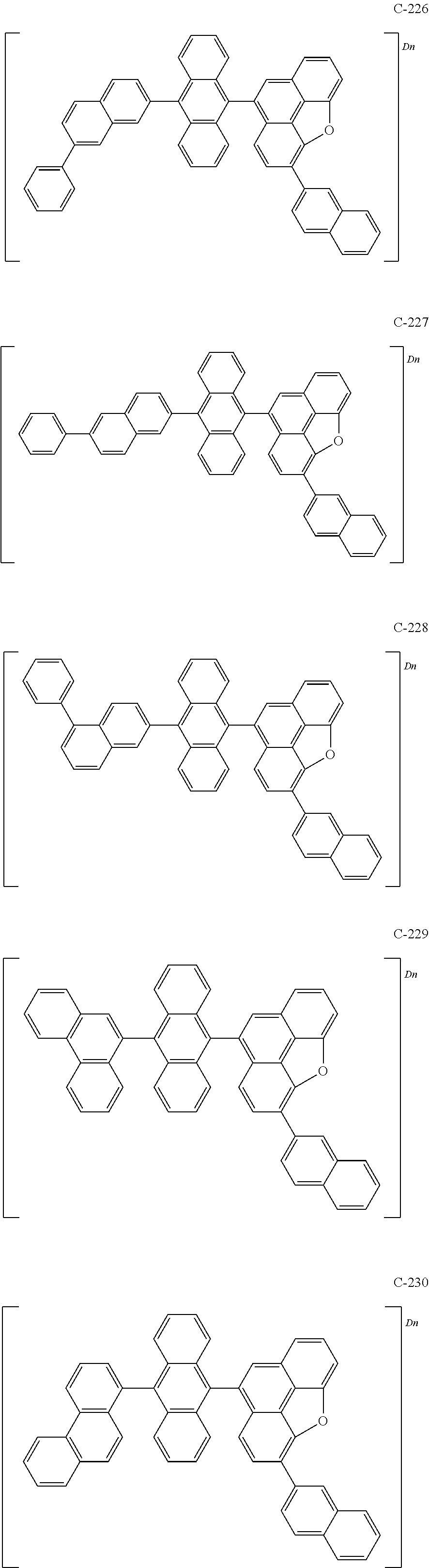 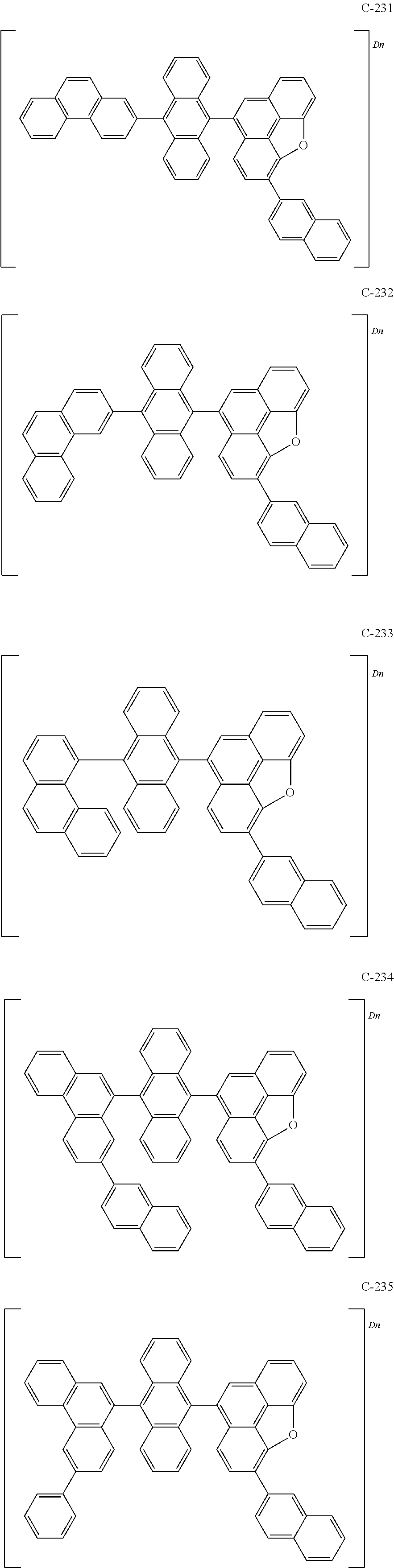 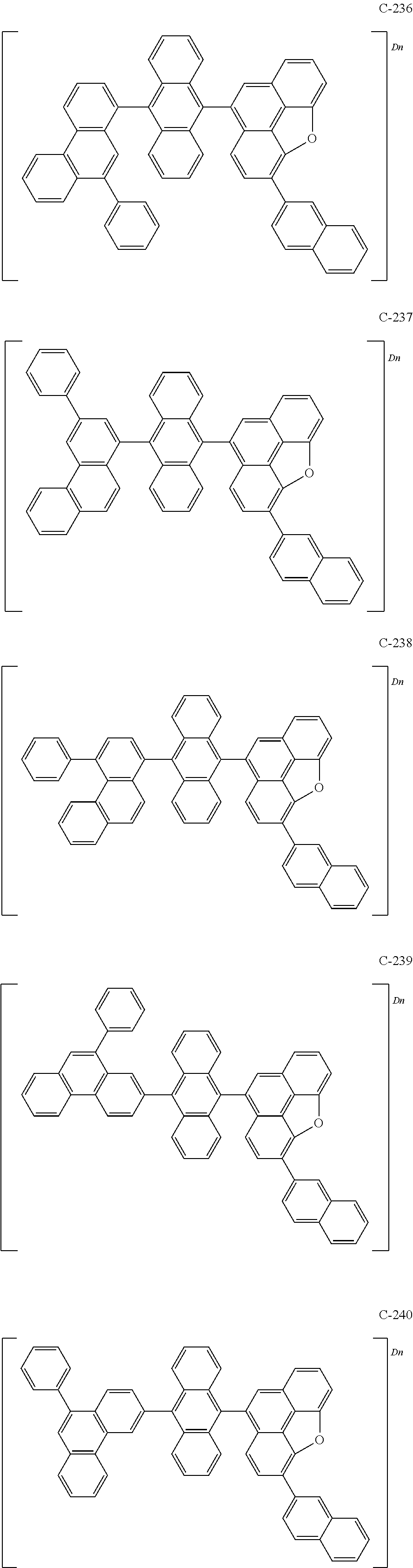 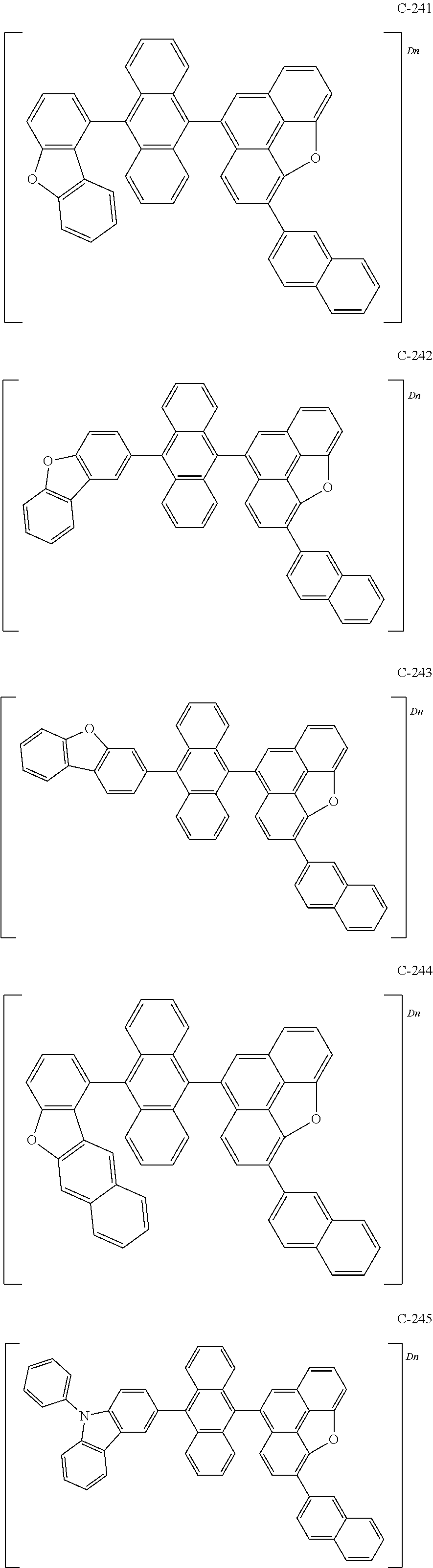 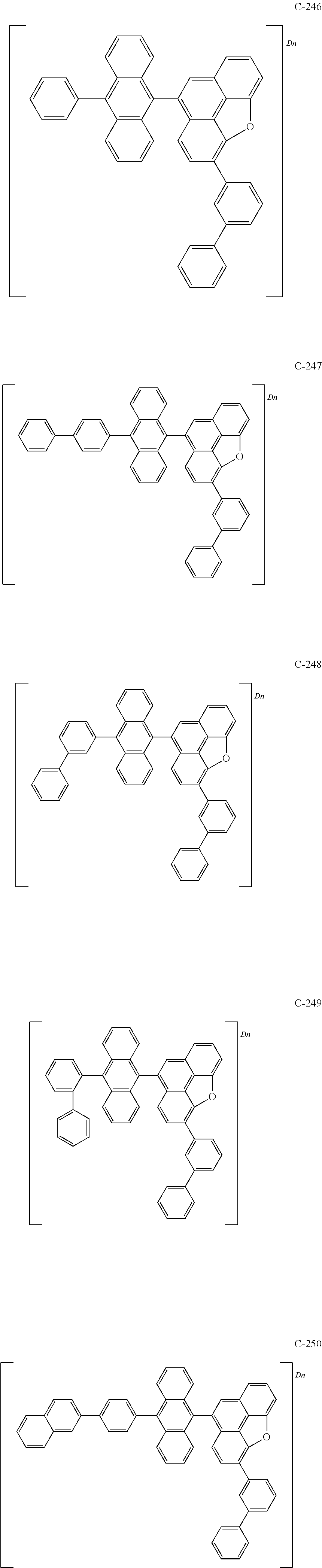 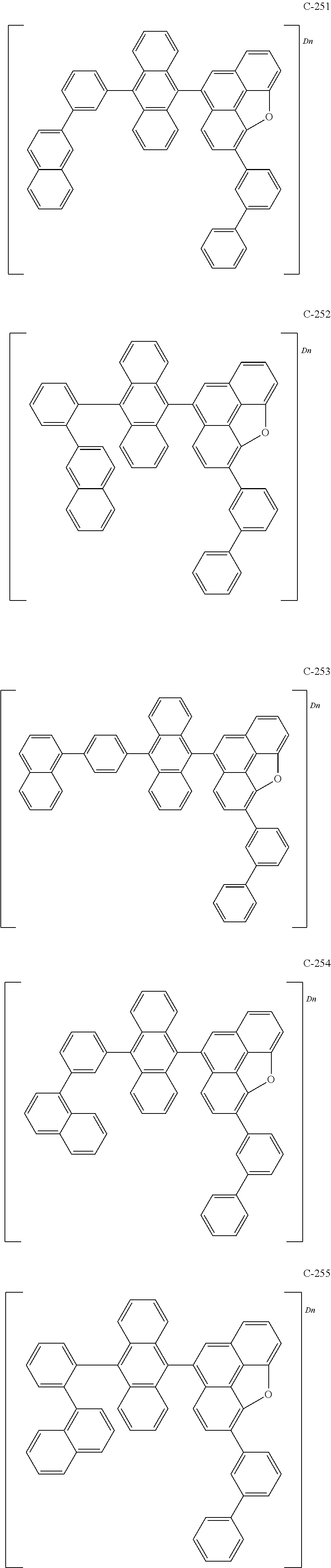 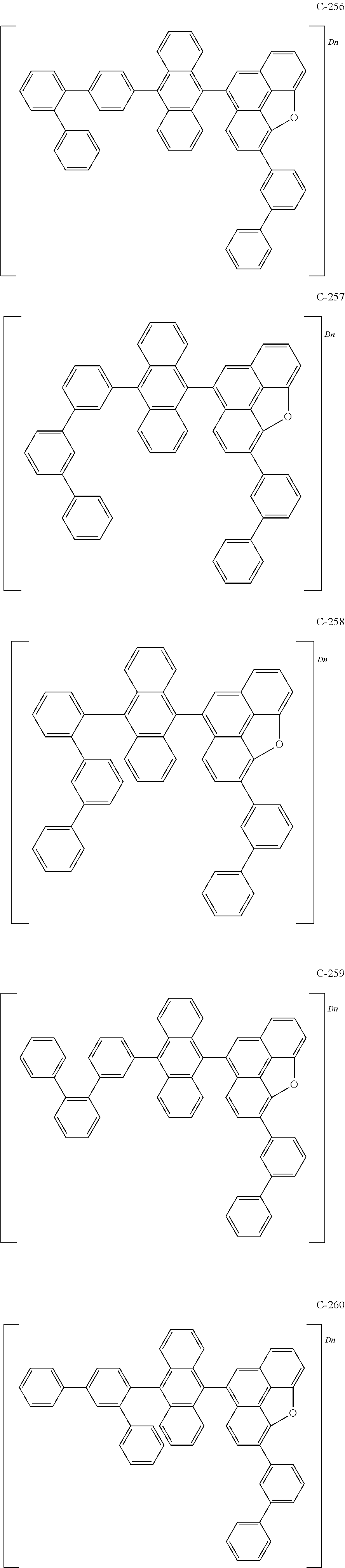 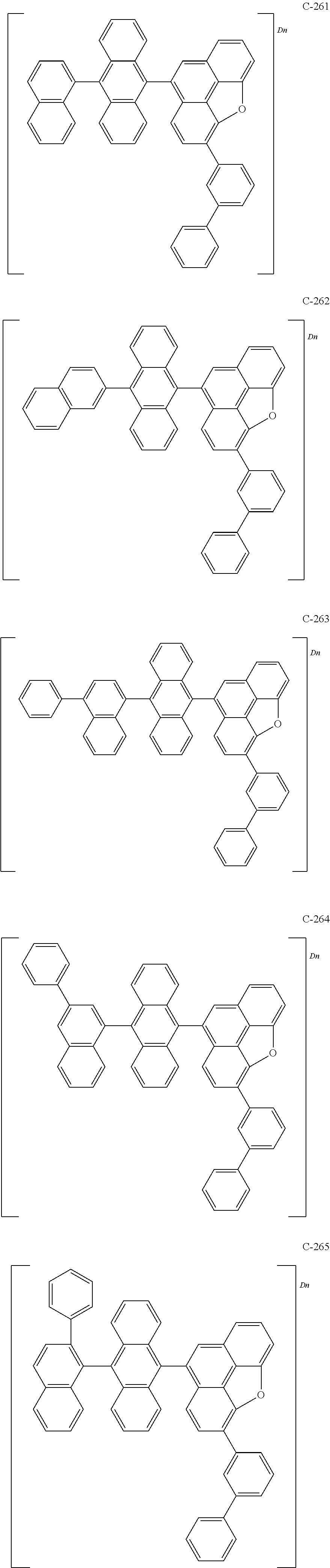 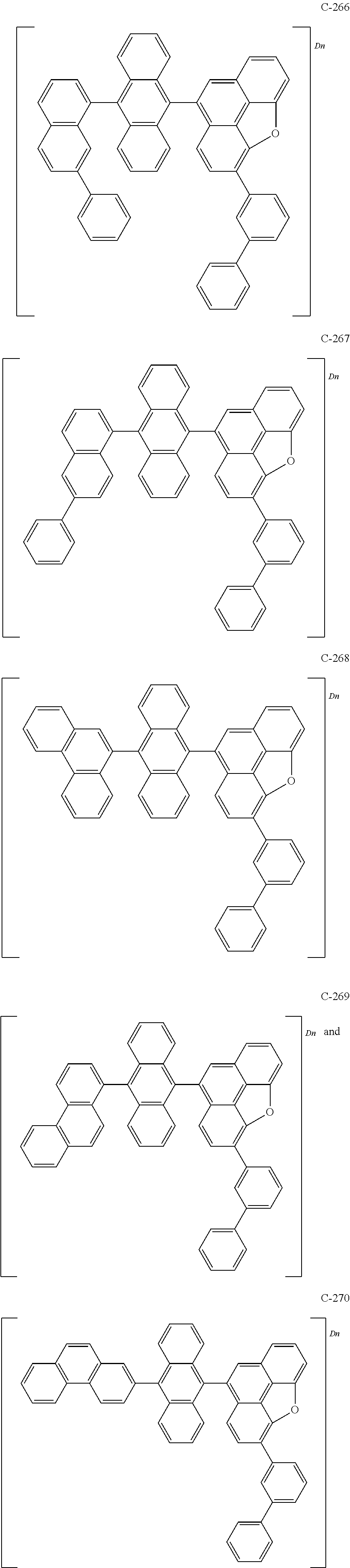
wherein Dn of compounds C-136 to C-270 means that n hydrogens are replaced by deuteriums, and n is between 1 and the maximum number of hydrogens in each compound.

9. A compound represented by the following formula 4: 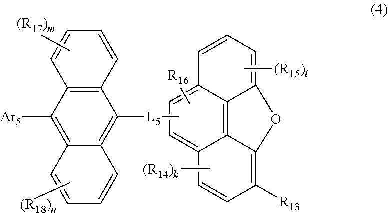
wherein,
Ar₅ represents a substituted or unsubstituted (C6-C30)aryl or a substituted or unsubstituted (3- to 30-membered)heteroaryl;
L₅ represents a single bond, a substituted or unsubstituted (C6-C30)arylene, or a substituted or unsubstituted (3- to 30-membered)heteroarylene;
R₁₃ represents hydrogen or deuterium n
R₁₄ to R₁₈ each independently represent: hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —N—(Ar₁₃)(Ar₁₄);
Ar₁₃ and Ar₁₄ each independently represent: hydrogen, deuterium, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
k represents an integer of 1 or 2, I represents an integer of 1 to 3, and m and n each independently represent an integer of 1 to 4, where if k to n are an integer of 2 or more, each of R₁₄, R₁₅, R₁₇, and R₁₈ may be the same or different;
provided that, at least one of R₁₄, R₁₅ and R₁₆ represents a substituted or unsubstituted (C6-C30)aryl or a substituted or unsubstituted (3- to 30-membered)heteroaryl; and at least one of R₁₃ to R₁₈ represents deuterium.

10. The compound of claim 9, wherein formula 4 is represented by the following formula 4-1 or 4-2: 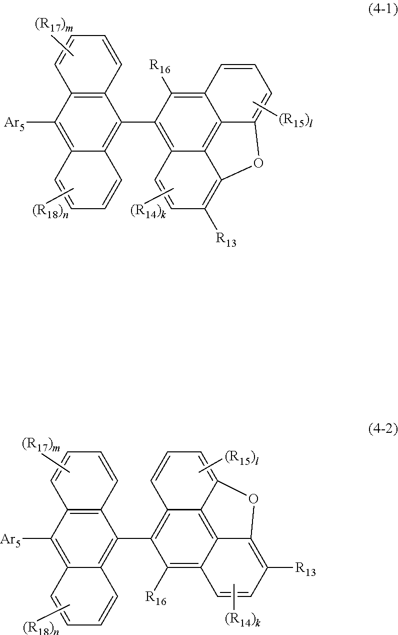
wherein Ar₅, R₁₃ to R₁₈, and k to n are the same as defined in claim 9.

11. The compound of claim 9, wherein the compound represented by formula 4 is selected from the following compounds: 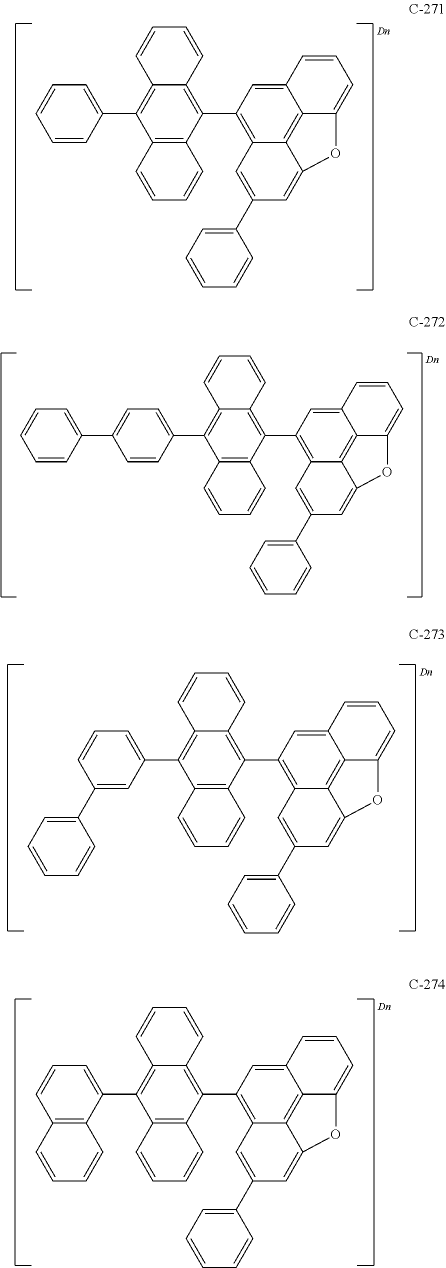 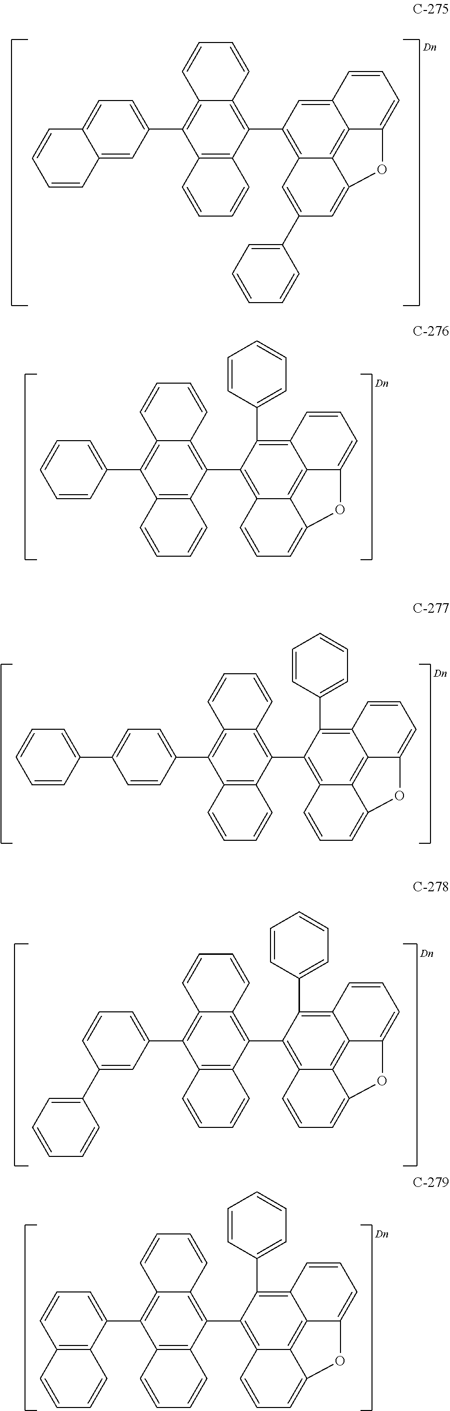 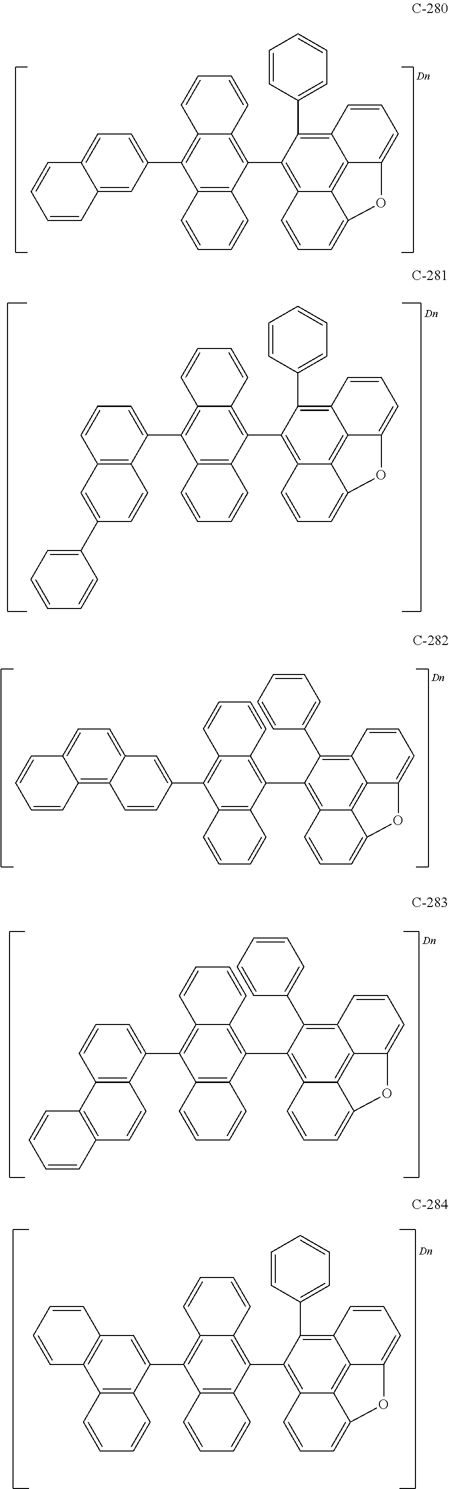 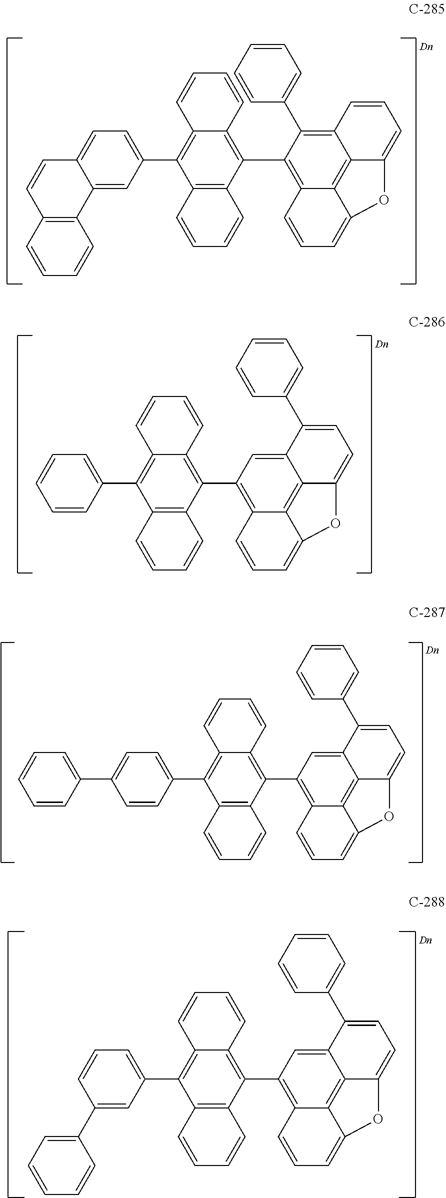 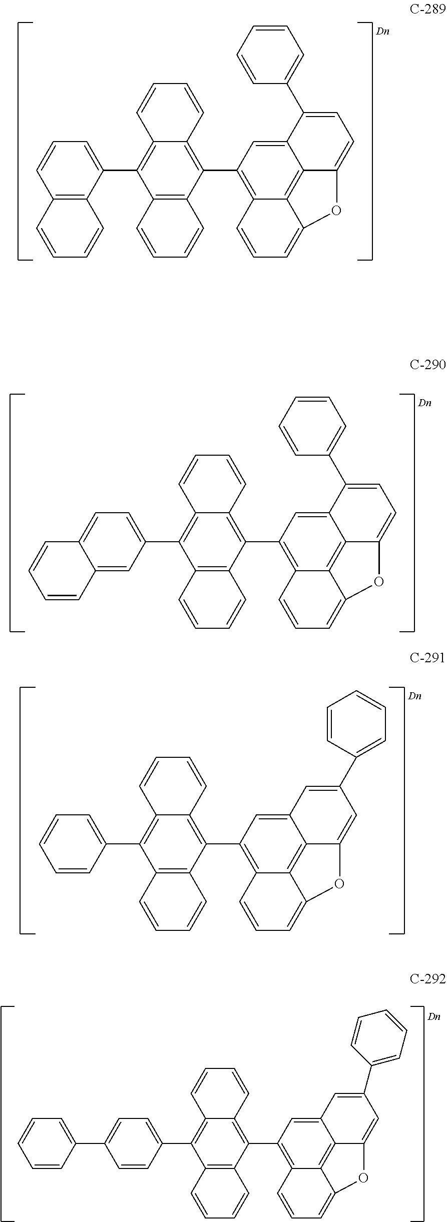 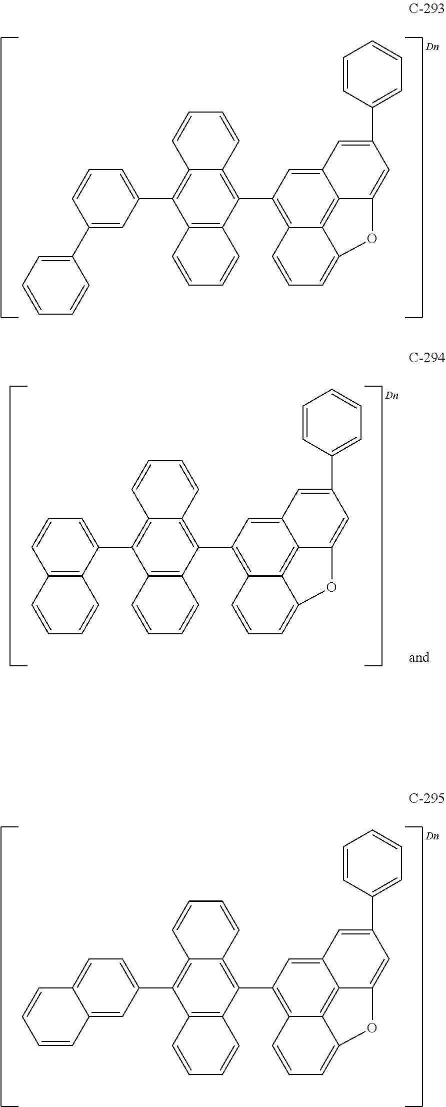
wherein Dn of compounds C-271 to C-295 means that n hydrogens are replaced by deuteriums, and n is between 1 and the maximum number of hydrogens in each compound.

12. An organic electroluminescence device comprising a first electrode, a second electrode, and at least one light-emitting layer(s) between the first electrode and the second electrode, wherein at least one layer of the light-emitting layer comprises the plurality of host materials of claim 1.
